(19) United States
(12) Patent Application Publication
(10) Pub. No.: US 2024/0224795 A1
(43) Pub. Date: Jul. 4, 2024

(54) LIGHT-EMITTING DEVICE INCLUDING AMINE-BASED COMPOUND, ELECTRONIC APPARATUS INCLUDING THE LIGHT-EMITTING DEVICE, LIGHT-EMITTING EQUIPMENT, AND THE AMINE-BASED COMPOUND

(71) Applicant: Samsung Display Co., Ltd., Yongin-si (KR)

(72) Inventors: Chaeyeong Kim, Yongin-si (KR); Dongjun Kim, Yongin-si (KR); Beomjoon Kim, Yongin-si (KR); Eunjae Jeong, Yongin-si (KR); Hanna Cha, Yongin-si (KR); Sanghyun Han, Yongin-si (KR)

(21) Appl. No.: 18/531,406

(22) Filed: Dec. 6, 2023

(30) Foreign Application Priority Data
Dec. 12, 2022 (KR) 10-2022-0173064

(51) Int. Cl. H10K 85/60; H10K 50/15

(52) U.S. Cl. CPC H10K 85/633; H10K 50/15; H10K 85/615; H10K 85/6574; H10K 85/6576

(57) ABSTRACT

An amine-based compound represented by Formula 1, a light-emitting device including the amine-based compound, and an electronic apparatus and an electronic equipment, each including the light-emitting device are provided. The light-emitting device includes a first electrode, a second electrode on the first electrode, and an interlayer between the first electrode and the second electrode and including the amine-based compound.

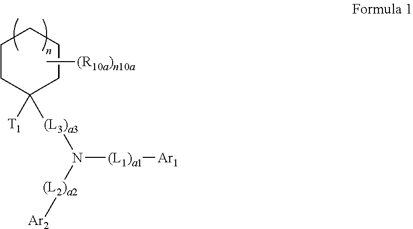

### CROSS-REFERENCE TO RELATED APPLICATION

This application claims priority to and the benefit of Korean Patent Application No. 10-2022-0173064, filed on Dec. 12, 2022, in the Korean Intellectual Property Office, the content of which is incorporated by reference herein in its entirety.

### BACKGROUND

### 1. Field

One or more embodiments of the present disclosure relate to a light-emitting device including an amine-based compound, an electronic apparatus including the light-emitting device, an electronic equipment including the light-emitting device, and the amine-based compound.

### 2. Description of the Related Art

Among light-emitting devices, self-emissive devices, such as organic light-emitting devices, have relatively wide viewing angles, high contrast ratios, short response times, and excellent or suitable characteristics in terms of luminance, driving voltage, and response speed.

In a light-emitting device, a first electrode is disposed on a substrate, and a hole transport region, an emission layer, an electron transport region, and a second electrode are sequentially disposed on the first electrode. Holes provided from the first electrode move toward the emission layer through the hole transport region, and electrons provided from the second electrode move toward the emission layer through the electron transport region. Carriers, such as holes and electrons, recombine in the emission layer to produce excitons. These excitons transition and relax from an excited state to a ground state to thus generate light.

### SUMMARY

One or more aspects of embodiments of the present disclosure are directed toward a light-emitting device including an amine-based compound, an electronic apparatus including the light-emitting device, and the amine-based compound.

Additional aspects will be set forth in part in the description which follows and, in part, will be apparent from the description, or may be learned by practice of the presented embodiments of the present disclosure.

According to one or more embodiments of the present disclosure, a light-emitting device includes:
a first electrode,
a second electrode facing the first electrode,
an interlayer between the first electrode and the second electrode and including an emission layer, and
an amine-based compound represented by Formula 1.

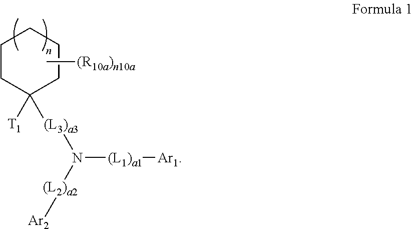

In Formula 1,
n may be an integer from 0 to 5,
L₁ to L₃ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
a₁ to a3 may each independently be an integer from 0 to 3,
*-(L₁)ₐ₁-* may be a single bond when a1 is 0,
*-(L₂)ₐ₂-*′ may be a single bond when a2 is 0,

*-(L₃)ₐ₃-*′ may be a single bond when a3 is 0,

Ar₁ and Ar₂ may each independently include a group represented by Formula 1-1, a group represented by Formula 1-2, a C₃-C₆₀ carbocyclic group unsubstituted or substituted with at least one R₁₀ₐ, or a C₁-C₆₀ heterocyclic group unsubstituted or substituted with at least one R₁₀ₐ,

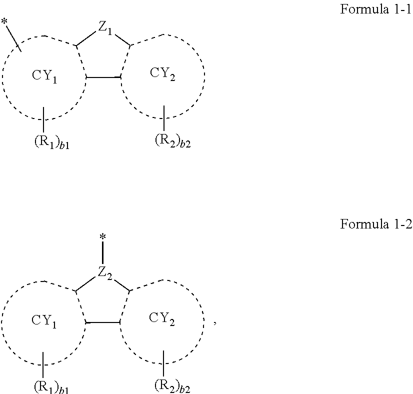

wherein, in Formulae 1-1 and 1-2,
Z₁ may be O, S, P(R₃), C(R₃)(R₄), C(R₃)H, CH₂, Si(R₃)(R₄), Si(R₃)H, or SiH₂,
Z₂ may be P, C(R₃), CH, Si(R₃), or SiH,
CY₁ and CY₂ may each independently be a C₃-C₆₀ carbocyclic group or a C₁-C₆₀ heterocyclic group,
b1 and b2 may each independently be an integer from 0 to 5,
* indicates a binding site to a neighboring atom,
T₁, R₁, R₂, R₃, R₄, and R₁₀ₐ may each independently be:
deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, —Si(Q₁₁)(Q₁₂)(Q₁₃), —B(Q₁₁)(Q₁₂), —C(═O)(Q₁₁), —S(═O)₂(Q₁₁), —P(═O)(Q₁₁)(Q₁₂), or a combination thereof;
a C₃-C₆₀ carbocyclic group, a C₁-C₆₀ heterocyclic group, a C₆-C₆₀ aryloxy group, or a C₆-C₆₀ arylthio group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, —Si(Q₂₁)(Q₂₂)(Q₂₃), —B(Q₂₁)(Q₂₂), —C(═O)(Q₂₁), —S(═O)₂(Q₂₁), —P(═O)(Q₂₁)(Q₂₂), or a combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O) (Q₃₁) (Q₃₂),
wherein Q₁₁ to Q¹³, Q²¹ to Q²³, and Q³¹ to Q³³ may each independently be:
hydrogen; deuterium; —F; —Cl; —Br; —I; a hydroxyl group; a cyano group; a nitro group; a C₁-C₆₀ alkyl group; a C₂-C₆₀ alkenyl group; a C₂-C₆₀ alkynyl group; a C₁-C₆₀ alkoxy group; or a C₃-C₆₀ carbocyclic group or a C₁-C₆₀ heterocyclic group, each unsubstituted or substituted with deuterium, —F, a cyano group, a C₁-C₆₀ alkyl group, a C₁-C₆₀ alkoxy group, a phenyl group, a biphenyl group, or a combination thereof, and
n10a may be an integer from 0 to 10.

According to one or more embodiments of the present disclosure, an electronic apparatus includes the light-emitting device according to one or more embodiments.

According to one or more embodiments of the present disclosure, an electronic equipment includes the light-emitting device according to one or more embodiments.

According to one or more embodiments of the present disclosure, provided is an amine-based compound represented by Formula 1.

### BRIEF DESCRIPTION OF THE DRAWINGS

The accompanying drawings are included to provide a further understanding of the present disclosure and are incorporated in and constitute a part of the present disclosure. The drawings illustrate embodiments of the present disclosure and, together with the description, serve to explain principles of the present disclosure. The above and other aspects, features, and advantages of certain embodiments of the disclosure will be more apparent from the following description taken in conjunction with the accompanying drawings, in which:

FIG. 1 is a schematic view of a light-emitting device according to one or more embodiments of the present disclosure;

FIG. 2 is a schematic view of an electronic apparatus according to one or more embodiments of the present disclosure;

FIG. 3 is a schematic view of an electronic apparatus according to one or more embodiments of the present disclosure;

FIG. 4 is a schematic view of electronic equipment according to one or more embodiments of the present disclosure; and

FIGS. 5 and 6A-6C are schematic views each being of an electronic equipment according to one or more embodiments of the present disclosure.

### DETAILED DESCRIPTION

Reference will now be made in more detail to embodiments, examples of which are illustrated in the accompanying drawings, wherein like reference numerals refer to like elements throughout the present disclosure, and duplicative descriptions thereof may not be provided for conciseness. In this regard, the embodiments of the present disclosure may have different forms and should not be construed as being limited to the descriptions set forth herein. Accordingly, the embodiments of the present disclosure are merely described, by referring to the drawings, to explain aspects of the present disclosure. As utilized herein, the term “and/or” or “or” may include any and all combinations of one or more of the associated listed items. Throughout the disclosure, the expressions such as “at least one of,” “one of,” and “selected from,” when preceding a list of elements, modify the entire list of elements and do not modify the individual elements of the list. For example, “at least one of a, b or c”, “at least one selected from a, b, and c”, “at least one selected from among a to c”, etc., may indicate only a, only b, only c, both (e.g., simultaneously) a and b, both (e.g., simultaneously) a and c, both (e.g., simultaneously) b and c, all of a, b, and c, or variations thereof. The ‘T’ utilized herein may be interpreted as “and” or as “or” depending on the situation.

In one or more embodiments of the present disclosure, a light-emitting device may include:
a first electrode;
a second electrode facing the first electrode;
an interlayer between the first electrode and the second electrode and including an emission layer; and
an amine-based compound represented by Formula 1:

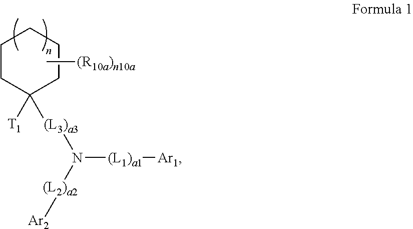

wherein, in Formula 1,
n may be an integer from 0 to 5 (for example, 0, 1, 2, 3, 4, 5),
L₁ to L₃ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
a1 to a3 may each independently be an integer from 0 to 3 (for example, 0, 1, 2, 3),
*-(L₁)ₐ₁-* may be a single bond when a1 is 0,
*-(L₂)ₐ₂-*′ may be a single bond when a2 is 0,
*-(L₃)ₐ₃-*′ may be a single bond when a3 is 0,
Ar₁ and Ar₂ may each independently include a group represented by Formula 1-1, a group represented by Formula 1-2, a C₃-C₆₀ carbocyclic group unsubstituted or substituted with at least one R₁₀ₐ, or a C₁-C₆₀ heterocyclic group unsubstituted or substituted with at least one R₁₀ₐ,

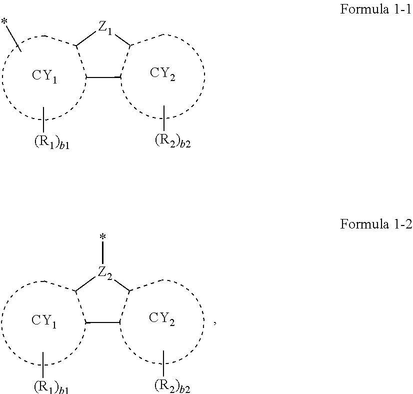

wherein, in Formulae 1-1 and 1-2,
Z₁ may be O, S, P(R₃), C(R₃)(R₄), C(R₃)H, CH₂, Si(R₃)(R₄), Si(R₃)H, or SiH₂,
Z₂ may be P, C(R₃), CH, Si(R₃), or SiH,
CY₁ and CY₂ may each independently be a C₃-C₆₀ carbocyclic group or a C₁-C₆₀ heterocyclic group,
b1 and b2 may each independently be an integer from 0 to 5 (for example, 0, 1,2,3,4,5),
* indicates a binding site to a neighboring atom,
T₁, R₁, R₂, R₃, R₄, and R₁₀ₐ may each independently be:
deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, —Si(Q₁₁)(Q₁₂)(Q₁₃), —B(Q₁₁)(Q₁₂), —C(═O)(Q₁₁), —S(═O)₂(Q₁₁), —P(═O)(Q₁₁)(Q₁₂), or a combination thereof;
a C₃-C₆₀ carbocyclic group, a C₁-C₆₀ heterocyclic group, a C₆-C₆₀ aryloxy group, or a C₆-C₆₀ arylthio group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, —Si(Q₂₁)(Q₂₂)(Q₂₃), —B(Q₂₁)(Q₂₂), —C(═O)(Q₂₁), —S(═O)₂(Q₂₁), —P(═O)(Q₂₁)(Q₂₂), or a combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂),
wherein Q₁₁ to Q₁₃, Q₂₁ to Q₂₃, and Q₃₁ to Q₃₃ may each independently be:
hydrogen; deuterium; —F; —C₁; —Br; —I; a hydroxyl group; a cyano group; a nitro group; a C₁-C₆₀ alkyl group; a C₂-C₆₀ alkenyl group; a C₂-C₆₀ alkynyl group; a C₁-C₆₀ alkoxy group; or a C₃-C₆₀ carbocyclic group or a C₁-C₆₀ heterocyclic group, each unsubstituted or substituted with deuterium, —F, a cyano group, a C₁-C₆₀ alkyl group, a C₁-C₆₀ alkoxy group, a phenyl group, a biphenyl group, or a combination thereof, and
n10a may be an integer from 0 to 10.

In the light-emitting device according to one or more embodiments, the interlayer may include the amine-based compound represented by Formula 1.

In the light-emitting device according to one or more embodiments,
the first electrode may be an anode,
the second electrode may be a cathode,
the interlayer may further include a hole transport region between the first electrode and the emission layer and an electron transport region between the emission layer and the second electrode,
the hole transport region may include a hole injection layer, a hole transport layer, an emission auxiliary layer, an electron blocking layer, or a combination thereof, and
the electron transport region may include a hole blocking layer, an electron transport layer, an electron injection layer, or a combination thereof.

In the light-emitting device according to one or more embodiments,
the hole transport region may include at least one amine-based compound represented by Formula 1.

In the light-emitting device according to one or more embodiments,
the hole transport layer may include at least one amine-based compound represented by Formula 1.

In the light-emitting device according to one or more embodiments,
the hole transport layer may be in direct contact with the emission layer.

The light-emitting device according to one or more embodiments may further include:
a first capping layer, a second capping layer, or a first capping layer and a second capping layer, wherein
the first capping layer may be on a surface of the first electrode, and
the second capping layer may be on a surface of the second electrode.

In the light-emitting device according to one or more embodiments,
at least one of the first capping layer or the second capping layer may include the amine-based compound represented by Formula 1.

In the light-emitting device according to one or more embodiments,
the emission layer may include a fluorescent dopant or a phosphorescent dopant.

In the light-emitting device according to one or more embodiments,
the emission layer may be to emit blue light.

In one or more embodiments, an electronic apparatus may include the light-emitting device according to one or more embodiments of the present disclosure.

The electronic apparatus according to one or more embodiments may further include:
a thin-film transistor; and
a color filter, a color conversion layer, a touch screen layer, a polarizing layer, or a combination thereof.

In one or more embodiments, the thin-film transistor may include a source electrode and a drain electrode, and
the first electrode of the light-emitting device may be electrically connected to the source electrode or the drain electrode of the thin-film transistor.

In one or more embodiments, electronic equipment may include any one of the light-emitting devices according to one or more embodiments.

The electronic equipment may be at least one selected from a flat panel display, a curved display, a computer monitor, a medical monitor, a television, an advertisement board, an indoor or outdoor lighting and/or signaling light, a head-up display, a fully or partially transparent display, a flexible display, a rollable display, a foldable display, a stretchable display, a laser printer, a telephone, a mobile phone, a tablet, a phablet, a personal digital assistant (FDA), a wearable device, a laptop computer, a digital camera, a camcorder, a viewfinder, a microdisplay, a 3D display, a virtual or augmented reality display, a vehicle, a video wall including multiple displays tiled together, a theater or stadium screen, a phototherapy device, and a sign.

In one or more embodiments of the present disclosure, an amine-based compound may be represented by Formula 1:

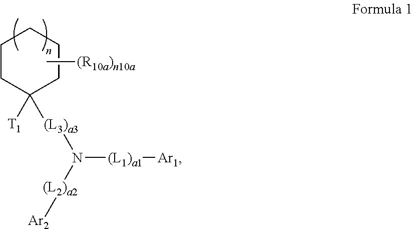

wherein, in Formula 1,
n may be an integer from 0 to 5 (for example, 0, 1, 2, 3, 4, 5),
L₁ to L₃ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
a1 to a3 may each independently be an integer from 0 to 3 (for example, 0, 1, 2, 3),
*-(L₁)ₐ₁-* may be a single bond when a1 is 0,
*-(L₂)ₐ₂-*′ may be a single bond when a2 is 0,
*-(L₃)ₐ₃-*′ may be a single bond when a3 is 0,

An and Are may each independently include a group represented by Formula 1-1, a group represented by Formula 1-2, a C₃-C₆₀ carbocyclic group unsubstituted or substituted with at least one R₁₀ₐ, or a C₁-C₆₀ heterocyclic group unsubstituted or substituted with at least one R₁₀ₐ,

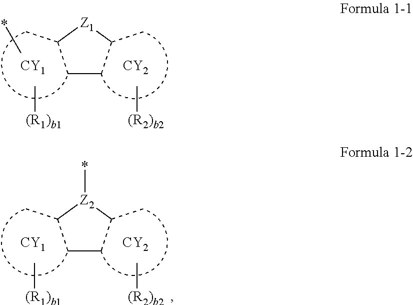

wherein, in Formulae 1-1 and 1-2,
Z₁ may be O, S, P(R₃), C(R₃)(R₄), C(R₃)H, CH₂, Si(R₃)(R₄), Si(R₃)H, or SiH₂,
Z₂ may be P, C(R₃), CH, Si(R₃), or SiH,
CY₁ and CY₂ may each independently be a C₃-C₆₀ carbocyclic group or a C₁-C₆₀ heterocyclic group,
b1 and b2 may each independently be an integer from 0 to 5 (for example, 0, 1,2,3,4,5),
* indicates a binding site to a neighboring atom,
T₁, R₁, R₂, R₃, R₄, and R₁₀ₐ may each independently be:
deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, —Si(Q₁₁)(Q₁₂)(Q₁₃), —B(Q₁₁)(Q₁₂), —C(═O)(Q₁₁), —S(═O)₂(Q₁₁), —P(═O)(Q₁₁)(Q₁₂), or a combination thereof;
a C₃-C₆₀ carbocyclic group, a C₁-C₆₀ heterocyclic group, a C₆-C₆₀ aryloxy group, or a C₆-C₆₀ arylthio group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, —Si(Q₂₁)(Q₂₂)(Q₂₃), —B(Q₂₁)(Q₂₂), —C(═O)(Q₂₁), —S(═O)₂(Q₂₁), —P(═O)(Q₂₁)(Q₂₂), or a combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O) (Q₃₁) (Q₃₂),
wherein Q₁₁ to Q₁₃, Q₂₁ to Q₂₃, and Q₃₁ to Q₃₃ may each independently be:
hydrogen; deuterium; —F; —CI; —Br; —I; a hydroxyl group; a cyano group; a nitro group; a C₁-C₆₀ alkyl group; a C₂-C₆₀ alkenyl group; a C₂-C₆₀ alkynyl group; a C₁-C₆₀ alkoxy group; or a C₃-C₆₀ carbocyclic group or a C₁-C₆₀ heterocyclic group, each unsubstituted or substituted with deuterium, —F, a cyano group, a C₁-C₆₀ alkyl group, a C₁-C₆₀ alkoxy group, a phenyl group, a biphenyl group, or a combination thereof, and
n10a may be an integer from 0 to 10.

In the amine-based compound according to one or more embodiments, n may be an integer from 0 to 2.

In the amine-based compound according to one or more embodiments, n may be 1.

In the amine-based compound according to one or more embodiments,
an A value of a moiety represented by

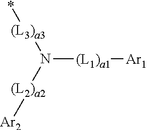

in Formula 1 may be equal to or less than an A value of T₁. The term “A value of a moiety” as utilized herein may refer to a numerical value utilized in the determination of the most stable orientation of the moiety in a cyclic conformation. A moiety on an alicyclic ring prefers to reside in the equatorial position to the axial. The difference in Gibbs free energy between the higher energy conformation (axial substitution) and the lower energy conformation (equatorial substitution) is the A-value of the moiety.

In the amine-based compound according to one or more embodiments, a sum of a1, a2, and a3 may be 1 or more.

In the amine-based compound according to one or more embodiments, a sum of a1, a2, and a3 may be 3 or more.

In the amine-based compound according to one or more embodiments, a1 may be 1 or more. As a result, an A value of a moiety represented by

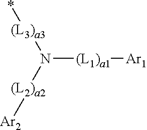

in Formula 1 may decrease even more.

In the amine-based compound according to one or more embodiments, at least one selected from among L₁, L₂, and L₃ may be a benzene group unsubstituted or substituted with at least one R₁₀ₐ, a naphthalene group unsubstituted or substituted with at least one R₁₀ₐ, a fluorene group unsubstituted or substituted with at least one R₁₀ₐ, a dibenzofuran group unsubstituted or substituted with at least one R₁₀ₐ, or a dibenzothiophene group unsubstituted or substituted with at least one R₁₀ₐ.

In the amine-based compound according to one or more embodiments, at least one selected from among L₁, L₂, and L₃ may be a group represented by one selected from Formulae 1-6-1 to 1-6-3:

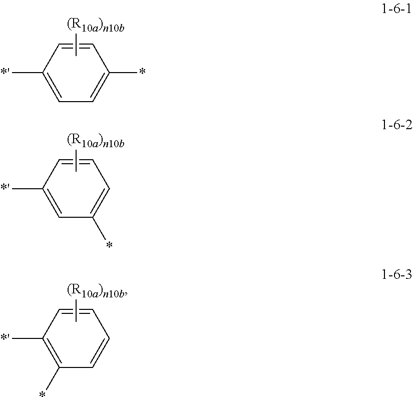

wherein, in Formulae 1-6-1 to 1-6-3,
R₁₀ₐ may be the same as described with respect to R₁₀ₐ in Formula 1,
n1 Ob may be an integer from 0 to 4, and
* and *′ may be a binding site to a neighboring atom.

The amine-based compound according to one or more embodiments may satisfy one of Condition i) or ii):
i) at least one selected from among Ar₁ and Ar₂ includes a group represented by Formula 1-1 or a group represented by Formula 1-2;
ii) Ar₁ and Ar₂ each do not include a group represented by Formula 1-1 and a group represented by Formula 1-2.

When the amine-based compound according to one or more embodiments satisfies Condition i) and includes the group represented by represented by Formula 1-1, a moiety represented by

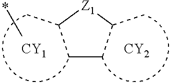

may be one selected from Formulae 1-1-1 to 1-1-4:

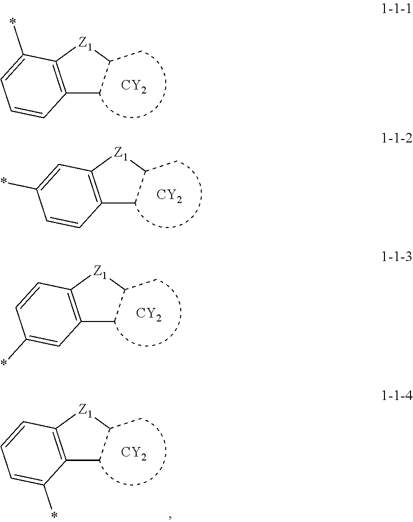

wherein, in Formulae 1-1-1 to 1-1-4,
Z₁ and CY₂ may each be the same as described with respect to Z₁ of Formula 1 and CY₂ of Formula 1-1, respectively, and
* indicates a binding site to a neighboring atom.

In the amine-group compound according to one or more embodiments, Ar₁ and Are may each independently include a cyclopentane group, a cyclohexane group, a norbornane group, a bicycloheptane group, an adamantane group, a benzene group, a pentalene group, a naphthalene group, an azulene group, an indacene group, an acenaphthylene group, a phenalene group, a phenanthrene group, an anthracene group, a fluoranthene group, a triphenylene group, a pyrene group, a chrysene group, a perylene group, a pentaphene group, a heptalene group, a naphthacene group, a picene group, a hexacene group, a pentacene group, a rubicene group, a coronene group, an ovalene group, an indene group, a fluorene group, a spiro-bifluorene group, a benzofluorene group, an indenophenanthrene group, an indenoanthracene group, a pyrrole group, a thiophene group, a furan group, an indole group, a benzoindole group, a naphthoindole group, an isoindole group, a benzoisoindole group, a naphthoisoindole group, a benzosilole group, a benzothiophene group, a benzofuran group, a dibenzosilole group, a dibenzothiophene group, a dibenzofuran group, a benzonaphthofuran group, a benzonaphthothiophene group, a benzonaphthosilole group, a benzofurodibenzofuran group, a benzofurodibenzothiophene group, a benzothienodibenzothiophene group, a pyrazole group, an imidazole group, a triazole group, an oxazole group, an isoxazole group, an oxadiazole group, a thiazole group, an isothiazole group, a thiadiazole group, a benzopyrazole group, a benzimidazole group, a benzoxazole group, a benzoisoxazole group, a benzothiazole group, a benzoisothiazole group, a pyridine group, a pyrimidine group, a pyrazine group, a pyridazine group, a triazine group, a quinoline group, an isoquinoline group, a benzoquinoline group, a benzoisoquinoline group, a quinoxaline group, a benzoquinoxaline group, a quinazoline group, a benzoquinazoline group, a phenanthroline group, a cinnoline group, a phthalazine group, a naphthyridine group, an imidazopyridine group, an imidazopyrimidine group, an imidazotriazine group, an imidazopyrazine group, an imidazopyridazine group, an azafluorene group, an azadibenzosilole group, an azadibenzothiophene group, or an azadibenzofuran group.

In the amine-based compound according to one or more embodiments, Ar₁ and Are may each independently include a cyclopentane group, a cyclohexane group, a norbornane group, a bicycloheptane group, an adamantane group, a benzene group, a pentalene group, a naphthalene group, an azulene group, an indacene group, an acenaphthylene group, a phenalene group, a phenanthrene group, an anthracene group, a fluoranthene group, a triphenylene group, a pyrene group, a chrysene group, a perylene group, a pentaphene group, a heptalene group, a naphthacene group, a picene group, a hexacene group, a pentacene group, a rubicene group, a coronene group, an ovalene group, an indene group, a fluorene group, a spiro-bifluorene group, a benzofluorene group, an indenophenanthrene group, or an indenoanthracene group.

In the amine-based compound according to one or more embodiments, T₁ and L₃ may include identical groups identical to each other.

In the amine-based compound according to one or more embodiments, T₁ and L₃ may include a benzene group.

In the amine-based compound according to one or more embodiments, T₁ and L₃ may include different groups differing from each other.

In the amine-based compound according to one or more embodiments, T₁ may include an isopropyl group, an isobutyl group, a sec-butyl group, a tert-butyl group, an isopentyl group, a sec-pentyl group, a tert-pentyl group, a neopentyl group, a cyclopentane group, a cyclohexane group, a norbornane group, a bicycloheptane group, an adamantane group, a benzene group, a pentalene group, a naphthalene group, an azulene group, an indacene group, an acenaphthylene group, a phenalene group, a phenanthrene group, an anthracene group, a fluoranthene group, a triphenylene group, a pyrene group, a chrysene group, a perylene group, a pentaphene group, a heptalene group, a naphthacene group, a picene group, a hexacene group, a pentacene group, a rubicene group, a coronene group, an ovalene group, an indene group, a fluorene group, a spiro-bifluorene group, a benzofluorene group, an indenophenanthrene group, or an indenoanthracene group.

In the amine-based compound according to one or more embodiments, n10a in Formula 1 may be 0.

In the amine-based compound according to one or more embodiments, the compound represented by Formula 1 may be one selected from among Compounds 1 to 384:

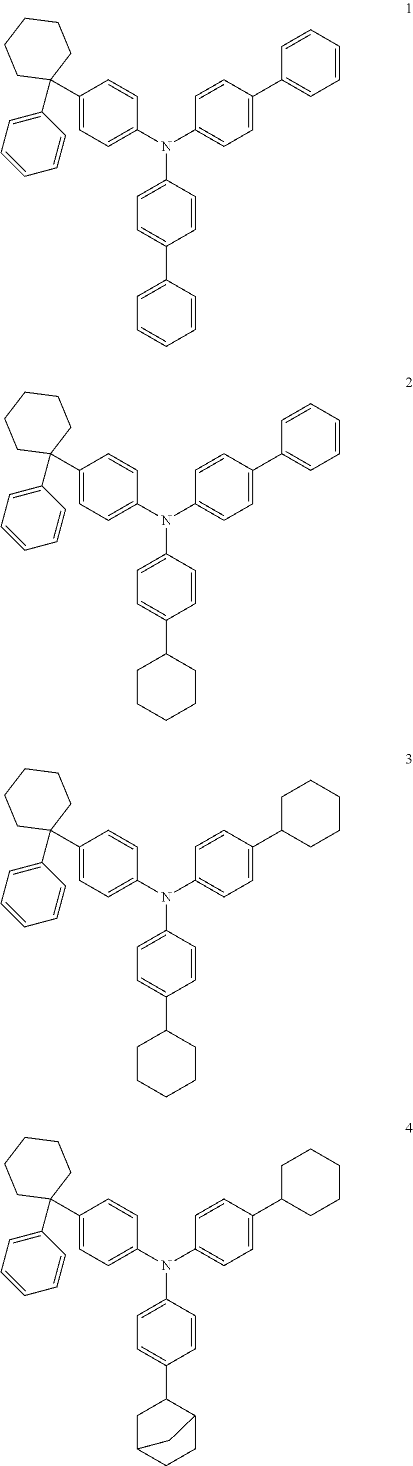 [SMILES: c1ccc(-c2ccc(N(c3ccc(-c4ccccc4)cc3)c3ccc(C4(c5ccccc5)CCCCC4)cc3)cc2)cc1 ; c1ccc(-c2ccc(N(c3ccc(C4CCCCC4)cc3)c3ccc(C4(c5ccccc5)CCCCC4)cc3)cc2)cc1 ; c1ccc(C2(c3ccc(N(c4ccc(C5CCCCC5)cc4)c4ccc(C5CCCCC5)cc4)cc3)CCCCC2)cc1 ; c1ccc(C2(c3ccc(N(c4ccc(C5CCCCC5)cc4)c4ccc(C5CC6CCC5C6)cc4)cc3)CCCCC2)cc1]

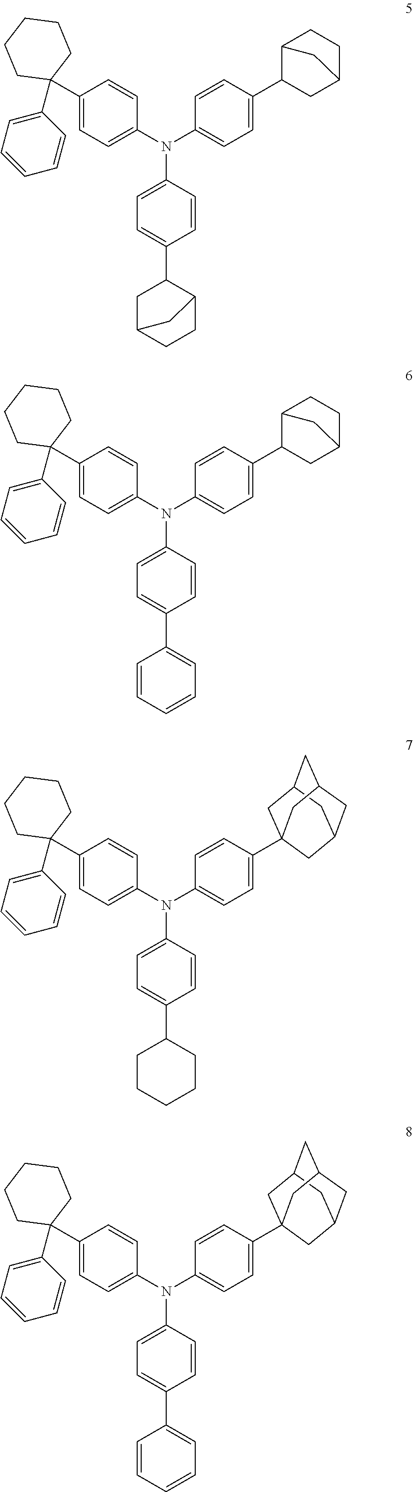 [SMILES: c1ccc(C2(c3ccc(N(c4ccc(C5CC6CCC5C6)cc4)c4ccc(C5CC6CCC5C6)cc4)cc3)CCCCC2)cc1 ; c1ccc(-c2ccc(N(c3ccc(C4CC5CCC4C5)cc3)c3ccc(C4(c5ccccc5)CCCCC4)cc3)cc2)cc1 ; c1ccc(C2(c3ccc(N(c4ccc(C5CCCCC5)cc4)c4ccc(C56CC7CC(CC(C7)C5)C6)cc4)cc3)CCCCC2)cc1 ; c1ccc(-c2ccc(N(c3ccc(C45CC6CC(CC(C6)C4)C5)cc3)c3ccc(C4(c5ccccc5)CCCCC4)cc3)cc2)cc1]

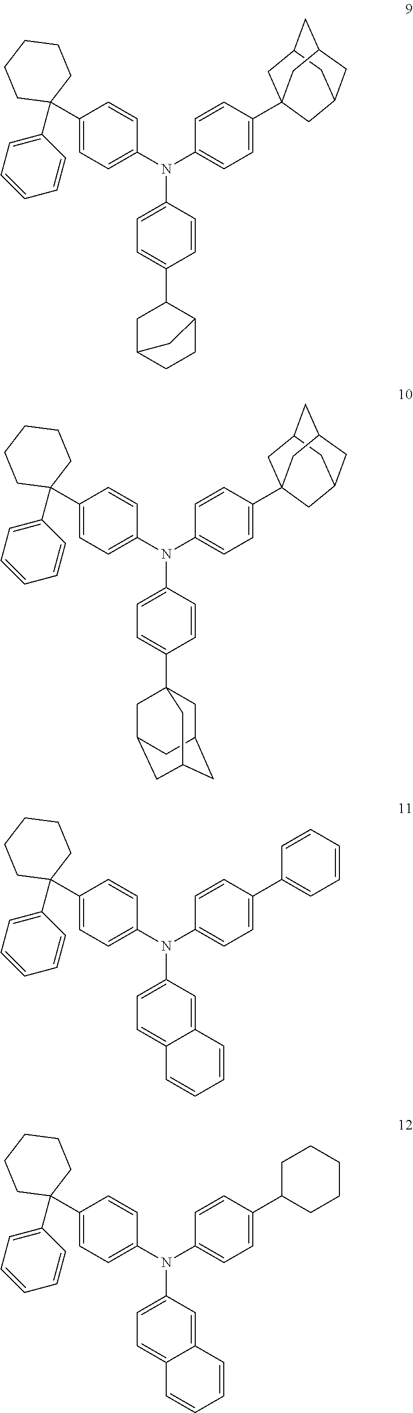 [SMILES: c1ccc(C2(c3ccc(N(c4ccc(C5CC6CCC5C6)cc4)c4ccc(C56CC7CC(CC(C7)C5)C6)cc4)cc3)CCCCC2)cc1 ; c1ccc(C2(c3ccc(N(c4ccc(C56CC7CC(CC(C7)C5)C6)cc4)c4ccc(C56CC7CC(CC(C7)C5)C6)cc4)cc3)CCCCC2)cc1 ; c1ccc(-c2ccc(N(c3ccc(C4(c5ccccc5)CCCCC4)cc3)c3ccc4ccccc4c3)cc2)cc1 ; c1ccc(C2(c3ccc(N(c4ccc(C5CCCCC5)cc4)c4ccc5ccccc5c4)cc3)CCCCC2)cc1]

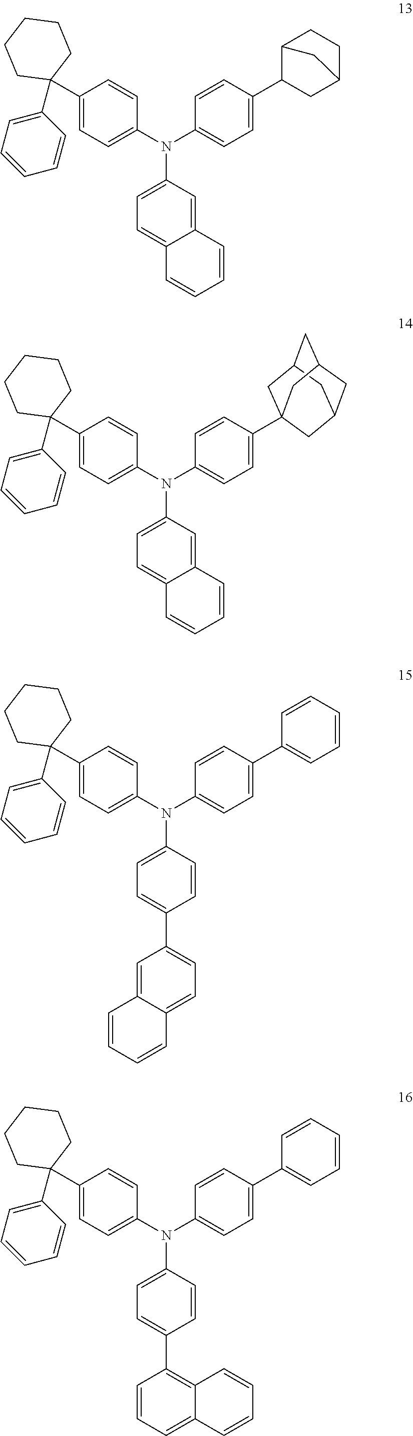 [SMILES: c1ccc(C2(c3ccc(N(c4ccc(C5CC6CCC5C6)cc4)c4ccc5ccccc5c4)cc3)CCCCC2)cc1 ; c1ccc(C2(c3ccc(N(c4ccc(C56CC7CC(CC(C7)C5)C6)cc4)c4ccc5ccccc5c4)cc3)CCCCC2)cc1 ; c1ccc(-c2ccc(N(c3ccc(-c4ccc5ccccc5c4)cc3)c3ccc(C4(c5ccccc5)CCCCC4)cc3)cc2)cc1 ; c1ccc(-c2ccc(N(c3ccc(-c4cccc5ccccc45)cc3)c3ccc(C4(c5ccccc5)CCCCC4)cc3)cc2)cc1]

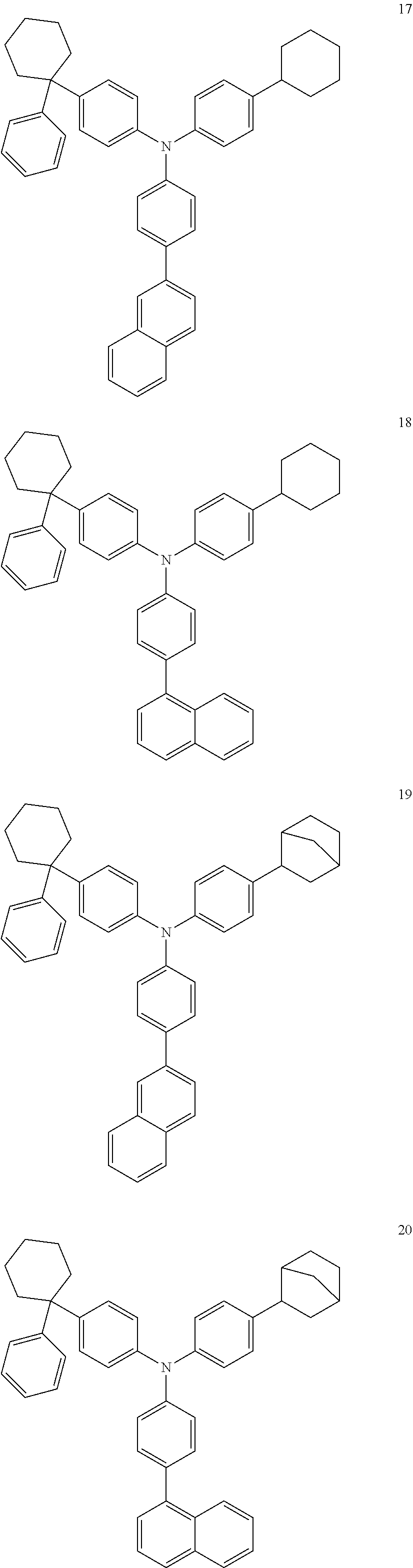 [SMILES: c1ccc(C2(c3ccc(N(c4ccc(-c5ccc6ccccc6c5)cc4)c4ccc(C5CCCCC5)cc4)cc3)CCCCC2)cc1 ; c1ccc(C2(c3ccc(N(c4ccc(-c5cccc6ccccc56)cc4)c4ccc(C5CCCCC5)cc4)cc3)CCCCC2)cc1 ; c1ccc(C2(c3ccc(N(c4ccc(-c5ccc6ccccc6c5)cc4)c4ccc(C5CC6CCC5C6)cc4)cc3)CCCCC2)cc1 ; c1ccc(C2(c3ccc(N(c4ccc(-c5cccc6ccccc56)cc4)c4ccc(C5CC6CCC5C6)cc4)cc3)CCCCC2)cc1]

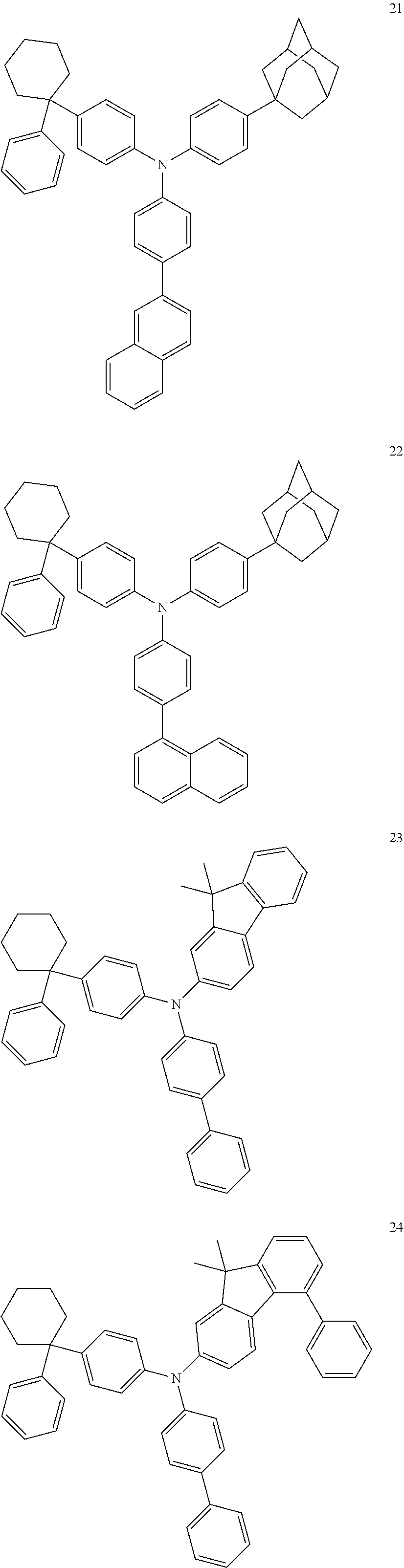 [SMILES: c1ccc(C2(c3ccc(N(c4ccc(-c5ccc6ccccc6c5)cc4)c4ccc(C56CC7CC(CC(C7)C5)C6)cc4)cc3)CCCCC2)cc1 ; c1ccc(C2(c3ccc(N(c4ccc(-c5cccc6ccccc56)cc4)c4ccc(C56CC7CC(CC(C7)C5)C6)cc4)cc3)CCCCC2)cc1 ; CC1(C)c2ccccc2-c2ccc(N(c3ccc(-c4ccccc4)cc3)c3ccc(C4(c5ccccc5)CCCCC4)cc3)cc21 ; CC1(C)c2cc(N(c3ccc(-c4ccccc4)cc3)c3ccc(C4(c5ccccc5)CCCCC4)cc3)ccc2-c2c(-c3ccccc3)cccc21]

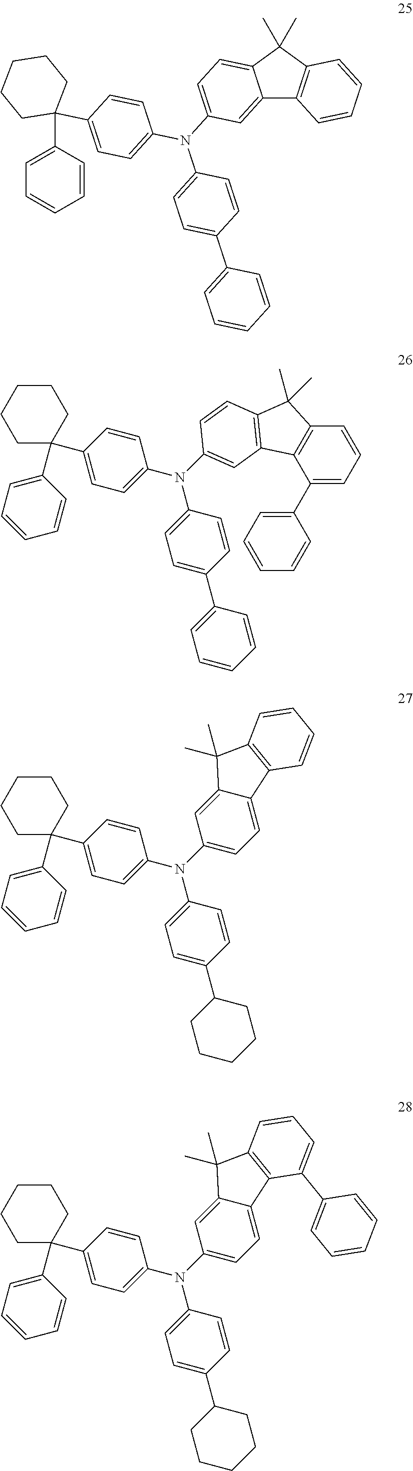 [SMILES: CC1(C)c2ccccc2-c2cc(N(c3ccc(-c4ccccc4)cc3)c3ccc(C4(c5ccccc5)CCCCC4)cc3)ccc21 ; CC1(C)c2ccc(N(c3ccc(-c4ccccc4)cc3)c3ccc(C4(c5ccccc5)CCCCC4)cc3)cc2-c2c(-c3ccccc3)cccc21 ; CC1(C)c2ccccc2-c2ccc(N(c3ccc(C4CCCCC4)cc3)c3ccc(C4(c5ccccc5)CCCCC4)cc3)cc21 ; CC1(C)c2cc(N(c3ccc(C4CCCCC4)cc3)c3ccc(C4(c5ccccc5)CCCCC4)cc3)ccc2-c2c(-c3ccccc3)cccc21]

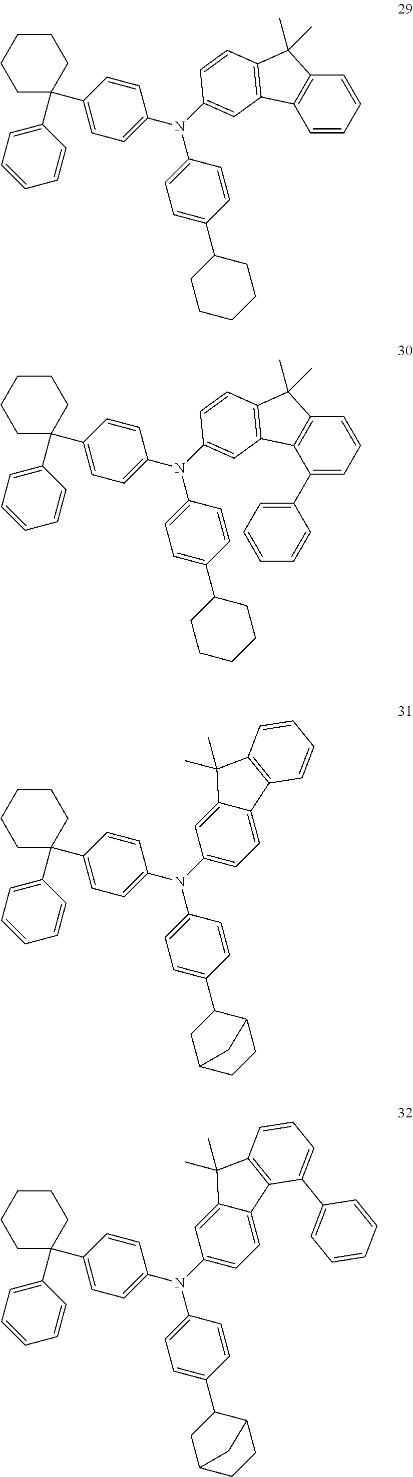 [SMILES: CC1(C)c2ccccc2-c2cc(N(c3ccc(C4CCCCC4)cc3)c3ccc(C4(c5ccccc5)CCCCC4)cc3)ccc21 ; CC1(C)c2ccc(N(c3ccc(C4CCCCC4)cc3)c3ccc(C4(c5ccccc5)CCCCC4)cc3)cc2-c2c(-c3ccccc3)cccc21 ; CC1(C)c2ccccc2-c2ccc(N(c3ccc(C4CC5CCC4C5)cc3)c3ccc(C4(c5ccccc5)CCCCC4)cc3)cc21 ; CC1(C)c2cc(N(c3ccc(C4CC5CCC4C5)cc3)c3ccc(C4(c5ccccc5)CCCCC4)cc3)ccc2-c2c(-c3ccccc3)cccc21]

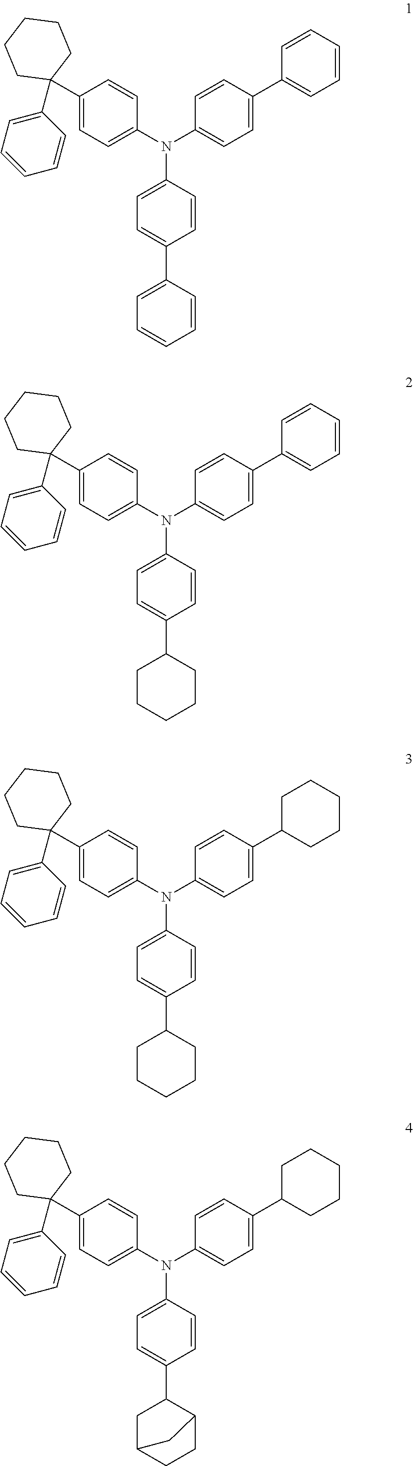 [SMILES: c1ccc(-c2ccc(N(c3ccc(-c4ccccc4)cc3)c3ccc(C4(c5ccccc5)CCCCC4)cc3)cc2)cc1 ; c1ccc(-c2ccc(N(c3ccc(C4CCCCC4)cc3)c3ccc(C4(c5ccccc5)CCCCC4)cc3)cc2)cc1 ; c1ccc(C2(c3ccc(N(c4ccc(C5CCCCC5)cc4)c4ccc(C5CCCCC5)cc4)cc3)CCCCC2)cc1 ; c1ccc(C2(c3ccc(N(c4ccc(C5CCCCC5)cc4)c4ccc(C5CC6CCC5C6)cc4)cc3)CCCCC2)cc1]

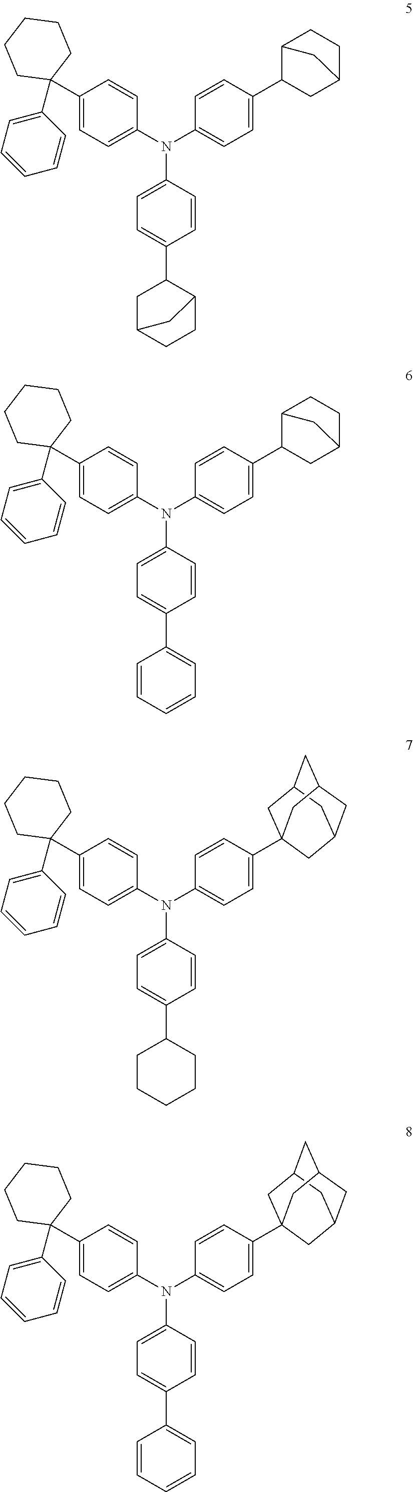 [SMILES: c1ccc(C2(c3ccc(N(c4ccc(C5CC6CCC5C6)cc4)c4ccc(C5CC6CCC5C6)cc4)cc3)CCCCC2)cc1 ; c1ccc(-c2ccc(N(c3ccc(C4CC5CCC4C5)cc3)c3ccc(C4(c5ccccc5)CCCCC4)cc3)cc2)cc1 ; c1ccc(C2(c3ccc(N(c4ccc(C5CCCCC5)cc4)c4ccc(C56CC7CC(CC(C7)C5)C6)cc4)cc3)CCCCC2)cc1 ; c1ccc(-c2ccc(N(c3ccc(C45CC6CC(CC(C6)C4)C5)cc3)c3ccc(C4(c5ccccc5)CCCCC4)cc3)cc2)cc1]

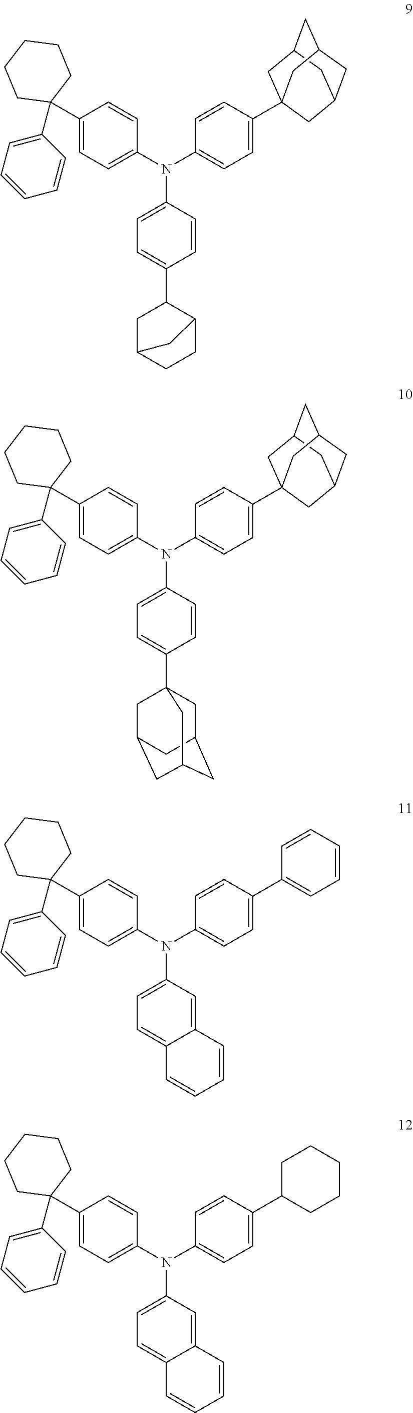 [SMILES: c1ccc(C2(c3ccc(N(c4ccc(C5CC6CCC5C6)cc4)c4ccc(C56CC7CC(CC(C7)C5)C6)cc4)cc3)CCCCC2)cc1 ; c1ccc(C2(c3ccc(N(c4ccc(C56CC7CC(CC(C7)C5)C6)cc4)c4ccc(C56CC7CC(CC(C7)C5)C6)cc4)cc3)CCCCC2)cc1 ; c1ccc(-c2ccc(N(c3ccc(C4(c5ccccc5)CCCCC4)cc3)c3ccc4ccccc4c3)cc2)cc1 ; c1ccc(C2(c3ccc(N(c4ccc(C5CCCCC5)cc4)c4ccc5ccccc5c4)cc3)CCCCC2)cc1]

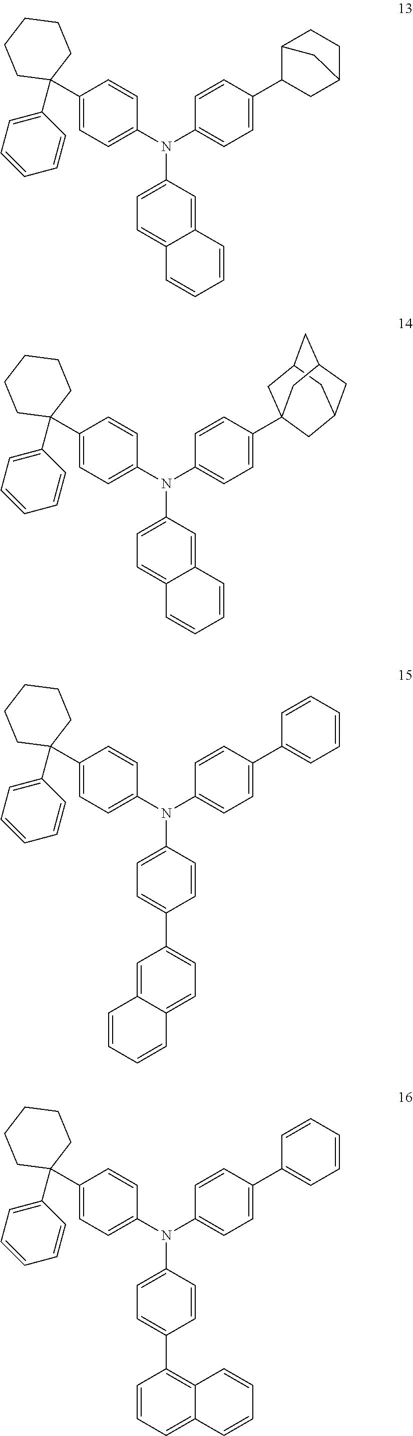 [SMILES: c1ccc(C2(c3ccc(N(c4ccc(C5CC6CCC5C6)cc4)c4ccc5ccccc5c4)cc3)CCCCC2)cc1 ; c1ccc(C2(c3ccc(N(c4ccc(C56CC7CC(CC(C7)C5)C6)cc4)c4ccc5ccccc5c4)cc3)CCCCC2)cc1 ; c1ccc(-c2ccc(N(c3ccc(-c4ccc5ccccc5c4)cc3)c3ccc(C4(c5ccccc5)CCCCC4)cc3)cc2)cc1 ; c1ccc(-c2ccc(N(c3ccc(-c4cccc5ccccc45)cc3)c3ccc(C4(c5ccccc5)CCCCC4)cc3)cc2)cc1]

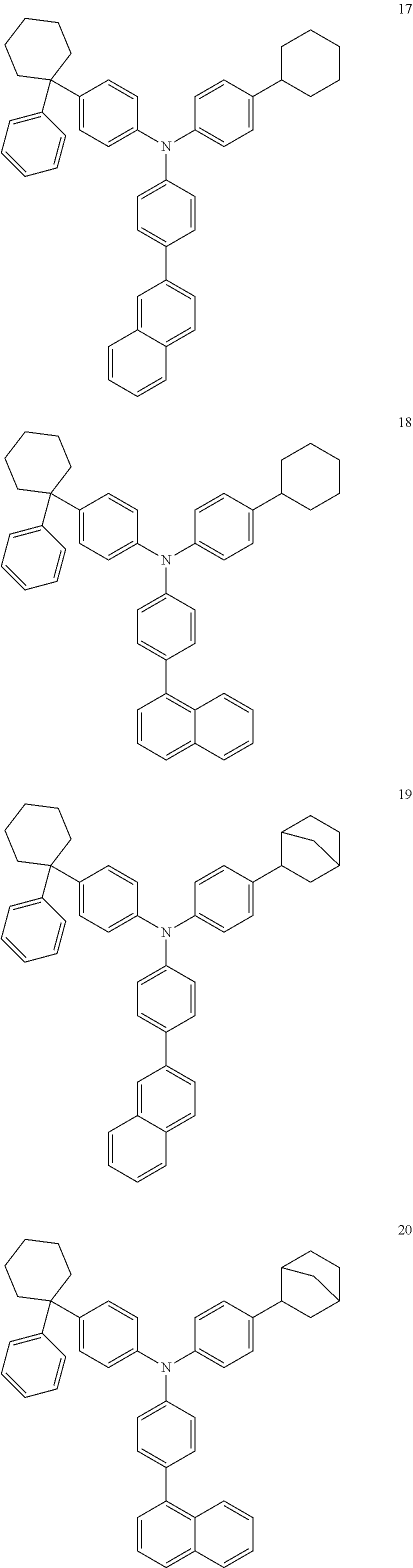 [SMILES: c1ccc(C2(c3ccc(N(c4ccc(-c5ccc6ccccc6c5)cc4)c4ccc(C5CCCCC5)cc4)cc3)CCCCC2)cc1 ; c1ccc(C2(c3ccc(N(c4ccc(-c5cccc6ccccc56)cc4)c4ccc(C5CCCCC5)cc4)cc3)CCCCC2)cc1 ; c1ccc(C2(c3ccc(N(c4ccc(-c5ccc6ccccc6c5)cc4)c4ccc(C5CC6CCC5C6)cc4)cc3)CCCCC2)cc1 ; c1ccc(C2(c3ccc(N(c4ccc(-c5cccc6ccccc56)cc4)c4ccc(C5CC6CCC5C6)cc4)cc3)CCCCC2)cc1]

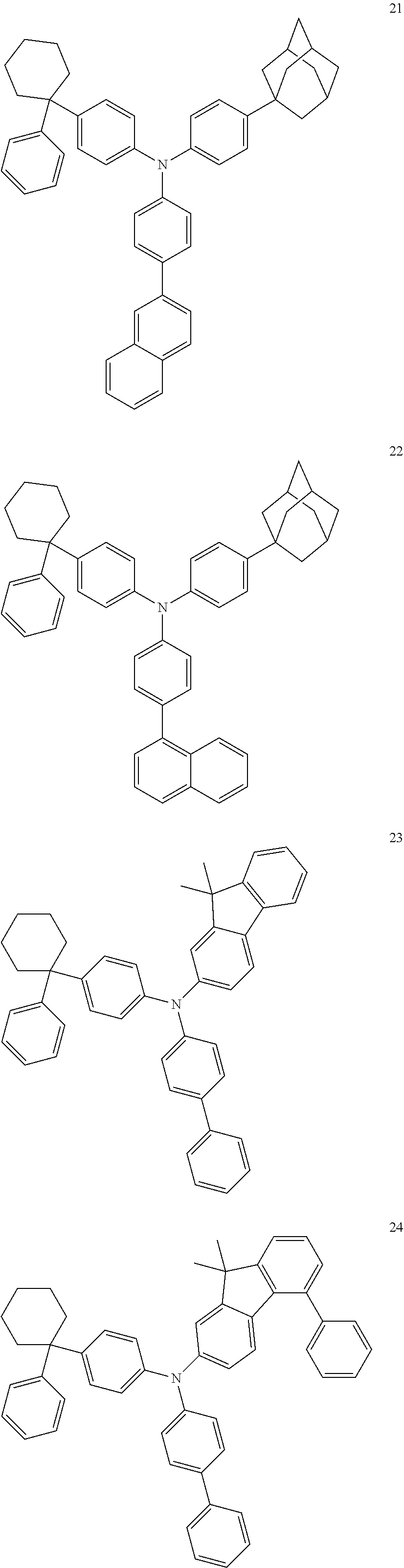 [SMILES: c1ccc(C2(c3ccc(N(c4ccc(-c5ccc6ccccc6c5)cc4)c4ccc(C56CC7CC(CC(C7)C5)C6)cc4)cc3)CCCCC2)cc1 ; c1ccc(C2(c3ccc(N(c4ccc(-c5cccc6ccccc56)cc4)c4ccc(C56CC7CC(CC(C7)C5)C6)cc4)cc3)CCCCC2)cc1 ; CC1(C)c2ccccc2-c2ccc(N(c3ccc(-c4ccccc4)cc3)c3ccc(C4(c5ccccc5)CCCCC4)cc3)cc21 ; CC1(C)c2cc(N(c3ccc(-c4ccccc4)cc3)c3ccc(C4(c5ccccc5)CCCCC4)cc3)ccc2-c2c(-c3ccccc3)cccc21]

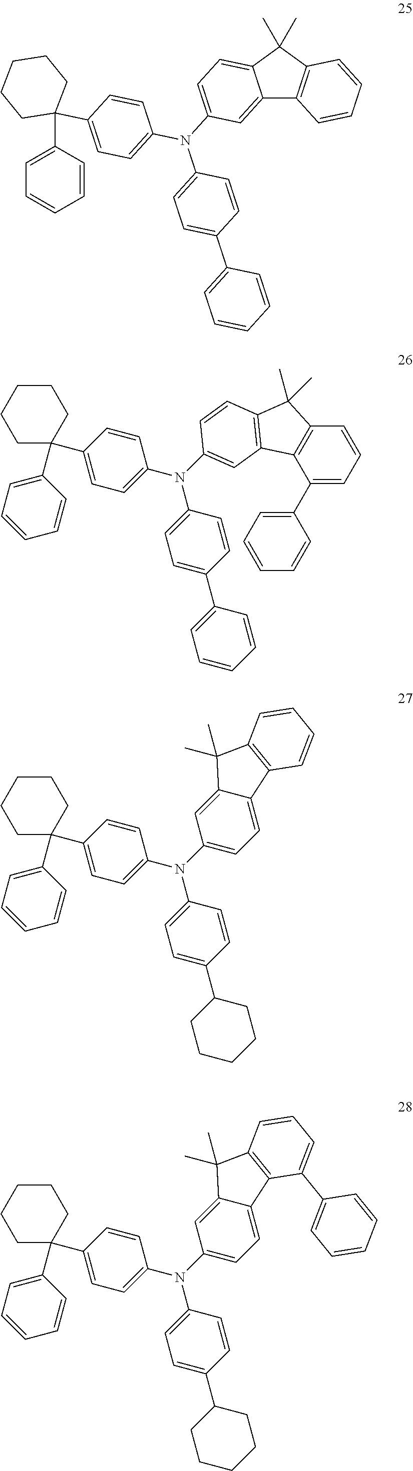 [SMILES: CC1(C)c2ccccc2-c2cc(N(c3ccc(-c4ccccc4)cc3)c3ccc(C4(c5ccccc5)CCCCC4)cc3)ccc21 ; CC1(C)c2ccc(N(c3ccc(-c4ccccc4)cc3)c3ccc(C4(c5ccccc5)CCCCC4)cc3)cc2-c2c(-c3ccccc3)cccc21 ; CC1(C)c2ccccc2-c2ccc(N(c3ccc(C4CCCCC4)cc3)c3ccc(C4(c5ccccc5)CCCCC4)cc3)cc21 ; CC1(C)c2cc(N(c3ccc(C4CCCCC4)cc3)c3ccc(C4(c5ccccc5)CCCCC4)cc3)ccc2-c2c(-c3ccccc3)cccc21]

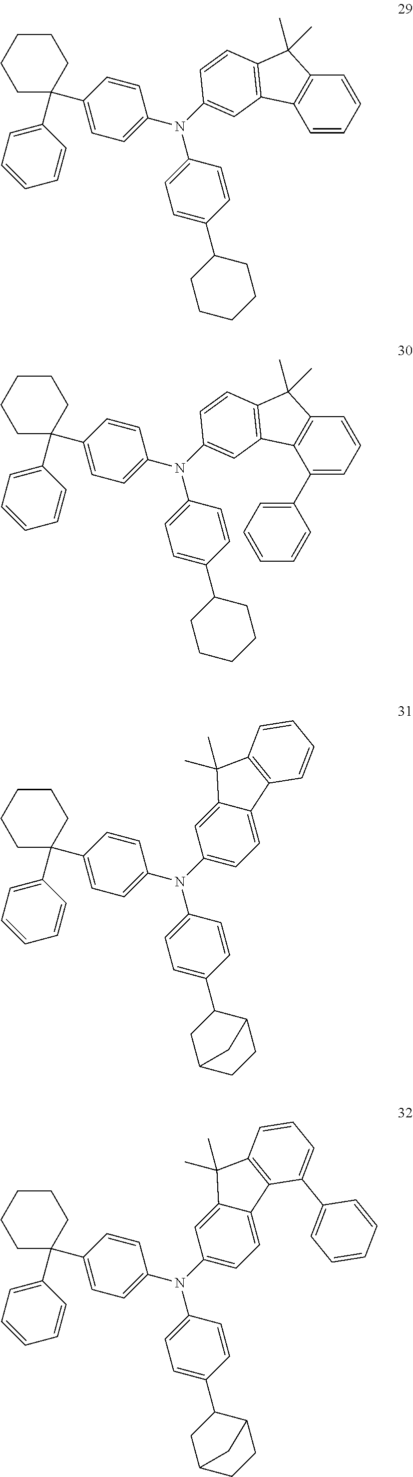 [SMILES: CC1(C)c2ccccc2-c2cc(N(c3ccc(C4CCCCC4)cc3)c3ccc(C4(c5ccccc5)CCCCC4)cc3)ccc21 ; CC1(C)c2ccc(N(c3ccc(C4CCCCC4)cc3)c3ccc(C4(c5ccccc5)CCCCC4)cc3)cc2-c2c(-c3ccccc3)cccc21 ; CC1(C)c2ccccc2-c2ccc(N(c3ccc(C4CC5CCC4C5)cc3)c3ccc(C4(c5ccccc5)CCCCC4)cc3)cc21 ; CC1(C)c2cc(N(c3ccc(C4CC5CCC4C5)cc3)c3ccc(C4(c5ccccc5)CCCCC4)cc3)ccc2-c2c(-c3ccccc3)cccc21]

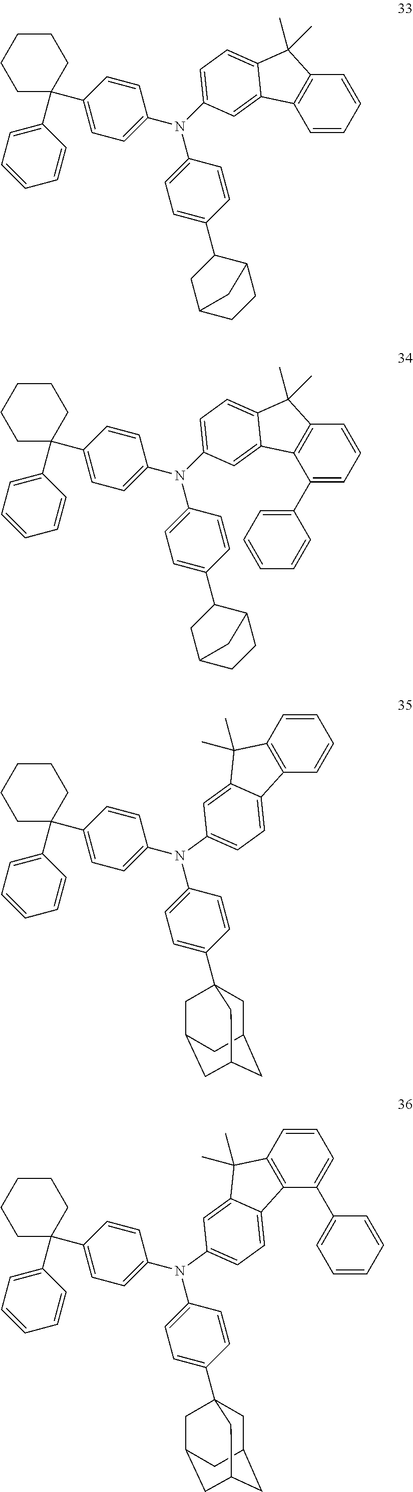 [SMILES: CC1(C)c2ccccc2-c2cc(N(c3ccc(C4CC5CCC4C5)cc3)c3ccc(C4(c5ccccc5)CCCCC4)cc3)ccc21 ; CC1(C)c2ccc(N(c3ccc(C4CC5CCC4C5)cc3)c3ccc(C4(c5ccccc5)CCCCC4)cc3)cc2-c2c(-c3ccccc3)cccc21 ; CC1(C)c2ccccc2-c2ccc(N(c3ccc(C45CC6CC(CC(C6)C4)C5)cc3)c3ccc(C4(c5ccccc5)CCCCC4)cc3)cc21 ; CC1(C)c2cc(N(c3ccc(C45CC6CC(CC(C6)C4)C5)cc3)c3ccc(C4(c5ccccc5)CCCCC4)cc3)ccc2-c2c(-c3ccccc3)cccc21]

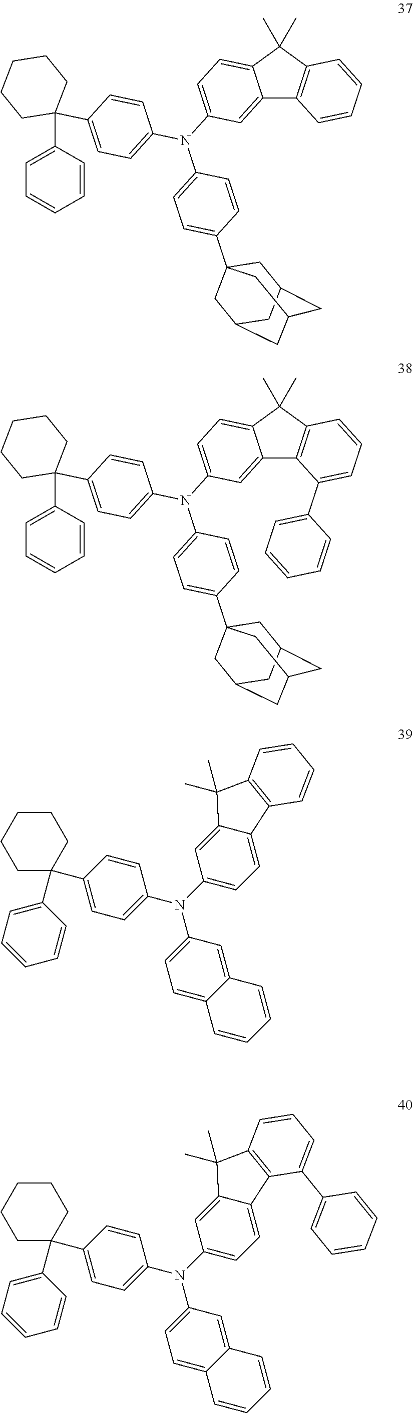

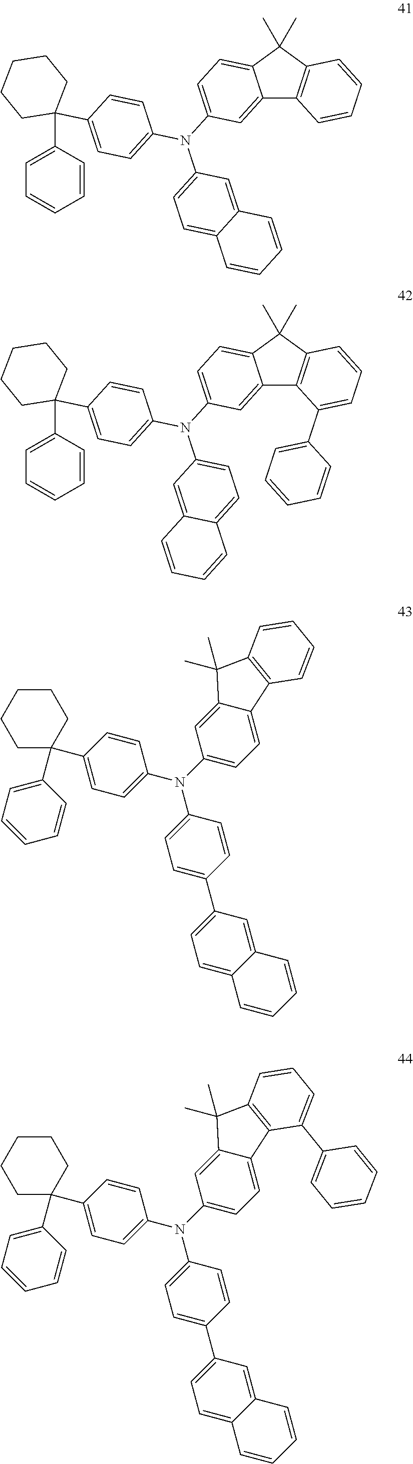

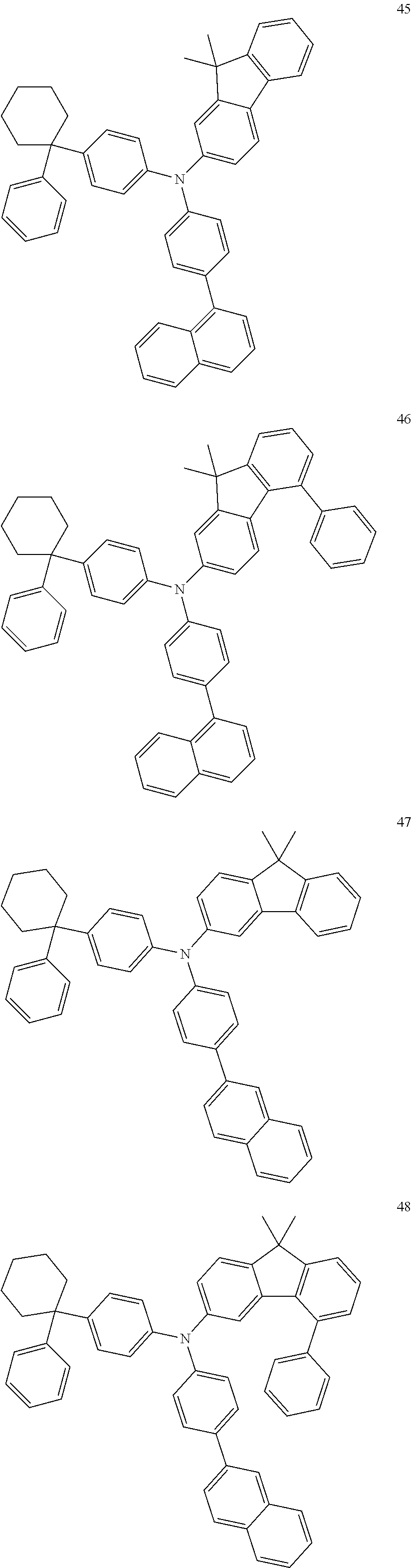

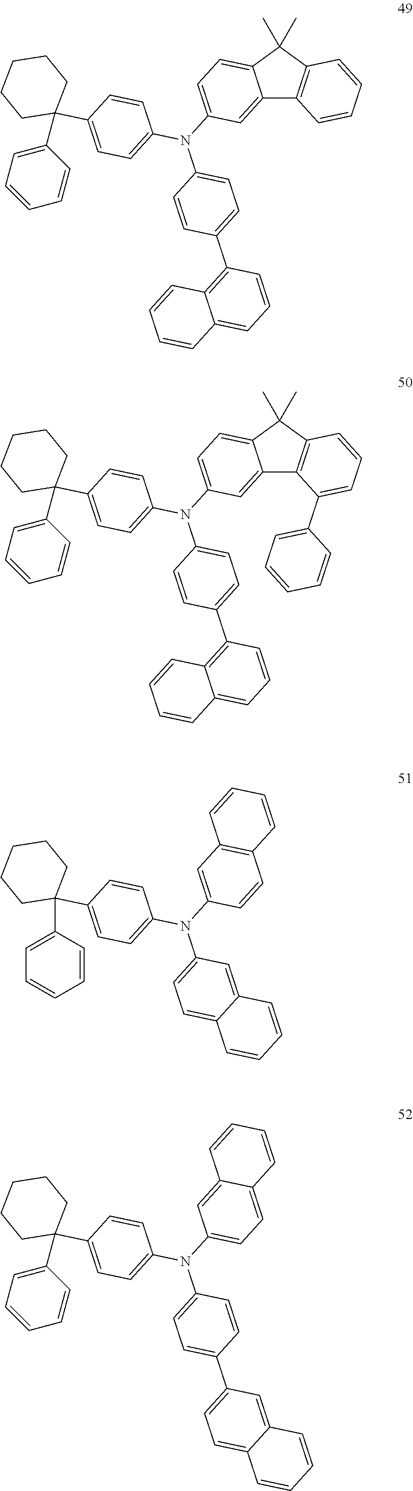 [SMILES: CC1(C)c2ccccc2-c2cc(N(c3ccc(-c4cccc5ccccc45)cc3)c3ccc(C4(c5ccccc5)CCCCC4)cc3)ccc21 ; CC1(C)c2ccc(N(c3ccc(-c4cccc5ccccc45)cc3)c3ccc(C4(c5ccccc5)CCCCC4)cc3)cc2-c2c(-c3ccccc3)cccc21 ; c1ccc(C2(c3ccc(N(c4ccc5ccccc5c4)c4ccc5ccccc5c4)cc3)CCCCC2)cc1 ; c1ccc(C2(c3ccc(N(c4ccc(-c5ccc6ccccc6c5)cc4)c4ccc5ccccc5c4)cc3)CCCCC2)cc1]

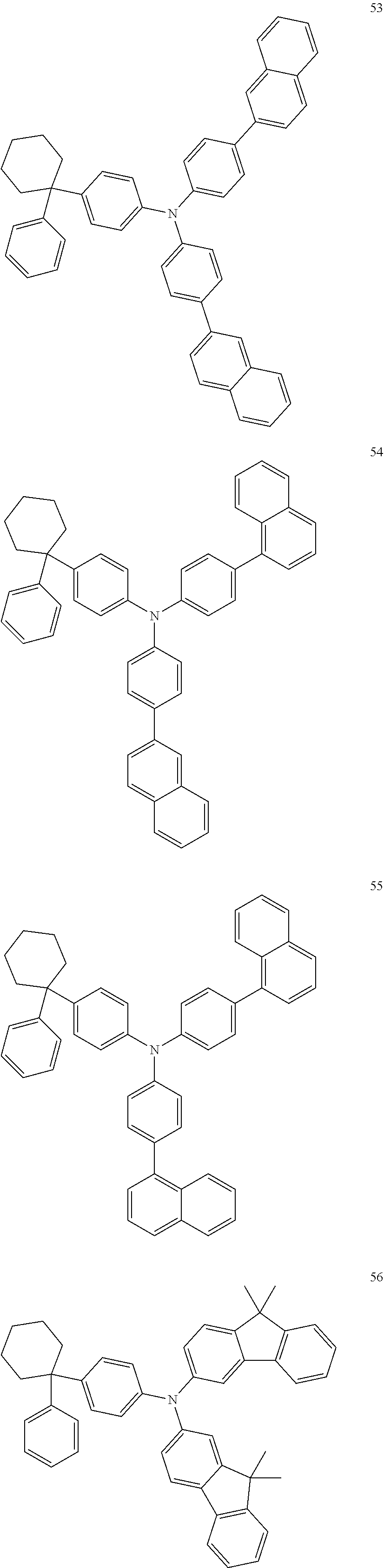

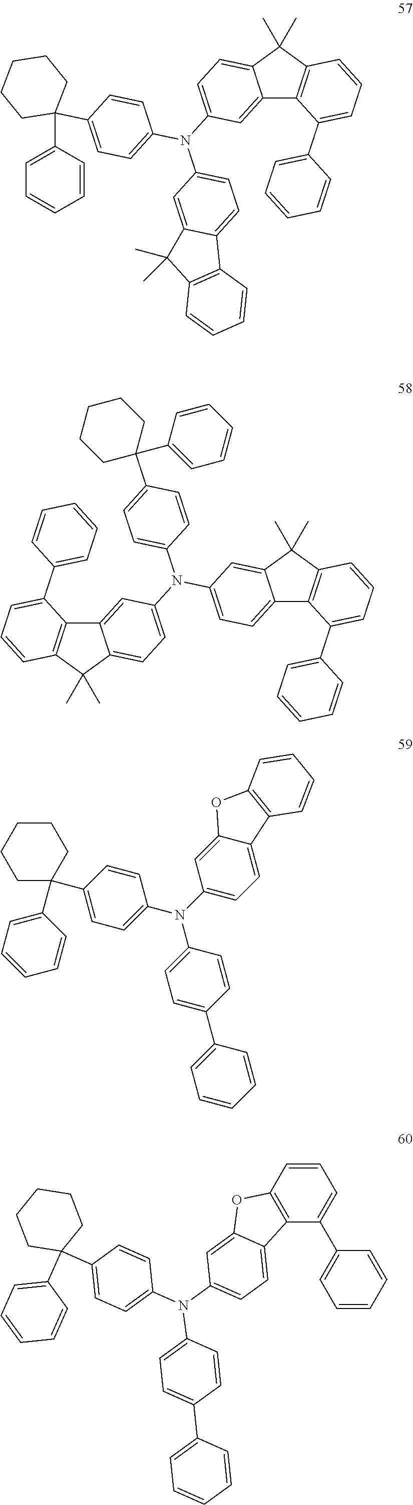

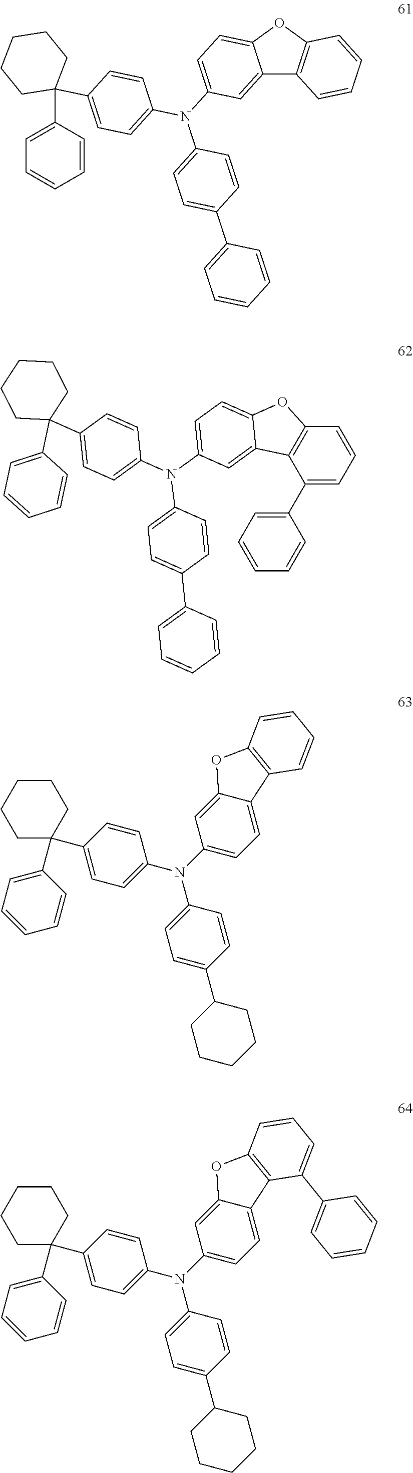 [SMILES: c1ccc(-c2ccc(N(c3ccc(C4(c5ccccc5)CCCCC4)cc3)c3ccc4oc5ccccc5c4c3)cc2)cc1 ; c1ccc(-c2ccc(N(c3ccc(C4(c5ccccc5)CCCCC4)cc3)c3ccc4oc5cccc(-c6ccccc6)c5c4c3)cc2)cc1 ; c1ccc(C2(c3ccc(N(c4ccc(C5CCCCC5)cc4)c4ccc5c(c4)oc4ccccc45)cc3)CCCCC2)cc1 ; c1ccc(-c2cccc3oc4cc(N(c5ccc(C6CCCCC6)cc5)c5ccc(C6(c7ccccc7)CCCCC6)cc5)ccc4c23)cc1]

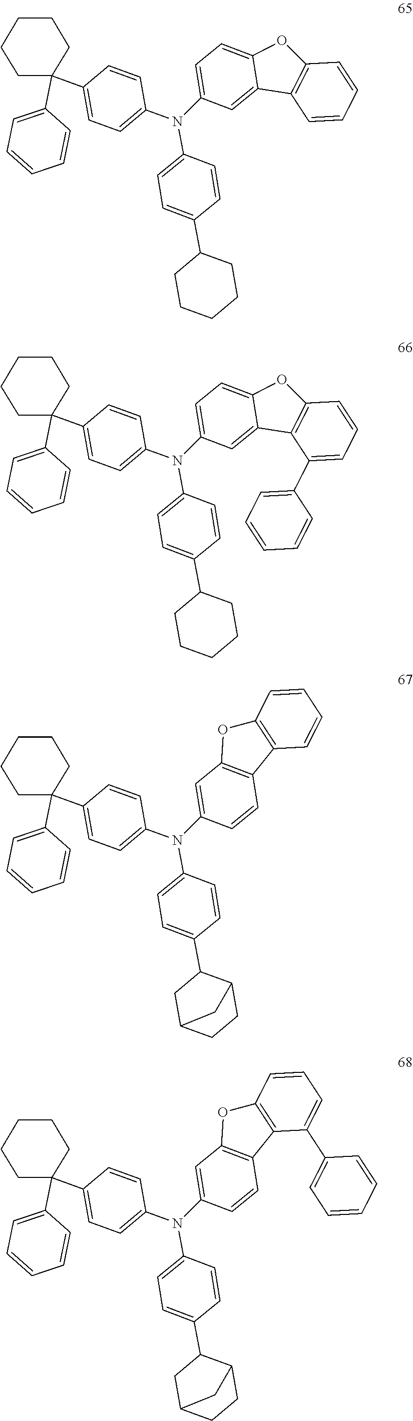 [SMILES: c1ccc(C2(c3ccc(N(c4ccc(C5CCCCC5)cc4)c4ccc5oc6ccccc6c5c4)cc3)CCCCC2)cc1 ; c1ccc(-c2cccc3oc4ccc(N(c5ccc(C6CCCCC6)cc5)c5ccc(C6(c7ccccc7)CCCCC6)cc5)cc4c23)cc1 ; c1ccc(C2(c3ccc(N(c4ccc(C5CC6CCC5C6)cc4)c4ccc5c(c4)oc4ccccc45)cc3)CCCCC2)cc1 ; c1ccc(-c2cccc3oc4cc(N(c5ccc(C6CC7CCC6C7)cc5)c5ccc(C6(c7ccccc7)CCCCC6)cc5)ccc4c23)cc1]

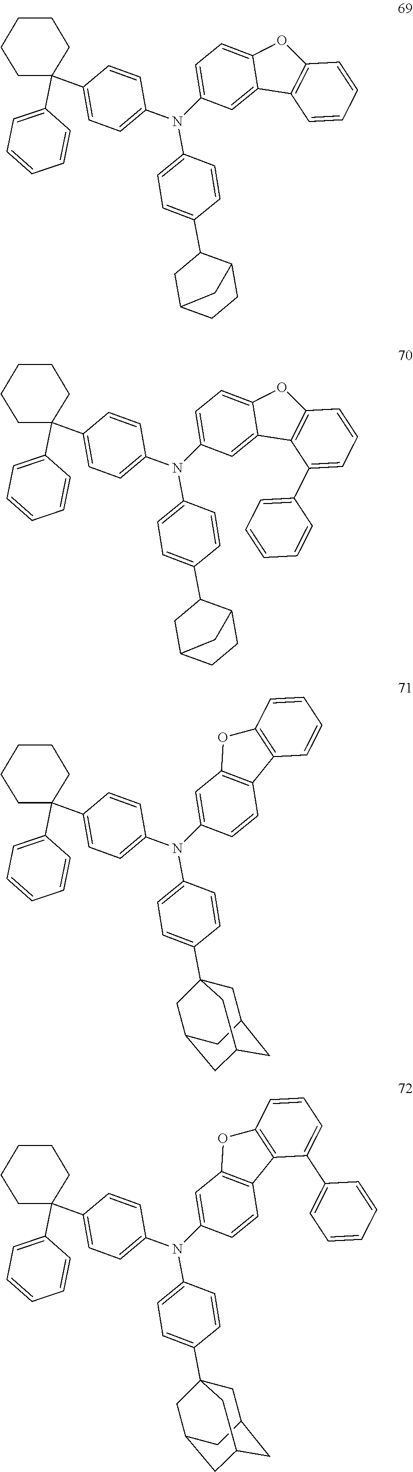

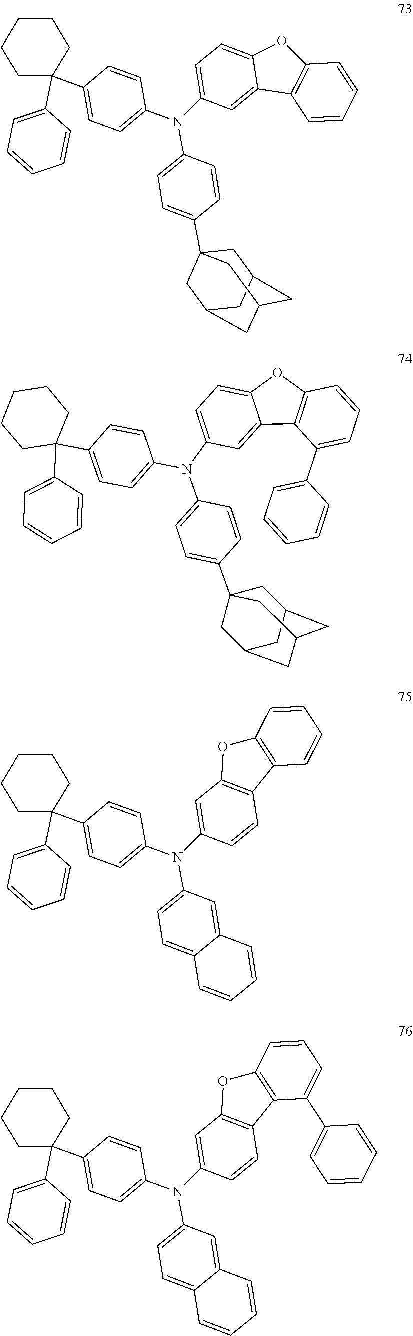

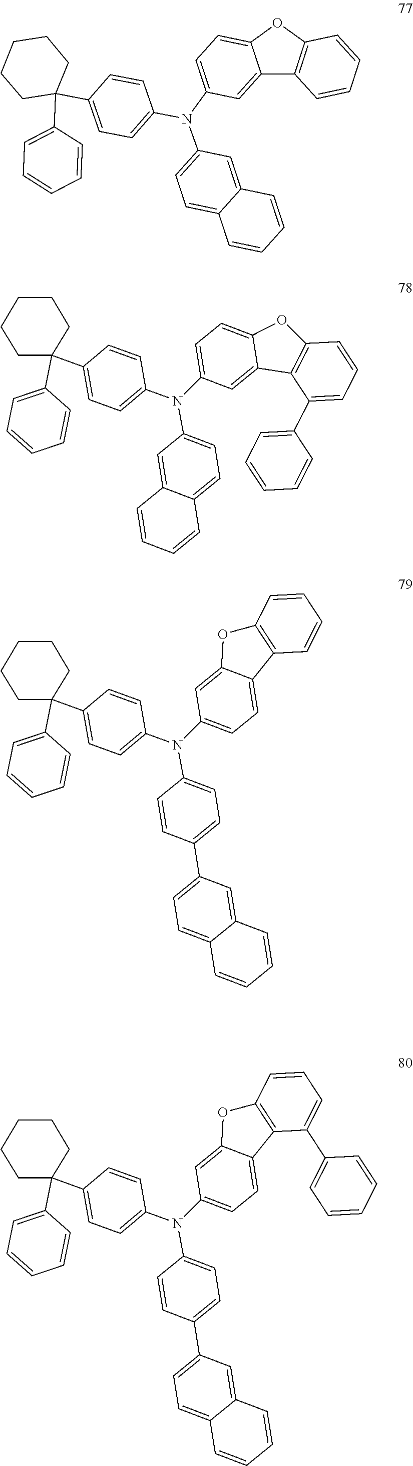 [SMILES: c1ccc(C2(c3ccc(N(c4ccc5ccccc5c4)c4ccc5oc6ccccc6c5c4)cc3)CCCCC2)cc1 ; c1ccc(-c2cccc3oc4ccc(N(c5ccc(C6(c7ccccc7)CCCCC6)cc5)c5ccc6ccccc6c5)cc4c23)cc1 ; c1ccc(C2(c3ccc(N(c4ccc(-c5ccc6ccccc6c5)cc4)c4ccc5c(c4)oc4ccccc45)cc3)CCCCC2)cc1 ; c1ccc(-c2cccc3oc4cc(N(c5ccc(-c6ccc7ccccc7c6)cc5)c5ccc(C6(c7ccccc7)CCCCC6)cc5)ccc4c23)cc1]

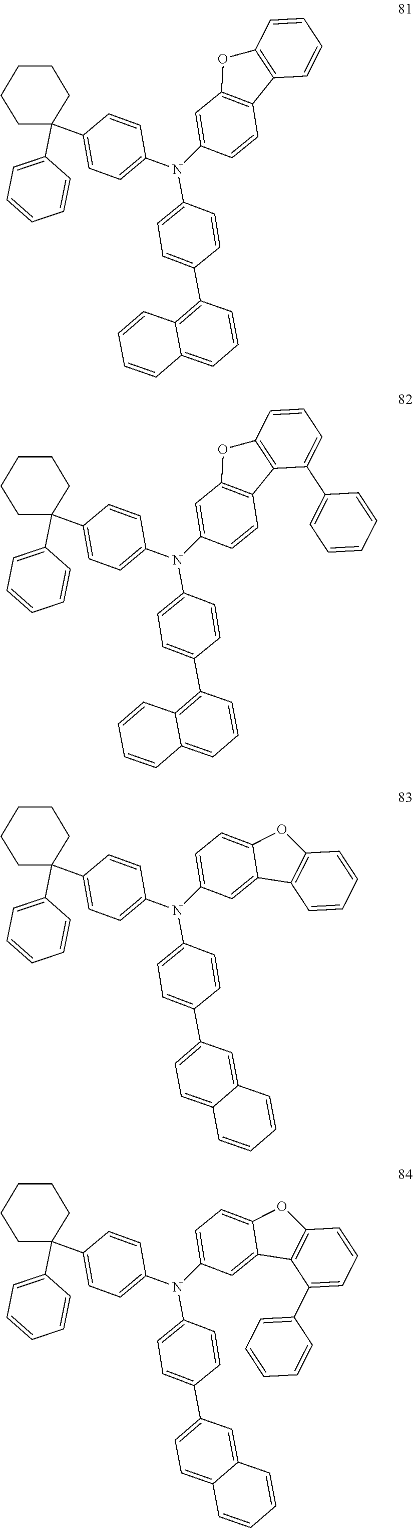 [SMILES: c1ccc(C2(c3ccc(N(c4ccc(-c5cccc6ccccc56)cc4)c4ccc5c(c4)oc4ccccc45)cc3)CCCCC2)cc1 ; c1ccc(-c2cccc3oc4cc(N(c5ccc(-c6cccc7ccccc67)cc5)c5ccc(C6(c7ccccc7)CCCCC6)cc5)ccc4c23)cc1 ; c1ccc(C2(c3ccc(N(c4ccc(-c5ccc6ccccc6c5)cc4)c4ccc5oc6ccccc6c5c4)cc3)CCCCC2)cc1 ; c1ccc(-c2cccc3oc4ccc(N(c5ccc(-c6ccc7ccccc7c6)cc5)c5ccc(C6(c7ccccc7)CCCCC6)cc5)cc4c23)cc1]

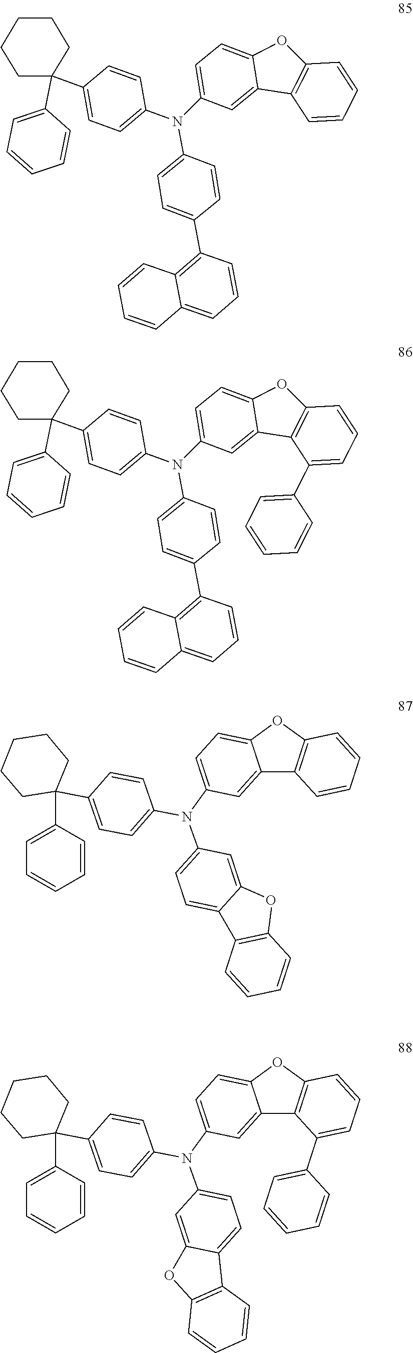 [SMILES: c1ccc(C2(c3ccc(N(c4ccc(-c5cccc6ccccc56)cc4)c4ccc5oc6ccccc6c5c4)cc3)CCCCC2)cc1 ; c1ccc(-c2cccc3oc4ccc(N(c5ccc(-c6cccc7ccccc67)cc5)c5ccc(C6(c7ccccc7)CCCCC6)cc5)cc4c23)cc1 ; c1ccc(C2(c3ccc(N(c4ccc5c(c4)oc4ccccc45)c4ccc5oc6ccccc6c5c4)cc3)CCCCC2)cc1 ; c1ccc(-c2cccc3oc4ccc(N(c5ccc(C6(c7ccccc7)CCCCC6)cc5)c5ccc6c(c5)oc5ccccc56)cc4c23)cc1]

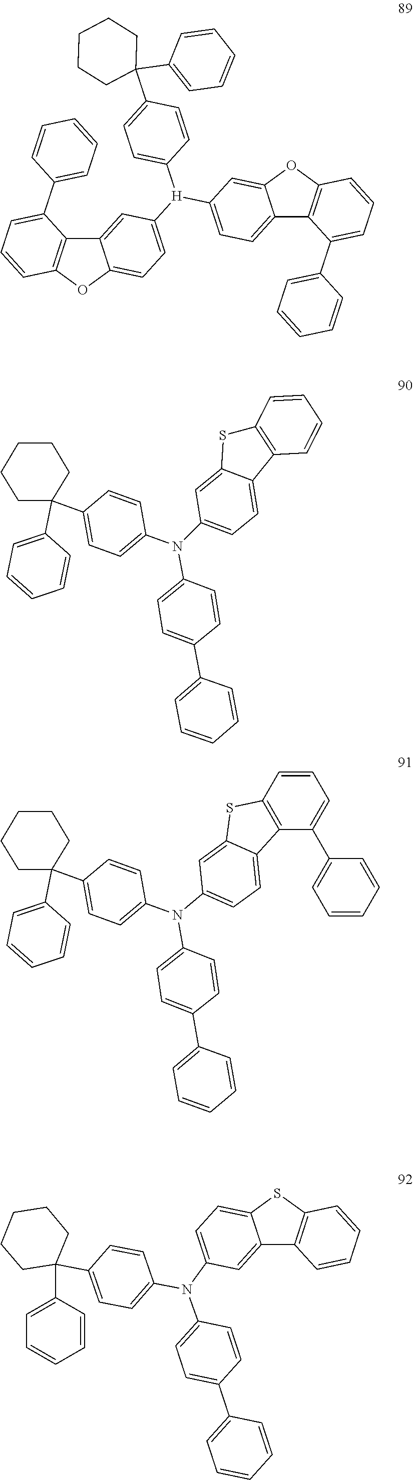

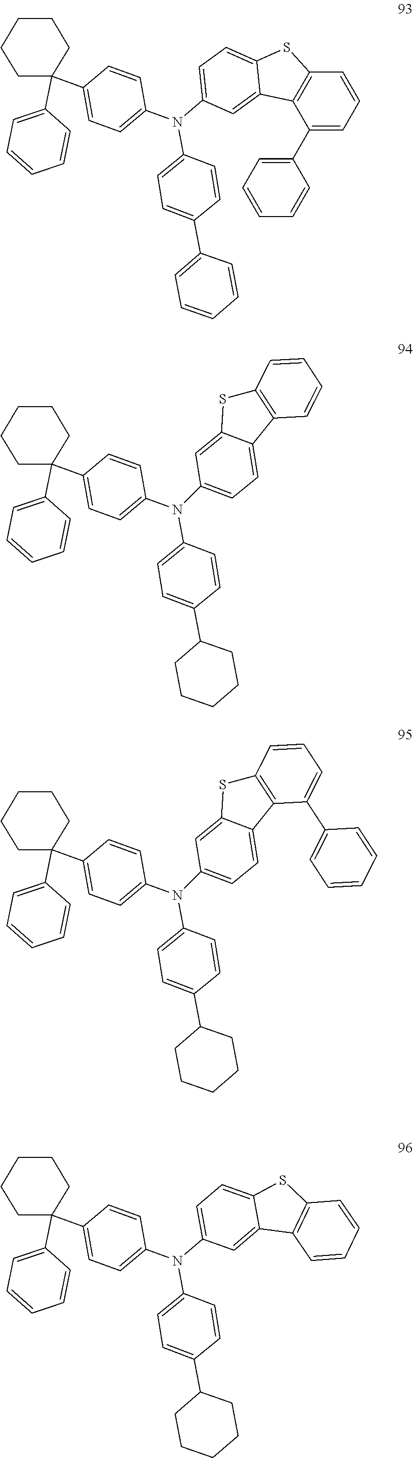 [SMILES: c1ccc(-c2ccc(N(c3ccc(C4(c5ccccc5)CCCCC4)cc3)c3ccc4sc5cccc(-c6ccccc6)c5c4c3)cc2)cc1 ; c1ccc(C2(c3ccc(N(c4ccc(C5CCCCC5)cc4)c4ccc5c(c4)sc4ccccc45)cc3)CCCCC2)cc1 ; c1ccc(-c2cccc3sc4cc(N(c5ccc(C6CCCCC6)cc5)c5ccc(C6(c7ccccc7)CCCCC6)cc5)ccc4c23)cc1 ; c1ccc(C2(c3ccc(N(c4ccc(C5CCCCC5)cc4)c4ccc5sc6ccccc6c5c4)cc3)CCCCC2)cc1]

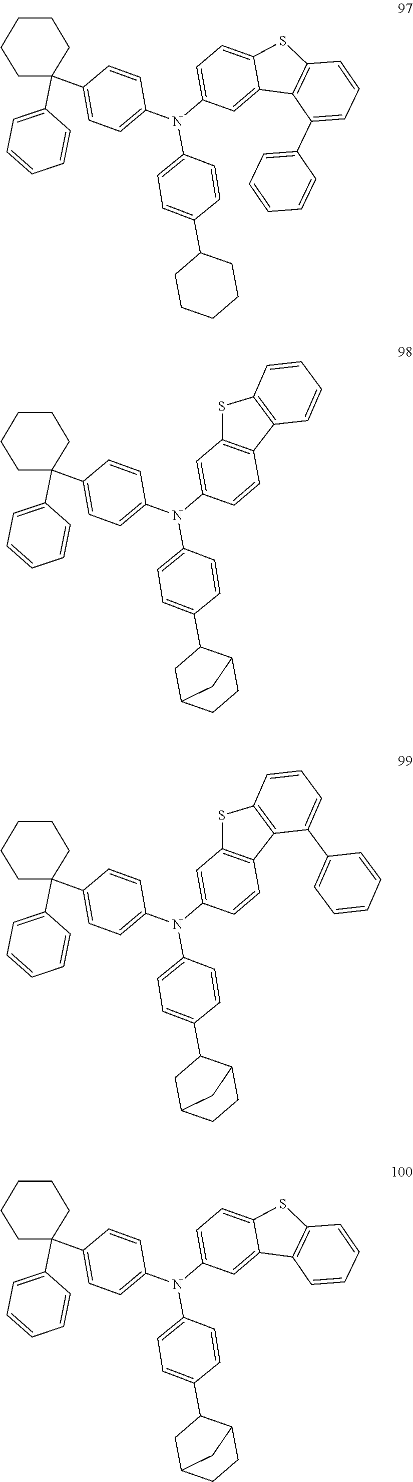 [SMILES: c1ccc(-c2cccc3sc4ccc(N(c5ccc(C6CCCCC6)cc5)c5ccc(C6(c7ccccc7)CCCCC6)cc5)cc4c23)cc1 ; c1ccc(C2(c3ccc(N(c4ccc(C5CC6CCC5C6)cc4)c4ccc5c(c4)sc4ccccc45)cc3)CCCCC2)cc1 ; c1ccc(-c2cccc3sc4cc(N(c5ccc(C6CC7CCC6C7)cc5)c5ccc(C6(c7ccccc7)CCCCC6)cc5)ccc4c23)cc1 ; c1ccc(C2(c3ccc(N(c4ccc(C5CC6CCC5C6)cc4)c4ccc5sc6ccccc6c5c4)cc3)CCCCC2)cc1]

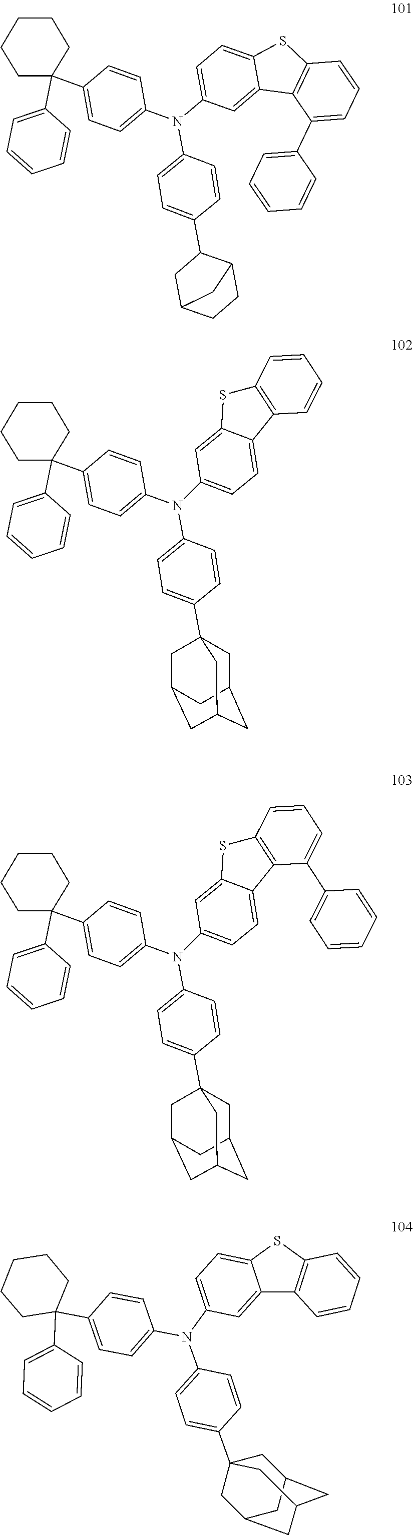 [SMILES: c1ccc(-c2cccc3sc4ccc(N(c5ccc(C6CC7CCC6C7)cc5)c5ccc(C6(c7ccccc7)CCCCC6)cc5)cc4c23)cc1 ; c1ccc(C2(c3ccc(N(c4ccc(C56CC7CC(CC(C7)C5)C6)cc4)c4ccc5c(c4)sc4ccccc45)cc3)CCCCC2)cc1 ; c1ccc(-c2cccc3sc4cc(N(c5ccc(C67CC8CC(CC(C8)C6)C7)cc5)c5ccc(C6(c7ccccc7)CCCCC6)cc5)ccc4c23)cc1 ; c1ccc(C2(c3ccc(N(c4ccc(C56CC7CC(CC(C7)C5)C6)cc4)c4ccc5sc6ccccc6c5c4)cc3)CCCCC2)cc1]

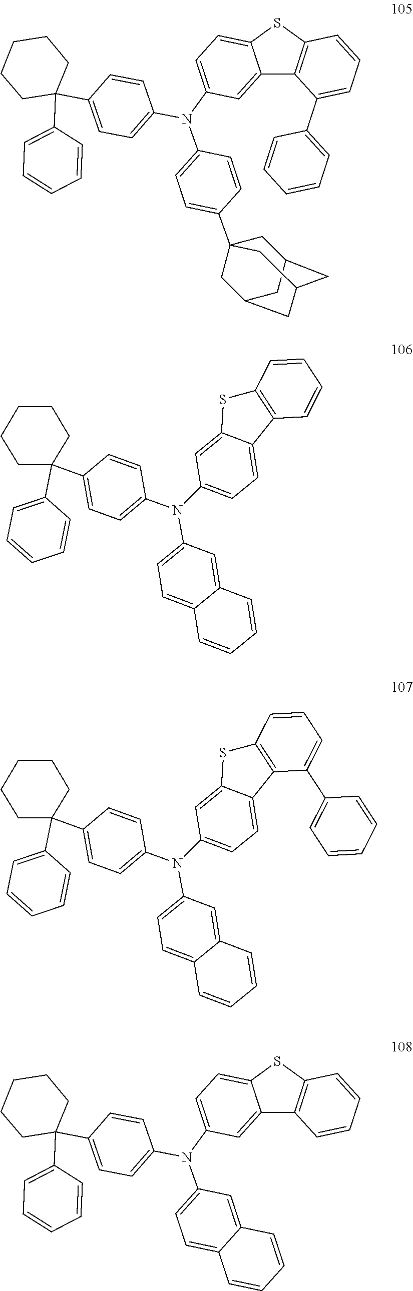

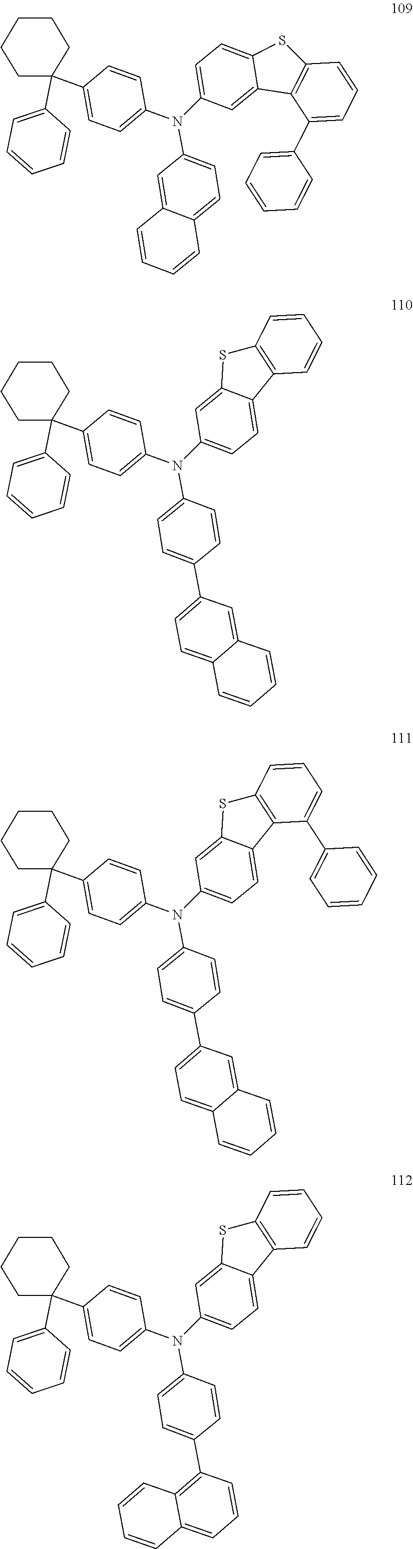

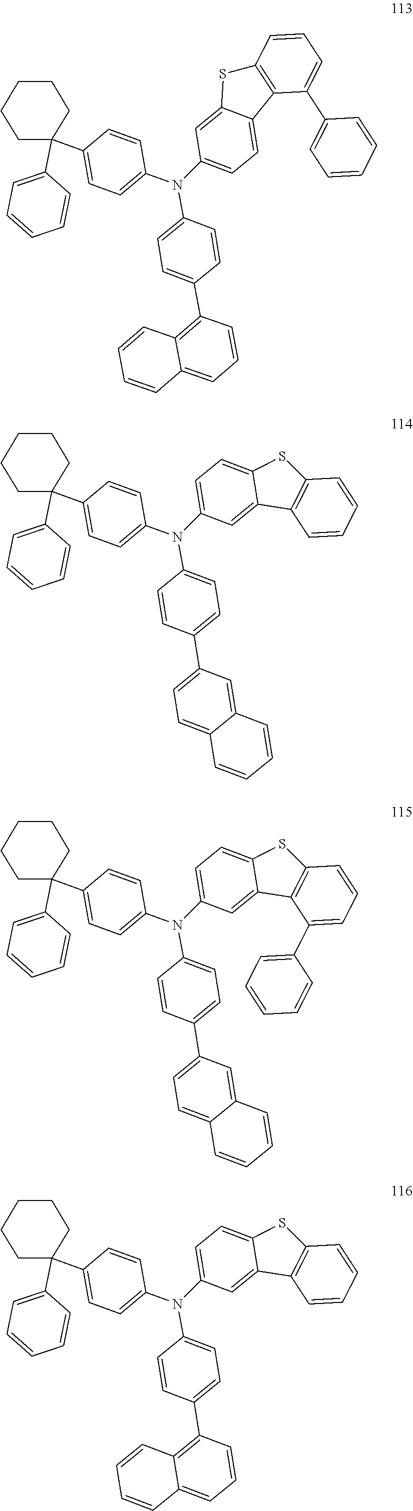 [SMILES: c1ccc(-c2cccc3sc4cc(N(c5ccc(-c6cccc7ccccc67)cc5)c5ccc(C6(c7ccccc7)CCCCC6)cc5)ccc4c23)cc1 ; c1ccc(C2(c3ccc(N(c4ccc(-c5ccc6ccccc6c5)cc4)c4ccc5sc6ccccc6c5c4)cc3)CCCCC2)cc1 ; c1ccc(-c2cccc3sc4ccc(N(c5ccc(-c6ccc7ccccc7c6)cc5)c5ccc(C6(c7ccccc7)CCCCC6)cc5)cc4c23)cc1 ; c1ccc(C2(c3ccc(N(c4ccc(-c5cccc6ccccc56)cc4)c4ccc5sc6ccccc6c5c4)cc3)CCCCC2)cc1]

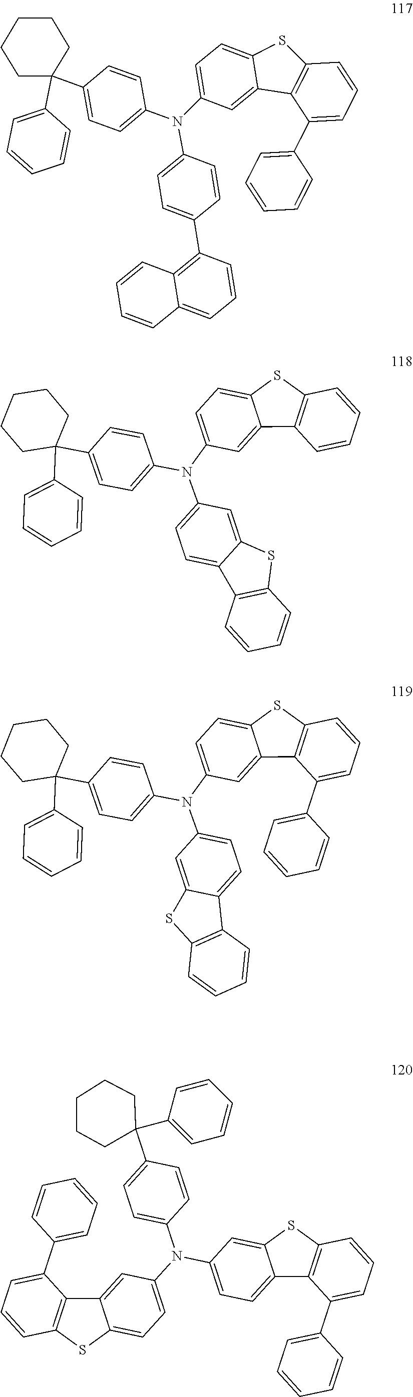 [SMILES: c1ccc(-c2cccc3sc4ccc(N(c5ccc(-c6cccc7ccccc67)cc5)c5ccc(C6(c7ccccc7)CCCCC6)cc5)cc4c23)cc1 ; c1ccc(C2(c3ccc(N(c4ccc5c(c4)sc4ccccc45)c4ccc5sc6ccccc6c5c4)cc3)CCCCC2)cc1 ; c1ccc(-c2cccc3sc4ccc(N(c5ccc(C6(c7ccccc7)CCCCC6)cc5)c5ccc6c(c5)sc5ccccc56)cc4c23)cc1 ; c1ccc(-c2cccc3sc4cc(N(c5ccc(C6(c7ccccc7)CCCCC6)cc5)c5ccc6sc7cccc(-c8ccccc8)c7c6c5)ccc4c23)cc1]

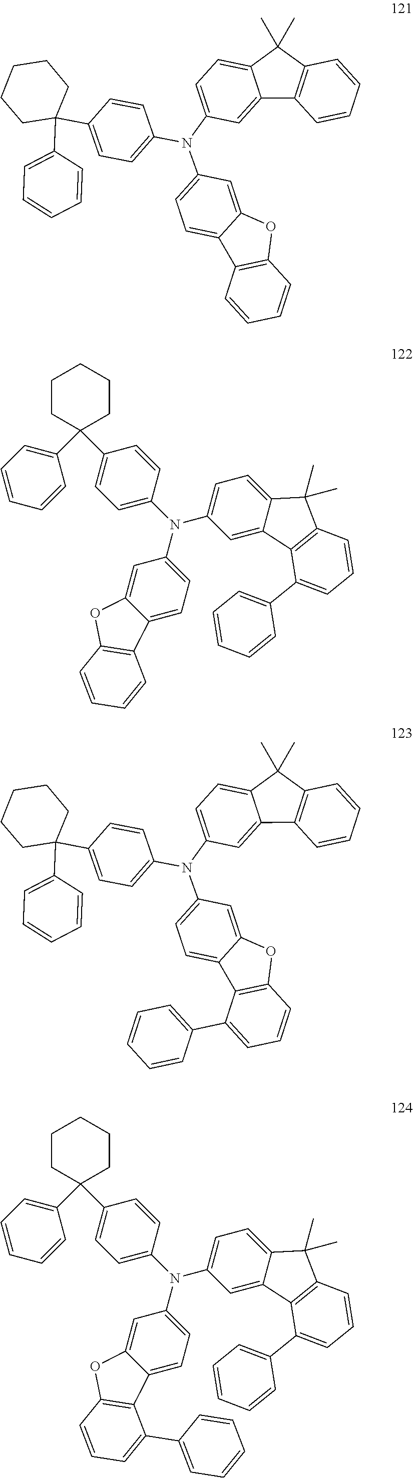 [SMILES: CC1(C)c2ccccc2-c2cc(N(c3ccc(C4(c5ccccc5)CCCCC4)cc3)c3ccc4c(c3)oc3ccccc34)ccc21 ; CC1(C)c2ccc(N(c3ccc(C4(c5ccccc5)CCCCC4)cc3)c3ccc4c(c3)oc3ccccc34)cc2-c2c(-c3ccccc3)cccc21 ; CC1(C)c2ccccc2-c2cc(N(c3ccc(C4(c5ccccc5)CCCCC4)cc3)c3ccc4c(c3)oc3cccc(-c5ccccc5)c34)ccc21 ; CC1(C)c2ccc(N(c3ccc(C4(c5ccccc5)CCCCC4)cc3)c3ccc4c(c3)oc3cccc(-c5ccccc5)c34)cc2-c2c(-c3ccccc3)cccc21]

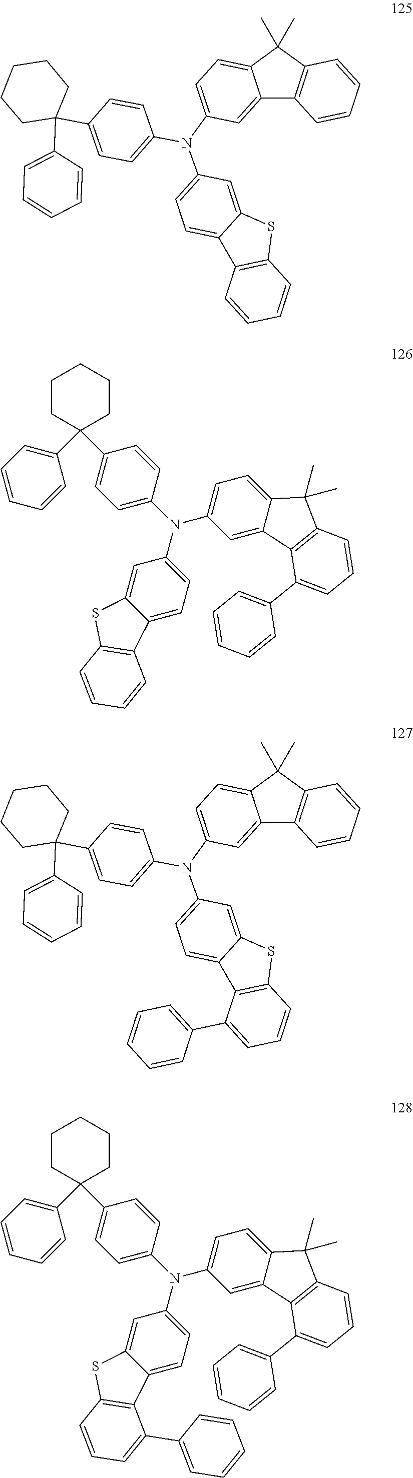 [SMILES: CC1(C)c2ccccc2-c2cc(N(c3ccc(C4(c5ccccc5)CCCCC4)cc3)c3ccc4c(c3)sc3ccccc34)ccc21 ; CC1(C)c2ccc(N(c3ccc(C4(c5ccccc5)CCCCC4)cc3)c3ccc4c(c3)sc3ccccc34)cc2-c2c(-c3ccccc3)cccc21 ; CC1(C)c2ccccc2-c2cc(N(c3ccc(C4(c5ccccc5)CCCCC4)cc3)c3ccc4c(c3)sc3cccc(-c5ccccc5)c34)ccc21 ; CC1(C)c2ccc(N(c3ccc(C4(c5ccccc5)CCCCC4)cc3)c3ccc4c(c3)sc3cccc(-c5ccccc5)c34)cc2-c2c(-c3ccccc3)cccc21]

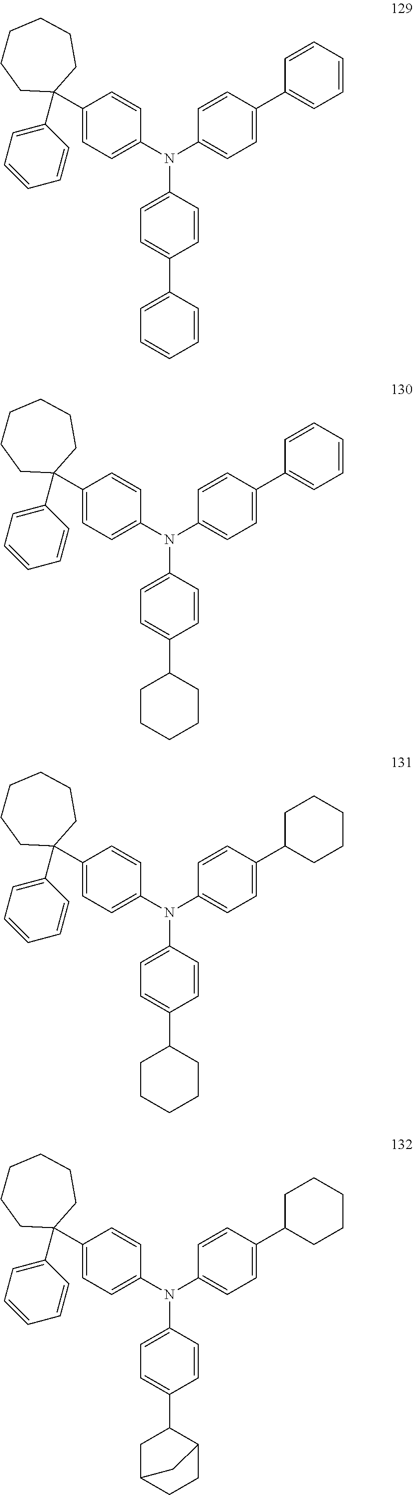 [SMILES: c1ccc(-c2ccc(N(c3ccc(-c4ccccc4)cc3)c3ccc(C4(c5ccccc5)CCCCCC4)cc3)cc2)cc1 ; c1ccc(-c2ccc(N(c3ccc(C4CCCCC4)cc3)c3ccc(C4(c5ccccc5)CCCCCC4)cc3)cc2)cc1 ; c1ccc(C2(c3ccc(N(c4ccc(C5CCCCC5)cc4)c4ccc(C5CCCCC5)cc4)cc3)CCCCCC2)cc1 ; c1ccc(C2(c3ccc(N(c4ccc(C5CCCCC5)cc4)c4ccc(C5CC6CCC5C6)cc4)cc3)CCCCCC2)cc1]

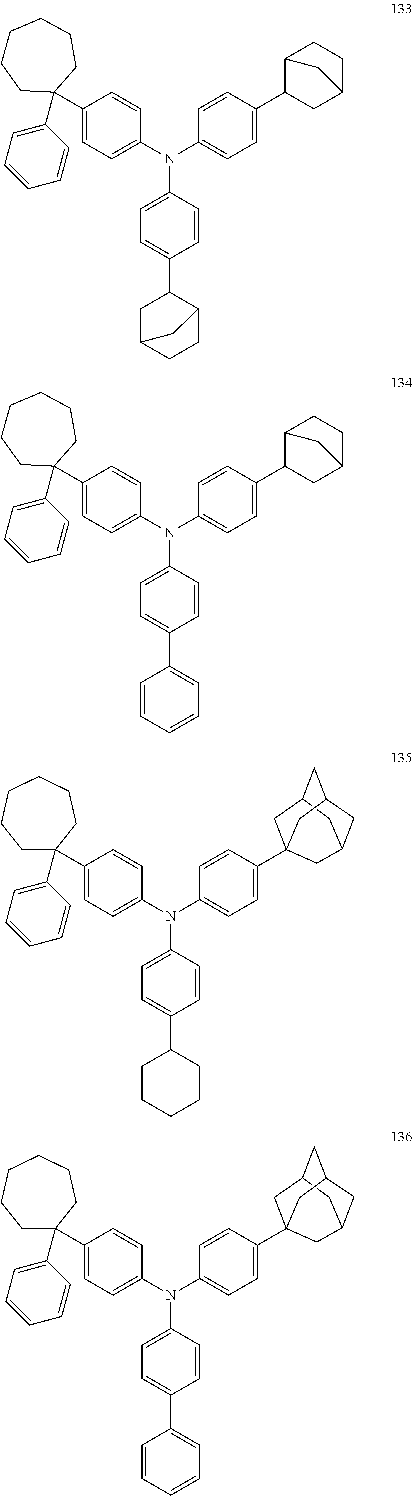 [SMILES: c1ccc(C2(c3ccc(N(c4ccc(C5CC6CCC5C6)cc4)c4ccc(C5CC6CCC5C6)cc4)cc3)CCCCCC2)cc1 ; c1ccc(-c2ccc(N(c3ccc(C4CC5CCC4C5)cc3)c3ccc(C4(c5ccccc5)CCCCCC4)cc3)cc2)cc1 ; c1ccc(C2(c3ccc(N(c4ccc(C5CCCCC5)cc4)c4ccc(C56CC7CC(CC(C7)C5)C6)cc4)cc3)CCCCCC2)cc1 ; c1ccc(-c2ccc(N(c3ccc(C45CC6CC(CC(C6)C4)C5)cc3)c3ccc(C4(c5ccccc5)CCCCCC4)cc3)cc2)cc1]

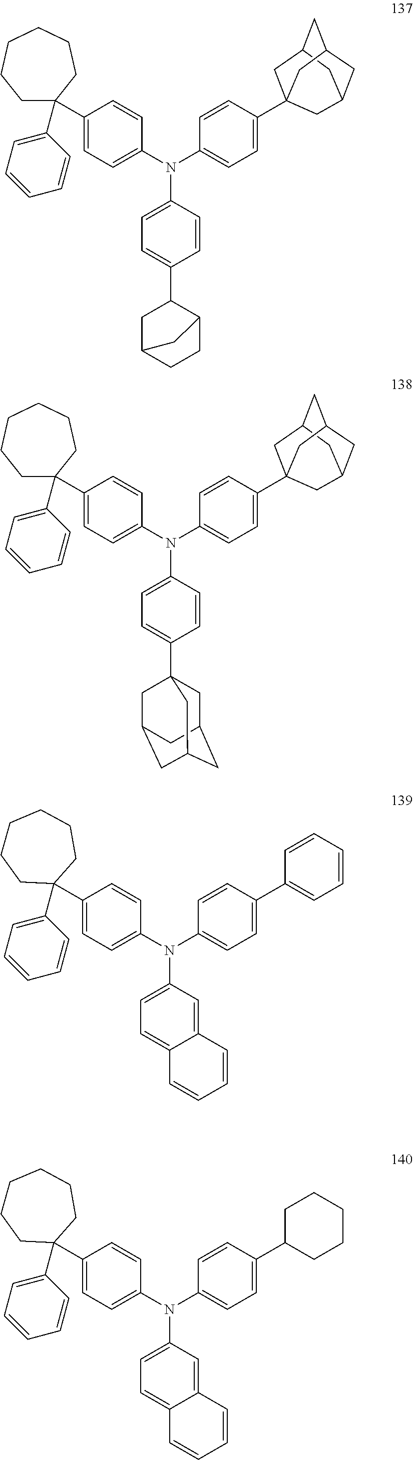 [SMILES: c1ccc(C2(c3ccc(N(c4ccc(C5CC6CCC5C6)cc4)c4ccc(C56CC7CC(CC(C7)C5)C6)cc4)cc3)CCCCCC2)cc1 ; c1ccc(C2(c3ccc(N(c4ccc(C56CC7CC(CC(C7)C5)C6)cc4)c4ccc(C56CC7CC(CC(C7)C5)C6)cc4)cc3)CCCCCC2)cc1 ; c1ccc(-c2ccc(N(c3ccc(C4(c5ccccc5)CCCCCC4)cc3)c3ccc4ccccc4c3)cc2)cc1 ; c1ccc(C2(c3ccc(N(c4ccc(C5CCCCC5)cc4)c4ccc5ccccc5c4)cc3)CCCCCC2)cc1]

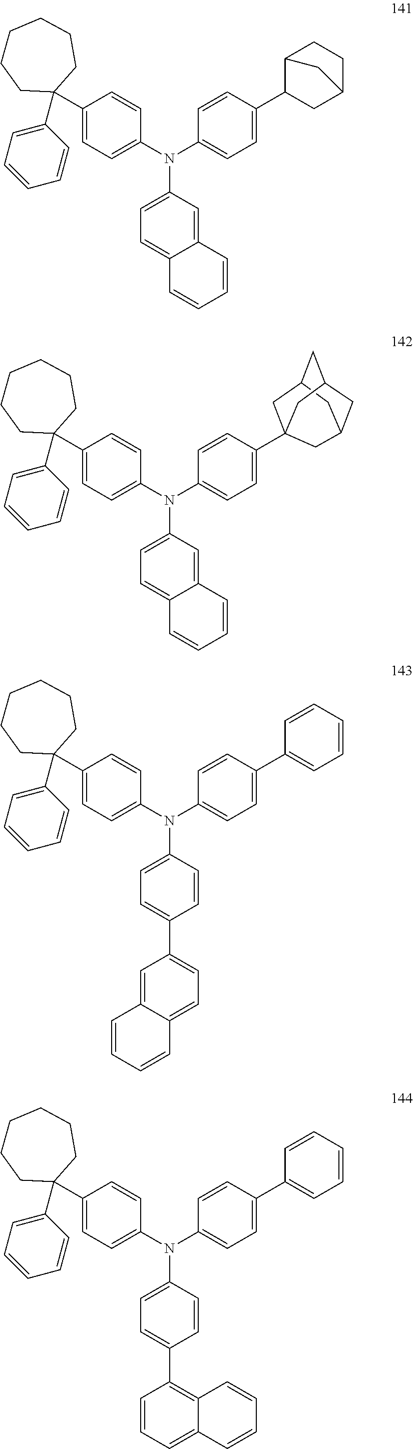 [SMILES: c1ccc(C2(c3ccc(N(c4ccc(C5CC6CCC5C6)cc4)c4ccc5ccccc5c4)cc3)CCCCCC2)cc1 ; c1ccc(C2(c3ccc(N(c4ccc(C56CC7CC(CC(C7)C5)C6)cc4)c4ccc5ccccc5c4)cc3)CCCCCC2)cc1 ; c1ccc(-c2ccc(N(c3ccc(-c4ccc5ccccc5c4)cc3)c3ccc(C4(c5ccccc5)CCCCCC4)cc3)cc2)cc1 ; c1ccc(-c2ccc(N(c3ccc(-c4cccc5ccccc45)cc3)c3ccc(C4(c5ccccc5)CCCCCC4)cc3)cc2)cc1]

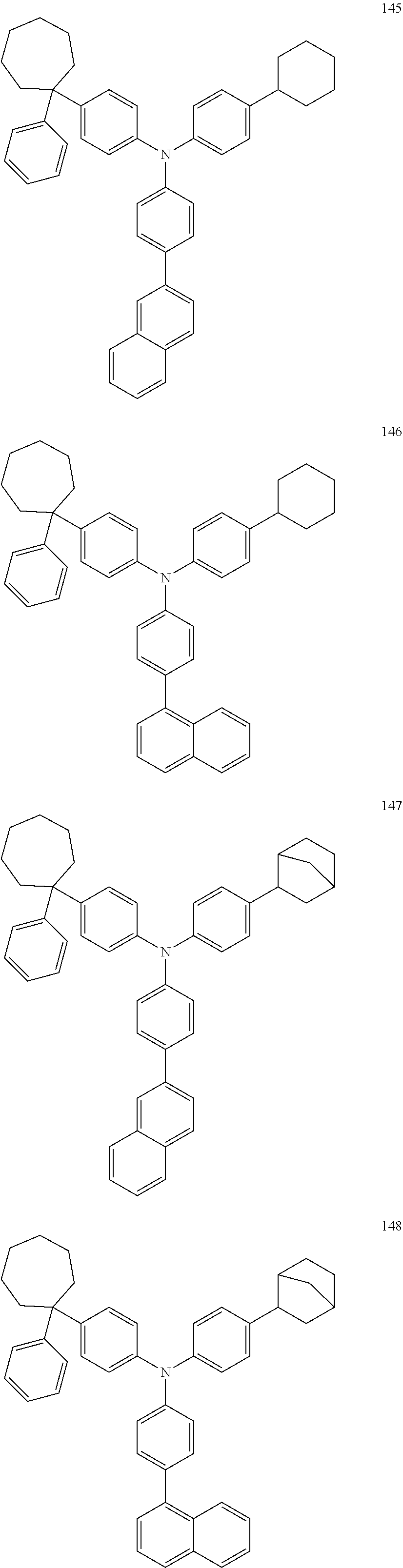 [SMILES: c1ccc(C2(c3ccc(N(c4ccc(-c5ccc6ccccc6c5)cc4)c4ccc(C5CCCCC5)cc4)cc3)CCCCCC2)cc1 ; c1ccc(C2(c3ccc(N(c4ccc(-c5cccc6ccccc56)cc4)c4ccc(C5CCCCC5)cc4)cc3)CCCCCC2)cc1 ; c1ccc(C2(c3ccc(N(c4ccc(-c5ccc6ccccc6c5)cc4)c4ccc(C5CC6CCC5C6)cc4)cc3)CCCCCC2)cc1 ; c1ccc(C2(c3ccc(N(c4ccc(-c5cccc6ccccc56)cc4)c4ccc(C5CC6CCC5C6)cc4)cc3)CCCCCC2)cc1]

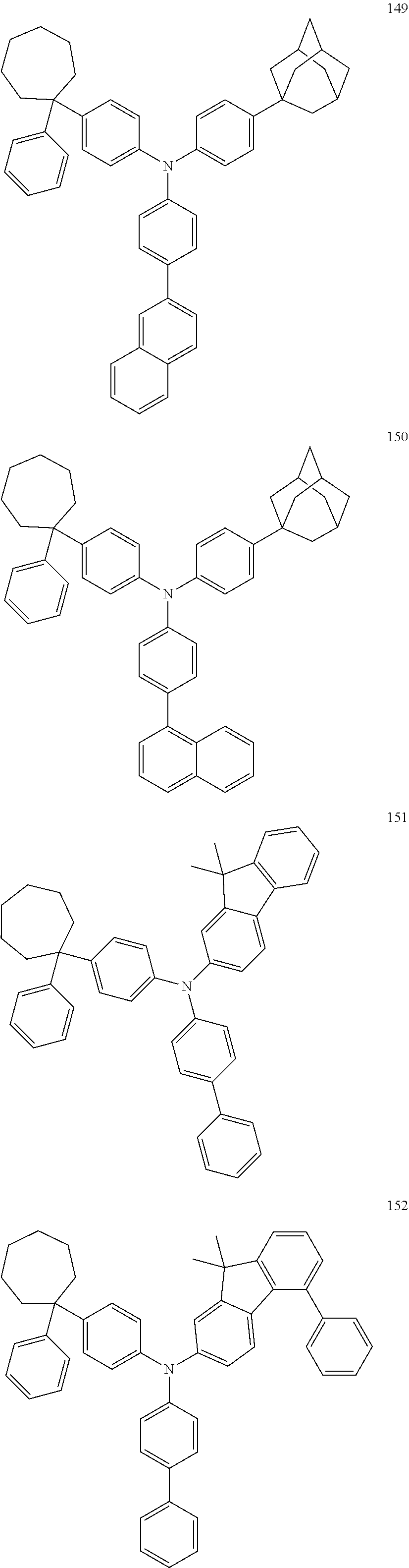 [SMILES: c1ccc(C2(c3ccc(N(c4ccc(-c5ccc6ccccc6c5)cc4)c4ccc(C56CC7CC(CC(C7)C5)C6)cc4)cc3)CCCCCC2)cc1 ; c1ccc(C2(c3ccc(N(c4ccc(-c5cccc6ccccc56)cc4)c4ccc(C56CC7CC(CC(C7)C5)C6)cc4)cc3)CCCCCC2)cc1 ; CC1(C)c2ccccc2-c2ccc(N(c3ccc(-c4ccccc4)cc3)c3ccc(C4(c5ccccc5)CCCCCC4)cc3)cc21 ; CC1(C)c2cc(N(c3ccc(-c4ccccc4)cc3)c3ccc(C4(c5ccccc5)CCCCCC4)cc3)ccc2-c2c(-c3ccccc3)cccc21]

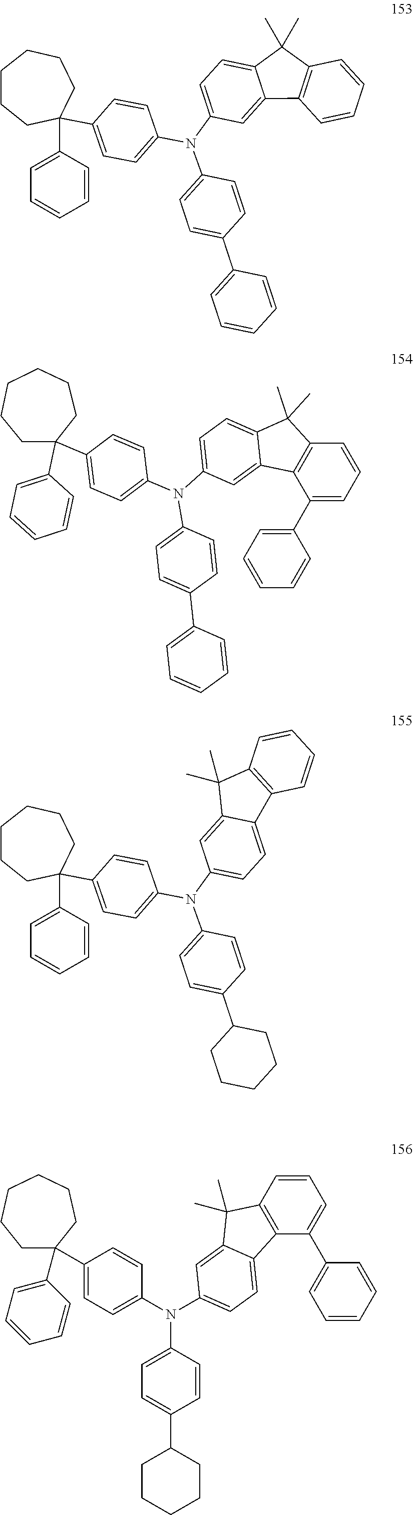 [SMILES: CC1(C)c2ccccc2-c2cc(N(c3ccc(-c4ccccc4)cc3)c3ccc(C4(c5ccccc5)CCCCCC4)cc3)ccc21 ; CC1(C)c2ccc(N(c3ccc(-c4ccccc4)cc3)c3ccc(C4(c5ccccc5)CCCCCC4)cc3)cc2-c2c(-c3ccccc3)cccc21 ; CC1(C)c2ccccc2-c2ccc(N(c3ccc(C4CCCCC4)cc3)c3ccc(C4(c5ccccc5)CCCCCC4)cc3)cc21 ; CC1(C)c2cc(N(c3ccc(C4CCCCC4)cc3)c3ccc(C4(c5ccccc5)CCCCCC4)cc3)ccc2-c2c(-c3ccccc3)cccc21]

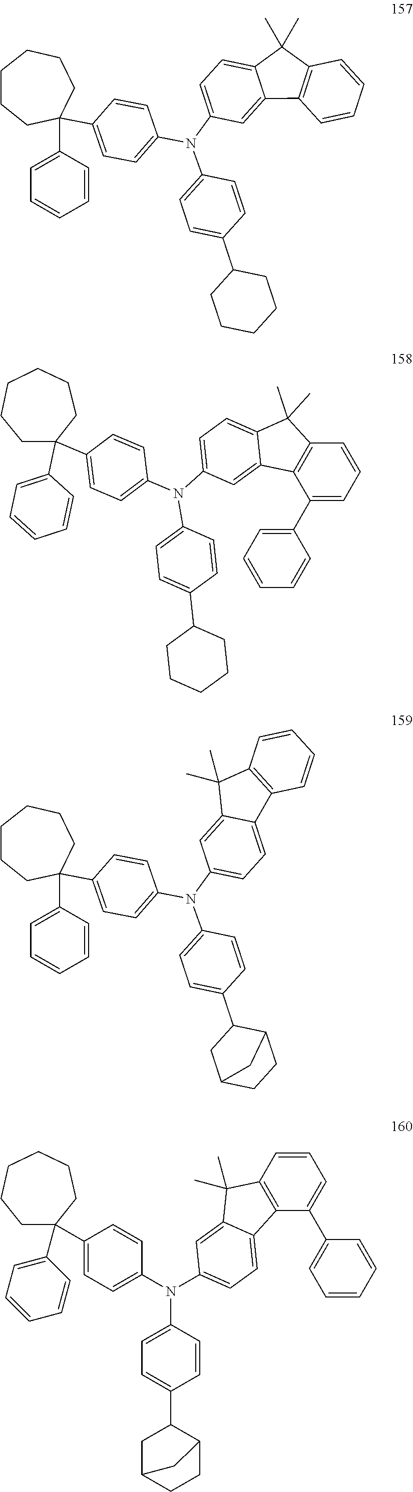 [SMILES: CC1(C)c2ccccc2-c2cc(N(c3ccc(C4CCCCC4)cc3)c3ccc(C4(c5ccccc5)CCCCCC4)cc3)ccc21 ; CC1(C)c2ccc(N(c3ccc(C4CCCCC4)cc3)c3ccc(C4(c5ccccc5)CCCCCC4)cc3)cc2-c2c(-c3ccccc3)cccc21 ; CC1(C)c2ccccc2-c2ccc(N(c3ccc(C4CC5CCC4C5)cc3)c3ccc(C4(c5ccccc5)CCCCCC4)cc3)cc21 ; CC1(C)c2cc(N(c3ccc(C4CC5CCC4C5)cc3)c3ccc(C4(c5ccccc5)CCCCCC4)cc3)ccc2-c2c(-c3ccccc3)cccc21]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

[chemical structure]

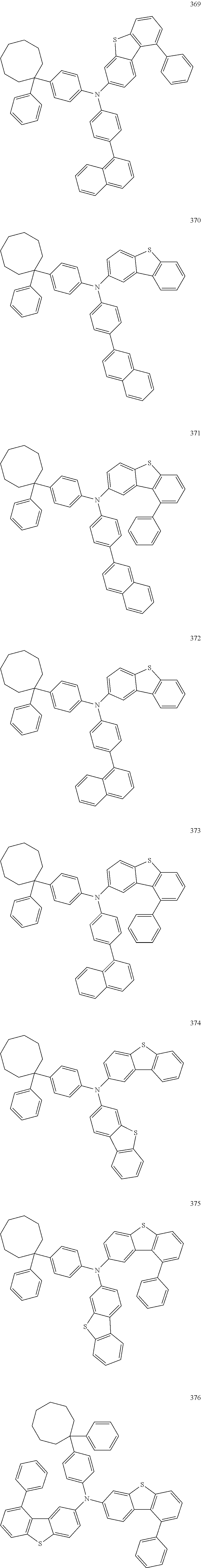

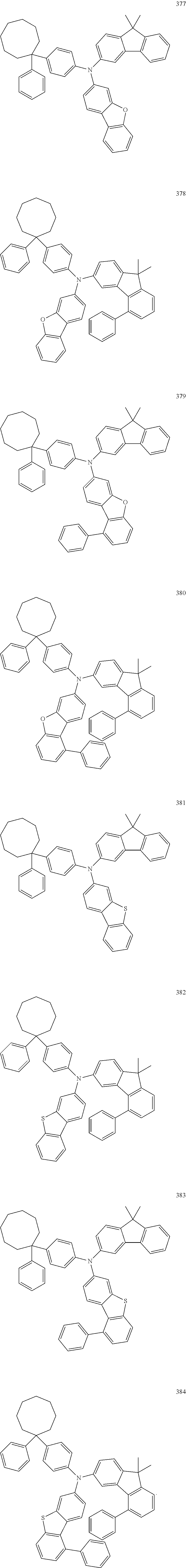

The amine-based compound according to one or more embodiments may include a core moiety described herein. Because n is an integer of 0 or more, the core moiety may be a 5- or more-membered saturated non-aromatic ring (e.g., saturated alicyclic ring). In one or more embodiments, the core moiety may be bonded to at least a first substituent and a second substituent, and the first substituent and the second substituent may each be bonded to same carbon atom of the core moiety:

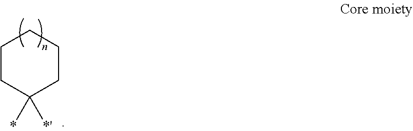

In the core moiety, * indicates a binding site to an atom included in the first substituent, and * indicates a binding site to an atom included in the second substituent. In one or more embodiments, in Formula 1, the first substituent is ted by T₁, and the second substituent is represented by

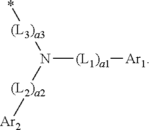

In one or more embodiments, an A value of the first substituent may be greater than an A value of the second substituent. For example, the first substituent prefers an equatorial position to an axial position, and the second substituent prefers an axial position to an equatorial position.

As a result, a hole transportability (e.g., hole mobility) of the amine-based compound according to one or more embodiments may be increased and, at the same time, a chemical reactivity of the amine group included in the second substituent may be decreased. Furthermore, the internal decomposition of the amine-based compound due to the formation of the exciplex may be inhibited, and the lifespan of the light-emitting device including the amine-based compound may be increased.

In one or more embodiments, the amine-based compound represented by Formula 1 may include a substituent represented by Ar₁ and Ar₂. As such, the amine-based compound according to one or more embodiments may have an even larger molecular structure and may maintain an optimal or suitable intermolecular density. Further, Ar₁ and Ar₂ may have electrochemical environments different from each other. As a result, energy levels such as the highest occupied molecular orbital (HOMO) energy level, the lowest unoccupied molecular orbital (LUMO) energy level, the first triplet energy level (T1), the first singlet energy level (Si), etc., the hole mobility, and the refractive index of the amine-based compound may be finely adjusted.

Thus, electronic devices including the amine-based compounds of the present disclosure, for example, organic light-emitting devices including the amine-based compounds, may have a low driving voltage, high efficiency, and long lifespan.

The wording “(interlayer and/or capping layer) includes an amine-based compound” as utilized herein may be understood as “(interlayer and/or capping layer) may include one kind or type of amine-based compound represented by Formula 1 or two different kinds or types of amine-based compounds, each represented by Formula 1.”

In some embodiments, the interlayer and/or the capping layer may include Compound 1 only as the amine-based compound. In this regard, Compound 1 may be present in the emission layer of the light-emitting device. In some embodiments, the interlayer may include, as the amine-based compound, Compound 1 and Compound 2. In this regard, Compound 1 and Compound 2 may be present in substantially the same layer (for example, all of Compound 1 and Compound 2 may be present in the emission layer), or may be present in different layers (for example, Compound 1 may be present in the emission layer, and Compound 2 may be present in the electron transport region).

The term “interlayer” as utilized herein refers to a single layer and/or all of a plurality of layers between the first electrode and the second electrode of the light-emitting device.

One or more aspects of embodiments of the present disclosure are directed toward an electronic apparatus including the light-emitting device. The electronic apparatus may further include a thin-film transistor. For example, in one or more embodiments, the electronic apparatus may further include a thin-film transistor including a source electrode and a drain electrode, wherein the first electrode of the light-emitting device may be electrically connected to the source electrode or the drain electrode. In one or more embodiments, the electronic apparatus may further include a color filter, a color conversion layer, a touch screen layer, a polarizing layer, or a combination thereof. More details of the electronic apparatus may be referred to the descriptions provided herein.

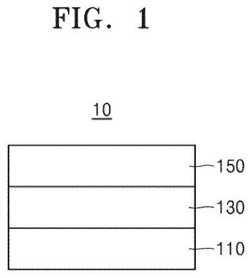

### Description of FIG. 1

FIG. 1 is a schematic cross-sectional view of a light-emitting device 10 according to one or more embodiments of the present disclosure. The light-emitting device 10 may include a first electrode 110, an interlayer 130, and a second electrode 150.

Hereinafter, the structure of the light-emitting device 10 according to one or more embodiments and a method of manufacturing the light-emitting device 10 will be described with reference to FIG. 1.

### First Electrode 110

In FIG. 1, in one or more embodiments, a substrate may be additionally provided and arranged under the first electrode 110 and/or on the second electrode 150. In some embodiments, as the substrate, a glass substrate or a plastic substrate may be utilized. In some embodiments, the substrate may be a flexible substrate, and may include plastics with excellent or suitable heat resistance and durability, such as polyimide, polyethylene terephthalate (PET), polycarbonate, polyethylene naphthalate, polyarylate (PAR), polyetherimide, or a combination thereof.

The first electrode 110 may be formed by, for example, depositing or sputtering a material for forming the first electrode 110 on the substrate. When the first electrode 110 is an anode, a material for forming the first electrode 110 may be a high-work function material that facilitates injection of holes.

The first electrode 110 may be a reflective electrode, a semi-transmissive electrode, or a transmissive electrode. In one or more embodiments, when the first electrode 110 is a transmissive electrode, a material for forming the first electrode 110 may include indium tin oxide (ITO), indium zinc oxide (IZO), tin oxide (SnO₂), zinc oxide (ZnO), or a combination thereof. In one or more embodiments, when the first electrode 110 is a semi-transmissive electrode or a reflective electrode, a material for forming the first electrode 110 may include magnesium (Mg), silver (Ag), aluminum (Al), aluminum-lithium (Al—Li), calcium (Ca), magnesium-indium (Mg—In), magnesium-silver (Mg—Ag), or a combination thereof.

The first electrode 110 may have a single-layer structure including (e.g., consisting of) a single layer or a multi-layer structure including multiple layers. For example, in some embodiments, the first electrode 110 may have a three-layer structure of ITO/Ag/ITO.

### Interlayer 130

The interlayer 130 may be arranged on the first electrode 110. The interlayer 130 may include an emission layer.

In one or more embodiments, the interlayer 130 may further include a hole transport region arranged between the first electrode 110 and the emission layer, and an electron transport region arranged between the emission layer and the second electrode 150.

In one or more embodiments, the interlayer 130 may further include, in addition to one or more suitable organic materials, a metal-containing compound such as an organometallic compound, an inorganic material such as quantum dots, and/or the like.

In one or more embodiments, the interlayer 130 may include, i) two or more emitting units sequentially stacked between the first electrode 110 and the second electrode 150, and ii) a charge generation layer between the two or more emitting units. When the interlayer 130 includes the two or more emitting units and the charge generation layer, the light-emitting device 10 may be a tandem light-emitting device.

### Hole Transport Region in Interlayer 130

The hole transport region may have: i) a single-layer structure including (e.g., consisting of) a single layer including (e.g., consisting of) a single material, ii) a single-layer structure including (e.g., consisting of) a single layer including (e.g., consisting of) multiple materials that are different from each other, or iii) a multi-layer structure including multiple materials including multiple materials that are different from each other.

The hole transport region may include a hole injection layer, a hole transport layer, an emission auxiliary layer, an electron blocking layer, or a combination thereof.

For example, in one or more embodiments, the hole transport region may have a multi-layer structure including a hole injection layer/hole transport layer structure, a hole injection layer/hole transport layer/emission auxiliary layer structure, a hole injection layer/emission auxiliary layer structure, a hole transport layer/emission auxiliary layer structure, or a hole injection layer/hole transport layer/electron blocking layer structure, wherein constituent layers of each structure are stacked sequentially from the first electrode 110 in each stated order.

In one or more embodiments, the hole transport region may include at least one amine-based compound represented by Formula 1. In some embodiments, the hole transport region may further include a compound represented by Formula 201, a compound represented by Formula 202, or a combination thereof:

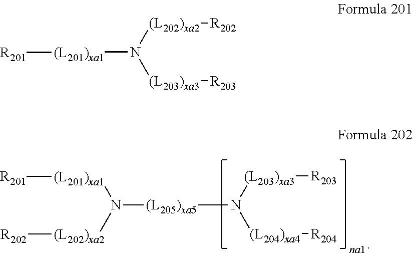

In Formulae 201 and 202,
L₂₀₁ to L₂₀₄ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
L₂₀₅ may be *—O—*′, *—S—*′, *—N(Q₂₀₁)-*′, a C₁-C₂₀ alkylene group unsubstituted or substituted with at least one R₁₀ₐ, a C₂-C₂₀ alkenylene group unsubstituted or substituted with at least one R₁₀ₐ, a C₃-C₆₀ carbocyclic group unsubstituted or substituted with at least one R₁₀ₐ, or a C₁-C₆₀ heterocyclic group unsubstituted or substituted with at least one R₁₀ₐ,
xa1 to xa4 may each independently be an integer from 0 to 5,
xa5 may be an integer from 1 to 10,
R₂₀₁ to R₂₀₄ and Q₂₀₁ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
R₂₀₁ and R₂₀₂ may optionally be bonded to each other via a single bond, a C₁-C₅ alkylene group unsubstituted or substituted with at least one R₁₀ₐ, or a C₂-C₅ alkenylene group unsubstituted or substituted with at least one R₁₀ₐ, to form a C₈-C₆₀ polycyclic group (for example, a carbazole group, etc.) unsubstituted or substituted with at least one R₁₀ₐ (for example, Compound HT16, etc.),
R₂₀₃ and R₂₀₄ may optionally be bonded to each other via a single bond, a C₁-C₅ alkylene group unsubstituted or substituted with at least one R₁₀ₐ, or a C₂-C₅ alkenylene group unsubstituted or substituted with at least one R₁₀ₐ, to form a C₈-C₆₀ polycyclic group unsubstituted or substituted with at least one R₁₀ₐ, and
na1 may be an integer from 1 to 4.

For example, in some embodiments, each of Formulae 201 and 202 may include at least one selected from groups represented by Formulae CY201 to CY217:

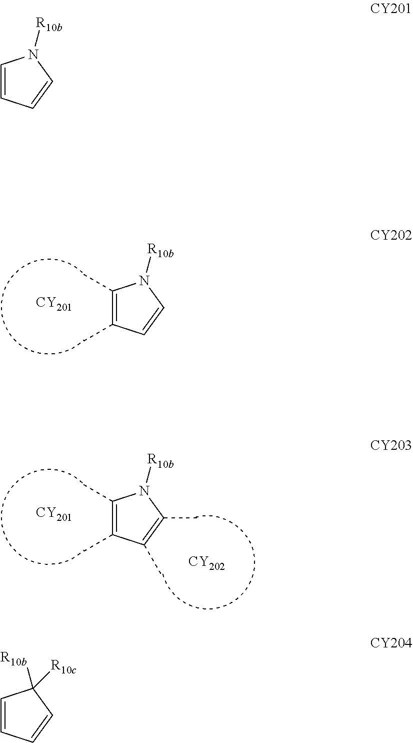

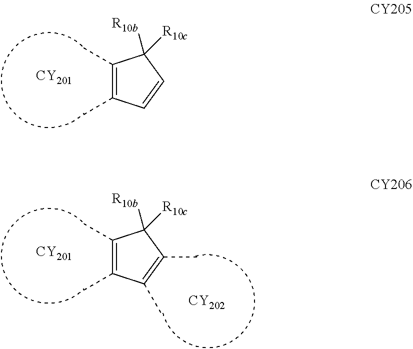

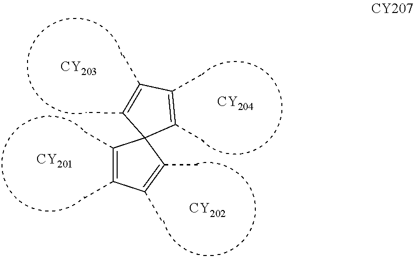

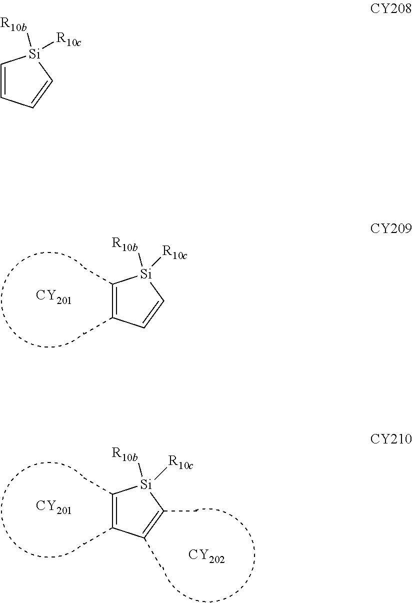

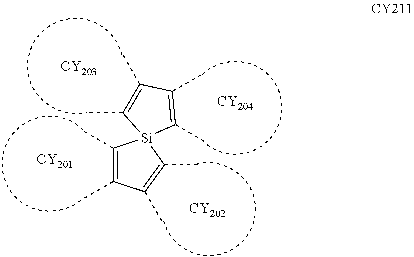

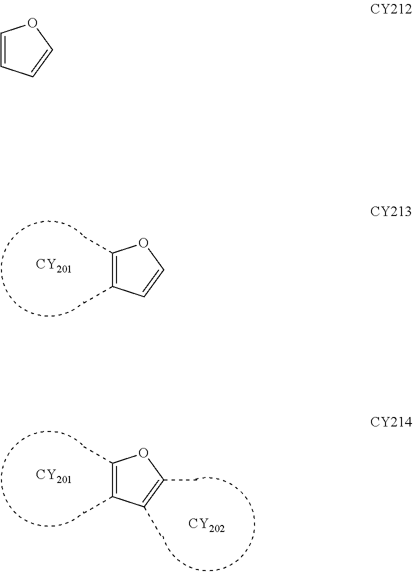

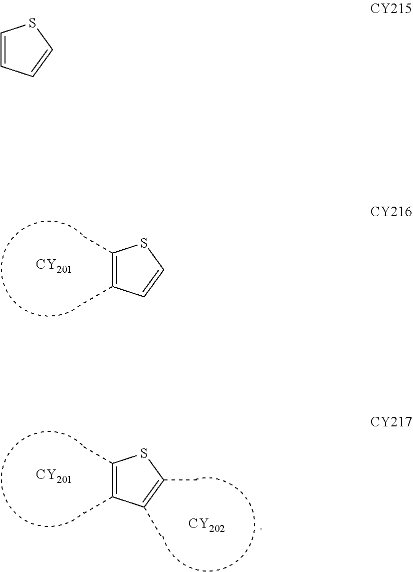

In Formulae CY201 to CY217, R_{10b} and R_{10c} may each be the same as described with respect to R₁₀ₐ, ring CY₂₀₁ to ring CY₂₀₄ may each independently be a C₃-C₂₀ carbocyclic group or a C₁-C₂₀ heterocyclic group, and at least one hydrogen in Formulae CY201 to CY217 may be unsubstituted or substituted with R₁₀ₐ.

In one or more embodiments, in Formulae CY201 to CY217, ring CY₂₀₁ to ring CY204 may each independently be a benzene group, a naphthalene group, a phenanthrene group, or an anthracene group.

In one or more embodiments, each of Formulae 201 and 202 may include at least one selected from the groups represented by Formulae CY201 to CY203.

In one or more embodiments, Formula 201 may include at least one selected from the groups represented by Formulae CY201 to CY203 and at least one selected from the groups represented by Formulae CY204 to CY217.

In one or more embodiments, in Formula 201, xa1 may be 1, R₂₀₁ may be one selected from the groups represented by Formulae CY201 to CY203, xa2 may be 0, and R₂₀₂ may be one selected from the groups represented by Formulae CY204 to CY207.

In one or more embodiments, each of Formulae 201 and 202 may not include (e.g., may exclude) the groups represented by Formulae CY201 to CY203.

In one or more embodiments, each of Formulae 201 and 202 may not include (e.g., may exclude) the groups represented by Formulae CY201 to CY203, and may include at least one selected from the groups represented by Formulae CY204 to CY217.

In one or more embodiments, each of Formulae 201 and 202 may not include (e.g., may exclude) the groups represented by Formulae CY201 to CY217.

For example, in some embodiments, the hole transport region may include at least one selected from Compounds HT1 to HT46, 4,4′,4″-[tris(3-methylphenyl)phenylamino] triphenylamine (m-MTDATA), 4,4′,4″-tris(N,N-diphenylamino)triphenylamine (TDATA), 4,4′,4″-tris[N-(2-naphthyl)-N-phenylamino]-triphenylamine (2-TNATA), N,N′-di(naphthalen-1-yl)-N,N′-diphenyl-benzidine (NPB(NPD)), β-NPB, N, N′-bis(3-methylphenyl)-N, N′-diphenyl-[1,1′-biphenyl]-4,4′-diamine (TPD), Spiro-TPD, Spiro-NPB, methylated NPB, 4,4′-cyclohexylidene bis[N,N-bis(4-methylphenyl)benzenamine] (TAPC), 4,4′-bis[N,N′-(3-tolyl)amino]-3,3′-dimethylbiphenyl (HMTPD), 4,4′,4″-tris(N-carbazolyl)triphenylamine (TCTA), polyaniline/dodecylbenzenesulfonic acid (PANI/DBSA), poly(3,4-ethylenedioxythiophene)/poly(4-styrenesulfonate) (PEDOT/PSS), polyaniline/camphor sulfonic acid (PANI/CSA), polyaniline/poly(4-styrenesulfonate) (PANI/PSS), and/or a combination thereof:

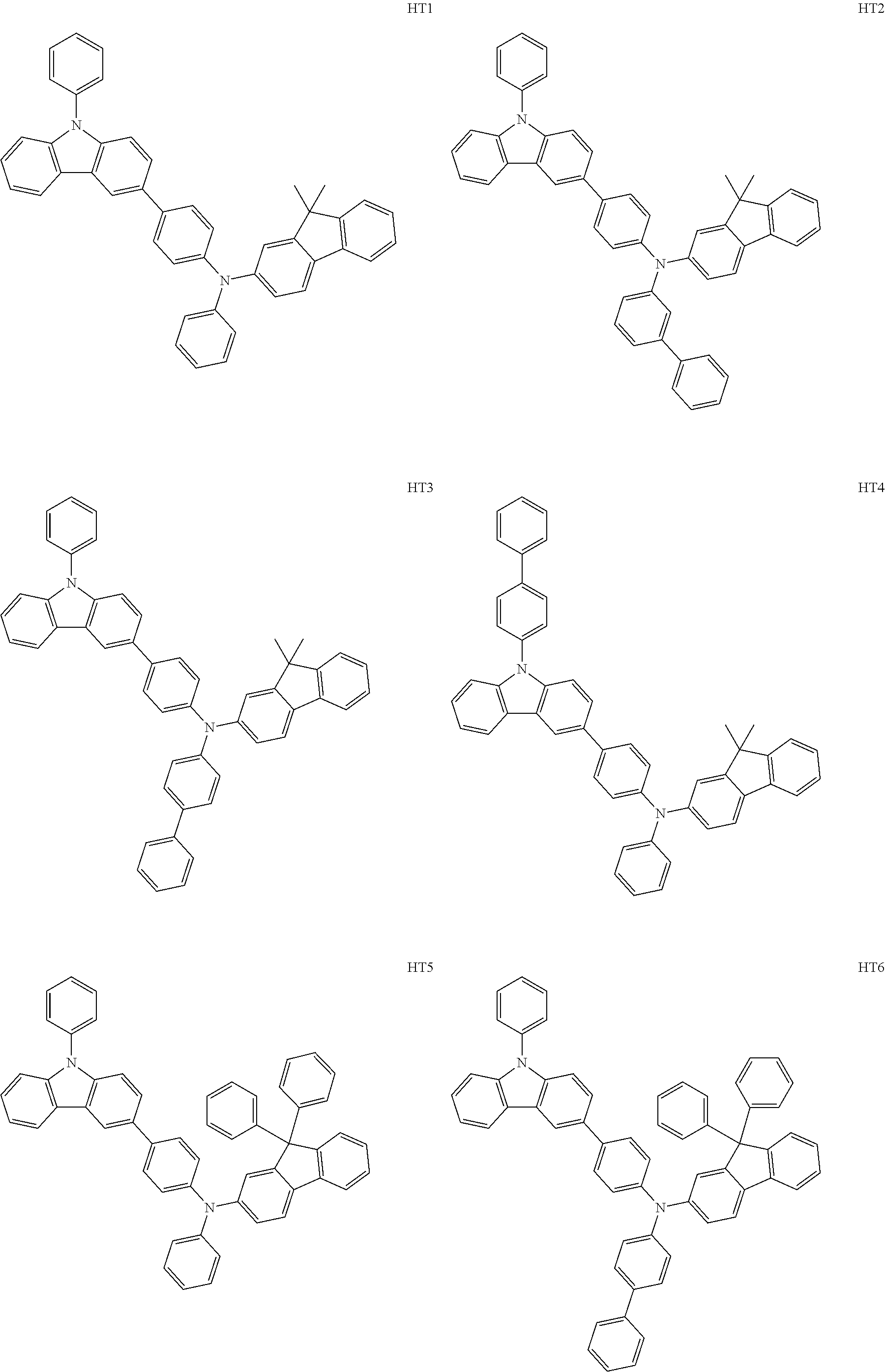

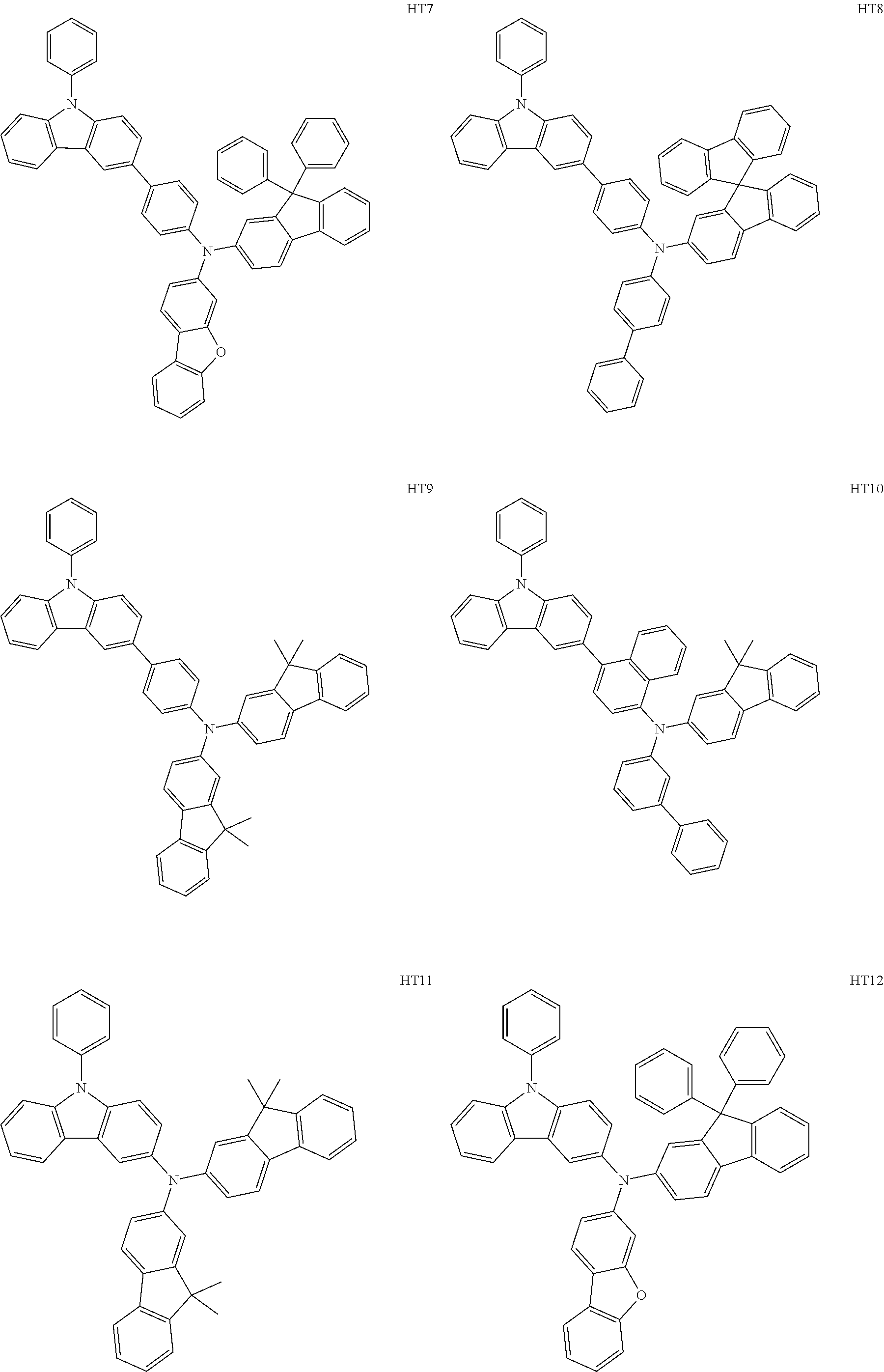

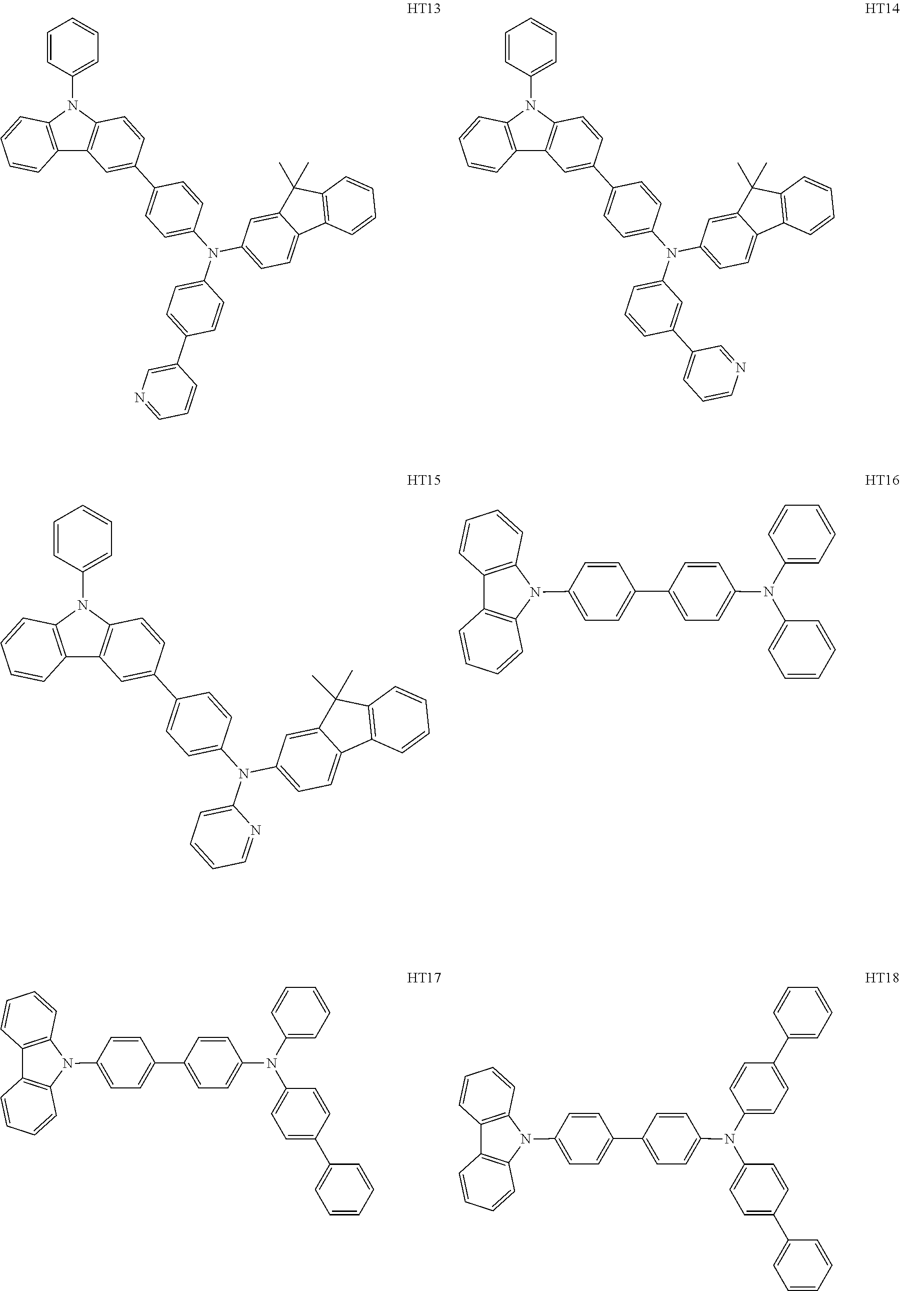 [SMILES: CC1(C)c2ccccc2-c2ccc(N(c3ccc(-c4cccnc4)cc3)c3ccc(-c4ccc5c(c4)c4ccccc4n5-c4ccccc4)cc3)cc21 ; CC1(C)c2ccccc2-c2ccc(N(c3ccc(-c4ccc5c(c4)c4ccccc4n5-c4ccccc4)cc3)c3cccc(-c4cccnc4)c3)cc21 ; CC1(C)c2ccccc2-c2ccc(N(c3ccc(-c4ccc5c(c4)c4ccccc4n5-c4ccccc4)cc3)c3ccccn3)cc21 ; c1ccc(N(c2ccccc2)c2ccc(-c3ccc(-n4c5ccccc5c5ccccc54)cc3)cc2)cc1 ; c1ccc(-c2ccc(N(c3ccccc3)c3ccc(-c4ccc(-n5c6ccccc6c6ccccc65)cc4)cc3)cc2)cc1 ; c1ccc(-c2ccc(N(c3ccc(-c4ccccc4)cc3)c3ccc(-c4ccc(-n5c6ccccc6c6ccccc65)cc4)cc3)cc2)cc1]

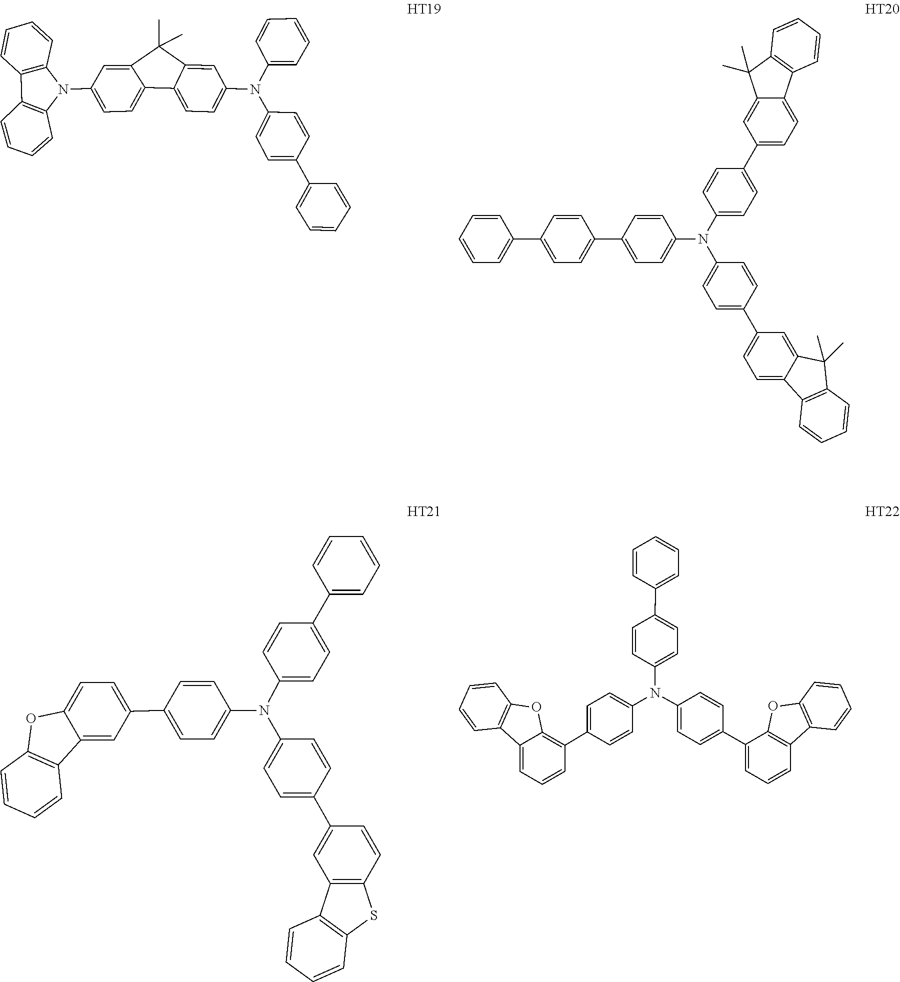 [SMILES: CC1(C)c2cc(N(c3ccccc3)c3ccc(-c4ccccc4)cc3)ccc2-c2ccc(-n3c4ccccc4c4ccccc43)cc21 ; CC1(C)c2ccccc2-c2ccc(-c3ccc(N(c4ccc(-c5ccc(-c6ccccc6)cc5)cc4)c4ccc(-c5ccc6c(c5)C(C)(C)c5ccccc5-6)cc4)cc3)cc21 ; c1ccc(-c2ccc(N(c3ccc(-c4ccc5oc6ccccc6c5c4)cc3)c3ccc(-c4ccc5sc6ccccc6c5c4)cc3)cc2)cc1 ; c1ccc(-c2ccc(N(c3ccc(-c4cccc5c4oc4ccccc45)cc3)c3ccc(-c4cccc5c4oc4ccccc45)cc3)cc2)cc1]

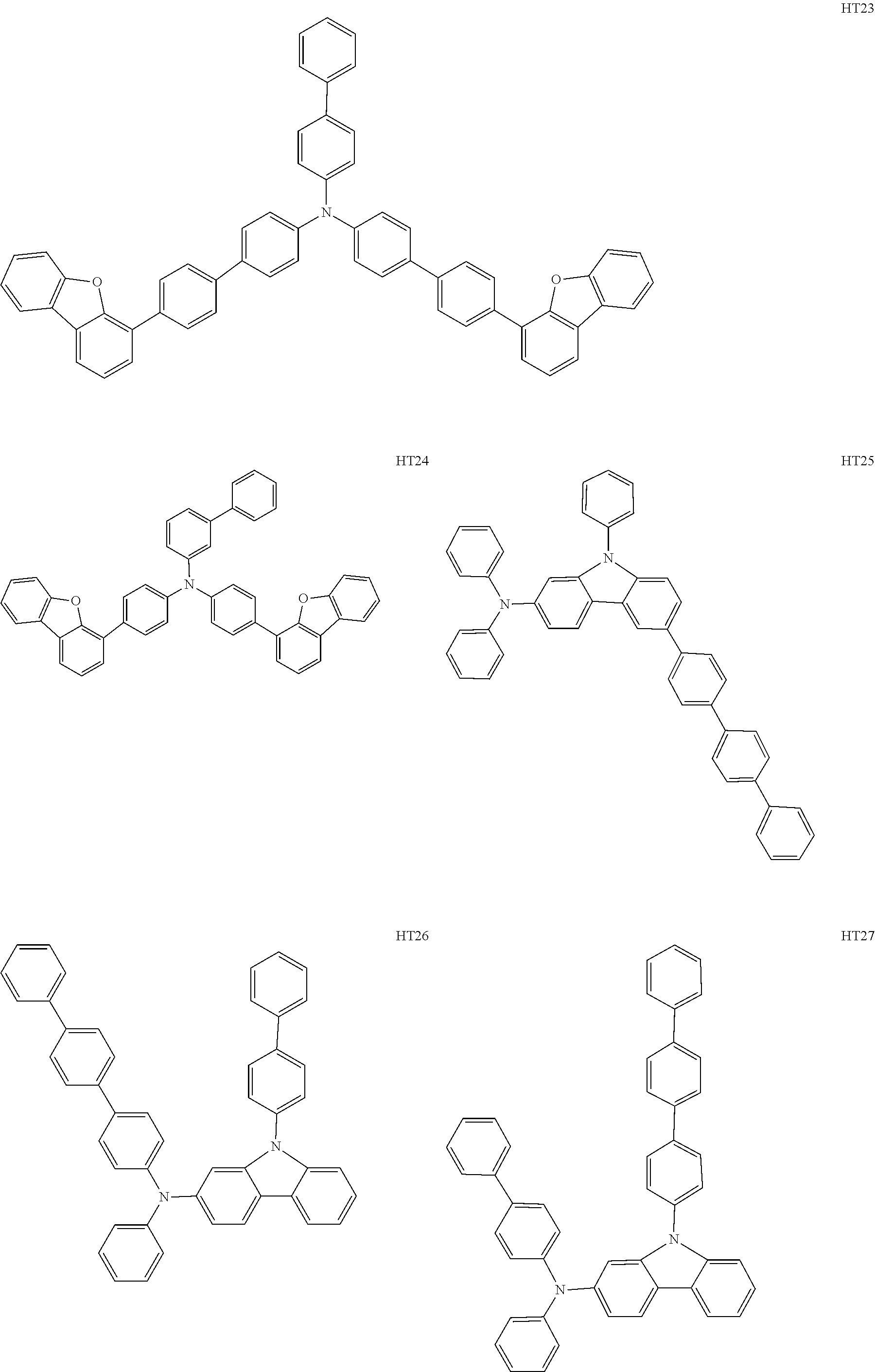 [SMILES: c1ccc(-c2ccc(N(c3ccc(-c4ccc(-c5cccc6c5oc5ccccc56)cc4)cc3)c3ccc(-c4ccc(-c5cccc6c5oc5ccccc56)cc4)cc3)cc2)cc1 ; c1ccc(-c2cccc(N(c3ccc(-c4cccc5c4oc4ccccc45)cc3)c3ccc(-c4cccc5c4oc4ccccc45)cc3)c2)cc1 ; c1ccc(-c2ccc(-c3ccc(-c4ccc5c(c4)c4ccc(N(c6ccccc6)c6ccccc6)cc4n5-c4ccccc4)cc3)cc2)cc1 ; c1ccc(-c2ccc(-c3ccc(N(c4ccccc4)c4ccc5c6ccccc6n(-c6ccc(-c7ccccc7)cc6)c5c4)cc3)cc2)cc1 ; c1ccc(-c2ccc(-c3ccc(-n4c5ccccc5c5ccc(N(c6ccccc6)c6ccc(-c7ccccc7)cc6)cc54)cc3)cc2)cc1]

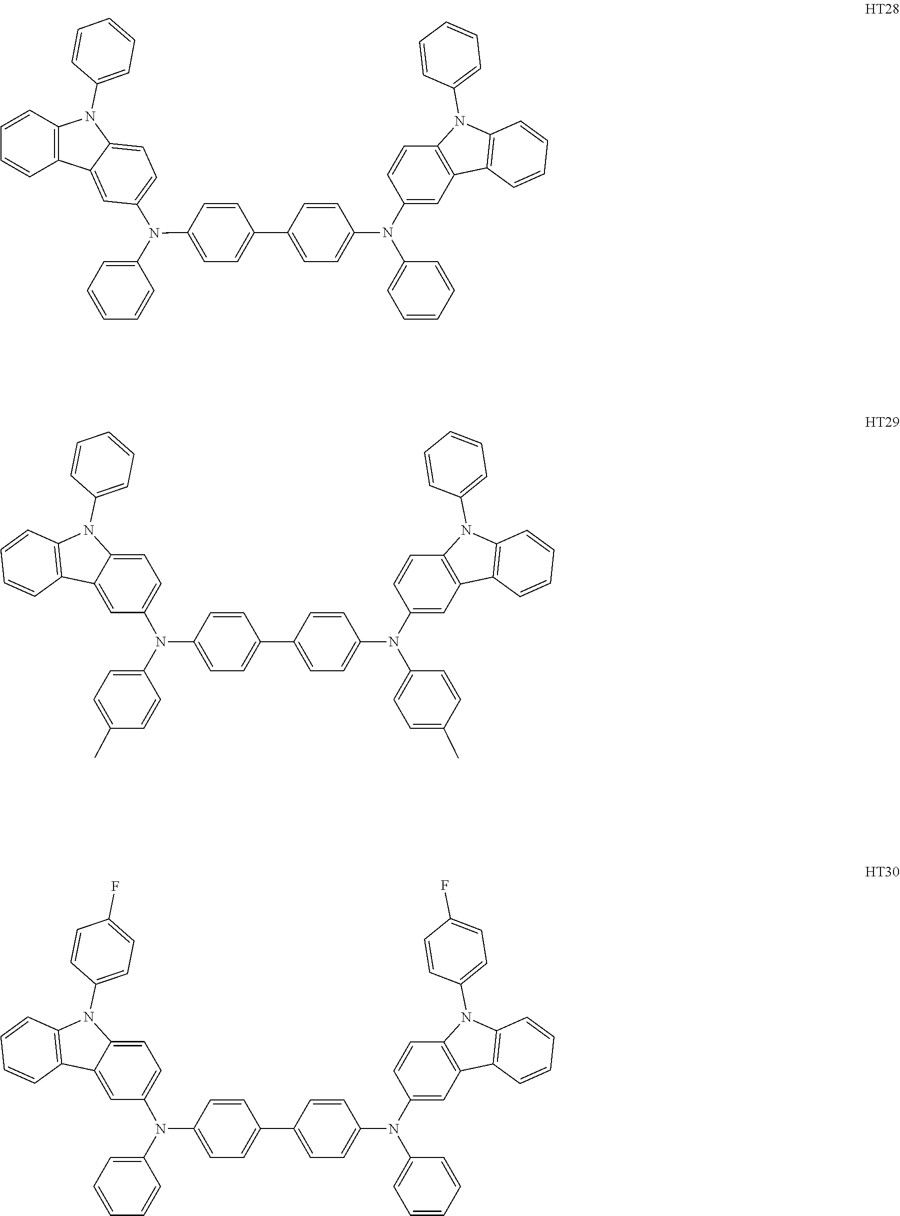 [SMILES: c1ccc(N(c2ccc(-c3ccc(N(c4ccccc4)c4ccc5c(c4)c4ccccc4n5-c4ccccc4)cc3)cc2)c2ccc3c(c2)c2ccccc2n3-c2ccccc2)cc1 ; Cc1ccc(N(c2ccc(-c3ccc(N(c4ccc(C)cc4)c4ccc5c(c4)c4ccccc4n5-c4ccccc4)cc3)cc2)c2ccc3c(c2)c2ccccc2n3-c2ccccc2)cc1 ; Fc1ccc(-n2c3ccccc3c3cc(N(c4ccccc4)c4ccc(-c5ccc(N(c6ccccc6)c6ccc7c(c6)c6ccccc6n7-c6ccc(F)cc6)cc5)cc4)ccc32)cc1]

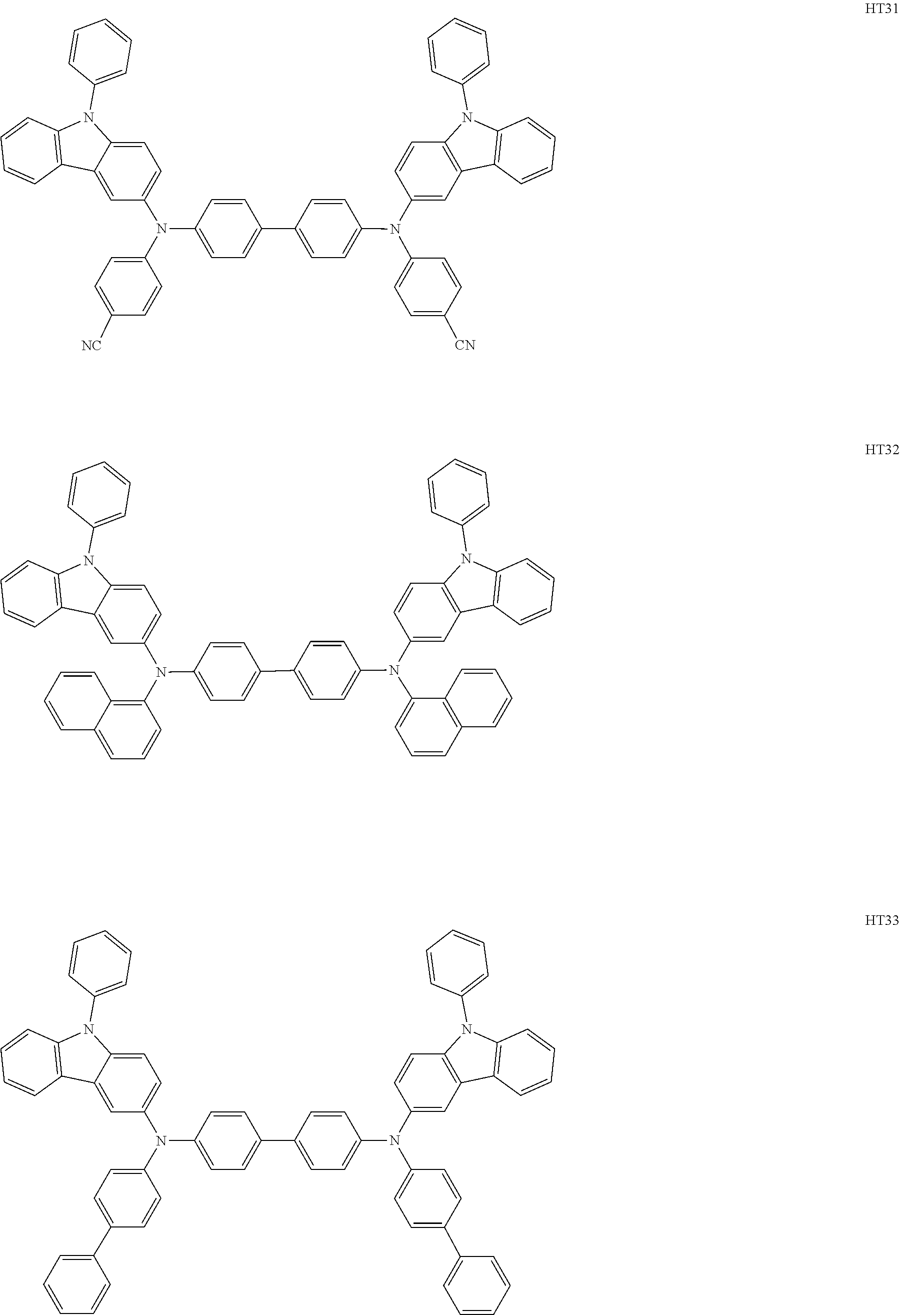 [SMILES: N#Cc1ccc(N(c2ccc(-c3ccc(N(c4ccc(C#N)cc4)c4ccc5c(c4)c4ccccc4n5-c4ccccc4)cc3)cc2)c2ccc3c(c2)c2ccccc2n3-c2ccccc2)cc1 ; c1ccc(-n2c3ccccc3c3cc(N(c4ccc(-c5ccc(N(c6ccc7c(c6)c6ccccc6n7-c6ccccc6)c6cccc7ccccc67)cc5)cc4)c4cccc5ccccc45)ccc32)cc1 ; c1ccc(-c2ccc(N(c3ccc(-c4ccc(N(c5ccc(-c6ccccc6)cc5)c5ccc6c(c5)c5ccccc5n6-c5ccccc5)cc4)cc3)c3ccc4c(c3)c3ccccc3n4-c3ccccc3)cc2)cc1]

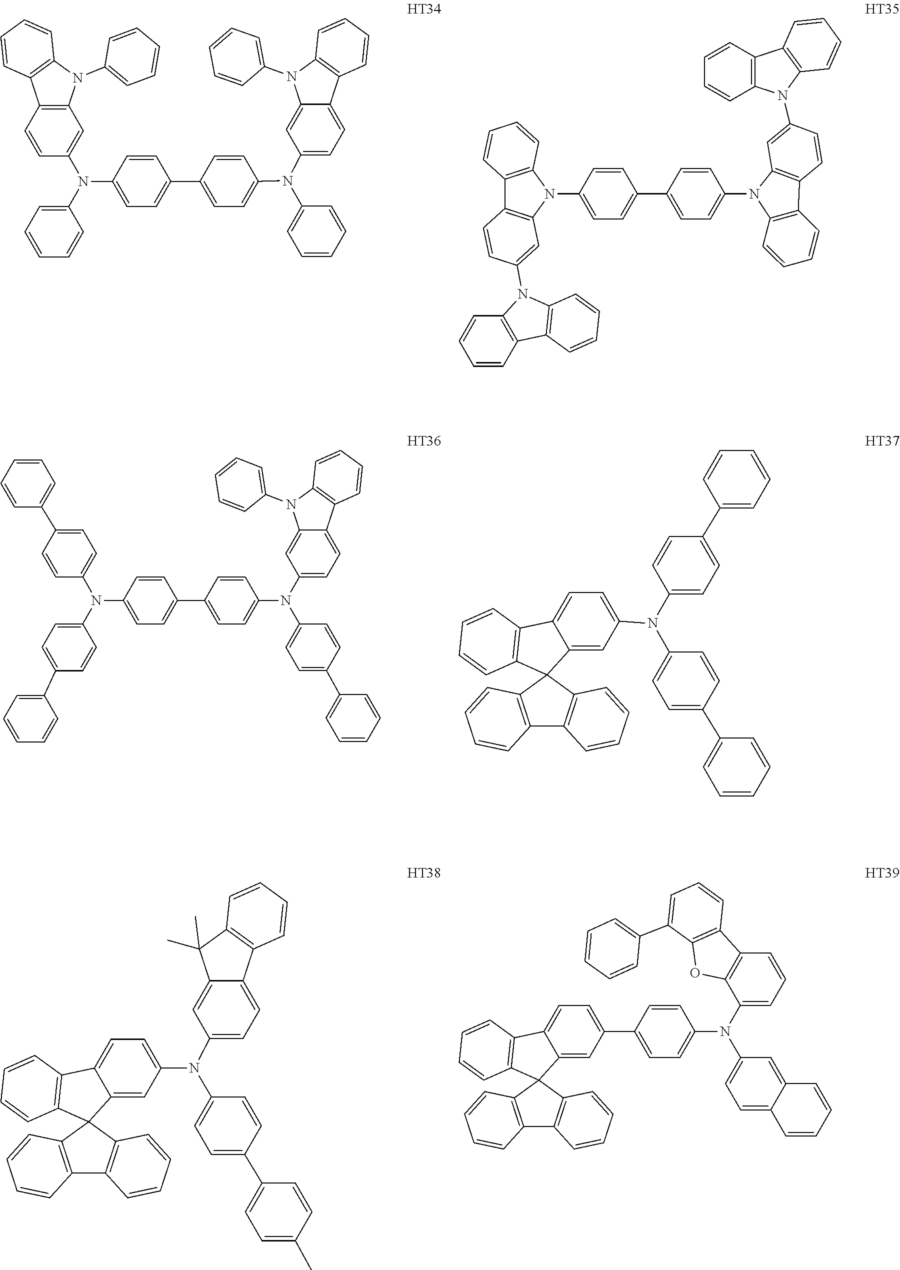

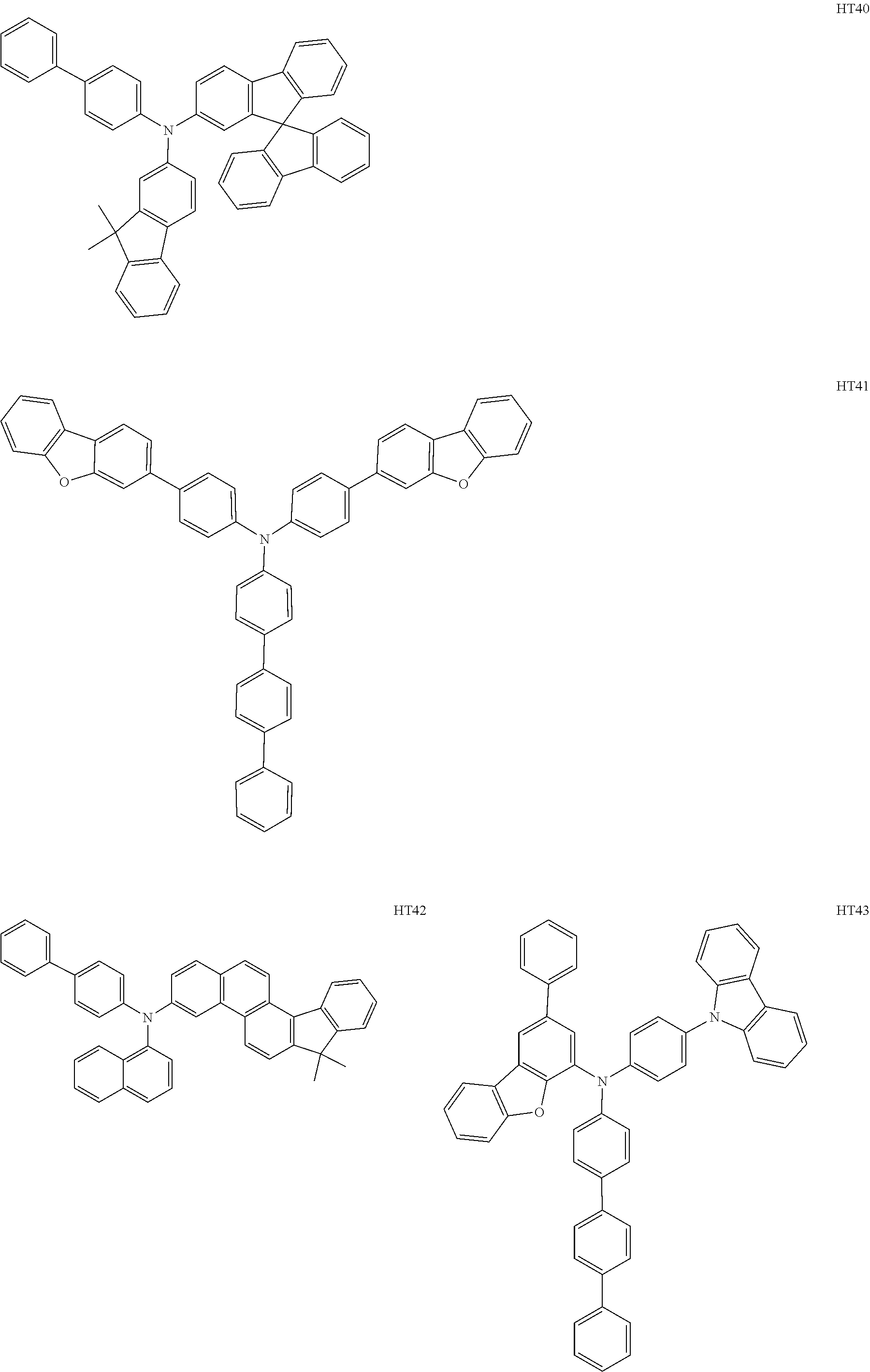 [SMILES: CC1(C)c2ccccc2-c2ccc(N(c3ccc(-c4ccccc4)cc3)c3ccc4c(c3)C3(c5ccccc5-c5ccccc53)c3ccccc3-4)cc21 ; c1ccc(-c2ccc(-c3ccc(N(c4ccc(-c5ccc6c(c5)oc5ccccc56)cc4)c4ccc(-c5ccc6c(c5)oc5ccccc56)cc4)cc3)cc2)cc1 ; CC1(C)c2ccccc2-c2c1ccc1c2ccc2ccc(N(c3ccc(-c4ccccc4)cc3)c3cccc4ccccc34)cc21 ; c1ccc(-c2ccc(-c3ccc(N(c4ccc(-n5c6ccccc6c6ccccc65)cc4)c4cc(-c5ccccc5)cc5c4oc4ccccc45)cc3)cc2)cc1]

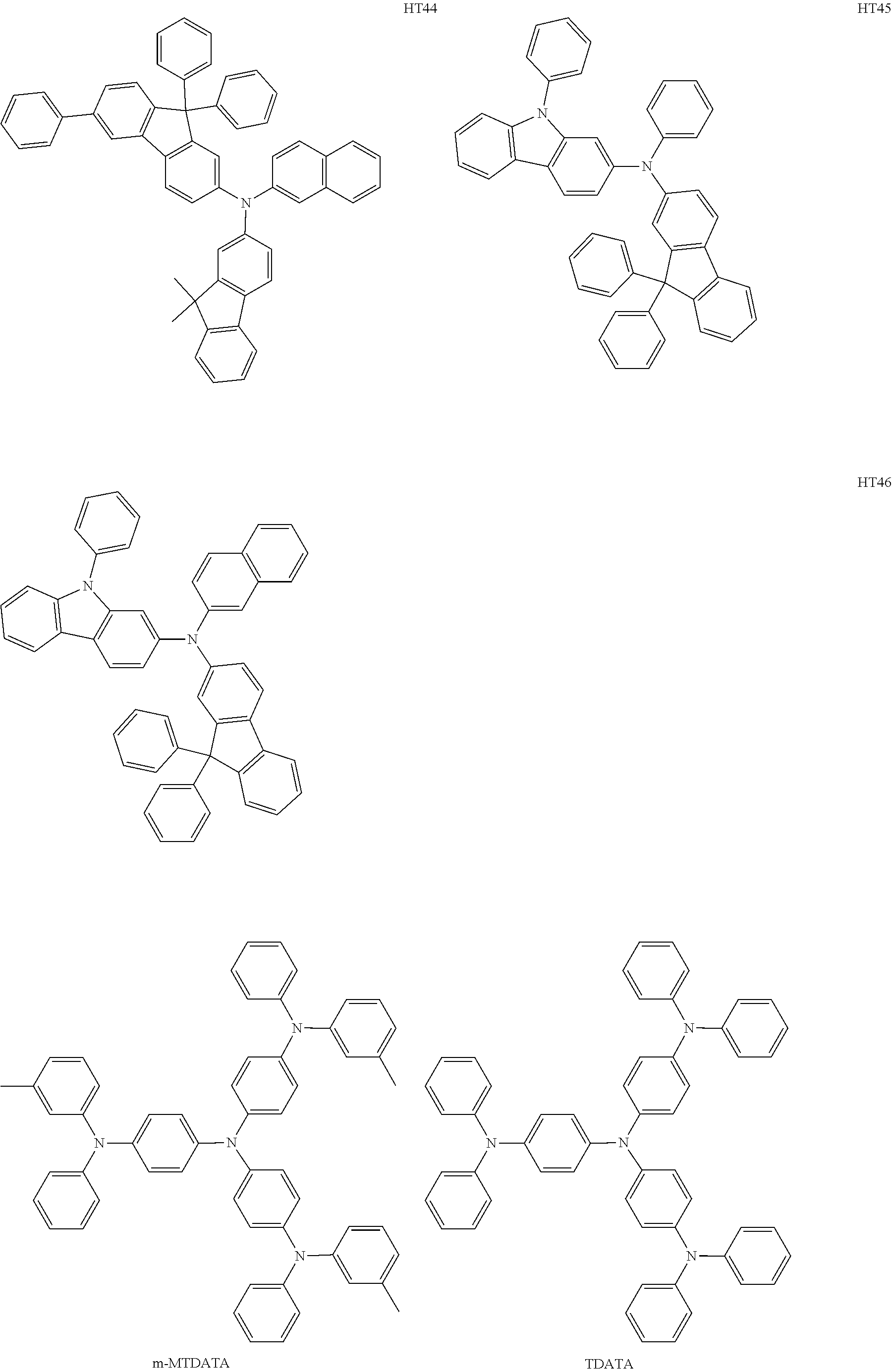

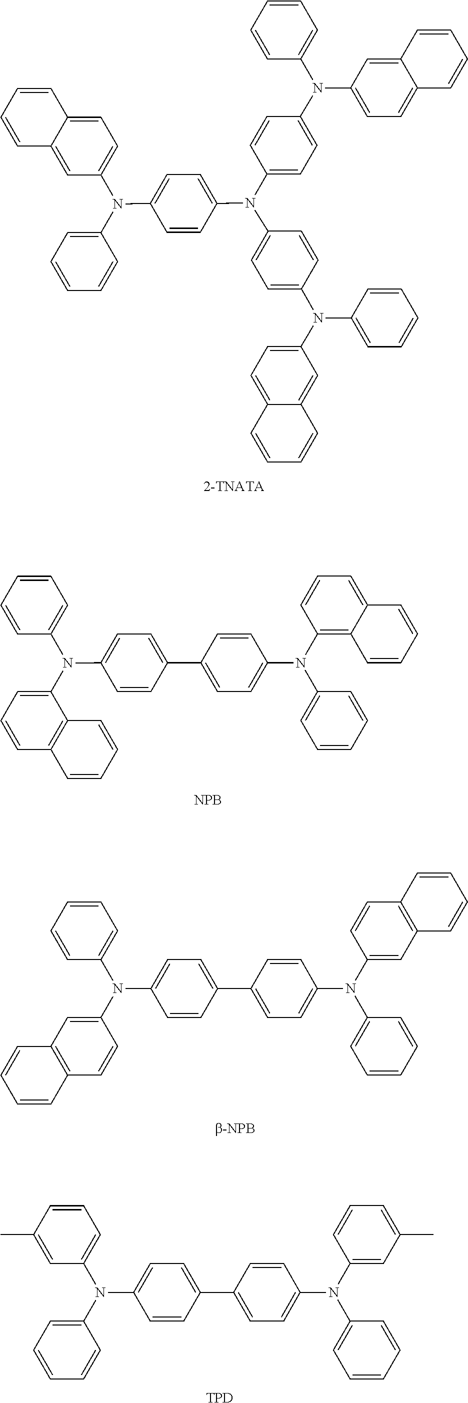

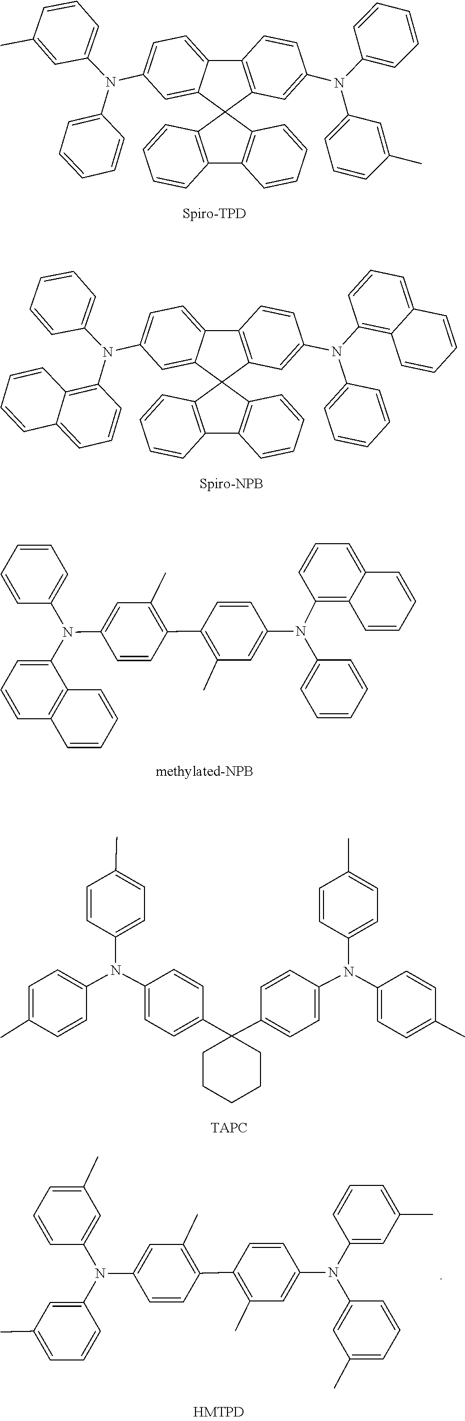

A thickness of the hole transport region may be in a range of about 50 Å to about 10,000 Å, for example, about 100 Å to about 4,000 Å. When the hole transport region includes a hole injection layer, a hole transport layer, or a combination thereof, a thickness of the hole injection layer may be in a range of about 100 Å to about 9,000 Å, for example, about 100 Å to about 1,000 Å, and a thickness of the hole transport layer may be in a range of about 50 Å to about 2,000 Å, for example, about 100 Å to about 1,500 Å. When the thicknesses of the hole transport region, the hole injection layer, and the hole transport layer are within these ranges, satisfactory hole transporting characteristics may be obtained without a substantial increase in driving voltage.

The emission auxiliary layer may increase light-emission efficiency by compensating for an optical resonance distance according to the wavelength of light emitted by the emission layer, and the electron blocking layer may block or reduce the leakage of electrons from the emission layer to the hole transport region. Materials that may be included in the hole transport region may be included in the emission auxiliary layer and the electron blocking layer.

p-Dopant

In one or more embodiments, the hole transport region may further include, in addition to these aforementioned materials, a charge-generation material for the improvement of conductive properties. The charge-generation material may be substantially uniformly or non-uniformly dispersed in the hole transport region (for example, in the form of a single layer including (e.g., consisting of) a charge-generation material).

The charge-generation material may be, for example, a p-dopant.

For example, in some embodiments, the p-dopant may have a lowest unoccupied molecular orbital (LUMO) energy level of less than or equal to about −3.5 eV.

In one or more embodiments, the p-dopant may include a quinone derivative, a cyano group-containing compound, a compound including element EL1 and element EL2, or a combination thereof.

Non-limiting examples of the quinone derivative are tetracyanoquinodimethane (TCNQ), 2,3,5,6-tetrafluoro-7,7,8,8-tetracyanoquinodimethane (F4-TCNQ), and/or the like.

Non-limiting examples of the cyano group-containing compound may be dipyrazino[2,3-f: 2′,3′-h] quinoxaline-2,3,6,7,10,11-hexacarbonitrile (HAT-CN), a compound represented by Formula 221, and/or the like:

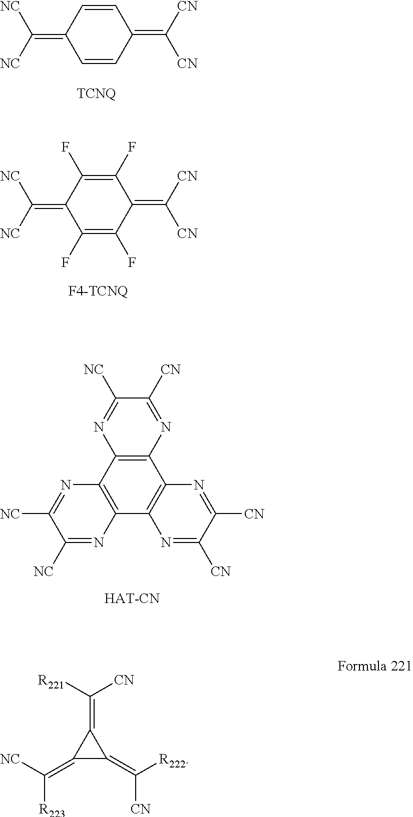

In Formula 221,
R₂₂₁ to R₂₂₃ may each independently be a C₃-C₆₀ carbocyclic group unsubstituted or substituted with at least one R₁₀ₐ or a C₁-C₆₀ heterocyclic group unsubstituted or substituted with at least one R₁₀ₐ, and
at least one selected from among R₂₂₁ to R₂₂₃ may each independently be a C₃-C₆₀ carbocyclic group or a C₁-C₆₀ heterocyclic group, each substituted with: a cyano group; —F; —C₁; —Br; —I; a C₁-C₂₀ alkyl group substituted with a cyano group, —F, —Cl, —Br, —I, or a combination thereof; or a combination thereof.

In the compound including element EL1 and element EL2, element EL1 may be metal, metalloid, or a combination thereof, and element EL2 may be non-metal, metalloid, or a combination thereof.

Non-limiting examples of the metal may be an alkali metal (for example, lithium (Li), sodium (Na), potassium (K), rubidium (Rb), cesium (Cs), etc.); an alkaline earth metal (for example, beryllium (Be), magnesium (Mg), calcium (Ca), strontium (Sr), barium (Ba), etc.); a transition metal (for example, titanium (Ti), zirconium (Zr), hafnium (Hf), vanadium (V), niobium (Nb), tantalum (Ta), chromium (Cr), molybdenum (Mo), tungsten (W), manganese (Mn), technetium (Tc), rhenium (Re), iron (Fe), ruthenium (Ru), osmium (Os), cobalt (Co), rhodium (Rh), iridium (Ir), nickel (Ni), palladium (Pd), platinum (Pt), copper (Cu), silver (Ag), gold (Au), etc.); a post-transition metal (for example, zinc (Zn), indium (In), tin (Sn), etc.); a lanthanide metal (for example, lanthanum (La), cerium (Ce), praseodymium (Pr), neodymium (Nd), promethium (Pm), samarium (Sm), europium (Eu), gadolinium (Gd), terbium (Tb), dysprosium (Dy), holmium (Ho), erbium (Er), thulium (Tm), ytterbium (Yb), lutetium (Lu), etc.); and/or the like.

Non-limiting examples of the metalloid may be silicon (Si), antimony (Sb), tellurium (Te), and/or the like.

Non-limiting examples of the non-metal may be oxygen (O), halogen (for example, F, Cl, Br, I, etc.), and/or the like.

Non-limiting examples of the compound including element EL1 and element EL2 may be metal oxide, metal halide (for example, metal fluoride, metal chloride, metal bromide, metal iodide, etc.), metalloid halide (for example, metalloid fluoride, metalloid chloride, metalloid bromide, metalloid iodide, etc.), metal telluride, or a combination thereof.

Non-limiting examples of the metal oxide may be tungsten oxide (for example, WO, W₂O₃, WO₂, WO₃, W₂O₅, etc.), vanadium oxide (for example, VO, V₂O₃, VO₂, V₂O₅, etc.), molybdenum oxide (MoO, Mo₂O₃, MoO₂, MoO₃, Mo₂O₅, etc.), rhenium oxide (for example, ReO₃, etc.), and/or the like.

Non-limiting examples of the metal halide may be alkali metal halide, alkaline earth metal halide, transition metal halide, post-transition metal halide, lanthanide metal halide, and/or the like.

Non-limiting examples of the alkali metal halide may be LiF, NaF, KF, RbF, CsF, LiCl, NaCl, KCl, RbCl, CsCl, LiBr, NaBr, KBr, RbBr, CsBr, LiI, NaI, KI, RbI, CsI, and/or the like.

Non-limiting examples of the alkaline earth metal halide may be BeF₂, MgF₂, CaF₂, SrF₂, BaF₂, BeCl₂, MgCl₂, CaCl₂), SrCl₂, BaCl₂, BeBr₂, MgBr₂, CaBr₂, SrBr₂, BaBr₂, BeI₂, MgI₂, CaI₂, SrI₂, Bale, and/or the like.

Non-limiting examples of the transition metal halide may be titanium halide (for example, TiF₄, TiCl₄, TiBr₄, TiI₄, etc.), zirconium halide (for example, ZrF₄, ZrCl₄, ZrBr₄, ZrI₄, etc.), hafnium halide (for example, HfF₄, HfCl₄, HfBr₄, HfI₄, etc.), vanadium halide (for example, VF₃, VCl₃, VBr₃, VI₃, etc.), niobium halide (for example, NbF₃, NbCl₃, NbBr₃, NbI₃, etc.), tantalum halide (for example, TaF₃, TaCl₃, TaBr₃, TaI₃, etc.), chromium halide (for example, CrF₃, CrCl₃, CrBr₃, CrI₃, etc.), molybdenum halide (for example, MoF₃, MoCl₃, MoBr₃, MoI₃, etc.), tungsten halide (for example, WF₃, WCl₃, WBr₃, WI₃, etc.), manganese halide (for example, MnF₂, MnCl₂, MnBr₂, MnI₂, etc.), technetium halide (for example, TcF₂, TcCl₂, TcBr₂, TcI₂, etc.), rhenium halide (for example, ReF₂, ReCl₂, ReBr₂, ReI₂, etc.), ferrous halide (for example, FeF₂, FeCl₂, FeBr₂, Felt, etc.), ruthenium halide (for example, RuF₂, RuCl₂, RuBr₂, Rule, etc.), osmium halide (for example, OsF₂, OsCl₂, OsBr₂, OsI₂, etc.), cobalt halide (for example, CoF₂, CoCl₂, CoBr₂, Cole, etc.), rhodium halide (for example, RhF₂, RhCl₂, RhBr₂, RhI₂, etc.), iridium halide (for example, IrF₂, IrCl₂, IrBr₂, IrI₂, etc.), nickel halide (for example, NiF₂, NiCl₂, NiBr₂, NiI₂, etc.), palladium halide (for example, PdF₂, PdCl₂, PdBr₂, PdI₂, etc.), platinum halide (for example, PtF₂, PtCl₂, PtBr₂, PtI₂, etc.), cuprous halide (for example, CuF, CuCl, CuBr, CuI, etc.), silver halide (for example, AgF, AgCl, AgBr, AgI, etc.), gold halide (for example, AuF, AuCI, AuBr, AuI, etc.), and/or the like.

Non-limiting examples of the post-transition metal halide may be zinc halide (for example, ZnF₂, ZnCl₂, ZnBr₂, ZnI₂, etc.), indium halide (for example, InI₃, etc.), tin halide (for example, SnI₂, etc.), and/or the like.

Non-limiting examples of the lanthanide metal halide may be YbF, YbF₂, YbF₃, SmF₃, YbCl, YbCl₂, YbCl₃, SmCl₃, YbBr, YbBr₂, YbBr₃, SmBr₃, YbI, YbI₂, YbI₃, SmI₃, and/or the like.

Non-limiting examples of the metalloid halide may be antimony halide (for example, SbCl₅, etc.) and/or the like.

Non-limiting examples of the metal telluride may be alkali metal telluride (for example, Li₂Te, Na₂Te, K₂Te, Rb₂Te, Cs₂Te, etc.), alkaline earth metal telluride (for example, BeTe, MgTe, CaTe, SrTe, BaTe, etc.), transition metal telluride (for example, TiTe₂, ZrTe₂, HfTe₂, V₂Te₃, Nb₂Te₃, Ta₂Te₃, Cr₂Te₃, Mo₂Te₃, W₂Te₃, MnTe, TcTe, ReTe, FeTe, RuTe, OsTe, CoTe, RhTe, IrTe, NiTe, PdTe, PtTe, Cu₂Te, CuTe, Ag₂Te, AgTe, Au₂Te, etc.), post-transition metal telluride (for example, ZnTe, etc.), lanthanide metal telluride (for example, LaTe, CeTe, PrTe, NdTe, PmTe, EuTe, GdTe, TbTe, DyTe, HoTe, ErTe, TmTe, YbTe, LuTe, etc.), and/or the like.

### Emission Layer in Interlayer 130

When the light-emitting device 10 is a full-color light-emitting device, the emission layer may be patterned into a red emission layer, a green emission layer, and/or a blue emission layer, according to a sub-pixel. In one or more embodiments, the emission layer may have a stacked structure of two or more layers of a red emission layer, a green emission layer, and/or a blue emission layer, in which the two or more layers contact each other or are separated from each other, to emit white light (e.g., combined white light). In one or more embodiments, the emission layer may include two or more materials of a red light-emitting material, a green light-emitting material, and/or a blue light-emitting material, in which the two or more materials are mixed with each other in a single layer, to emit white light (e.g., combined white light).

In one or more embodiments, the emission layer may include a host and a dopant. The dopant may include a phosphorescent dopant, a fluorescent dopant, or a combination thereof.

The amount of the dopant in the emission layer may be in a range of about 0.01 part by weight to about 15 parts by weight based on 100 parts by weight of the host.

In one or more embodiments, the emission layer may include a quantum dot.

In one or more embodiments, the emission layer may include a delayed fluorescence material. The delayed fluorescence material may act as a host and/or a dopant in the emission layer 120.

The thickness of the emission layer may be in a range of about 100 Å to about 1,000 Å, for example, about 200 Å to about 600 Å. When the thickness of the emission layer 120 is within these ranges, excellent or suitable luminescence characteristics may be obtained without a substantial increase in driving voltage.

### Host

In one or more embodiments, the host may include a compound represented by Formula 301:

[Ar₃₀₁]_{xb11}-[(L₃₀₁)_{xb1}-R₃₀₁]_{xb21}, Formula 301
wherein, in Formula 301,
Ar₃₀₁ and L₃₀₁ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,

xb11 may be 1, 2, or 3,

xb1 may be an integer from 0 to 5,

R₃₀₁ may be hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, —Si(Q₃₀₁)(Q₃₀₂)(Q₃₀₃), —N(Q₃₀₁)(Q₃₀₂), —B(Q₃₀₁)(Q₃₀₂), —C(═O)(Q₃₀₁), —S(═O)₂(Q₃₀₁), or —P(═O)(Q₃₀₁)(Q₃₀₂),

xb21 may be an integer from 1 to 5, and

0301 to 0303 may each be the same as described respect to Q₁.

For example, in some embodiments, when xb11 in Formula 301 is 2 or more, two or more of Ar₃₀₁ may be linked to each other via a single bond.

In one or more embodiments, the host may include a compound represented by Formula 301-1, a compound represented by Formula 301-2, or a combination thereof:

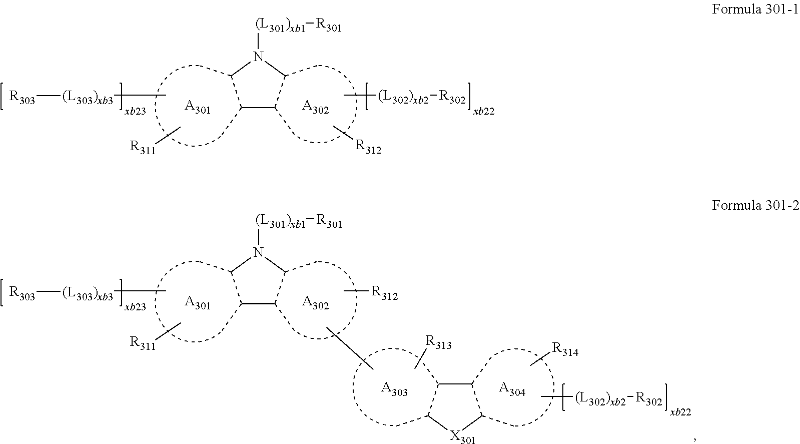

wherein, in Formulae 301-1 and 301-2,

ring A₃₀₁ to ring A₃₀₄ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
X₃₀₁ may be O, S, N-[(L₃₀₄)_{xb4}−R₃₀₄], C(R₃₀₄)(R₃₀₅), or Si(R₃₀₄)(R₃₀₅),
xb22 and xb23 may each independently be 0, 1, or 2,
L₃₀₁, xb1, and R₃₀₁ may each be the same as described herein,
L₃₀₂ to L₃₀₄ may each independently be the same as described with respect to L₃₀₁,
xb2 to xb4 may each independently be the same as described with respect to xb1, and
R₃₀₂ to R₃₀₅ and R₃₁₁ to R₃₁₄ may each be the same as described with respect to R₃₀₁.

In one or more embodiments, the host may include an alkaline earth metal complex, a post-transition metal complex, or a combination thereof. In one or more embodiments, the host may include a Be complex (for example, Compound H55), an Mg complex, a Zn complex, or a combination thereof.

In one or more embodiments, the host may include: at least one selected from among Compounds H1 to H128; 9,10-di(2-naphthyl)anthracene (ADN); 2-methyl-9,10-bis(naphthalen-2-yl)anthracene (MADN); 9,10-di-(2-naphthyl)-2-t-butyl-anthracene (TBADN); 4,4′-bis(N-carbazolyl)-1,1′-biphenyl (CBP); 1,3-di(carbazol-9-yl)benzene (mCP); 1,3,5-tri(carbazol-9-yl)benzene (TCP); and/or a combination thereof:

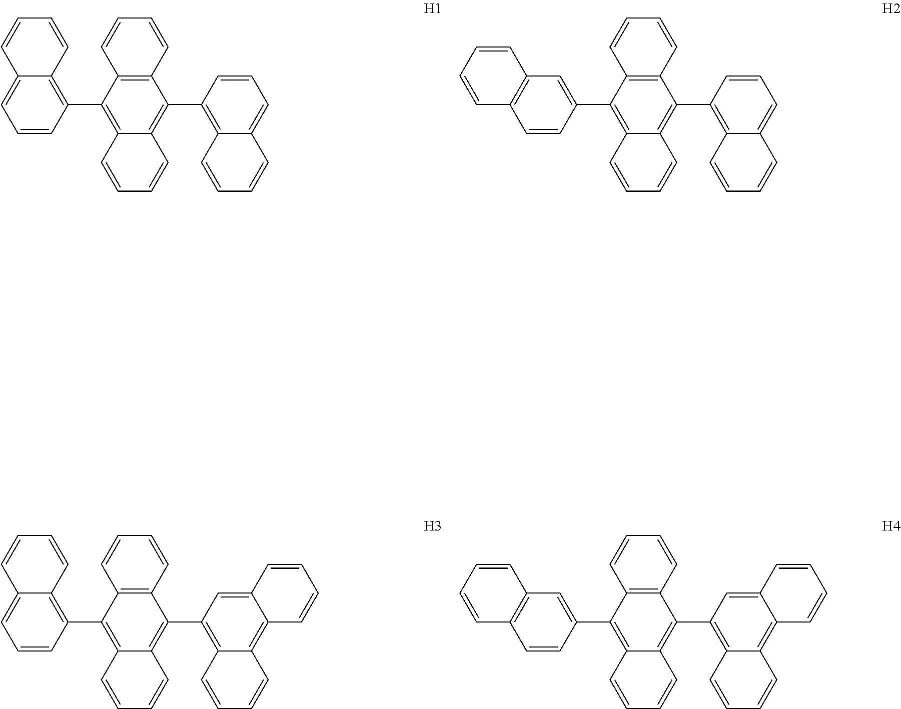

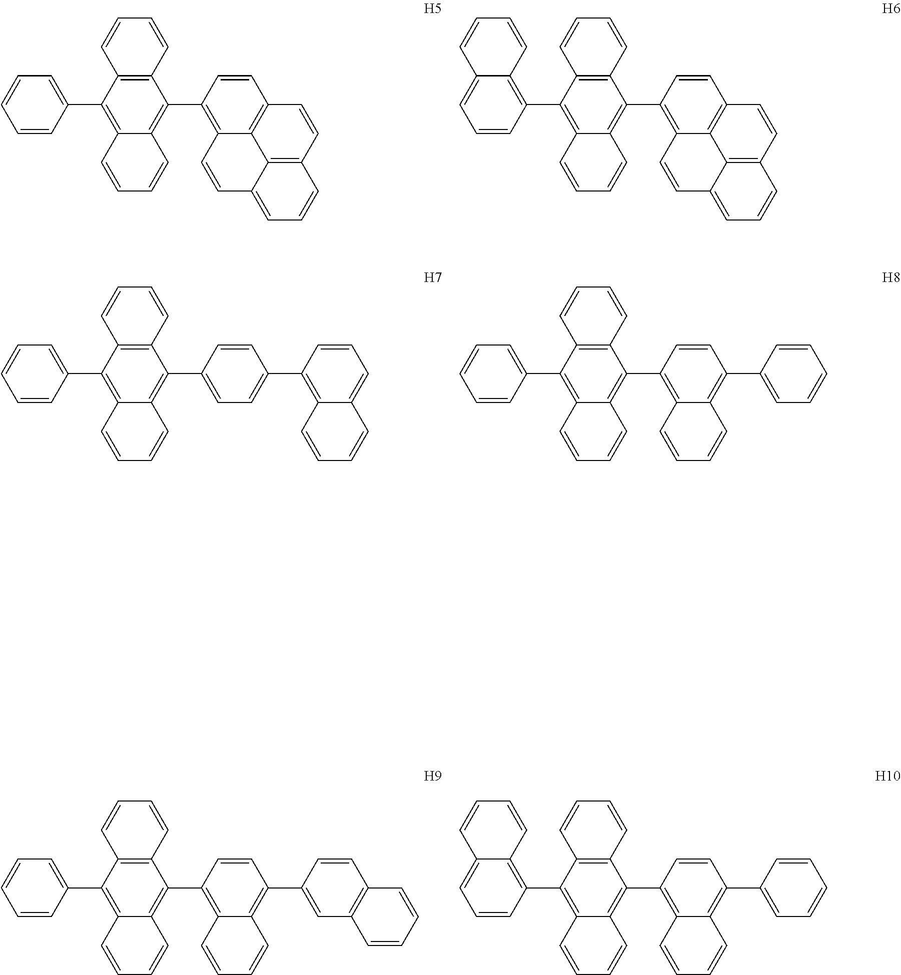

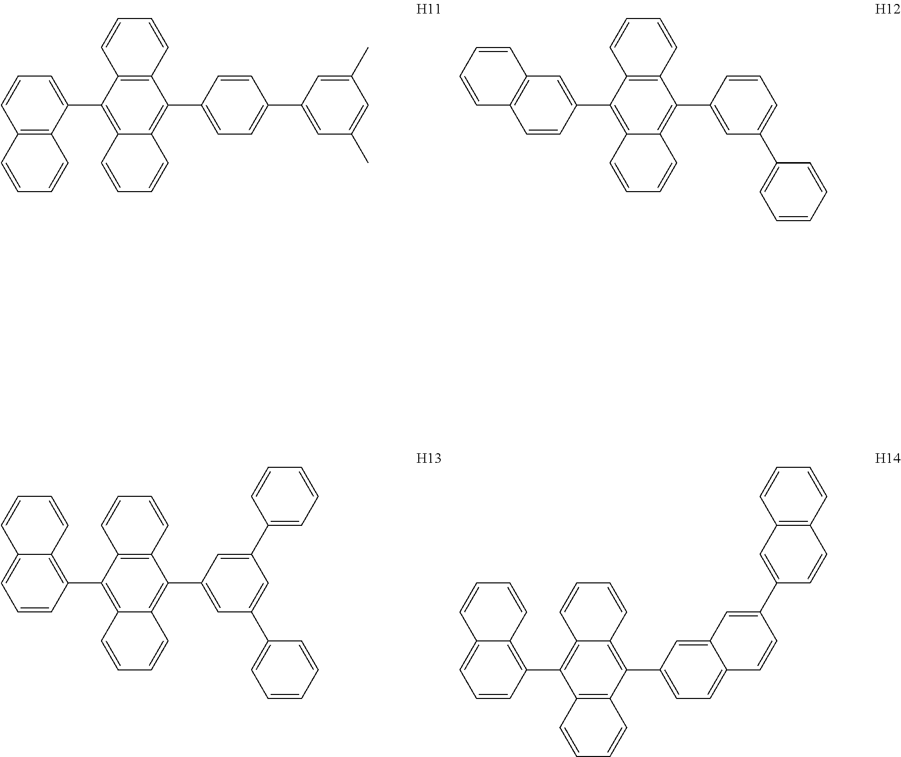

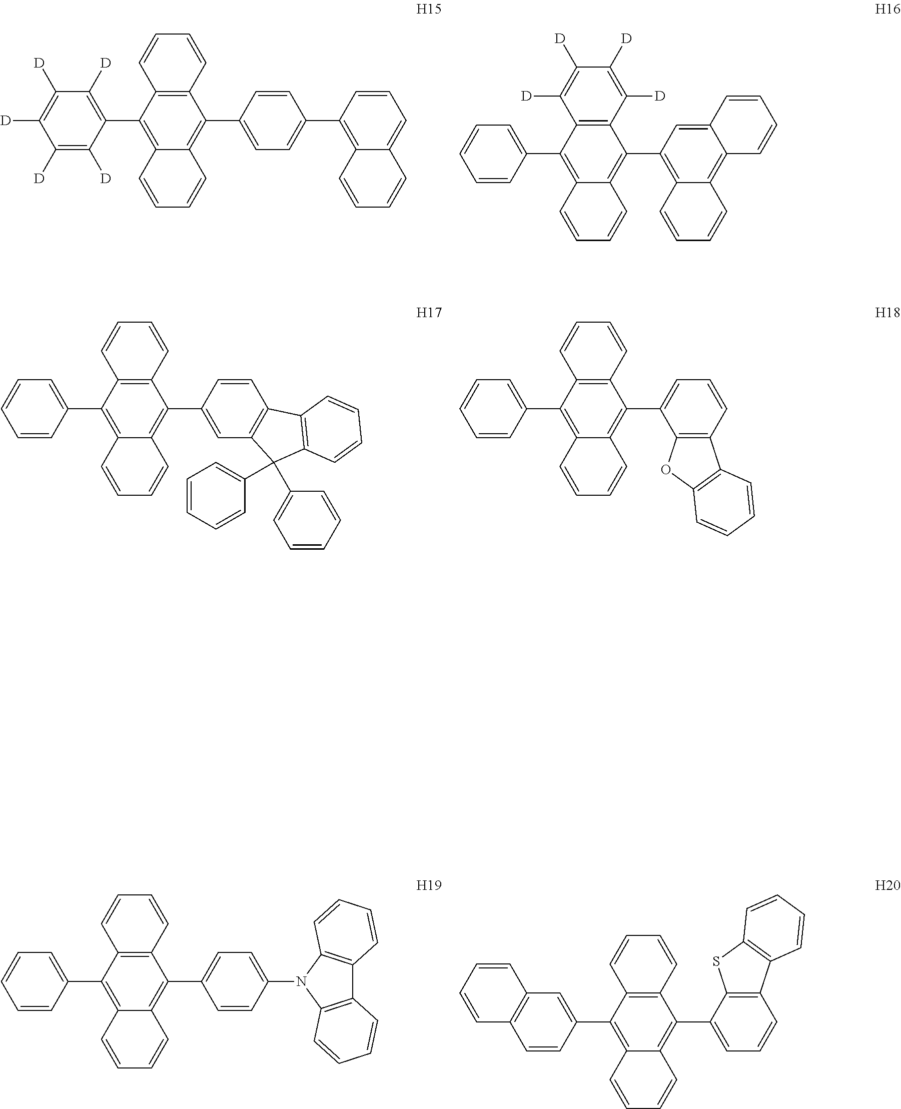

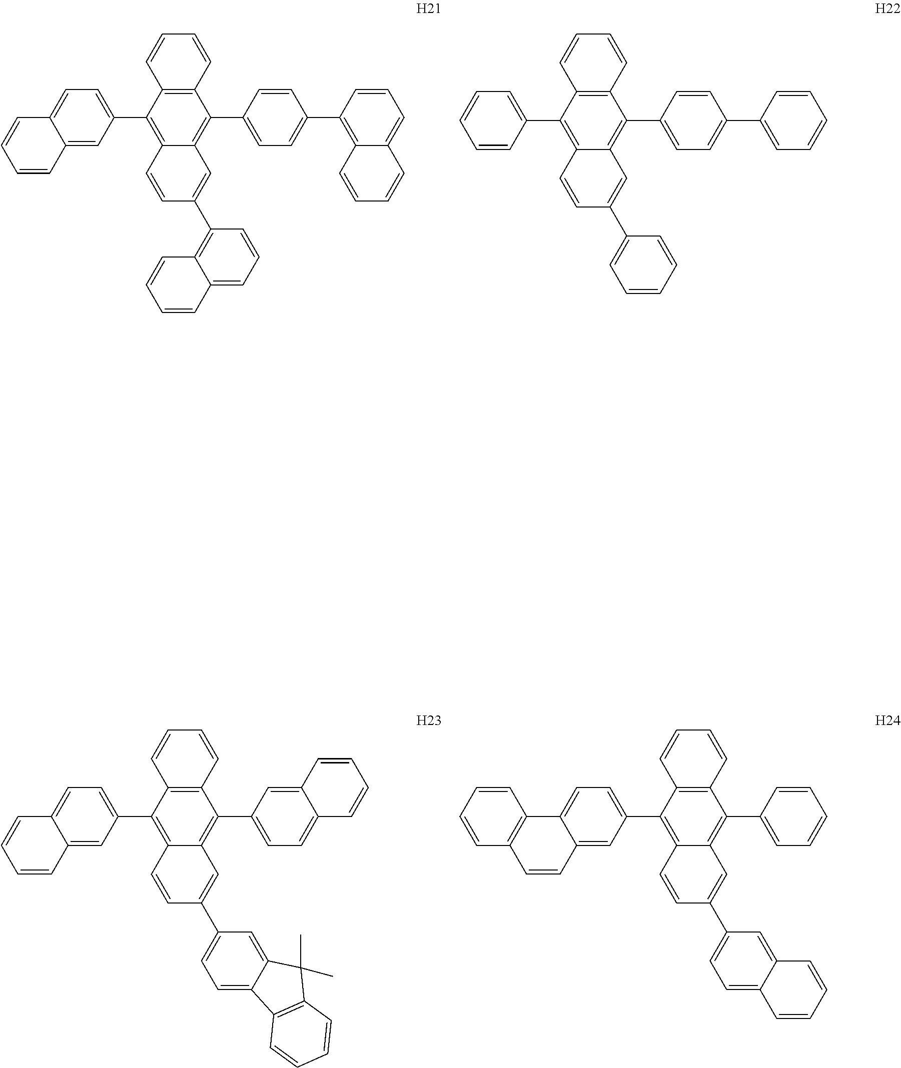

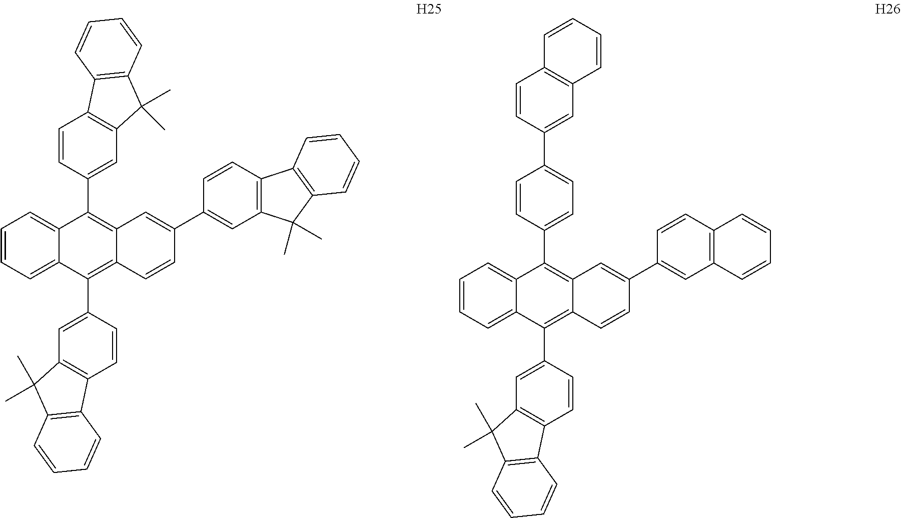

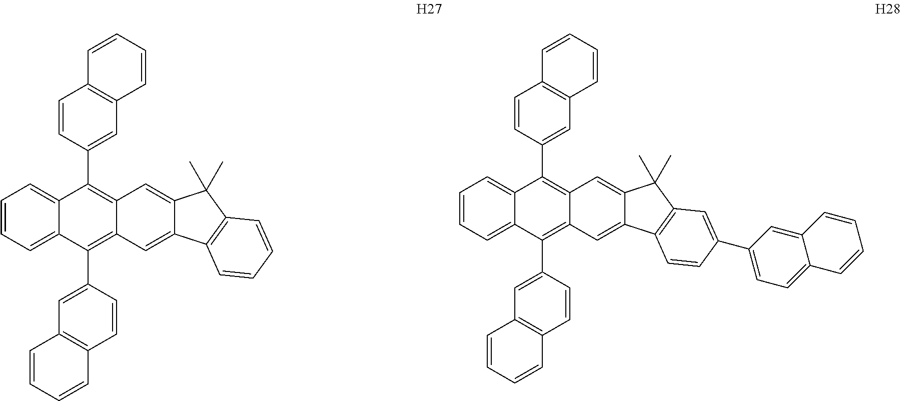

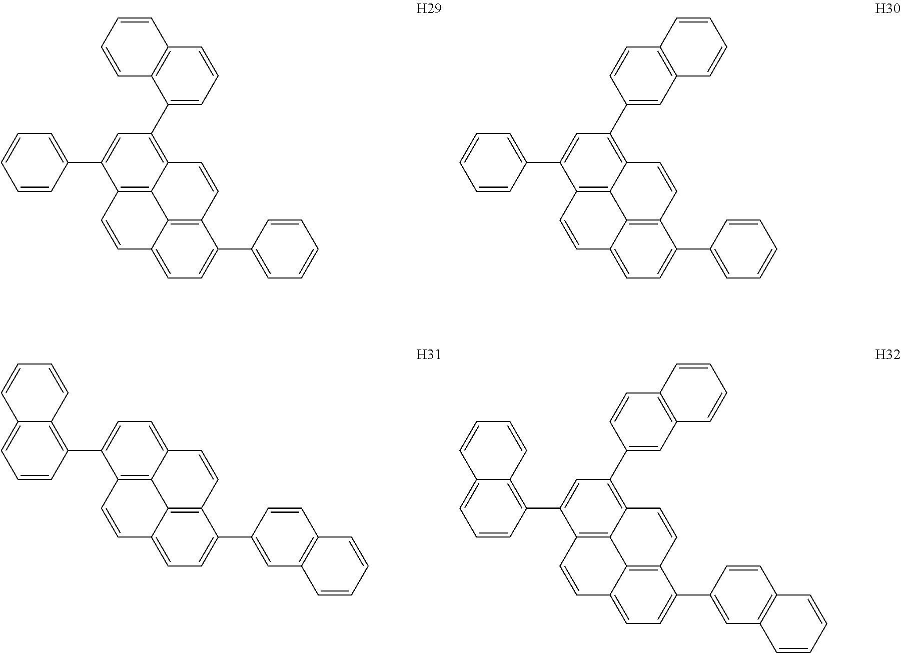

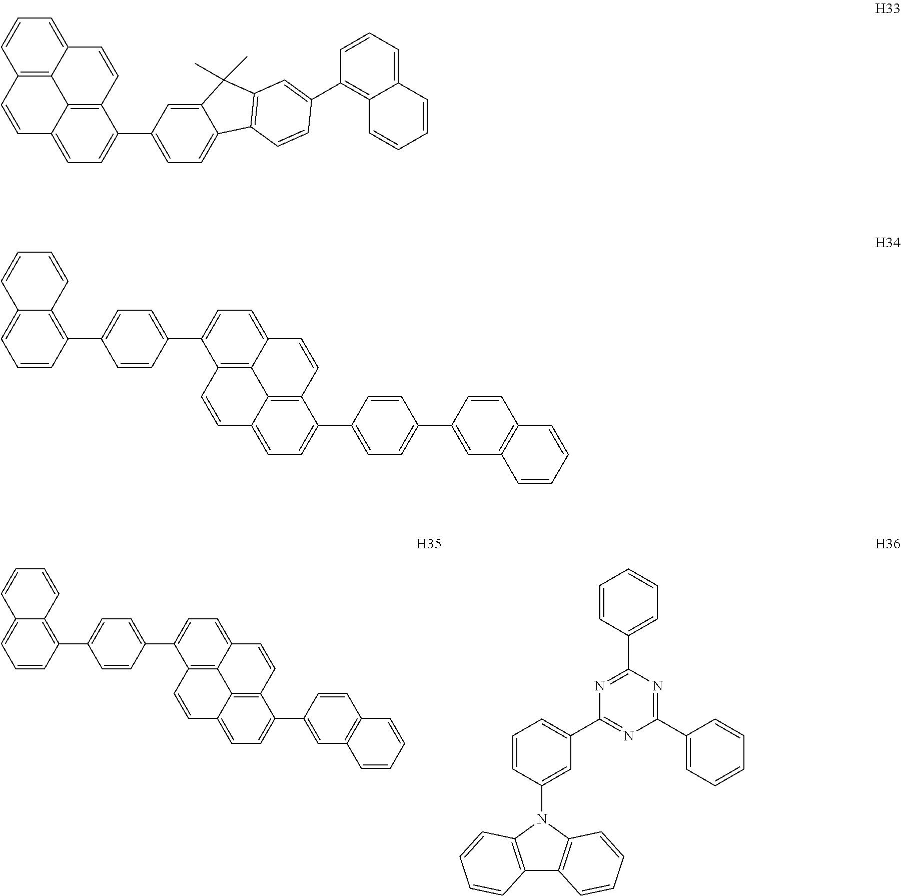

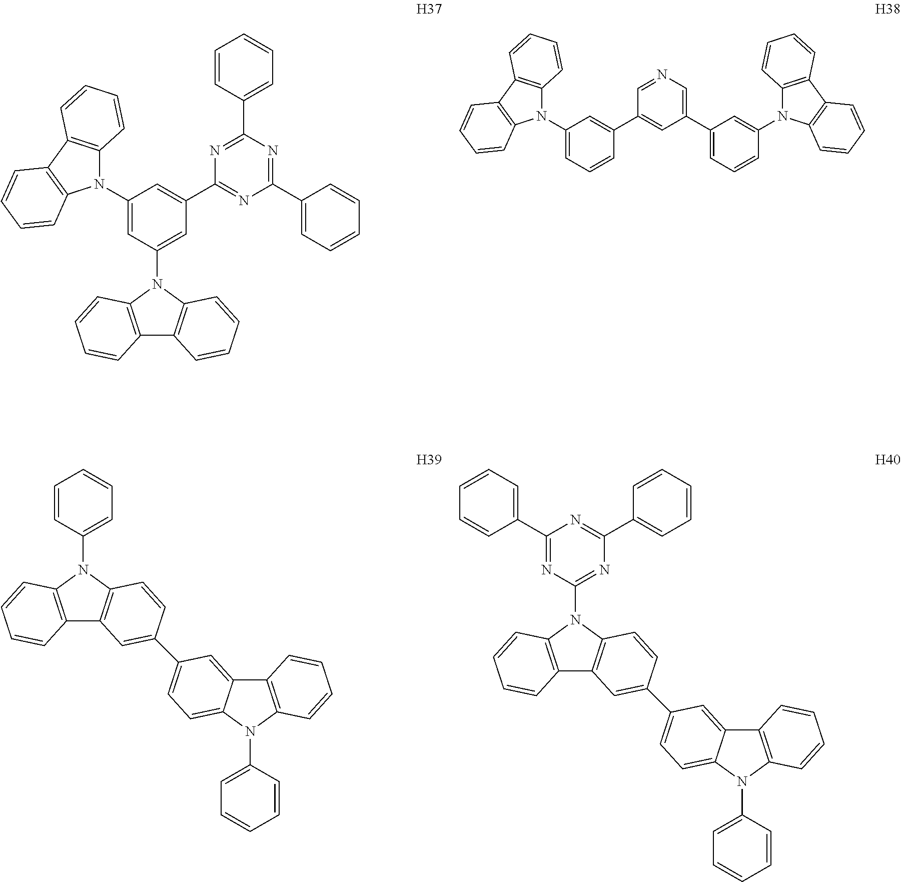 [SMILES: c1ccc(-c2nc(-c3ccccc3)nc(-c3cc(-n4c5ccccc5c5ccccc54)cc(-n4c5ccccc5c5ccccc54)c3)n2)cc1 ; c1cc(-c2cncc(-c3cccc(-n4c5ccccc5c5ccccc54)c3)c2)cc(-n2c3ccccc3c3ccccc32)c1 ; c1ccc(-n2c3ccccc3c3cc(-c4ccc5c(c4)c4ccccc4n5-c4ccccc4)ccc32)cc1 ; c1ccc(-c2nc(-c3ccccc3)nc(-n3c4ccccc4c4cc(-c5ccc6c(c5)c5ccccc5n6-c5ccccc5)ccc43)n2)cc1]

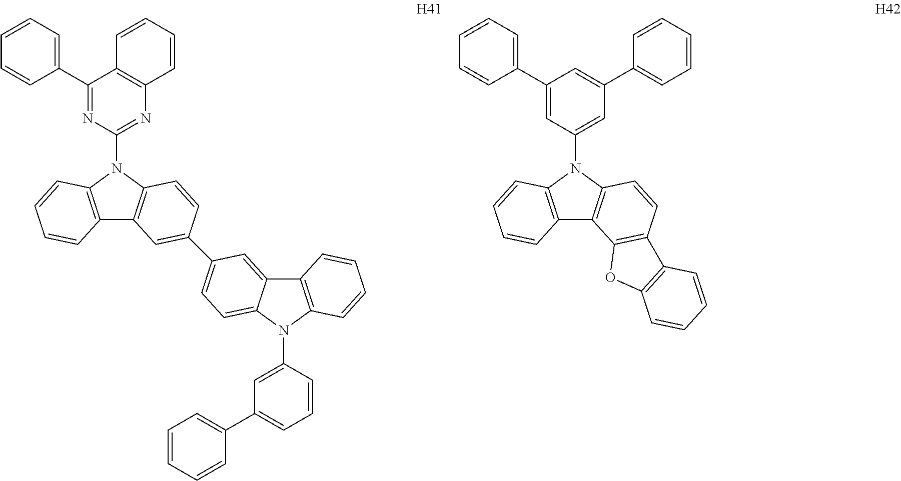 [SMILES: c1ccc(-c2cccc(-n3c4ccccc4c4cc(-c5ccc6c(c5)c5ccccc5n6-c5nc(-c6ccccc6)c6ccccc6n5)ccc43)c2)cc1 ; c1ccc(-c2cc(-c3ccccc3)cc(-n3c4ccccc4c4c5oc6ccccc6c5ccc43)c2)cc1]

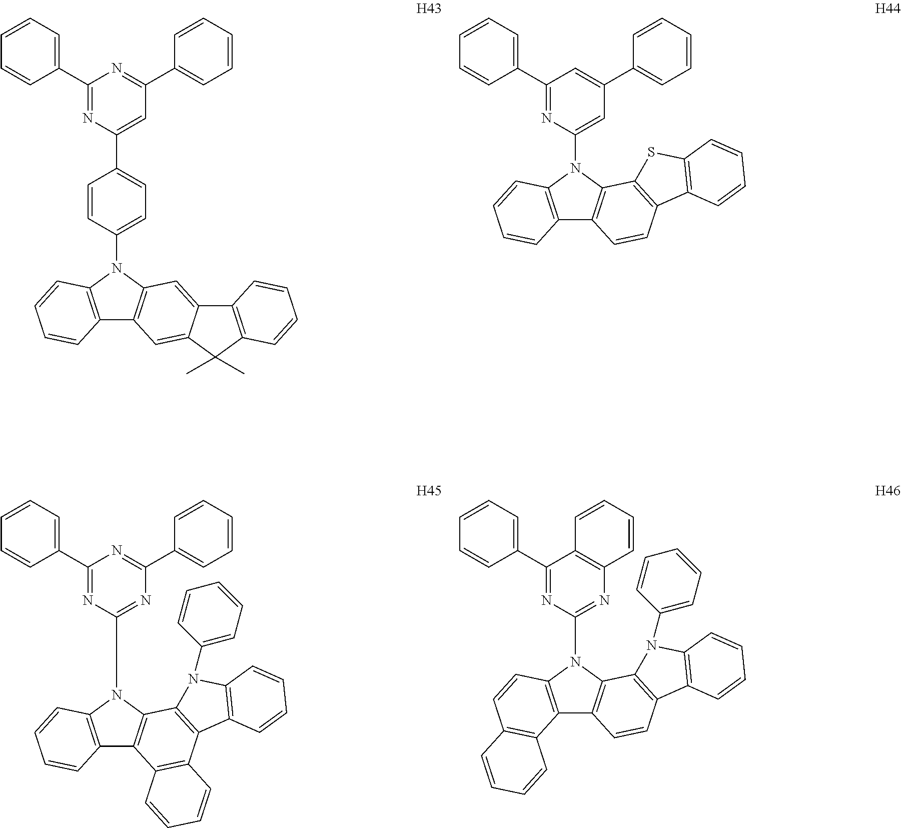 [SMILES: CC1(C)c2ccccc2-c2cc3c(cc21)c1ccccc1n3-c1ccc(-c2cc(-c3ccccc3)nc(-c3ccccc3)n2)cc1 ; c1ccc(-c2cc(-c3ccccc3)nc(-n3c4ccccc4c4ccc5c6ccccc6sc5c43)c2)cc1 ; c1ccc(-c2nc(-c3ccccc3)nc(-n3c4ccccc4c4c5ccccc5c5c6ccccc6n(-c6ccccc6)c5c43)n2)cc1 ; c1ccc(-c2nc(-n3c4ccc5ccccc5c4c4ccc5c6ccccc6n(-c6ccccc6)c5c43)nc3ccccc23)cc1]

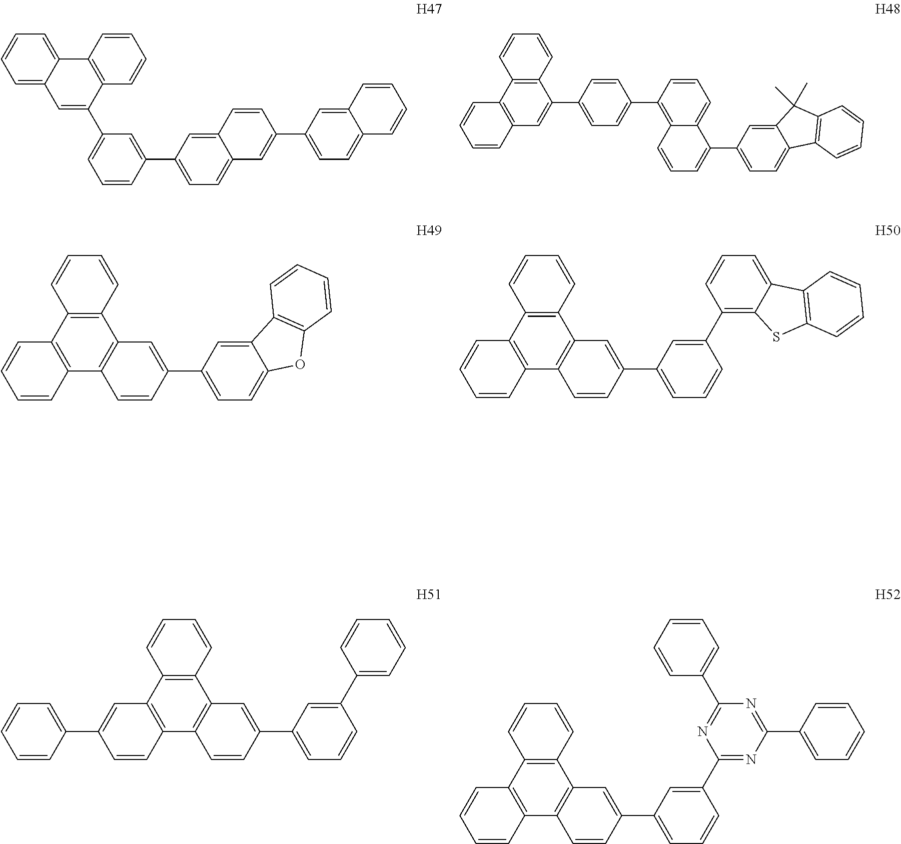

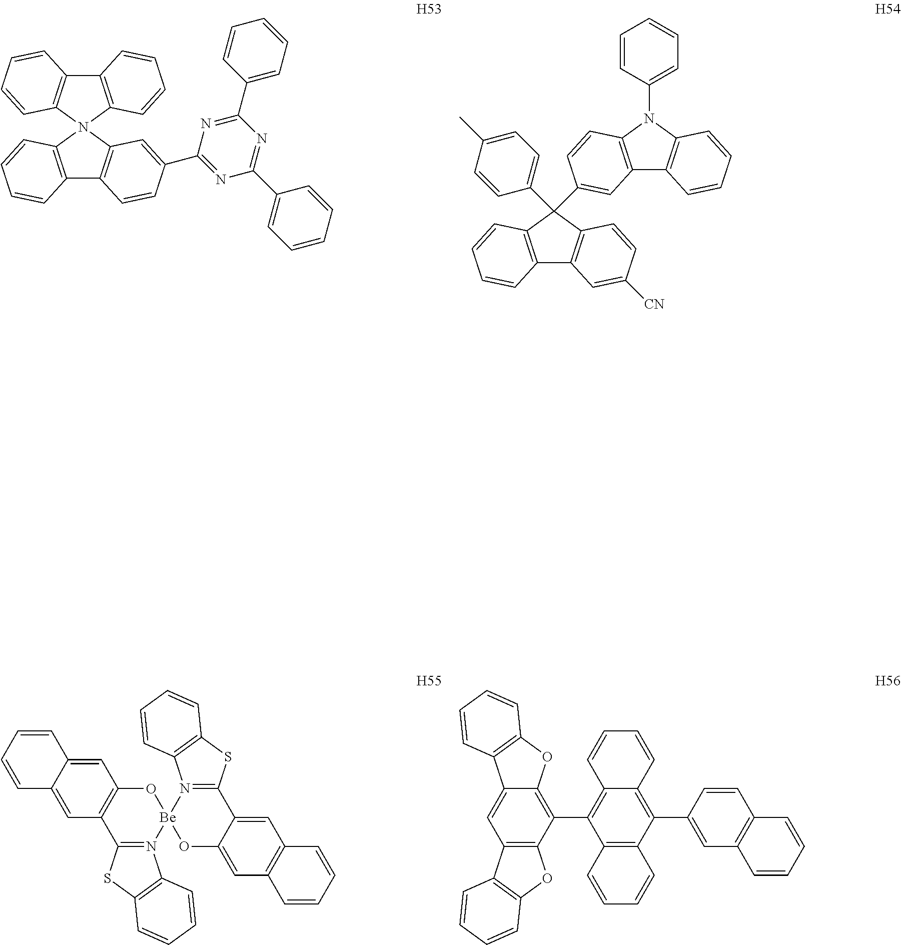

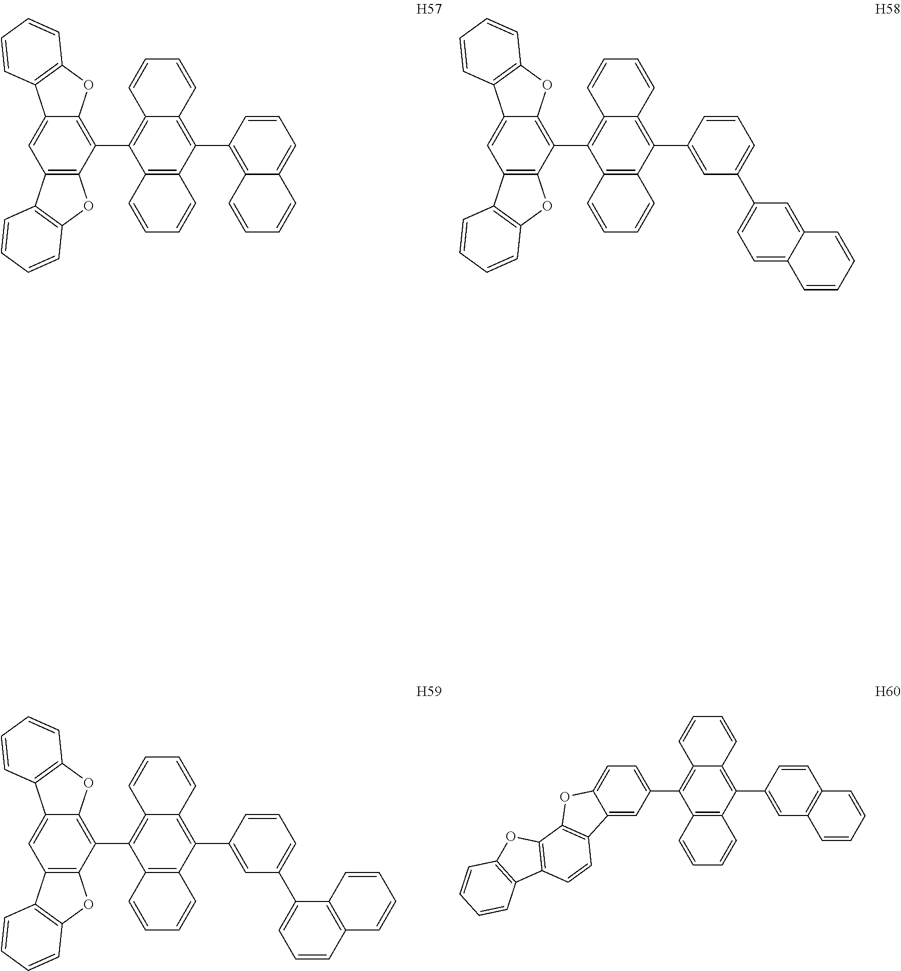 [SMILES: c1ccc2c(-c3c4ccccc4c(-c4c5oc6ccccc6c5cc5c4oc4ccccc45)c4ccccc34)cccc2c1 ; c1cc(-c2ccc3ccccc3c2)cc(-c2c3ccccc3c(-c3c4oc5ccccc5c4cc4c3oc3ccccc34)c3ccccc23)c1 ; c1cc(-c2cccc3ccccc23)cc(-c2c3ccccc3c(-c3c4oc5ccccc5c4cc4c3oc3ccccc34)c3ccccc23)c1 ; c1ccc2cc(-c3c4ccccc4c(-c4ccc5oc6c(ccc7c8ccccc8oc76)c5c4)c4ccccc34)ccc2c1]

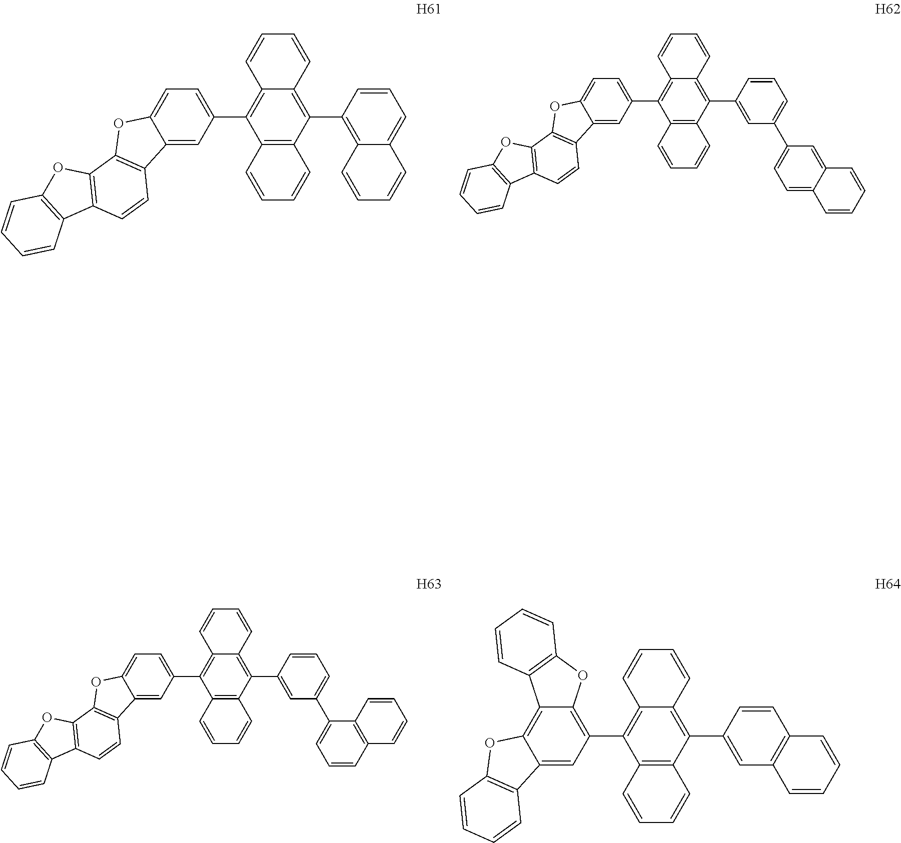 [SMILES: c1ccc2c(-c3c4ccccc4c(-c4ccc5oc6c(ccc7c8ccccc8oc76)c5c4)c4ccccc34)cccc2c1 ; c1cc(-c2ccc3ccccc3c2)cc(-c2c3ccccc3c(-c3ccc4oc5c(ccc6c7ccccc7oc65)c4c3)c3ccccc23)c1 ; c1cc(-c2cccc3ccccc23)cc(-c2c3ccccc3c(-c3ccc4oc5c(ccc6c7ccccc7oc65)c4c3)c3ccccc23)c1 ; c1ccc2cc(-c3c4ccccc4c(-c4cc5c6ccccc6oc5c5c4oc4ccccc45)c4ccccc34)ccc2c1]

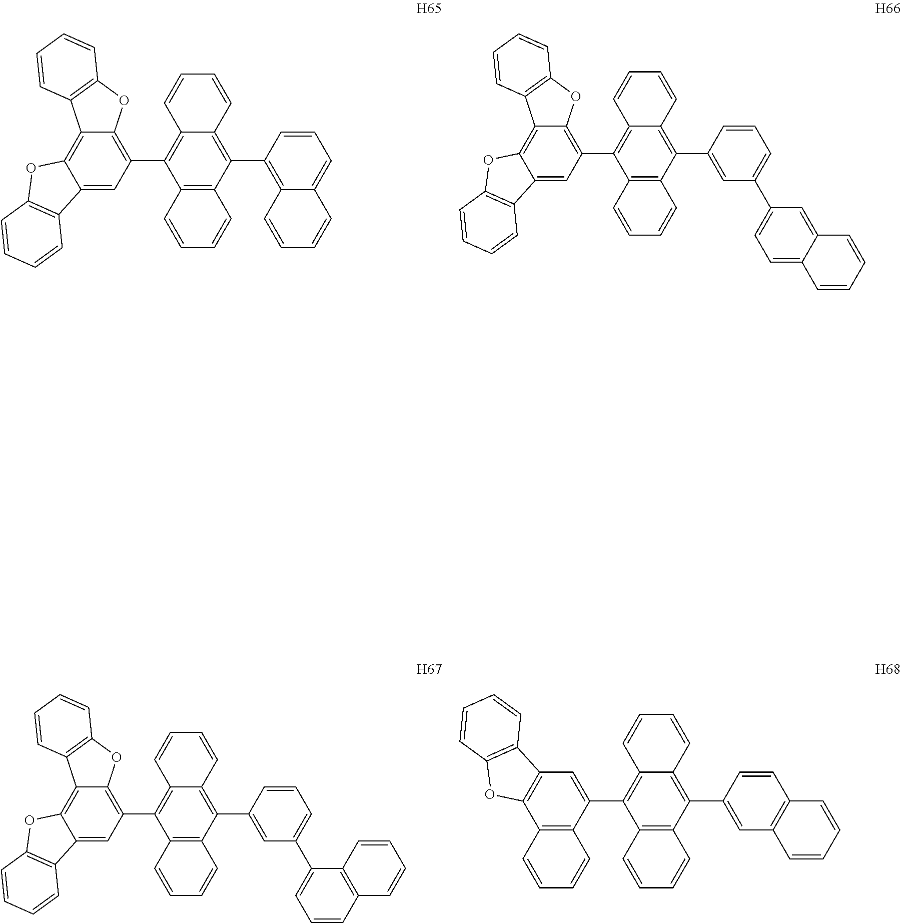 [SMILES: c1ccc2c(-c3c4ccccc4c(-c4cc5c6ccccc6oc5c5c4oc4ccccc45)c4ccccc34)cccc2c1 ; c1cc(-c2ccc3ccccc3c2)cc(-c2c3ccccc3c(-c3cc4c5ccccc5oc4c4c3oc3ccccc34)c3ccccc23)c1 ; c1cc(-c2cccc3ccccc23)cc(-c2c3ccccc3c(-c3cc4c5ccccc5oc4c4c3oc3ccccc34)c3ccccc23)c1 ; c1ccc2cc(-c3c4ccccc4c(-c4cc5c6ccccc6oc5c5ccccc45)c4ccccc34)ccc2c1]

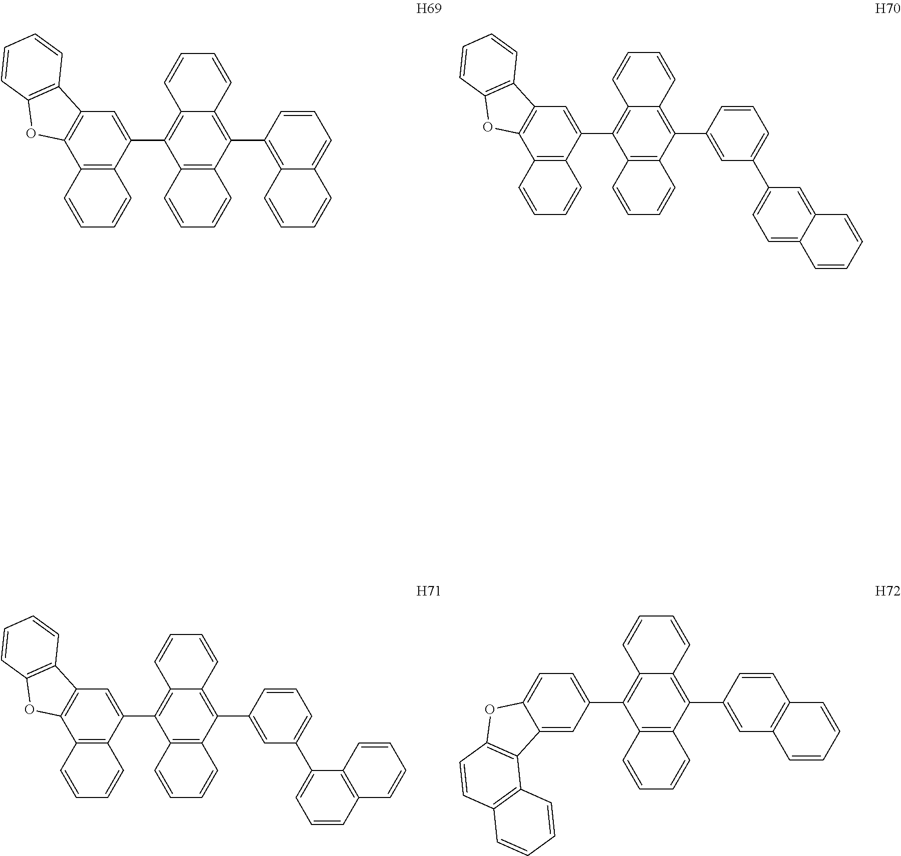 [SMILES: c1ccc2c(-c3c4ccccc4c(-c4cc5c6ccccc6oc5c5ccccc45)c4ccccc34)cccc2c1 ; c1cc(-c2ccc3ccccc3c2)cc(-c2c3ccccc3c(-c3cc4c5ccccc5oc4c4ccccc34)c3ccccc23)c1 ; c1cc(-c2cccc3ccccc23)cc(-c2c3ccccc3c(-c3cc4c5ccccc5oc4c4ccccc34)c3ccccc23)c1 ; c1ccc2cc(-c3c4ccccc4c(-c4ccc5oc6ccc7ccccc7c6c5c4)c4ccccc34)ccc2c1]

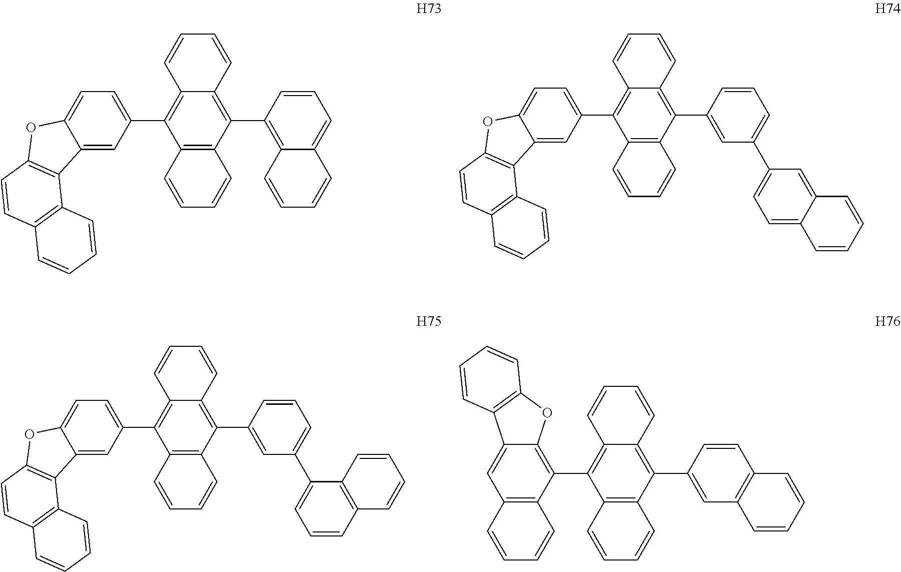 [SMILES: c1ccc2c(-c3c4ccccc4c(-c4ccc5oc6ccc7ccccc7c6c5c4)c4ccccc34)cccc2c1 ; c1cc(-c2ccc3ccccc3c2)cc(-c2c3ccccc3c(-c3ccc4oc5ccc6ccccc6c5c4c3)c3ccccc23)c1 ; c1cc(-c2cccc3ccccc23)cc(-c2c3ccccc3c(-c3ccc4oc5ccc6ccccc6c5c4c3)c3ccccc23)c1 ; c1ccc2cc(-c3c4ccccc4c(-c4c5ccccc5cc5c4oc4ccccc45)c4ccccc34)ccc2c1]

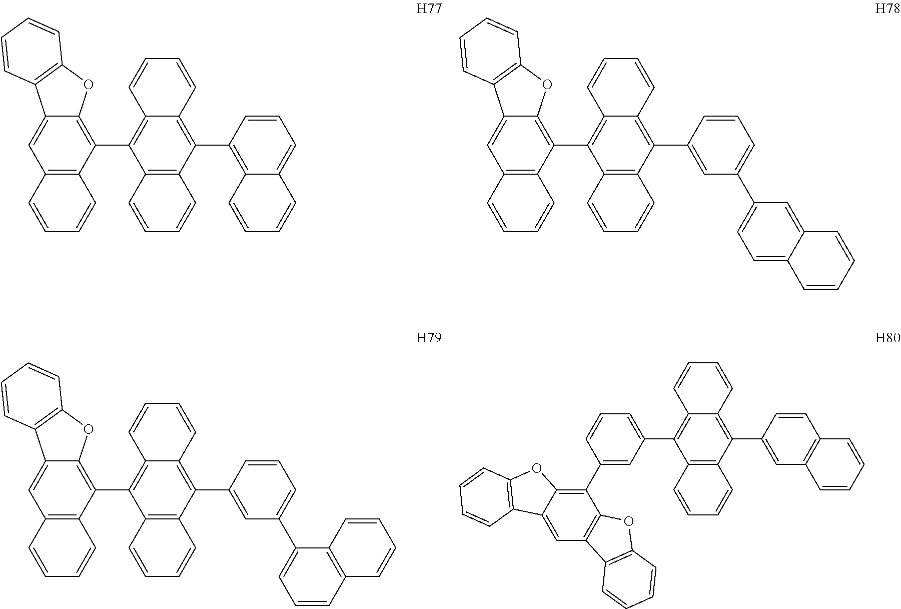 [SMILES: c1ccc2c(-c3c4ccccc4c(-c4c5ccccc5cc5c4oc4ccccc45)c4ccccc34)cccc2c1 ; c1cc(-c2ccc3ccccc3c2)cc(-c2c3ccccc3c(-c3c4ccccc4cc4c3oc3ccccc34)c3ccccc23)c1 ; c1cc(-c2cccc3ccccc23)cc(-c2c3ccccc3c(-c3c4ccccc4cc4c3oc3ccccc34)c3ccccc23)c1 ; c1cc(-c2c3ccccc3c(-c3ccc4ccccc4c3)c3ccccc23)cc(-c2c3oc4ccccc4c3cc3c2oc2ccccc23)c1]

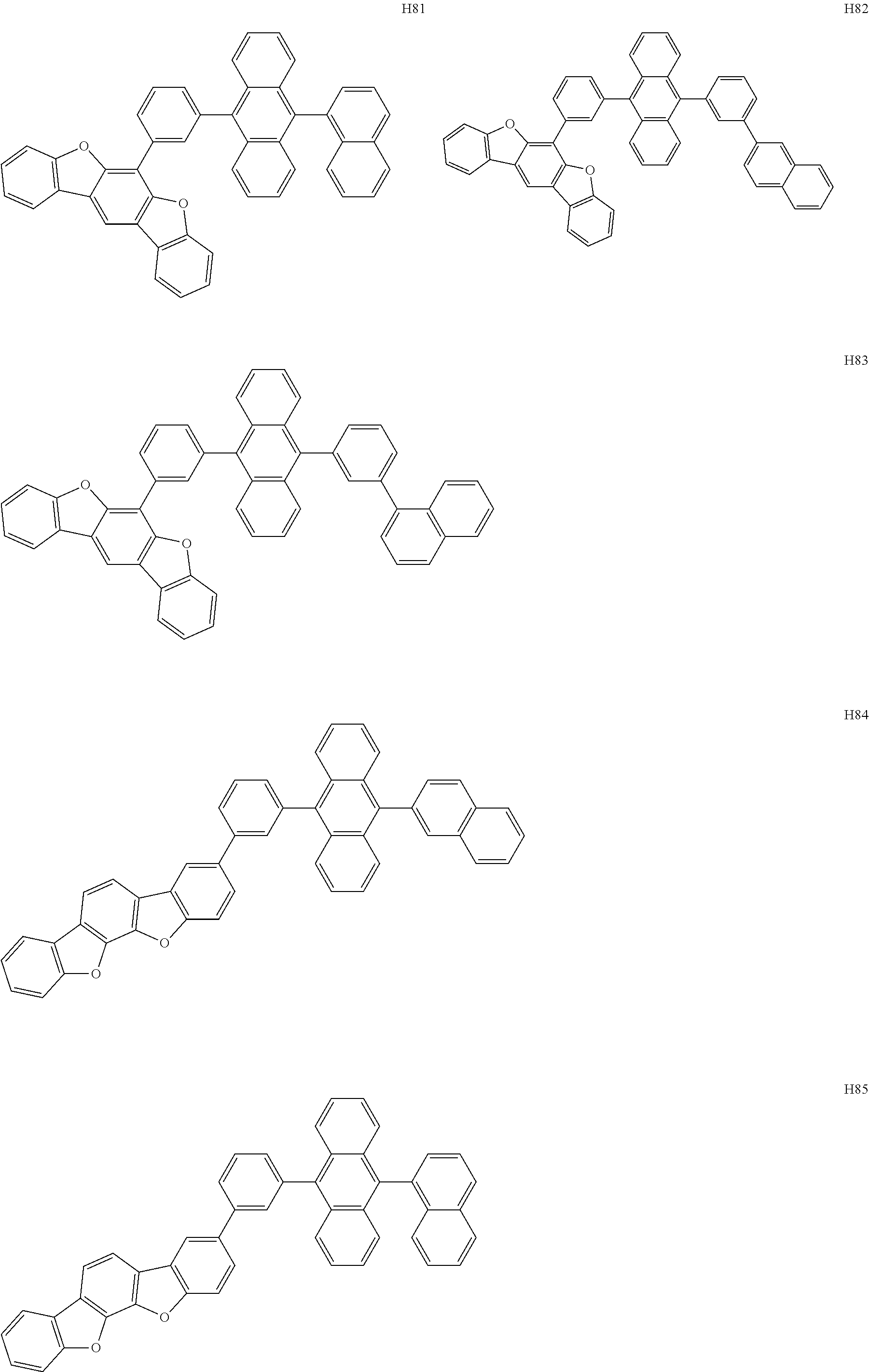 [SMILES: c1cc(-c2c3ccccc3c(-c3cccc4ccccc34)c3ccccc23)cc(-c2c3oc4ccccc4c3cc3c2oc2ccccc23)c1 ; c1cc(-c2ccc3ccccc3c2)cc(-c2c3ccccc3c(-c3cccc(-c4c5oc6ccccc6c5cc5c4oc4ccccc45)c3)c3ccccc23)c1 ; c1cc(-c2cccc3ccccc23)cc(-c2c3ccccc3c(-c3cccc(-c4c5oc6ccccc6c5cc5c4oc4ccccc45)c3)c3ccccc23)c1 ; c1cc(-c2ccc3oc4c(ccc5c6ccccc6oc54)c3c2)cc(-c2c3ccccc3c(-c3ccc4ccccc4c3)c3ccccc23)c1 ; c1cc(-c2ccc3oc4c(ccc5c6ccccc6oc54)c3c2)cc(-c2c3ccccc3c(-c3cccc4ccccc34)c3ccccc23)c1]

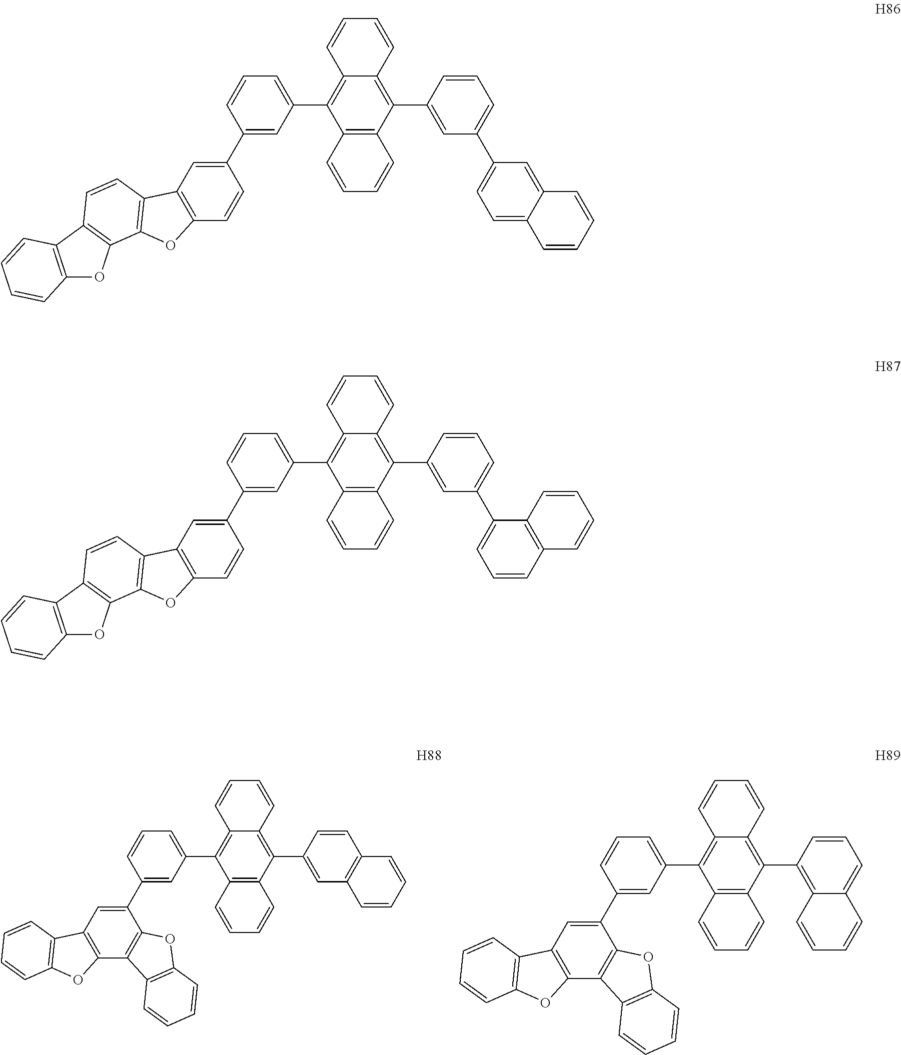 [SMILES: c1cc(-c2ccc3ccccc3c2)cc(-c2c3ccccc3c(-c3cccc(-c4ccc5oc6c(ccc7c8ccccc8oc76)c5c4)c3)c3ccccc23)c1 ; c1cc(-c2ccc3oc4c(ccc5c6ccccc6oc54)c3c2)cc(-c2c3ccccc3c(-c3cccc(-c4cccc5ccccc45)c3)c3ccccc23)c1 ; c1cc(-c2c3ccccc3c(-c3ccc4ccccc4c3)c3ccccc23)cc(-c2cc3c4ccccc4oc3c3c2oc2ccccc23)c1 ; c1cc(-c2c3ccccc3c(-c3cccc4ccccc34)c3ccccc23)cc(-c2cc3c4ccccc4oc3c3c2oc2ccccc23)c1]

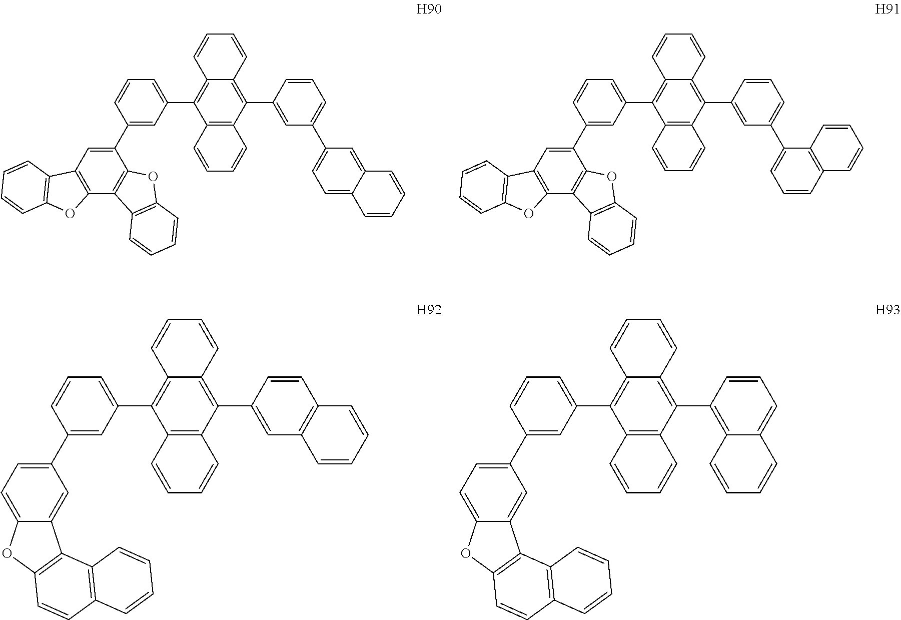 [SMILES: c1cc(-c2ccc3ccccc3c2)cc(-c2c3ccccc3c(-c3cccc(-c4cc5c6ccccc6oc5c5c4oc4ccccc45)c3)c3ccccc23)c1 ; c1cc(-c2cccc3ccccc23)cc(-c2c3ccccc3c(-c3cccc(-c4cc5c6ccccc6oc5c5c4oc4ccccc45)c3)c3ccccc23)c1 ; c1cc(-c2ccc3oc4ccc5ccccc5c4c3c2)cc(-c2c3ccccc3c(-c3ccc4ccccc4c3)c3ccccc23)c1 ; c1cc(-c2ccc3oc4ccc5ccccc5c4c3c2)cc(-c2c3ccccc3c(-c3cccc4ccccc34)c3ccccc23)c1]

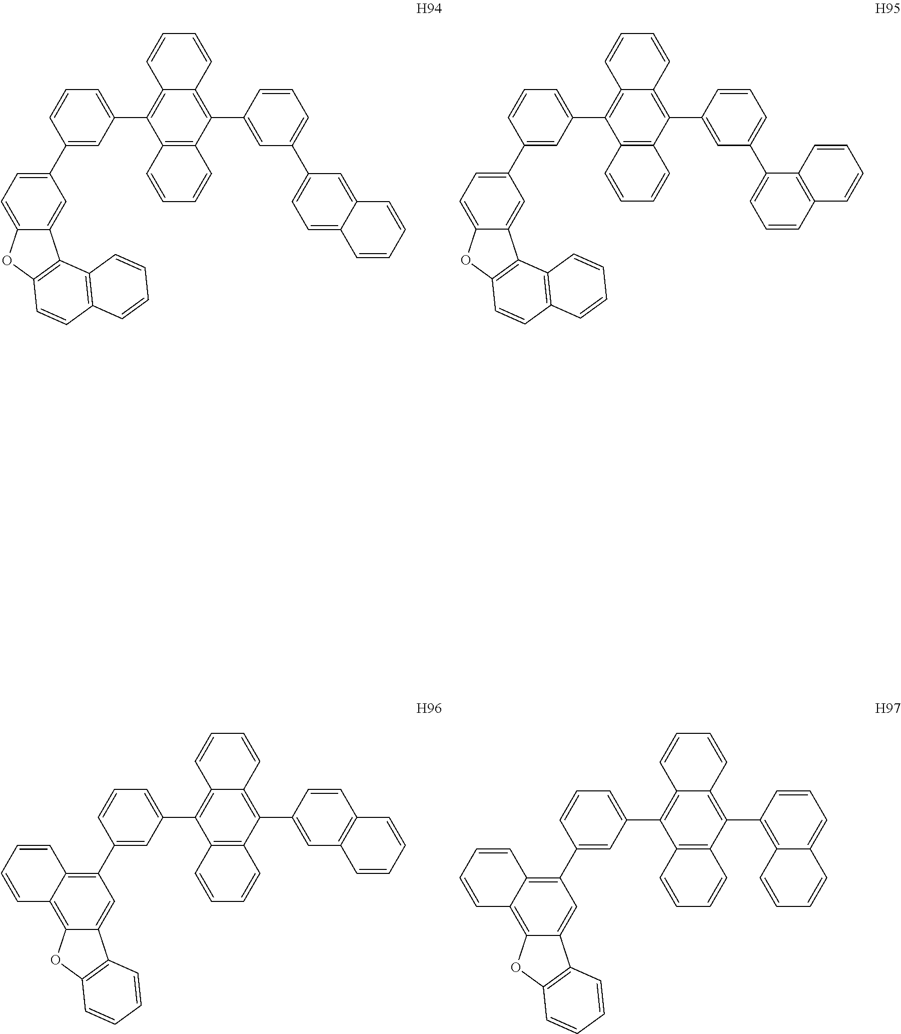 [SMILES: c1cc(-c2ccc3ccccc3c2)cc(-c2c3ccccc3c(-c3cccc(-c4ccc5oc6ccc7ccccc7c6c5c4)c3)c3ccccc23)c1 ; c1cc(-c2ccc3oc4ccc5ccccc5c4c3c2)cc(-c2c3ccccc3c(-c3cccc(-c4cccc5ccccc45)c3)c3ccccc23)c1 ; c1cc(-c2c3ccccc3c(-c3ccc4ccccc4c3)c3ccccc23)cc(-c2cc3c4ccccc4oc3c3ccccc23)c1 ; c1cc(-c2c3ccccc3c(-c3cccc4ccccc34)c3ccccc23)cc(-c2cc3c4ccccc4oc3c3ccccc23)c1]

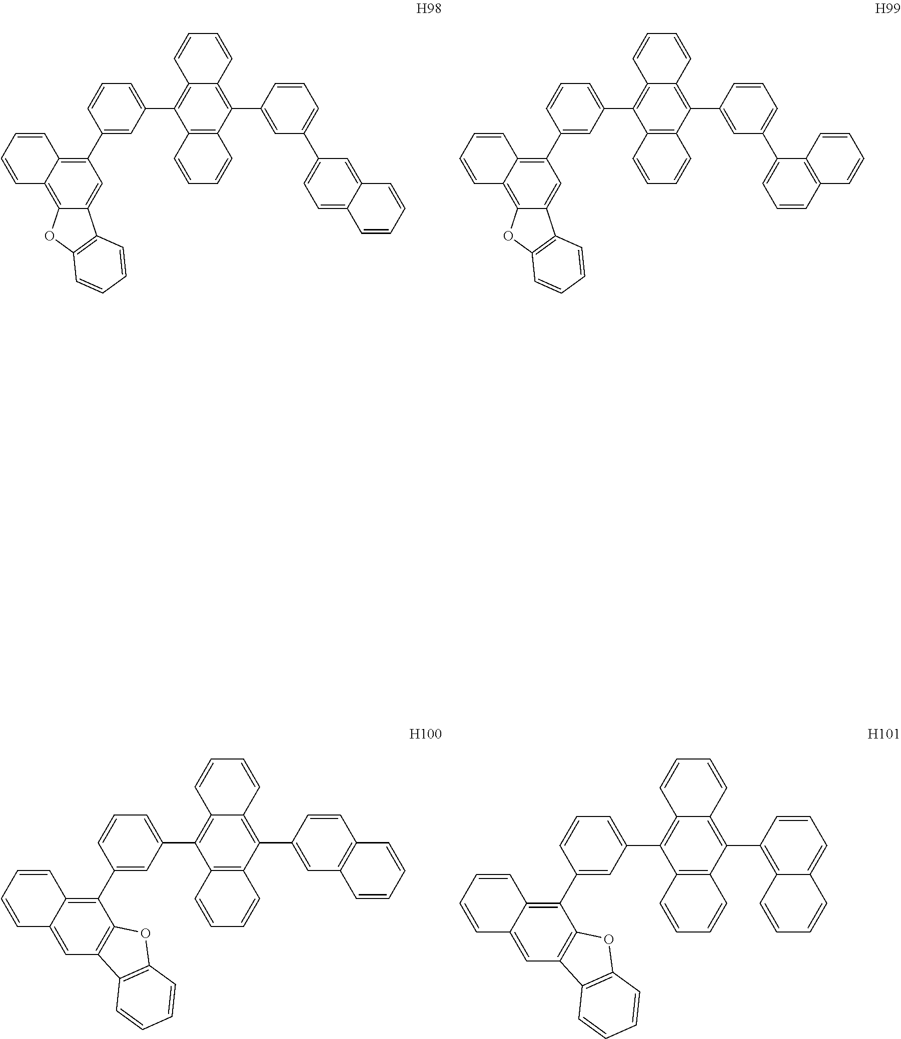 [SMILES: c1cc(-c2ccc3ccccc3c2)cc(-c2c3ccccc3c(-c3cccc(-c4cc5c6ccccc6oc5c5ccccc45)c3)c3ccccc23)c1 ; c1cc(-c2cccc3ccccc23)cc(-c2c3ccccc3c(-c3cccc(-c4cc5c6ccccc6oc5c5ccccc45)c3)c3ccccc23)c1 ; c1cc(-c2c3ccccc3c(-c3ccc4ccccc4c3)c3ccccc23)cc(-c2c3ccccc3cc3c2oc2ccccc23)c1 ; c1cc(-c2c3ccccc3c(-c3cccc4ccccc34)c3ccccc23)cc(-c2c3ccccc3cc3c2oc2ccccc23)c1]

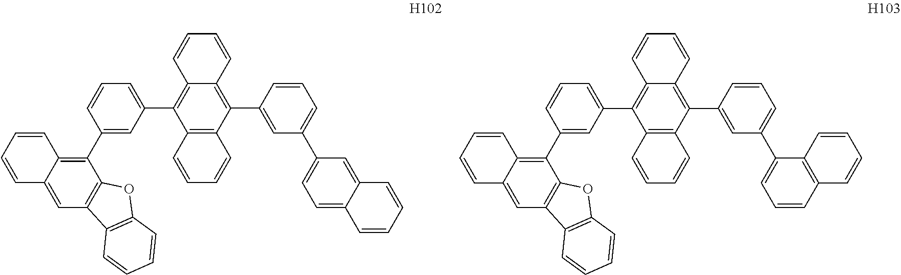 [SMILES: c1cc(-c2ccc3ccccc3c2)cc(-c2c3ccccc3c(-c3cccc(-c4c5ccccc5cc5c4oc4ccccc45)c3)c3ccccc23)c1 ; c1cc(-c2cccc3ccccc23)cc(-c2c3ccccc3c(-c3cccc(-c4c5ccccc5cc5c4oc4ccccc45)c3)c3ccccc23)c1]

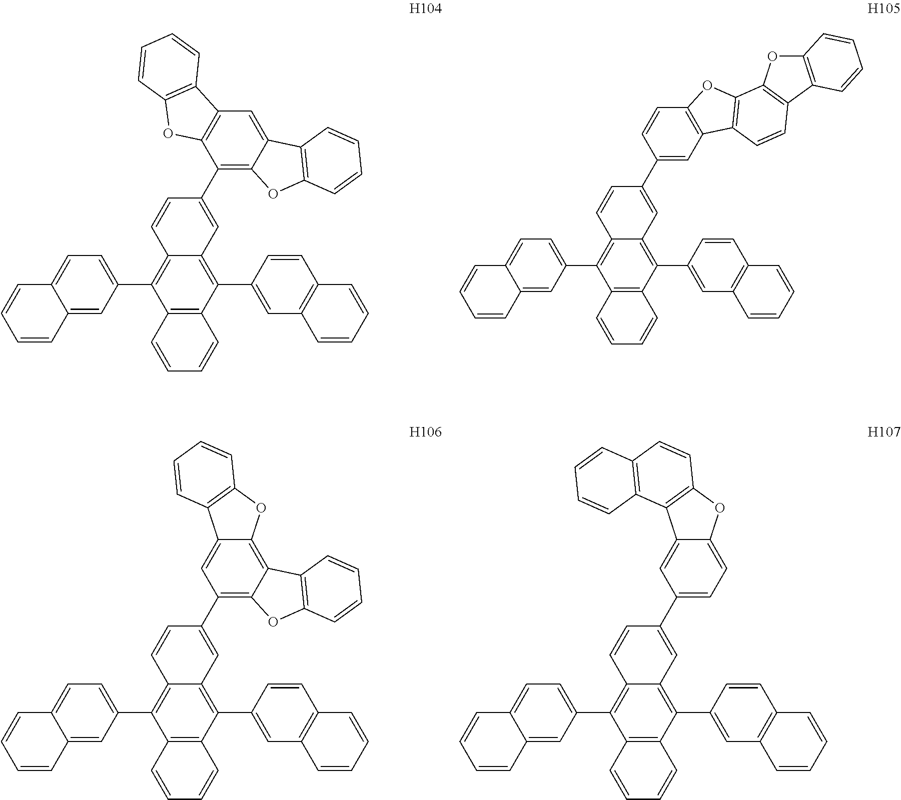 [SMILES: c1ccc2cc(-c3c4ccccc4c(-c4ccc5ccccc5c4)c4cc(-c5c6oc7ccccc7c6cc6c5oc5ccccc56)ccc34)ccc2c1 ; c1ccc2cc(-c3c4ccccc4c(-c4ccc5ccccc5c4)c4cc(-c5ccc6oc7c(ccc8c9ccccc9oc87)c6c5)ccc34)ccc2c1 ; c1ccc2cc(-c3c4ccccc4c(-c4ccc5ccccc5c4)c4cc(-c5cc6c7ccccc7oc6c6c5oc5ccccc56)ccc34)ccc2c1 ; c1ccc2cc(-c3c4ccccc4c(-c4ccc5ccccc5c4)c4cc(-c5ccc6oc7ccc8ccccc8c7c6c5)ccc34)ccc2c1]

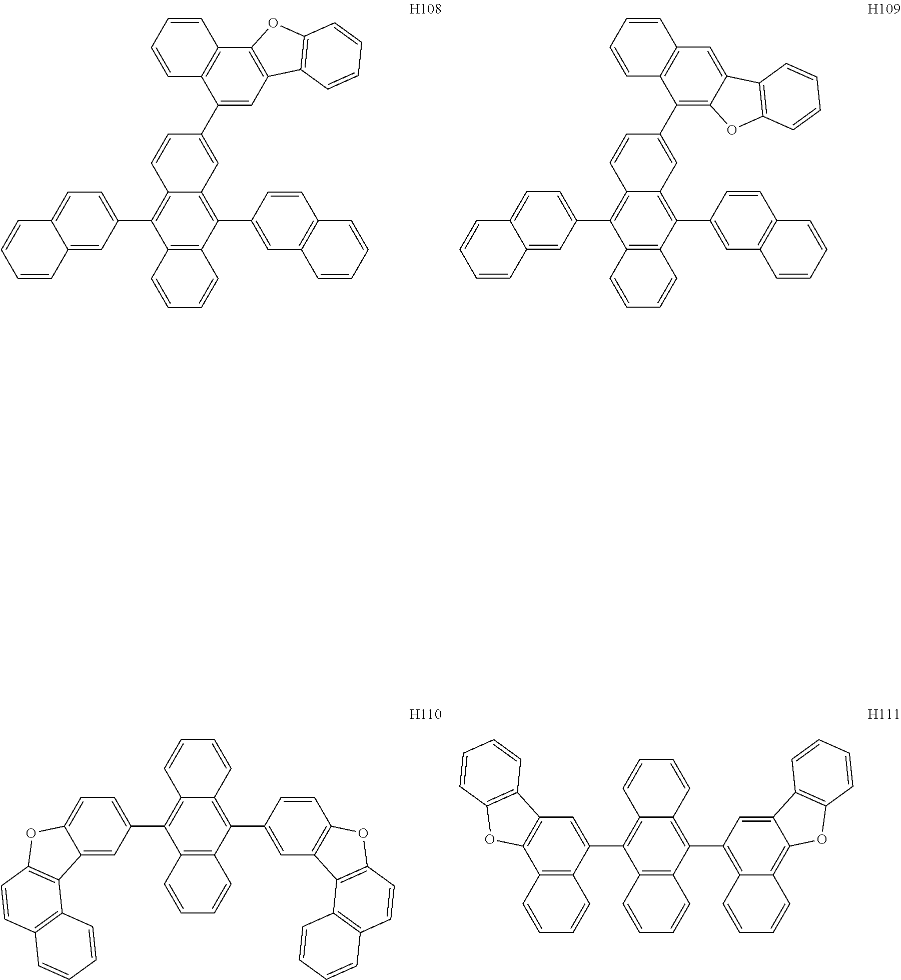 [SMILES: c1ccc2cc(-c3c4ccccc4c(-c4ccc5ccccc5c4)c4cc(-c5cc6c7ccccc7oc6c6ccccc56)ccc34)ccc2c1 ; c1ccc2cc(-c3c4ccccc4c(-c4ccc5ccccc5c4)c4cc(-c5c6ccccc6cc6c5oc5ccccc56)ccc34)ccc2c1 ; c1ccc2c(c1)ccc1oc3ccc(-c4c5ccccc5c(-c5ccc6oc7ccc8ccccc8c7c6c5)c5ccccc45)cc3c12 ; c1ccc2c(c1)oc1c3ccccc3c(-c3c4ccccc4c(-c4cc5c6ccccc6oc5c5ccccc45)c4ccccc34)cc21]

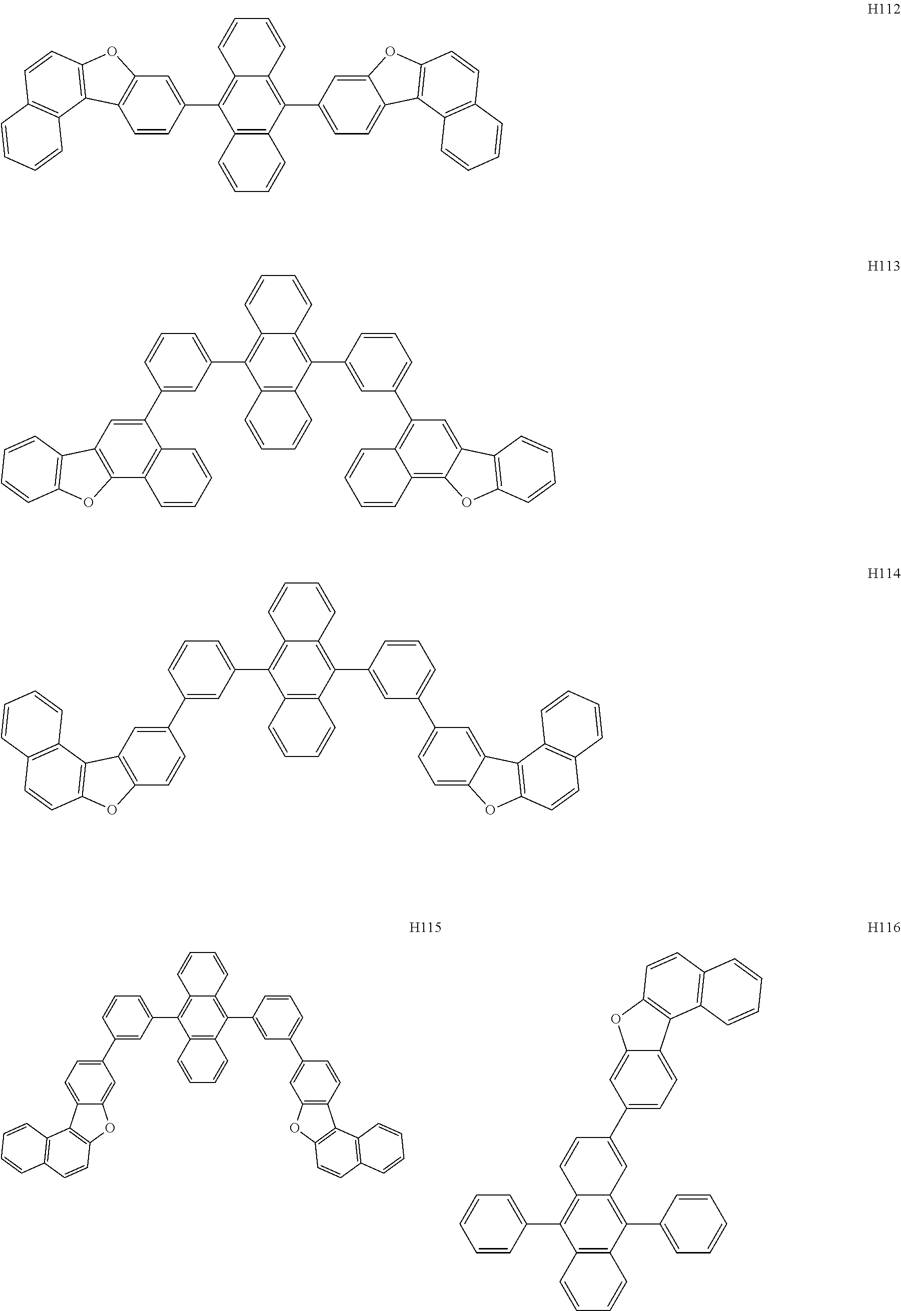 [SMILES: c1ccc2c(c1)ccc1oc3cc(-c4c5ccccc5c(-c5ccc6c(c5)oc5ccc7ccccc7c56)c5ccccc45)ccc3c12 ; c1cc(-c2c3ccccc3c(-c3cccc(-c4cc5c6ccccc6oc5c5ccccc45)c3)c3ccccc23)cc(-c2cc3c4ccccc4oc3c3ccccc23)c1 ; c1cc(-c2ccc3oc4ccc5ccccc5c4c3c2)cc(-c2c3ccccc3c(-c3cccc(-c4ccc5oc6ccc7ccccc7c6c5c4)c3)c3ccccc23)c1 ; c1cc(-c2ccc3c(c2)oc2ccc4ccccc4c23)cc(-c2c3ccccc3c(-c3cccc(-c4ccc5c(c4)oc4ccc6ccccc6c45)c3)c3ccccc23)c1 ; c1ccc(-c2c3ccccc3c(-c3ccccc3)c3cc(-c4ccc5c(c4)oc4ccc6ccccc6c45)ccc23)cc1]

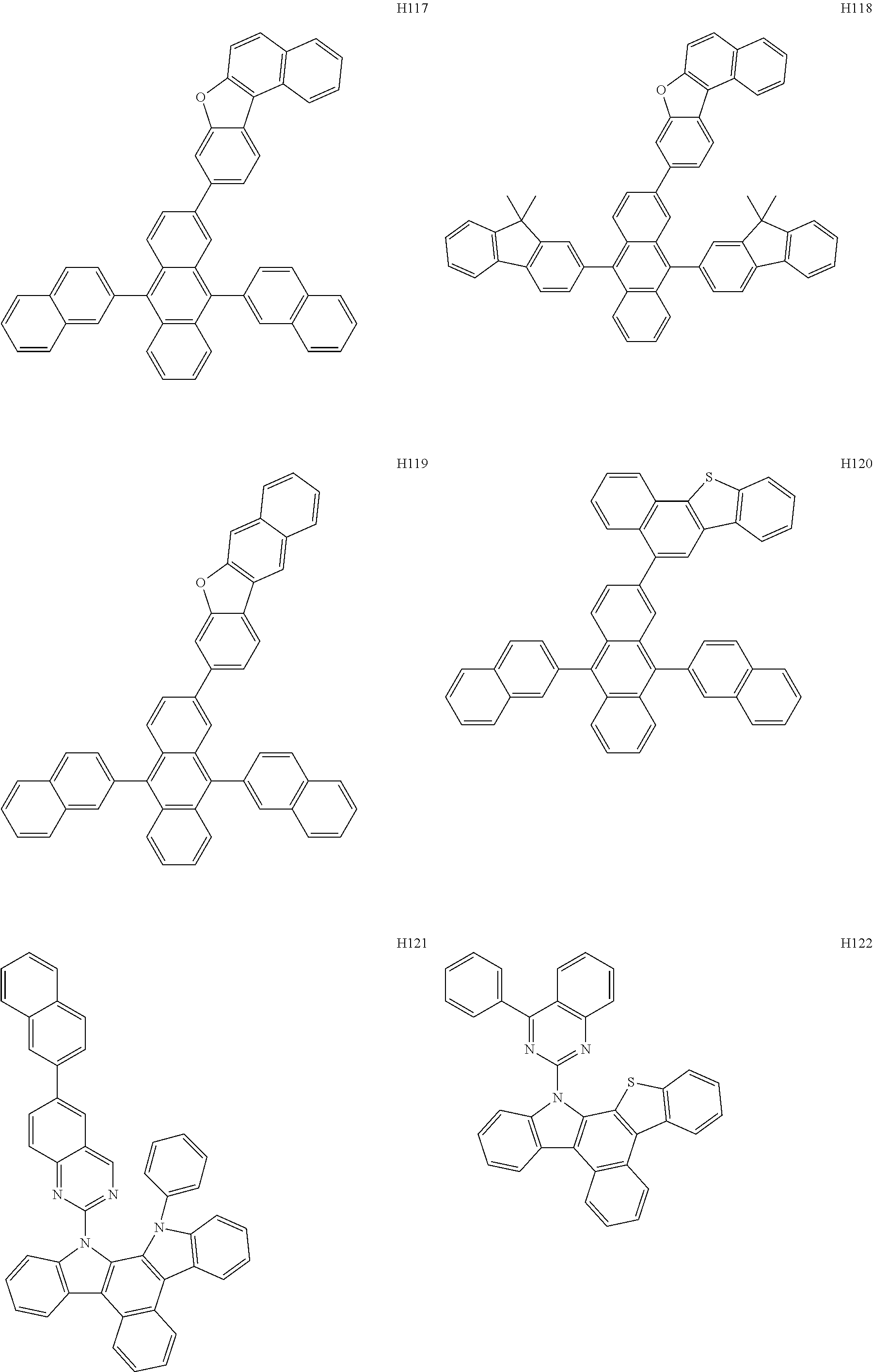

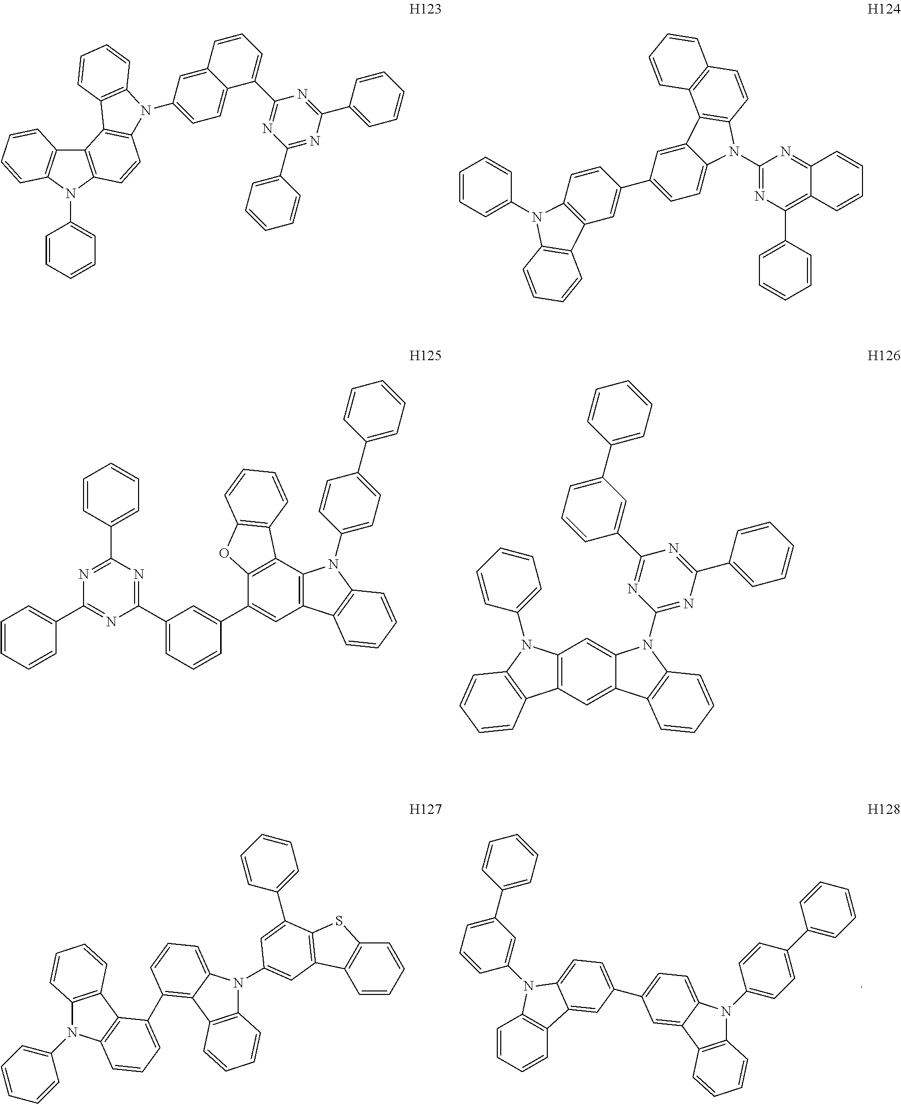

### Phosphorescent Dopant

The phosphorescent dopant may include at least one transition metal as a central metal.

The phosphorescent dopant may include a monodentate ligand, a bidentate ligand, a tridentate ligand, a tetradentate ligand, a pentadentate ligand, a hexadentate ligand, or a combination thereof.

In some embodiments, the phosphorescent dopant may be electrically neutral.

For example, in one or more embodiments, the phosphorescent dopant may include an organometallic compound represented by Formula 401:

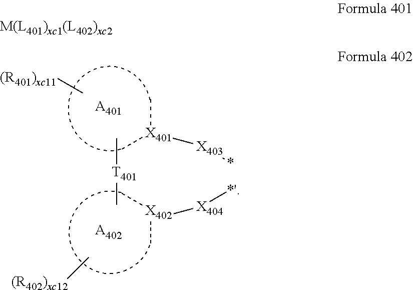

In Formulae 401 and 402,
M may be a transition metal (for example, iridium (Ir), platinum (Pt), palladium (Pd), osmium (Os), titanium (Ti), gold (Au), hafnium (Hf), europium (Eu), terbium (Tb), rhodium (Rh), rhenium (Re), or thulium (Tm)),
L₄₀₁ may be a ligand represented by Formula 402, and xc1 is 1, 2, or 3, wherein, when xc1 is 2 or more, two or more of L₄₀₁ may be identical to or different from each other,
L₄₀₂ may be an organic ligand, and xc2 may be 0, 1, 2, 3, or 4, wherein, when xc2 is 2 or more, two or more of L₄₀₂ may be identical to or different from each other,
X₄₀₁ and X₄₀₂ may each independently be N or C,
ring A₄₀₁ and ring A₄₀₂ may each independently be a C₃-C₆₀ carbocyclic group or a C₁-C₆₀ heterocyclic group,
T₄₀₁ may be a single bond, *—O—*′, *—S—*′, *—C(═O)—*′, *—N(Q₄₁₁)-*′, *—C(Q₄₁₁)(Q₄₁₂)-*′, *—C(Q₄₁₁)═C(Q₄₁₂)-*′, *—C(Q₄₁₁)=*′, or *=C=*′,
X₄₀₃ and X₄₀₄ may each independently be a chemical bond (for example, a covalent bond or a coordinate bond), O, S, N(Q₄₁₃), B(Q₄₁₃), P(Q₄₁₃), C(Q₄₁₃)(Q₄₁₄), or Si(Q₄₁₃) (Q₄₁₄),
Q₄₁₁ to Q₄₁₄ may each be the same as described respect to Q₁,
R₄₀₁ and R₄₀₂ may each independently be hydrogen, deuterium, —F, —Cl, —Br, —I, a hydroxyl group, a cyano group, a nitro group, a C₁-C₂₀ alkyl group unsubstituted or substituted with at least one R₁₀ₐ, a C₁-C₂₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, —Si(Q₄₀₁)(Q₄₀₂)(Q₄₀₃), —N(Q₄₀₁)(Q₄₀₂), —B(Q₄₀₁)(Q₄₀₂), —C(═O)(Q₄₀₁), —S(═O)₂(Q₄₀₁), or —P(═O)(Q₄₀₁)(Q₄₀₂),
Q₄₀₁ to Q₄₀₃ may each be the same as described with respect to Q₁,
xc11 and xc12 may each independently be an integer from 0 to 10, and
* and * in Formula 402 may each indicate a binding site to Min Formula 401.

For example, in one or more embodiments, in Formula 402, i) X₄₀₁ may be nitrogen and X₄₀₂ may be carbon, or ii) each of X₄₀₁ and X₄₀₂ may be nitrogen.

In one or more embodiments, when xc1 in Formula 401 is 2 or more, in two or more of L₄₀₁, two ring A₄₀₁(s) may optionally be linked to each other via T₄₀₂, which is a linking group, and/or two ring A₄₀₂(s) may optionally be linked to each other via T₄₀₃, which is a linking group. T₄₀₂ and T₄₀₃ may each be the same as described with respect to T₄₀₁.

In Formula 401, L₄₀₂ may be an organic ligand. For example, in one or more embodiments, L₄₀₂ may include a halogen, a diketone group (for example, an acetylacetonate group), a carboxylic acid group (for example, a picolinate group), —C(═O), an isonitrile group, a —CN group, a phosphorus group (for example, a phosphine group, a phosphite group, etc.), or a combination thereof.

In one or more embodiments, the phosphorescent dopant may include, for example, at least one selected from among Compounds PD1 to PD39, and/or a combination thereof:

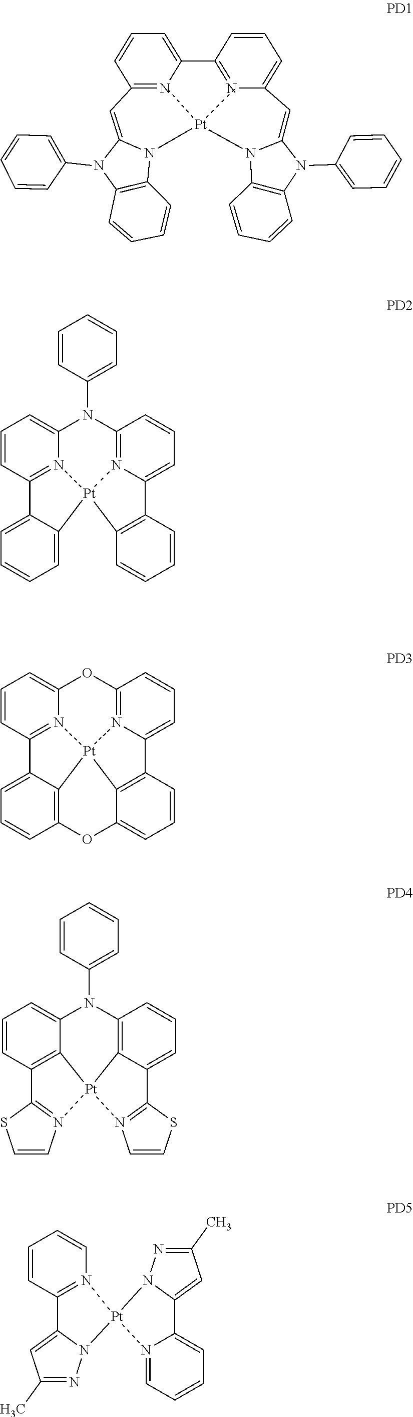

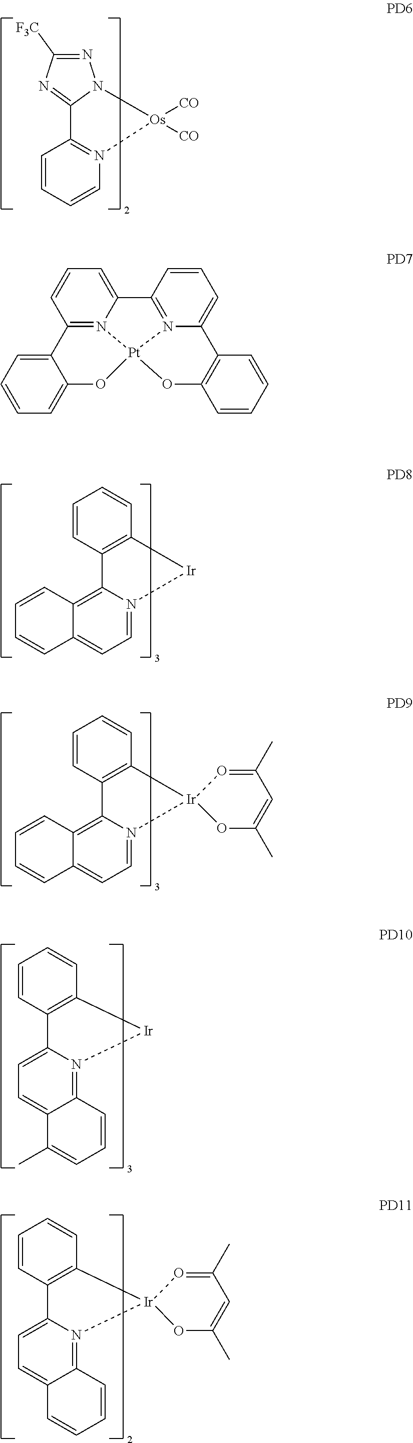

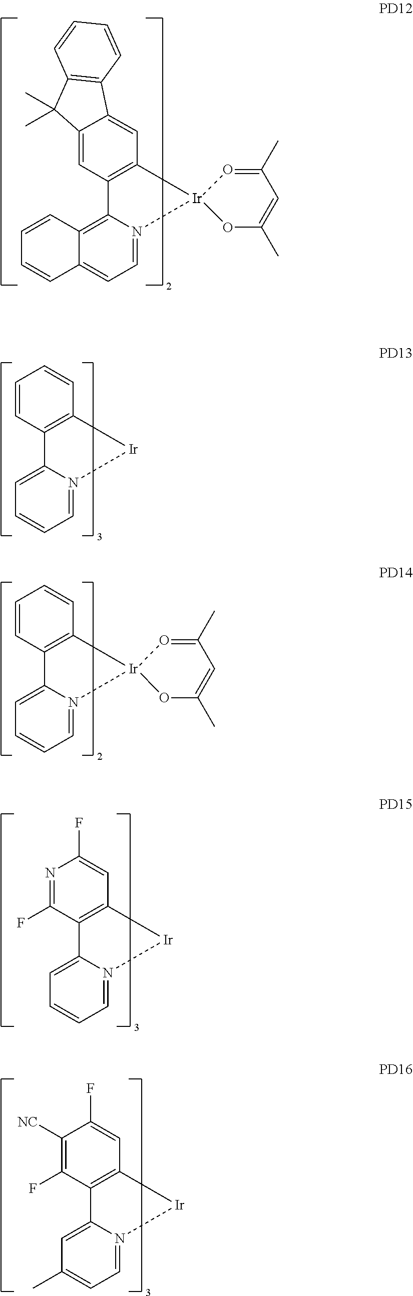

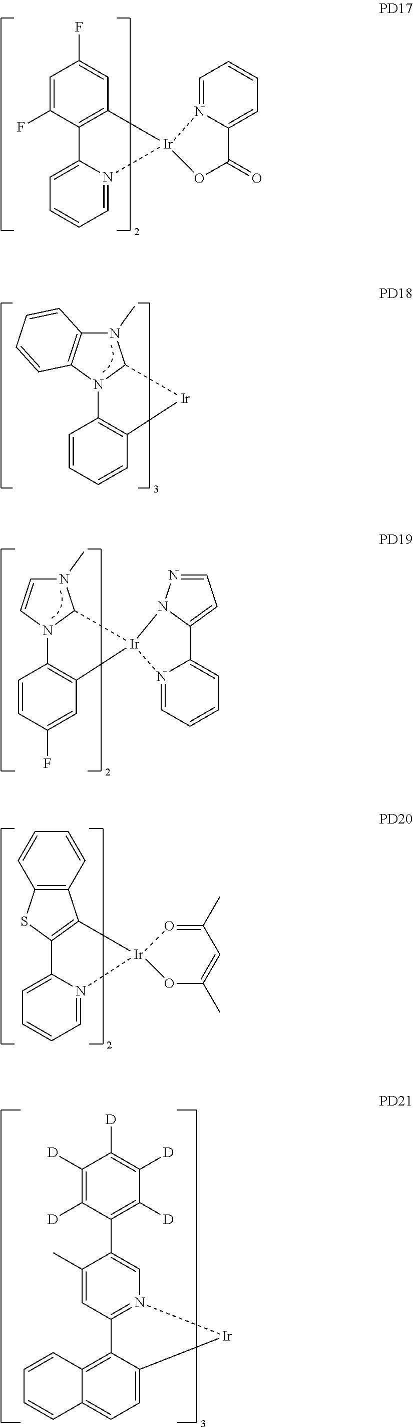

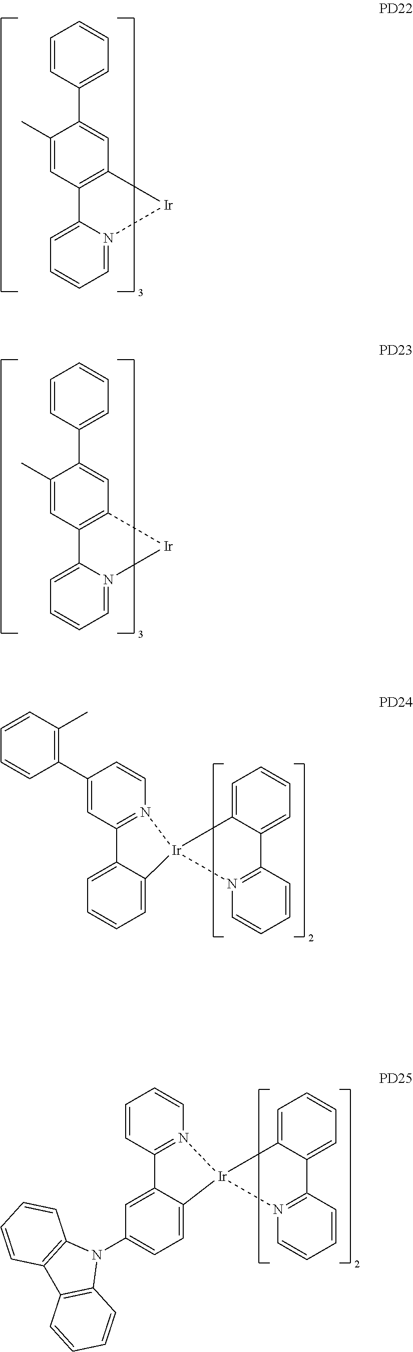

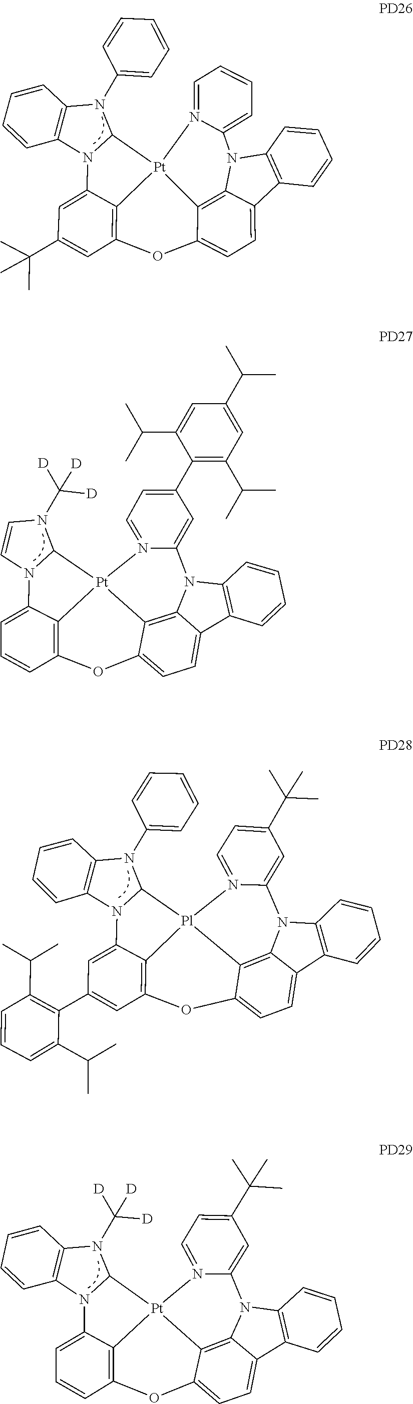

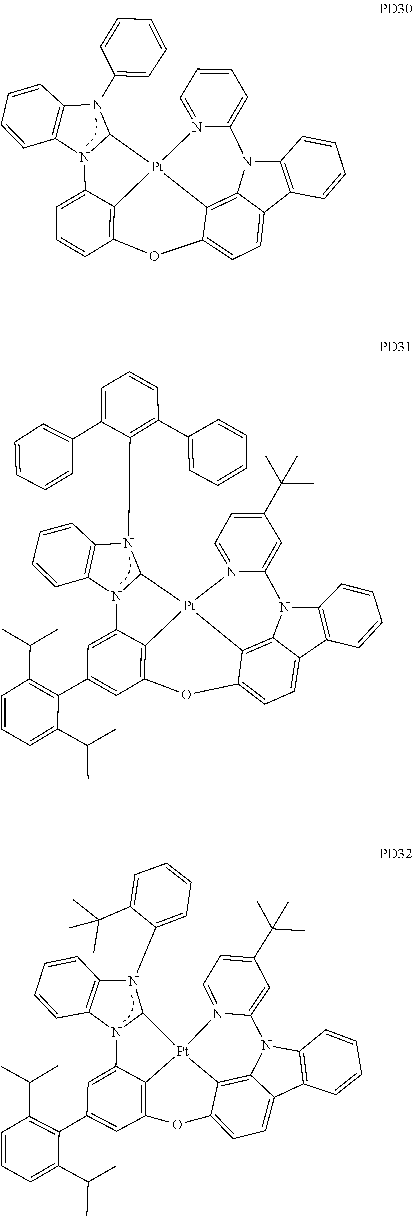

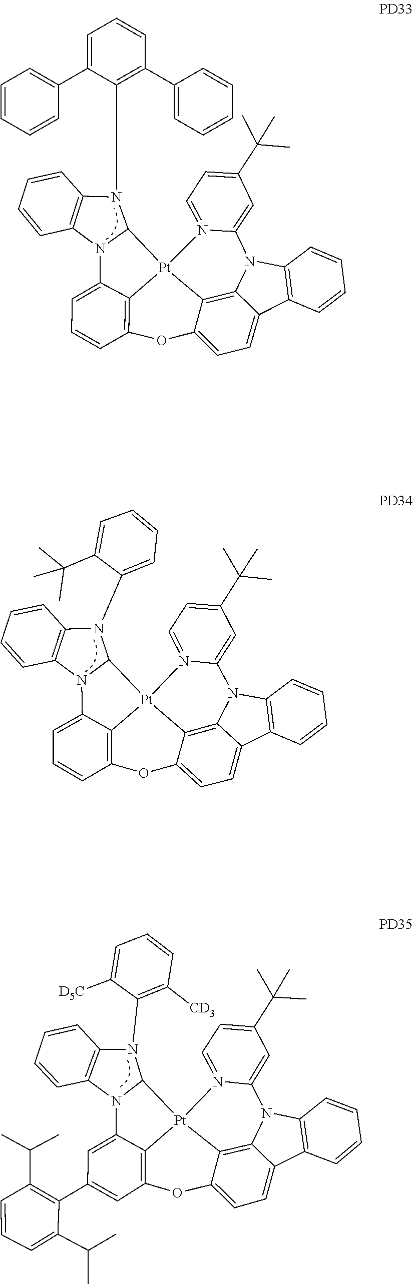

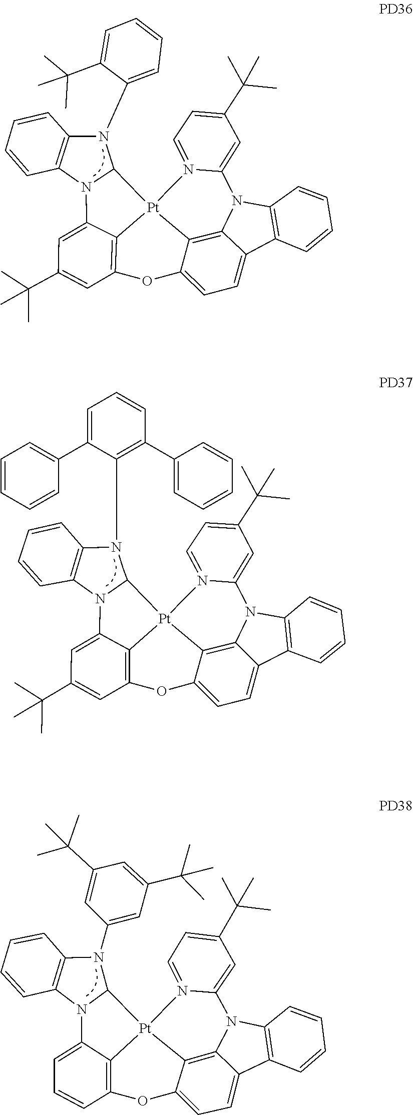

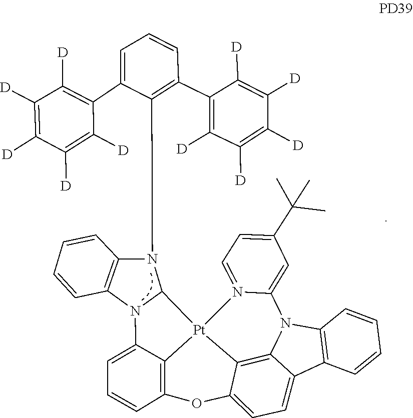

### Fluorescent Dopant

In one or more embodiments, the fluorescent dopant may include an amine group-containing compound, a styryl group-containing compound, or a combination thereof.

For example, in one or more embodiments, the fluorescent dopant may include a compound represented by Formula 501:

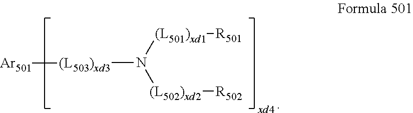

In Formula 501,
Ar₅₀₁, L₅₀₁ to L₅₀₃, R₅₀₁, and R₅₀₂ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
xd1 to xd3 may each independently be 0, 1, 2, or 3, and
xd4 may be 1, 2, 3, 4, 5, or 6.

In one or more embodiments, Ar₅₀₁ in Formula 501 may be a condensed cyclic group (for example, an anthracene group, a chrysene group, a pyrene group, etc.) in which three or more monocyclic groups are condensed together.

In one or more embodiments, xd4 in Formula 501 may be 2.

In one or more embodiments, the fluorescent dopant may include: at least one selected from among Compounds FD1 to FD37; 4,4′-bis(2,2-diphenylvinyl)-1,1′-biphenyl (DPVBi); 4,4′-bis[4-(N,N-diphenylamino)styryl]biphenyl (DPAVBi); and/or a combination thereof:

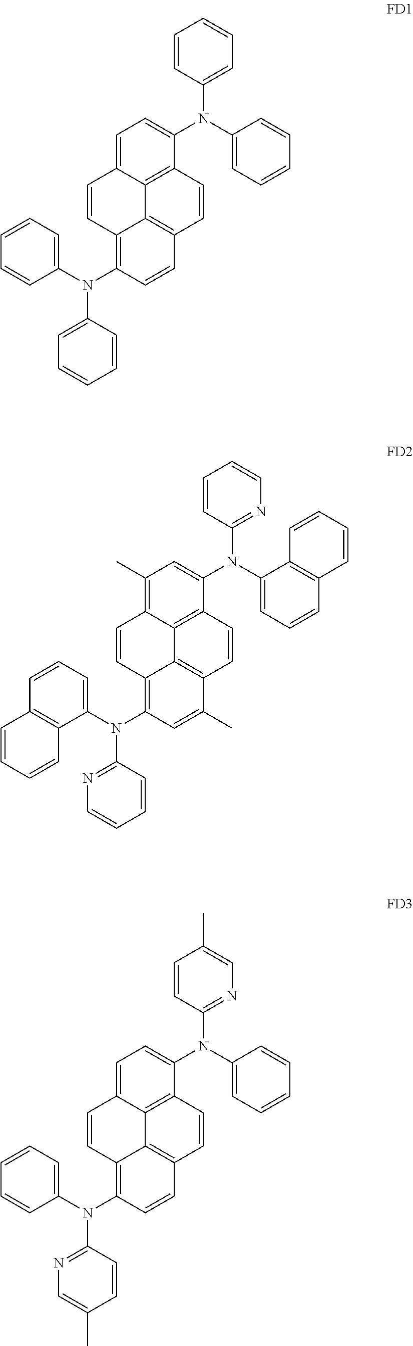 [SMILES: c1ccc(N(c2ccccc2)c2ccc3ccc4c(N(c5ccccc5)c5ccccc5)ccc5ccc2c3c54)cc1 ; Cc1cc(N(c2ccccn2)c2cccc3ccccc23)c2ccc3c(C)cc(N(c4ccccn4)c4cccc5ccccc45)c4ccc1c2c34 ; Cc1ccc(N(c2ccccc2)c2ccc3ccc4c(N(c5ccccc5)c5ccc(C)cn5)ccc5ccc2c3c54)nc1]

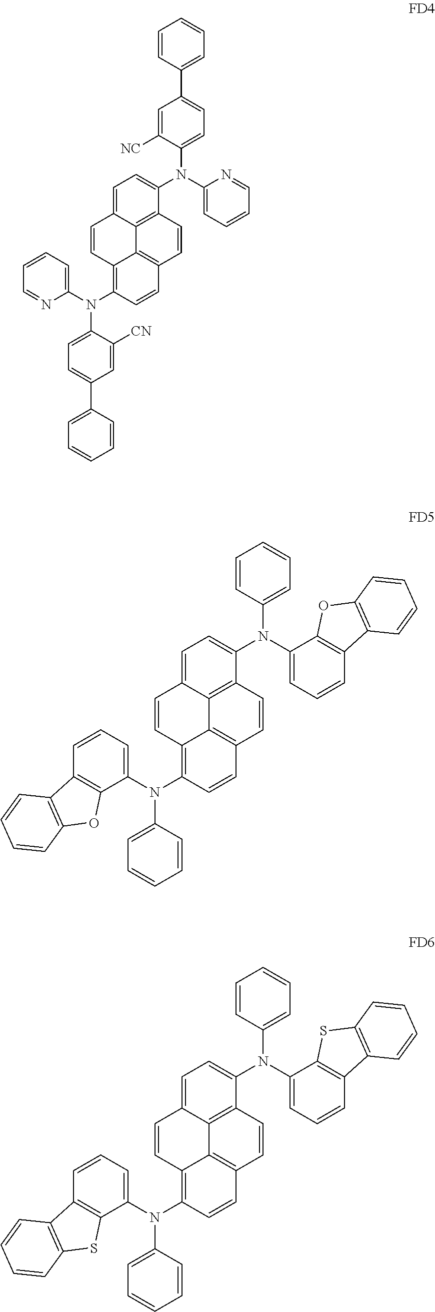 [SMILES: N#Cc1cc(-c2ccccc2)ccc1N(c1ccccn1)c1ccc2ccc3c(N(c4ccccn4)c4ccc(-c5ccccc5)cc4C#N)ccc4ccc1c2c43 ; c1ccc(N(c2ccc3ccc4c(N(c5ccccc5)c5cccc6c5oc5ccccc56)ccc5ccc2c3c54)c2cccc3c2oc2ccccc23)cc1 ; c1ccc(N(c2ccc3ccc4c(N(c5ccccc5)c5cccc6c5sc5ccccc56)ccc5ccc2c3c54)c2cccc3c2sc2ccccc23)cc1]

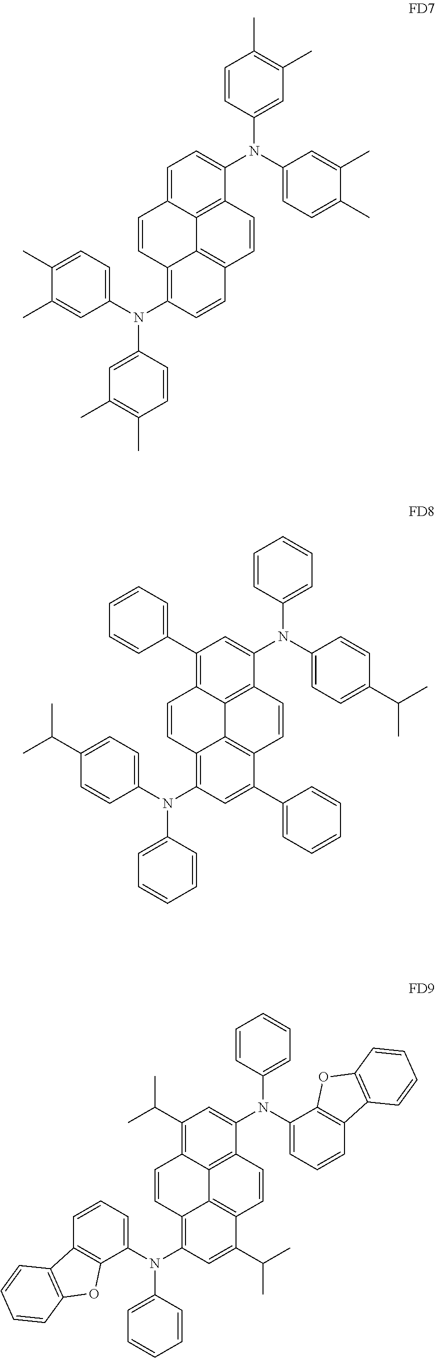

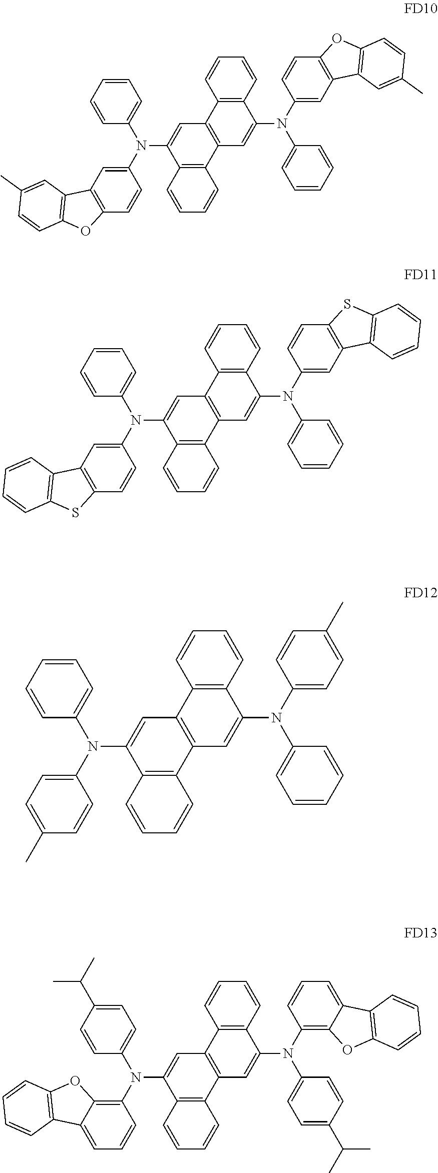

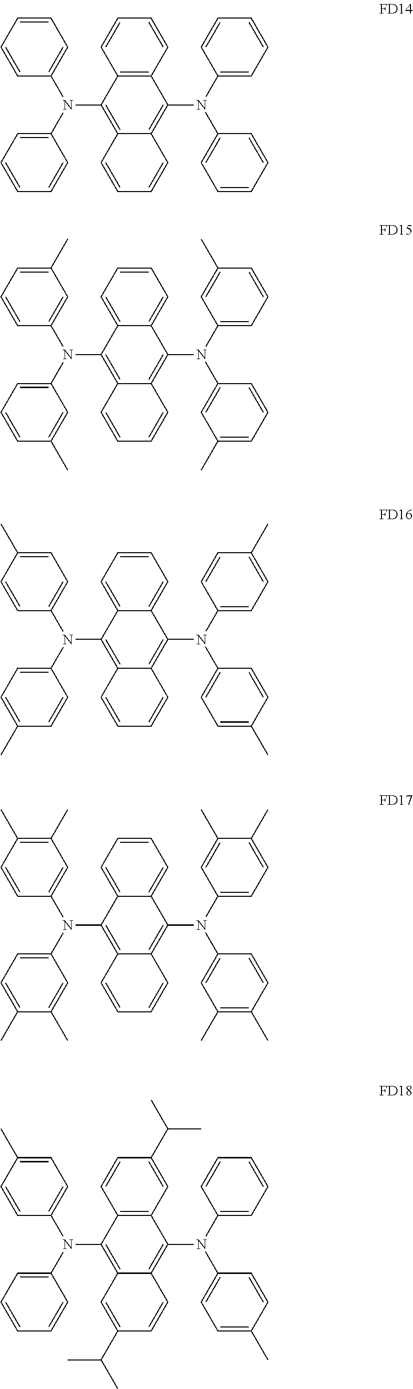 [SMILES: c1ccc(N(c2ccccc2)c2c3ccccc3c(N(c3ccccc3)c3ccccc3)c3ccccc23)cc1 ; Cc1cccc(N(c2cccc(C)c2)c2c3ccccc3c(N(c3cccc(C)c3)c3cccc(C)c3)c3ccccc23)c1 ; Cc1ccc(N(c2ccc(C)cc2)c2c3ccccc3c(N(c3ccc(C)cc3)c3ccc(C)cc3)c3ccccc23)cc1 ; Cc1ccc(N(c2ccc(C)c(C)c2)c2c3ccccc3c(N(c3ccc(C)c(C)c3)c3ccc(C)c(C)c3)c3ccccc23)cc1C ; Cc1ccc(N(c2ccccc2)c2c3ccc(C(C)C)cc3c(N(c3ccccc3)c3ccc(C)cc3)c3ccc(C(C)C)cc23)cc1]

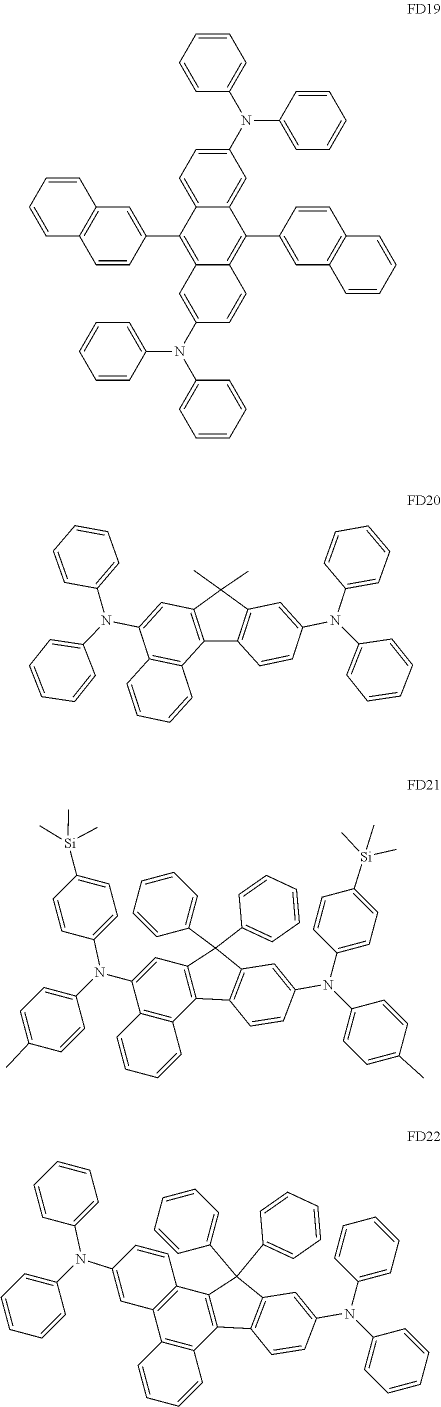

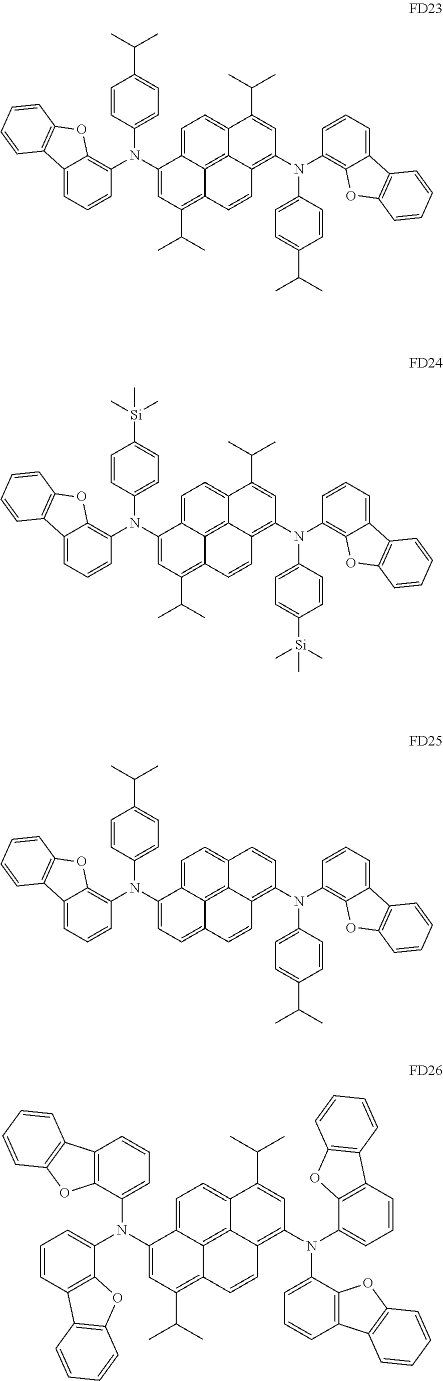 [SMILES: CC(C)c1ccc(N(c2cc(C(C)C)c3ccc4c(N(c5ccc(C(C)C)cc5)c5cccc6c5oc5ccccc56)cc(C(C)C)c5ccc2c3c54)c2cccc3c2oc2ccccc23)cc1 ; CC(C)c1cc(N(c2ccc([Si](C)(C)C)cc2)c2cccc3c2oc2ccccc23)c2ccc3c(C(C)C)cc(N(c4ccc([Si](C)(C)C)cc4)c4cccc5c4oc4ccccc45)c4ccc1c2c34 ; CC(C)c1ccc(N(c2ccc3ccc4c(N(c5ccc(C(C)C)cc5)c5cccc6c5oc5ccccc56)ccc5ccc2c3c54)c2cccc3c2oc2ccccc23)cc1 ; CC(C)c1cc(N(c2cccc3c2oc2ccccc23)c2cccc3c2oc2ccccc23)c2ccc3c(C(C)C)cc(N(c4cccc5c4oc4ccccc45)c4cccc5c4oc4ccccc45)c4ccc1c2c34]

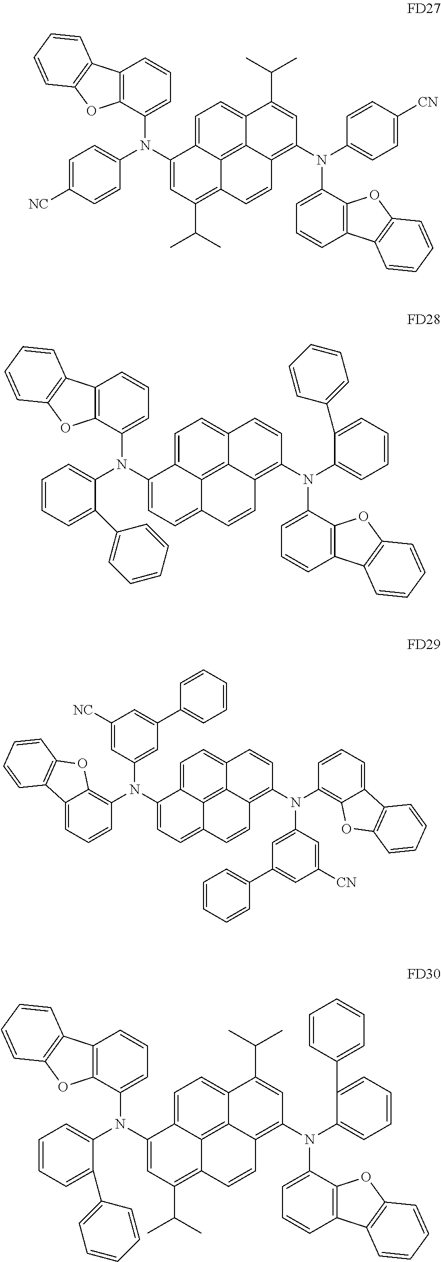 [SMILES: CC(C)c1cc(N(c2ccc(C#N)cc2)c2cccc3c2oc2ccccc23)c2ccc3c(C(C)C)cc(N(c4ccc(C#N)cc4)c4cccc5c4oc4ccccc45)c4ccc1c2c34 ; c1ccc(-c2ccccc2N(c2ccc3ccc4c(N(c5ccccc5-c5ccccc5)c5cccc6c5oc5ccccc56)ccc5ccc2c3c54)c2cccc3c2oc2ccccc23)cc1 ; N#Cc1cc(-c2ccccc2)cc(N(c2ccc3ccc4c(N(c5cc(C#N)cc(-c6ccccc6)c5)c5cccc6c5oc5ccccc56)ccc5ccc2c3c54)c2cccc3c2oc2ccccc23)c1 ; CC(C)c1cc(N(c2ccccc2-c2ccccc2)c2cccc3c2oc2ccccc23)c2ccc3c(C(C)C)cc(N(c4ccccc4-c4ccccc4)c4cccc5c4oc4ccccc45)c4ccc1c2c34]

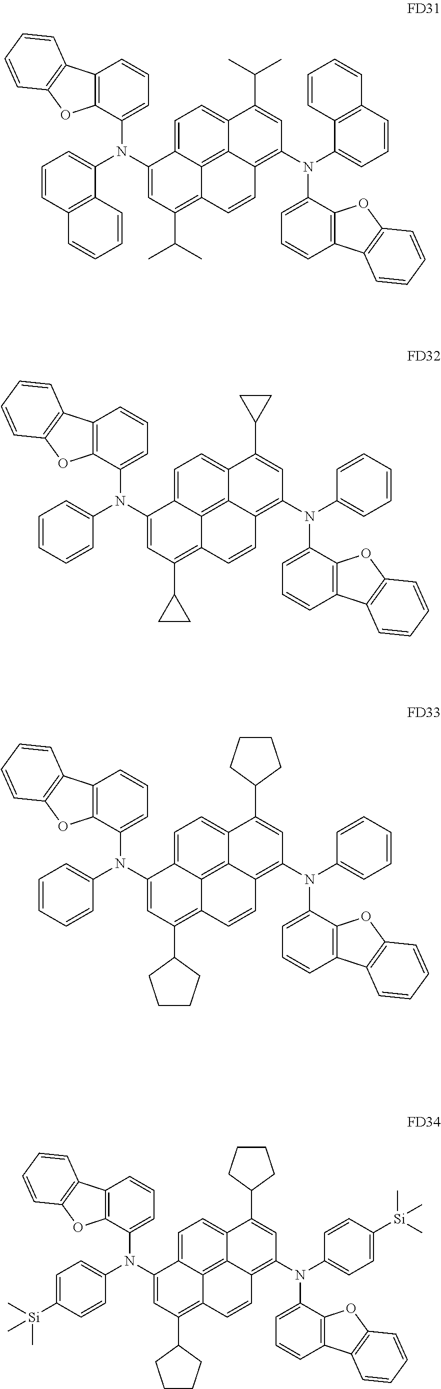

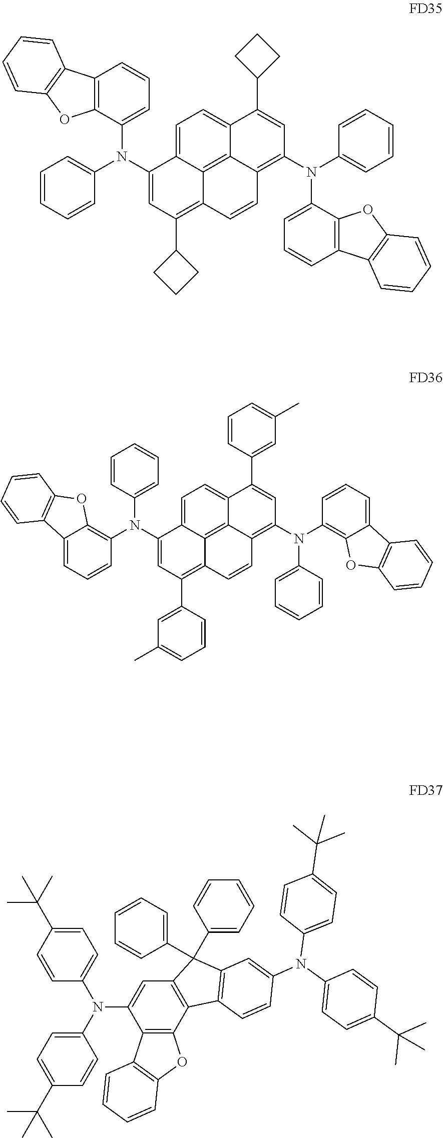 [SMILES: c1ccc(N(c2cc(C3CCC3)c3ccc4c(N(c5ccccc5)c5cccc6c5oc5ccccc56)cc(C5CCC5)c5ccc2c3c54)c2cccc3c2oc2ccccc23)cc1 ; Cc1cccc(-c2cc(N(c3ccccc3)c3cccc4c3oc3ccccc34)c3ccc4c(-c5cccc(C)c5)cc(N(c5ccccc5)c5cccc6c5oc5ccccc56)c5ccc2c3c45)c1 ; CC(C)(C)c1ccc(N(c2ccc(C(C)(C)C)cc2)c2ccc3c(c2)C(c2ccccc2)(c2ccccc2)c2cc(N(c4ccc(C(C)(C)C)cc4)c4ccc(C(C)(C)C)cc4)c4c(oc5ccccc54)c2-3)cc1]

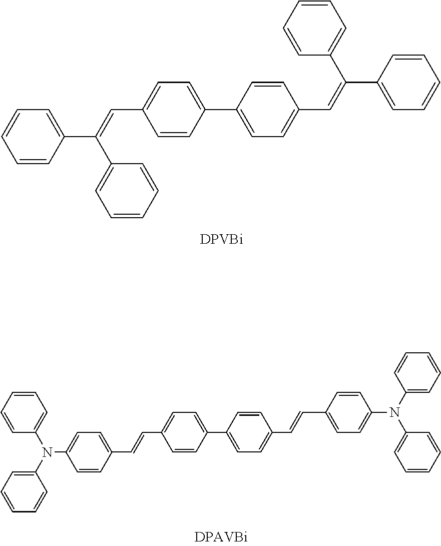

### Delayed Fluorescence Material

In one or more embodiments, the emission layer may include a delayed fluorescence material.

In the present disclosure, the delayed fluorescence material may be selected from compounds capable of emitting delayed fluorescence based on a delayed fluorescence emission mechanism.

The delayed fluorescence material included in the emission layer may act as a host or a dopant depending on the type or kind of other materials included in the emission layer.

In one or more embodiments, a difference between a triplet energy level (eV) of the delayed fluorescence material and a singlet energy level (eV) of the delayed fluorescence material may be greater than or equal to 0 eV and less than or equal to 0.5 eV. When the difference between the triplet energy level (eV) of the delayed fluorescence material and the singlet energy level (eV) of the delayed fluorescence material is satisfied within the range above, up-conversion from the triplet state to the singlet state of the delayed fluorescence materials may effectively occur, and thus, the light-emitting device 10 may have improved luminescence efficiency.

For example, in some embodiments, the delayed fluorescence material may include: i) a material including at least one electron donor (for example, a π electron-rich C₃-C₆₀ cyclic group and/or the like, such as a carbazole group) and at least one electron acceptor (for example, a sulfoxide group, a cyano group, a π electron-deficient nitrogen-containing C₁-C₆₀ cyclic group, and/or the like), ii) a material including a C₈-C₆₀ polycyclic group including at least two cyclic groups condensed to each other while sharing boron (B), and/or the like.

Non-limiting examples of the delayed fluorescence material may include at least one selected from among Compounds DF1 to DF14:

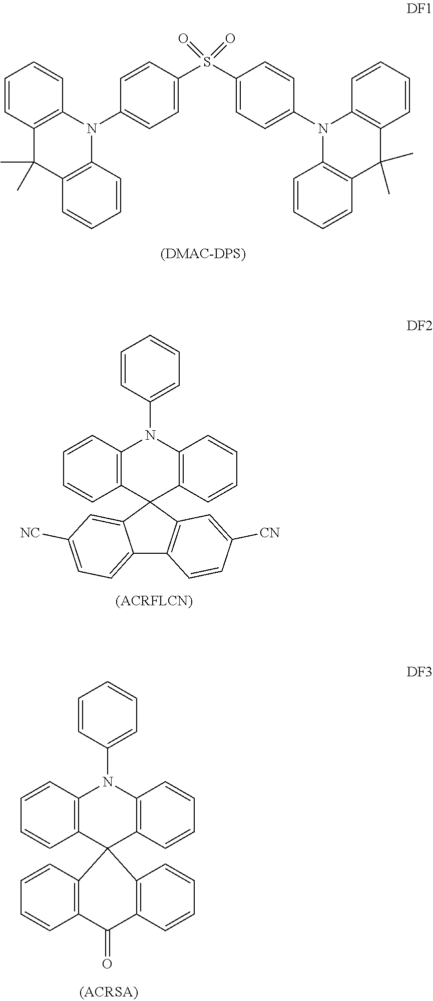

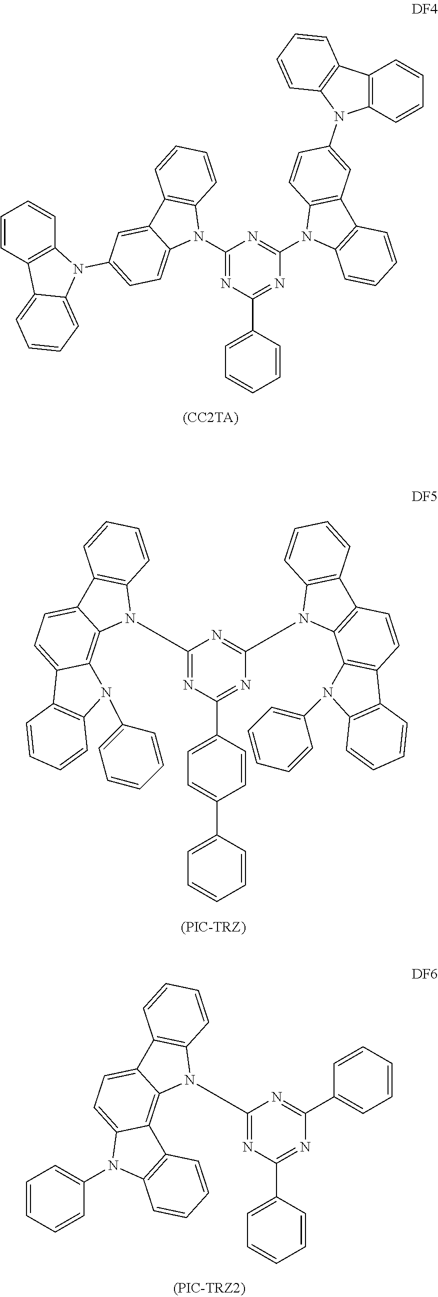 [SMILES: c1ccc(-c2nc(-n3c4ccccc4c4cc(-n5c6ccccc6c6ccccc65)ccc43)nc(-n3c4ccccc4c4cc(-n5c6ccccc6c6ccccc65)ccc43)n2)cc1 ; c1ccc(-c2ccc(-c3nc(-n4c5ccccc5c5ccc6c7ccccc7n(-c7ccccc7)c6c54)nc(-n4c5ccccc5c5ccc6c7ccccc7n(-c7ccccc7)c6c54)n3)cc2)cc1 ; c1ccc(-c2nc(-c3ccccc3)nc(-n3c4ccccc4c4ccc5c(c6ccccc6n5-c5ccccc5)c43)n2)cc1]

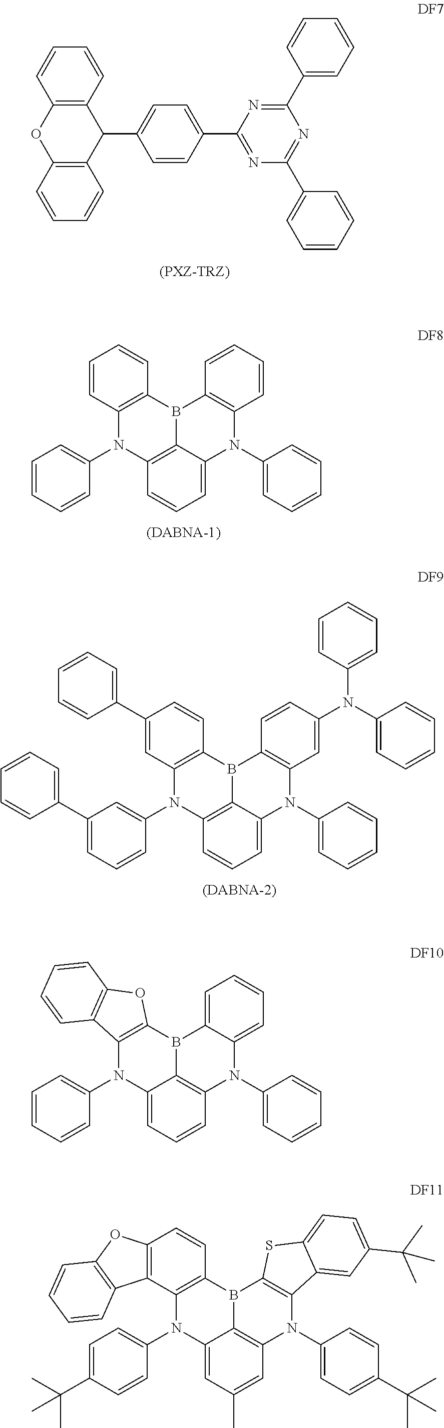

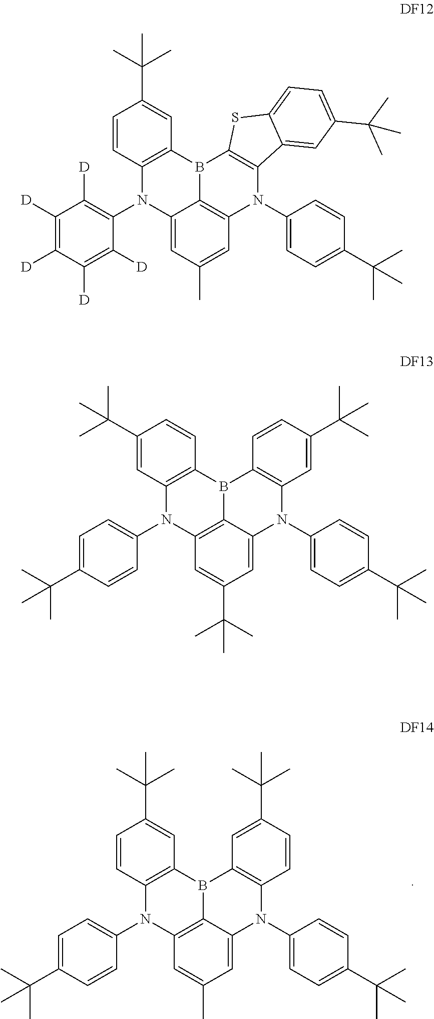

### Quantum Dot

In one or more embodiments, the emission layer may include a quantum dot.

The term “quantum dot” as utilized herein may refer to a crystal of a semiconductor compound, and may include any material capable of emitting light of one or more suitable emission wavelengths according to the size of the crystal.

A diameter of the quantum dot may be, for example, in a range of about 1 nm to about 10 nm.

The quantum dot may be synthesized by a wet chemical process, a metal organic chemical vapor deposition process, a molecular beam epitaxy process, or any process similar thereto.

The wet chemical process is a method including mixing a precursor material with an organic solvent and then growing quantum dot particle crystals. When the crystal grows, the organic solvent naturally acts as a dispersant coordinated on the surface of the quantum dot crystal and controls the growth of the crystal so that the growth of quantum dot particles can be controlled or selected through a process which costs lower, and is easier than vapor deposition methods, such as metal organic chemical vapor deposition (MOCVD) or molecular beam epitaxy (MBE).

The quantum dot may include: a Group II-VI semiconductor compound; a Group III-V semiconductor compound; a Group III-VI semiconductor compound; a Group I-III-VI semiconductor compound; a Group IV-VI semiconductor compound; a Group IV element or compound; or a combination thereof.

Non-limiting examples of the Group II-VI semiconductor compound may be: a binary compound, such as CdS, CdSe, CdTe, ZnS, ZnSe, ZnTe, ZnO, HgS, HgSe, HgTe, MgSe, MgS, and/or the like; a ternary compound, such as CdSeS, CdSeTe, CdSTe, ZnSeS, ZnSeTe, ZnSTe, HgSeS, HgSeTe, HgSTe, CdZnS, CdZnSe, CdZnTe, CdHgS, CdHgSe, CdHgTe, HgZnS, HgZnSe, HgZnTe, MgZnSe, MgZnS, and/or the like; a quaternary compound, such as CdZnSeS, CdZnSeTe, CdZnSTe, CdHgSeS, CdHgSeTe, CdHgSTe, HgZnSeS, HgZnSeTe, HgZnSTe, and/or the like; and/or a combination thereof.

Non-limiting examples of the Group III-V semiconductor compound may be: a binary compound, such as GaN, GaP, GaAs, GaSb, AlN, AlP, AlAs, AlSb, InN, InP, InAs, InSb, and/or the like; a ternary compound, such as GaNP, GaNAs, GaNSb, GaPAs, GaPSb, AlNP, AlNAs, AlNSb, AIPAs, AIPSb, InGaP, InNP, InAIP, InNAs, InNSb, InPAs, InPSb, and/or the like; a quaternary compound, such as GaAINP, GaAlNAs, GaAlNSb, GaAlPAs, GaAlPSb, GaInNP, GaInNAs, GaInNSb, GaInPAs, GaInPSb, InAlNP, InAlNAs, InAlNSb, InAlPAs, InAlPSb, and/or the like; and/or a combination thereof. In one or more embodiments, the Group III-V semiconductor compound may further include a Group II element. Non-limiting examples of the Group III-V semiconductor compound further including the Group II element may be InZnP, InGaZnP, InAIZnP, and/or the like.

Non-limiting examples of the Group III-VI semiconductor compound may be: a binary compound, such as GaS, GaSe, Ga₂Se₃, GaTe, InS, InSe, In₂S₃, In₂Se₃, InTe, and/or the like; a ternary compound, such as InGaS₃, InGaSe₃, and/or the like; and/or a combination thereof.

Non-limiting examples of the Group I-III-VI semiconductor compound may be: a ternary compound, such as AgInS, AgInS₂, CuInS, CuInS₂, CuGaO₂, AgGaO₂, AgAlO₂, and/or the like; and/or a combination thereof.

Non-limiting examples of the Group IV-VI semiconductor compound may be: a binary compound, such as SnS, SnSe, SnTe, PbS, PbSe, PbTe, and/or the like; a ternary compound, such as SnSeS, SnSeTe, SnSTe, PbSeS, PbSeTe, PbSTe, SnPbS, SnPbSe, SnPbTe, and/or the like; a quaternary compound, such as SnPbSSe, SnPbSeTe, SnPbSTe, and/or the like; and/or a combination thereof.

Non-limiting examples of the Group IV element or compound may be: a single element compound, such as Si, Ge, and/or the like; a binary compound, such as SiC, SiGe, and/or the like; and/or a combination thereof.

Each element included in a multi-element compound, such as in the binary compound, the ternary compound, and the quaternary compound, may be present at a substantially uniform concentration or non-substantially uniform concentration in a particle.

In one or more embodiments, the quantum dot may have a single structure in which the concentration of each element in the quantum dot is substantially uniform, or may have a core-shell dual structure. For example, a material included in the core and a material included in the shell may be different from each other.

The shell of the quantum dot may act as a protective layer which prevents or reduce chemical denaturation of the core to maintain semiconductor characteristics, and/or as a charging layer which impart electrophoretic characteristics to the quantum dot. The shell may be single-layered or multi-layered. The interface between the core and the shell may have a concentration gradient in which the concentration of an element existing in the shell decreases toward the center of the core.

Non-limiting examples of the shell of the quantum dot may be an oxide of metal, metalloid, or non-metal, a semiconductor compound, or a combination thereof. Non-limiting examples of the oxide of metal, metalloid, or non-metal may be: a binary compound, such as SiO₂, Al₂O₃, TiO₂, ZnO, MnO, Mn₂O₃, Mn₃O₄, CuO, FeO, Fe₂O₃, Fe₃O₄, CoO, Co₃O₄, NiO, and/or the like; a ternary compound, such as MgAl₂O₄, CoFe₂O₄, NiFe₂O₄, CoMn₂O₄, and/or the like; and/or a combination thereof. Non-limiting examples of the semiconductor compound may be: as described herein, a Group II-VI semiconductor compound; a Group III-V semiconductor compound; a Group III-VI semiconductor compound; a Group I-III-VI semiconductor compound; a Group IV-VI semiconductor compound; and/or a combination thereof. Non-limiting examples of the semiconductor compound suitable for the shell may be CdS, CdSe, CdTe, ZnS, ZnSe, ZnTe, ZnSeS, ZnTeS, GaAs, GaP, GaSb, HgS, HgSe, HgTe, InAs, InP, InGaP, InSb, AlAs, AlP, AlSb, or a combination thereof.

The quantum dot may have a full width at half maximum (FWHM) of the emission spectrum of less than or equal to about 45 nm, less than or equal to about 40 nm, or for example, less than or equal to about 30 nm. When the FWHM of the emission spectrum of the quantum dot is within these ranges, the quantum dot may have improved color purity or improved color reproducibility. In some embodiments, because light emitted through the quantum dot is emitted in all directions, the wide viewing angle may be improved.

In some embodiments, the quantum dot may be in the form of substantially spherical, pyramidal, multi-arm, or cubic nanoparticles, nanotubes, nanowires, nanofibers, and/or nanoplate particles.

Because the energy band gap may be adjusted by controlling or selecting the size of the quantum dot, light having one or more suitable wavelength bands may be obtained from a quantum dot emission layer. Accordingly, by utilizing quantum dots of different sizes, a light-emitting device that emits light of one or more suitable wavelengths may be implemented. In one or more embodiments, the size of the quantum dots may be selected to emit red light, green light, and/or blue light. In some embodiments, the size of the quantum dots may be configured to emit white light by combination of light of one or more suitable colors.

### Electron Transport Region in Interlayer 130

The electron transport region may have: i) a single-layer structure including (e.g., consisting of) a single layer including (e.g., consisting of) a single material, ii) a single-layer structure including (e.g., consisting of) a single layer including (e.g., consisting of) multiple different materials, or iii) a multi-layer structure including multiple layers including different materials.

The electron transport region may include a buffer layer, a hole blocking layer, an electron control layer, an electron transport layer, an electron injection layer, or a combination thereof.

For example, in one or more embodiments, the electron transport region may have an electron transport layer/electron injection layer structure, a hole blocking layer/electron transport layer/electron injection layer structure, an electron control layer/electron transport layer/electron injection layer structure, or a buffer layer/electron transport layer/electron injection layer structure, wherein constituent layers of each structure are sequentially stacked from the emission layer in each stated order.

In one or more embodiments, the electron transport region (for example, the buffer layer, the hole blocking layer, the electron control layer, or the electron transport layer in the electron transport region) may include a metal-free compound including at least one π electron-deficient nitrogen-containing C₁-C₆₀ cyclic group.

For example, in some embodiments, the electron transport region may include a compound represented by Formula 601:

[Ar₆₀₁]ₓₑ₁₁-[(L₆₀₁)ₓₑ₁-R₆₀₁]ₓₑ₂₁ Formula 601

In Formula 601,
Ar₆₀₁ and L₆₀₁ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
xe11 may be 1, 2, or 3,
xe1 may be 0, 1, 2, 3, 4, or 5,
R₆₀₁ may be a C₃-C₆₀ carbocyclic group unsubstituted or substituted with at least one R₁₀ₐ, a C₁-C₆₀ heterocyclic group unsubstituted or substituted with at least one R₁₀ₐ, —Si(Q₆₀₁)(Q₆₀₂)(Q₆₀₃), —C(═O)(Q₆₀₁), —S(═O)₂(Q₆₀₁), or —P(═O)(Q₆₀₁)(Q₆₀₂),
Q₆₀₁ to Q₆₀₃ may each be the same as described with respect to Q₁,
xe21 may be 1, 2, 3, 4, or 5, and
at least one selected from among Ar₆₀₁, L₆₀₁, and R₆₀₁ may each independently be a π electron-deficient nitrogen-containing C₁-C₆₀ cyclic group unsubstituted or substituted with at least one R₁₀ₐ.

In one or more embodiments, when xe11 in Formula 601 is 2 or more, two or more of Ar₆₀₁(s) may be linked to each other via a single bond.

In some embodiments, Ar₆₀₁ in Formula 601 may be a substituted or unsubstituted anthracene group.

In one or more embodiments, the electron transport region may include a compound represented by Formula 601-1:

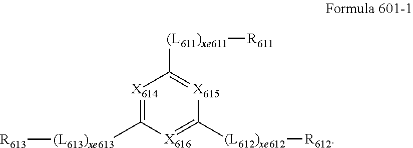

In Formula 601-1,
X₆₁₄ may be N or C(R₆₁₄), X₆₁₅ may be N or C(R₆₁₅), X₆₁₆ may be N or C(R₆₁₆), and at least one selected from among X₆₁₄ to X₆₁₆ may be N,
L₆₁₁ to L₆₁₃ may each be the same as described with respect to L₆₀₁,
xe611 to xe613 may each be the same as described with respect to xe1,
R₆₁₁ to R₆₁₃ may each be the same as described with respect to R₆₀₁, and
R₆₁₄ to R₆₁₆ may each independently be hydrogen, deuterium, —F, —Cl, —Br, —I, a hydroxyl group, a cyano group, a nitro group, a C₁-C₂₀ alkyl group, a C₁-C₂₀ alkoxy group, a C₃-C₆₀ carbocyclic group unsubstituted or substituted with at least one R₁₀ₐ, or a C₁-C₆₀ heterocyclic group unsubstituted or substituted with at least one R₁₀ₐ.

For example, in some embodiments, xe1 and xe611 to xe613 in Formulae 601 and 601-1 may each independently be 0, 1, or 2.

In one or more embodiments, the electron transport region may include at least one selected from among Compounds ET1 to ET45, 2,9-dimethyl-4,7-diphenyl-1,10-phenanthroline (BCP), 4,7-diphenyl-1,10-phenanthroline (Bphen), tris(8-hydroxyquinolinato)aluminum (Alq₃), bis(2-methyl-8-quinolinolato-N1,O8)-(1,1′-biphenyl-4-olato)aluminum (BAlq), 3-(4-biphenyl)-4-phenyl-5-tert-butylphenyl-1,2,4-triazole (TAZ), 4-(naphthalen-1-yl)-3,5-diphenyl-4H-1,2,4-triazole (NTAZ), and/or a combination thereof:

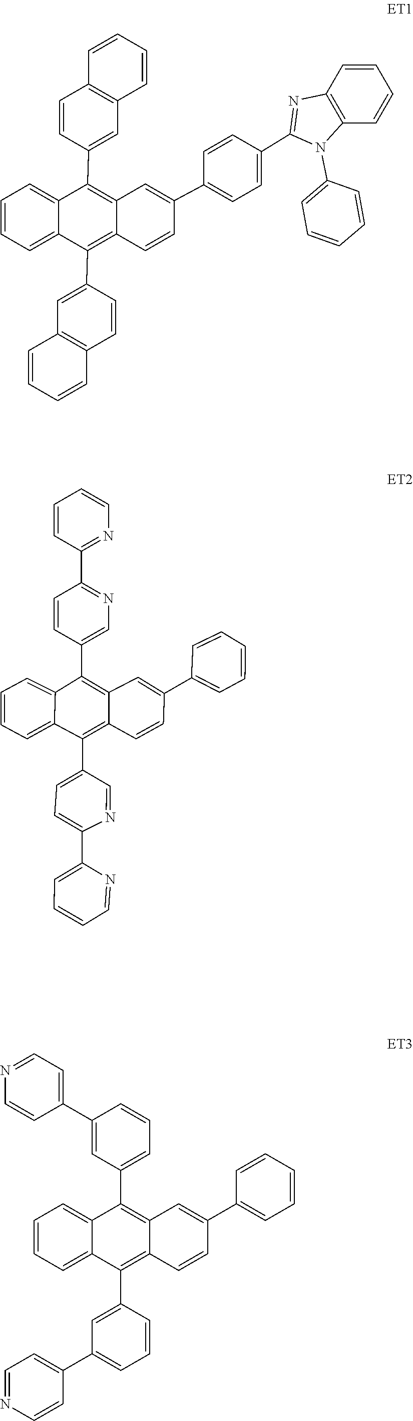 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1 ; c1ccc(-c2ccc3c(-c4ccc(-c5ccccn5)nc4)c4ccccc4c(-c4ccc(-c5ccccn5)nc4)c3c2)cc1 ; c1ccc(-c2ccc3c(-c4cccc(-c5ccncc5)c4)c4ccccc4c(-c4cccc(-c5ccncc5)c4)c3c2)cc1]

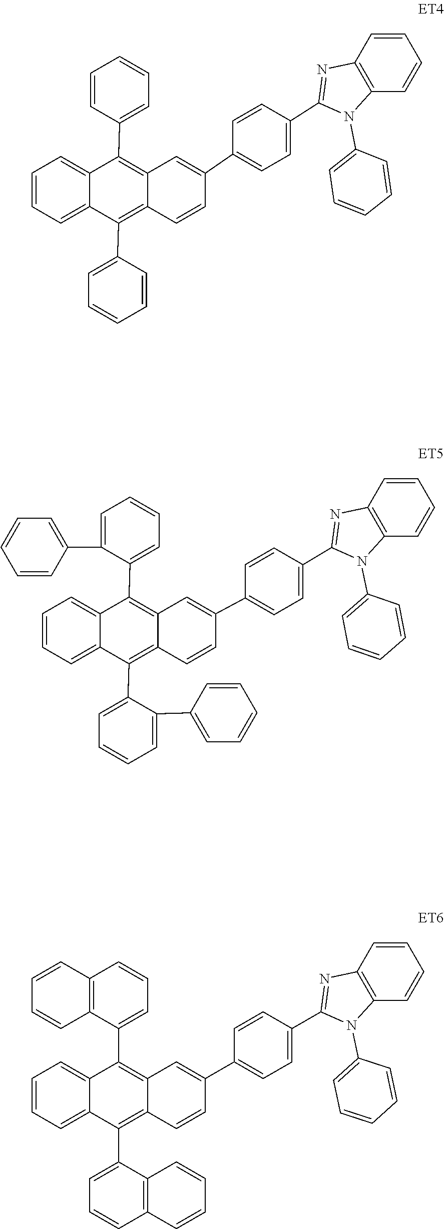 [SMILES: c1ccc(-c2c3ccccc3c(-c3ccccc3)c3cc(-c4ccc(-c5nc6ccccc6n5-c5ccccc5)cc4)ccc23)cc1 ; c1ccc(-c2ccccc2-c2c3ccccc3c(-c3ccccc3-c3ccccc3)c3cc(-c4ccc(-c5nc6ccccc6n5-c5ccccc5)cc4)ccc23)cc1 ; c1ccc(-n2c(-c3ccc(-c4ccc5c(-c6cccc7ccccc67)c6ccccc6c(-c6cccc7ccccc67)c5c4)cc3)nc3ccccc32)cc1]

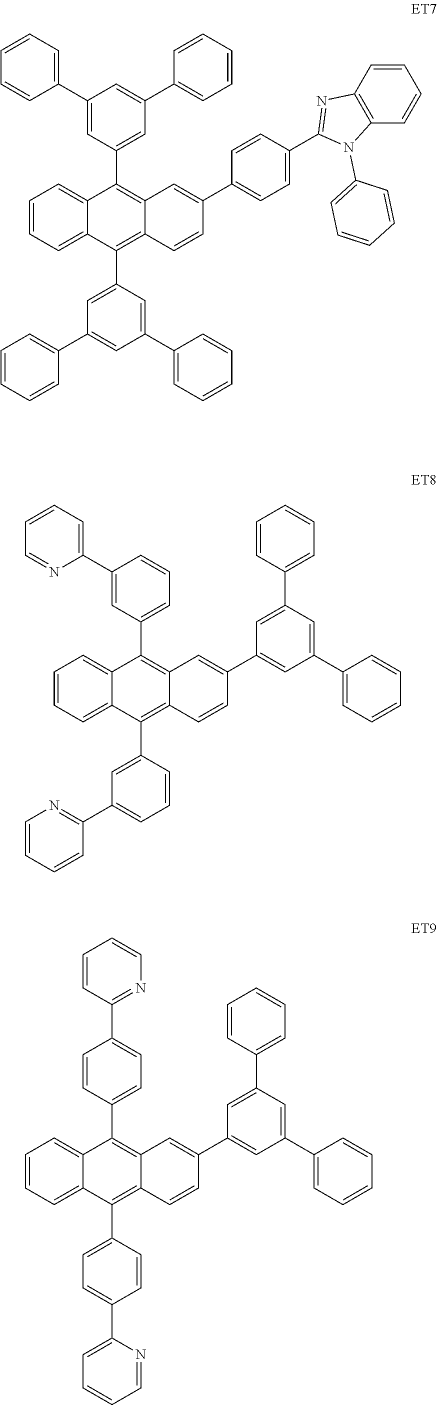 [SMILES: c1ccc(-c2cc(-c3ccccc3)cc(-c3c4ccccc4c(-c4cc(-c5ccccc5)cc(-c5ccccc5)c4)c4cc(-c5ccc(-c6nc7ccccc7n6-c6ccccc6)cc5)ccc34)c2)cc1 ; c1ccc(-c2cc(-c3ccccc3)cc(-c3ccc4c(-c5cccc(-c6ccccn6)c5)c5ccccc5c(-c5cccc(-c6ccccn6)c5)c4c3)c2)cc1 ; c1ccc(-c2cc(-c3ccccc3)cc(-c3ccc4c(-c5ccc(-c6ccccn6)cc5)c5ccccc5c(-c5ccc(-c6ccccn6)cc5)c4c3)c2)cc1]

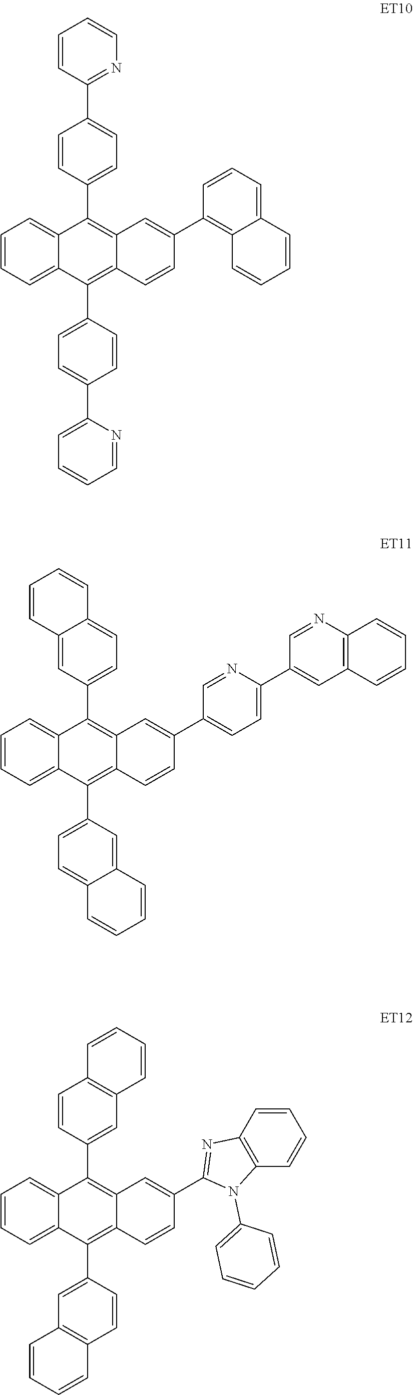 [SMILES: c1ccc(-c2ccc(-c3c4ccccc4c(-c4ccc(-c5ccccn5)cc4)c4cc(-c5cccc6ccccc56)ccc34)cc2)nc1 ; c1ccc2cc(-c3c4ccccc4c(-c4ccc5ccccc5c4)c4cc(-c5ccc(-c6cnc7ccccc7c6)nc5)ccc34)ccc2c1 ; c1ccc(-n2c(-c3ccc4c(-c5ccc6ccccc6c5)c5ccccc5c(-c5ccc6ccccc6c5)c4c3)nc3ccccc32)cc1]

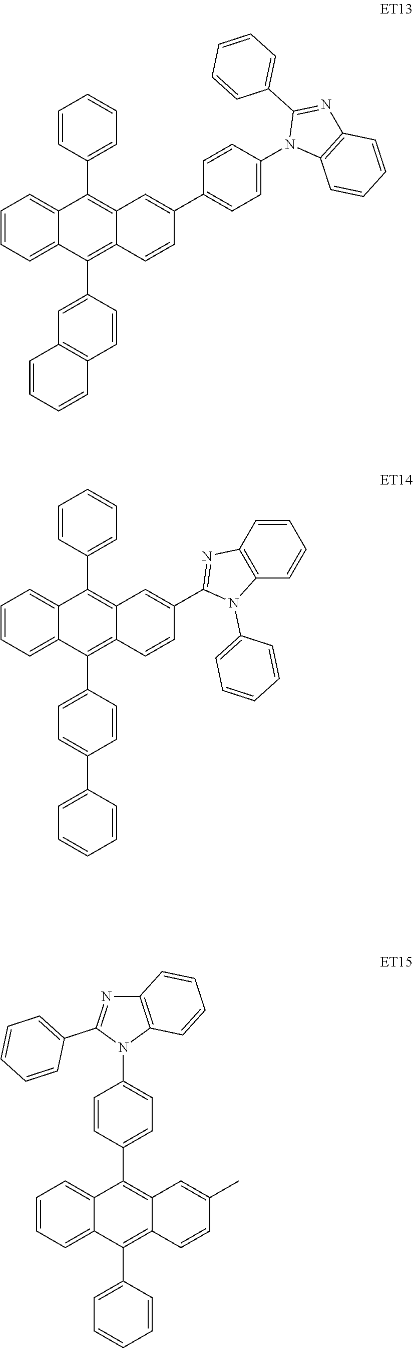 [SMILES: c1ccc(-c2c3ccccc3c(-c3ccc4ccccc4c3)c3ccc(-c4ccc(-n5c(-c6ccccc6)nc6ccccc65)cc4)cc23)cc1 ; c1ccc(-c2ccc(-c3c4ccccc4c(-c4ccccc4)c4cc(-c5nc6ccccc6n5-c5ccccc5)ccc34)cc2)cc1 ; Cc1ccc2c(-c3ccccc3)c3ccccc3c(-c3ccc(-n4c(-c5ccccc5)nc5ccccc54)cc3)c2c1]

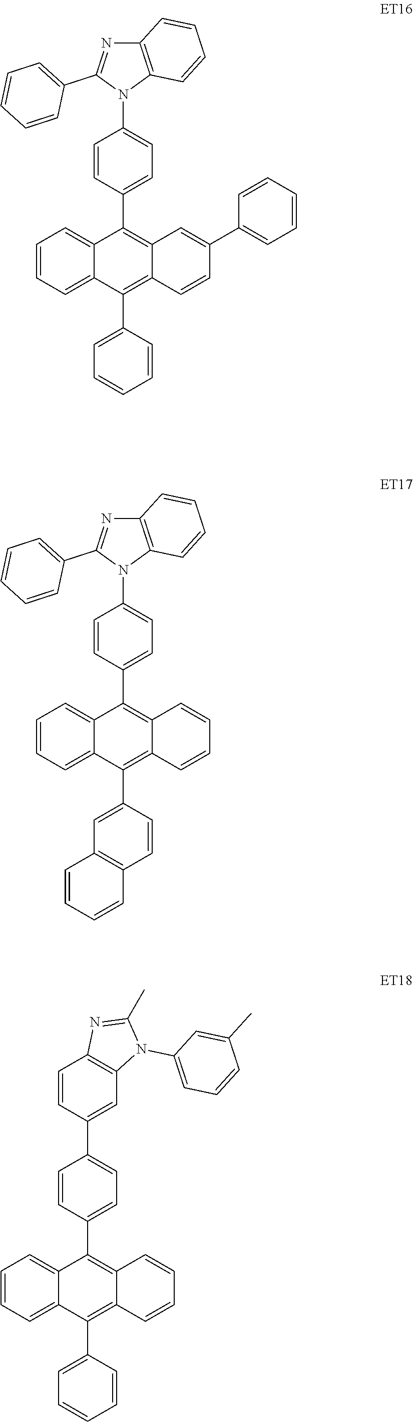 [SMILES: c1ccc(-c2ccc3c(-c4ccccc4)c4ccccc4c(-c4ccc(-n5c(-c6ccccc6)nc6ccccc65)cc4)c3c2)cc1 ; c1ccc(-c2nc3ccccc3n2-c2ccc(-c3c4ccccc4c(-c4ccc5ccccc5c4)c4ccccc34)cc2)cc1 ; Cc1cccc(-n2c(C)nc3ccc(-c4ccc(-c5c6ccccc6c(-c6ccccc6)c6ccccc56)cc4)cc32)c1]

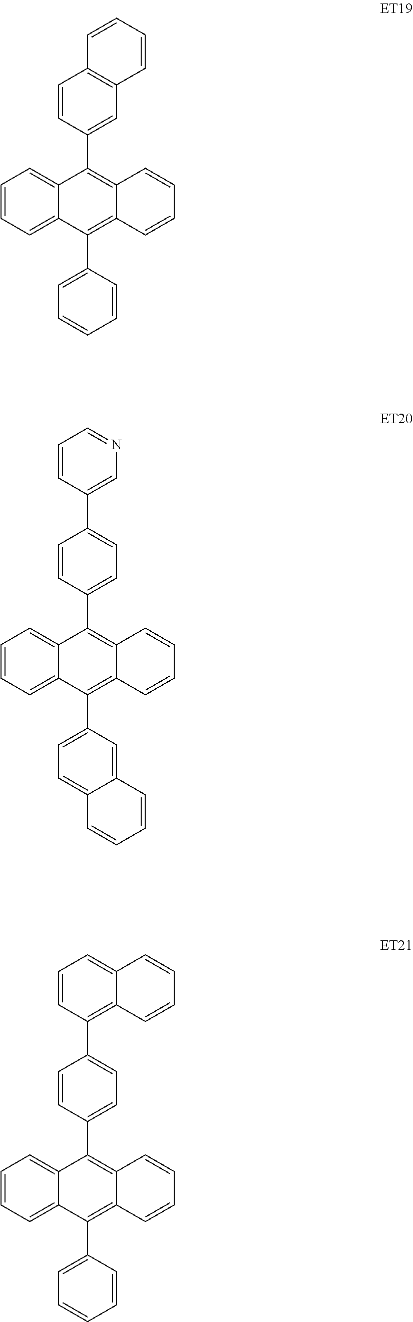

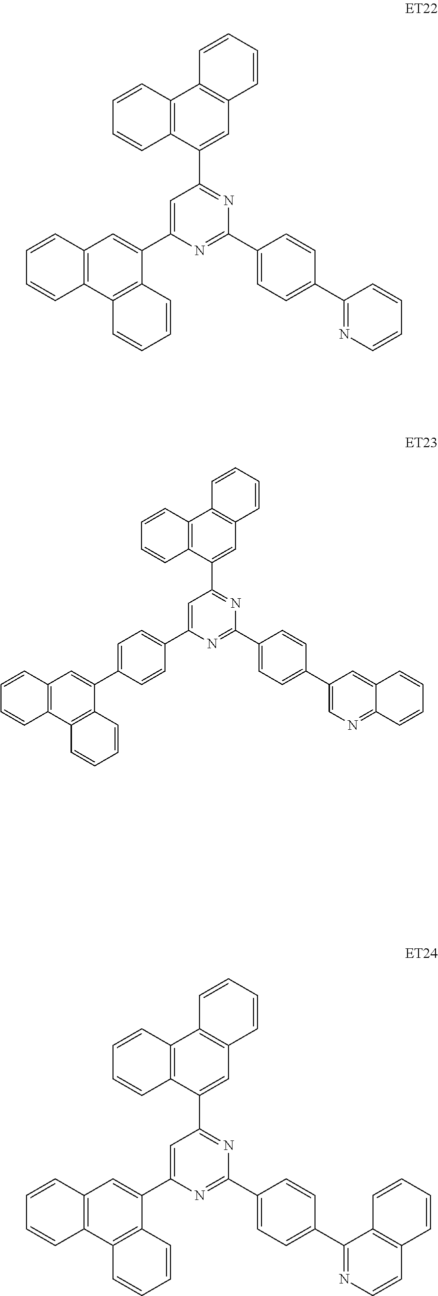 [SMILES: c1ccc(-c2ccc(-c3nc(-c4cc5ccccc5c5ccccc45)cc(-c4cc5ccccc5c5ccccc45)n3)cc2)nc1 ; c1ccc2ncc(-c3ccc(-c4nc(-c5ccc(-c6cc7ccccc7c7ccccc67)cc5)cc(-c5cc6ccccc6c6ccccc56)n4)cc3)cc2c1 ; c1ccc2c(-c3ccc(-c4nc(-c5cc6ccccc6c6ccccc56)cc(-c5cc6ccccc6c6ccccc56)n4)cc3)nccc2c1]

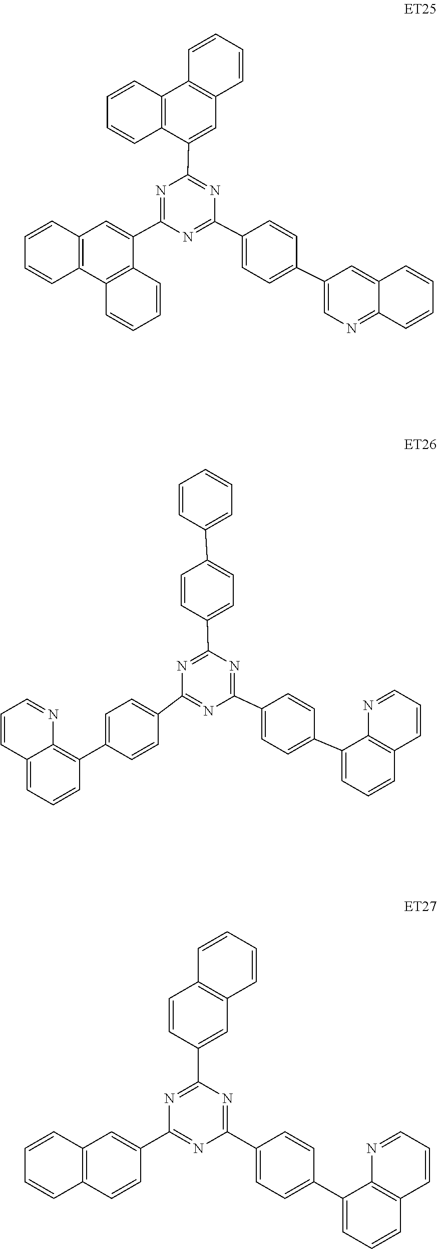 [SMILES: c1ccc2ncc(-c3ccc(-c4nc(-c5cc6ccccc6c6ccccc56)nc(-c5cc6ccccc6c6ccccc56)n4)cc3)cc2c1 ; c1ccc(-c2ccc(-c3nc(-c4ccc(-c5cccc6cccnc56)cc4)nc(-c4ccc(-c5cccc6cccnc56)cc4)n3)cc2)cc1 ; c1ccc2cc(-c3nc(-c4ccc(-c5cccc6cccnc56)cc4)nc(-c4ccc5ccccc5c4)n3)ccc2c1]

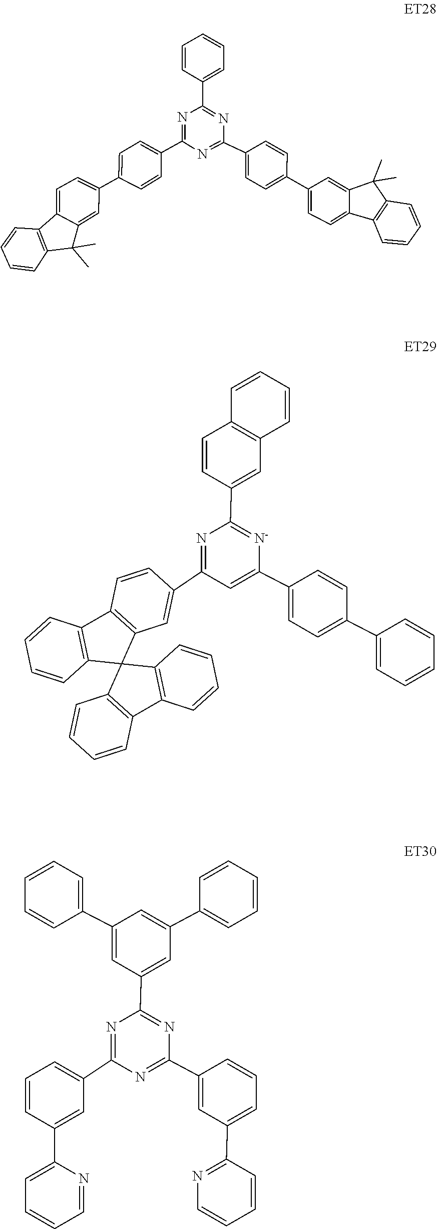

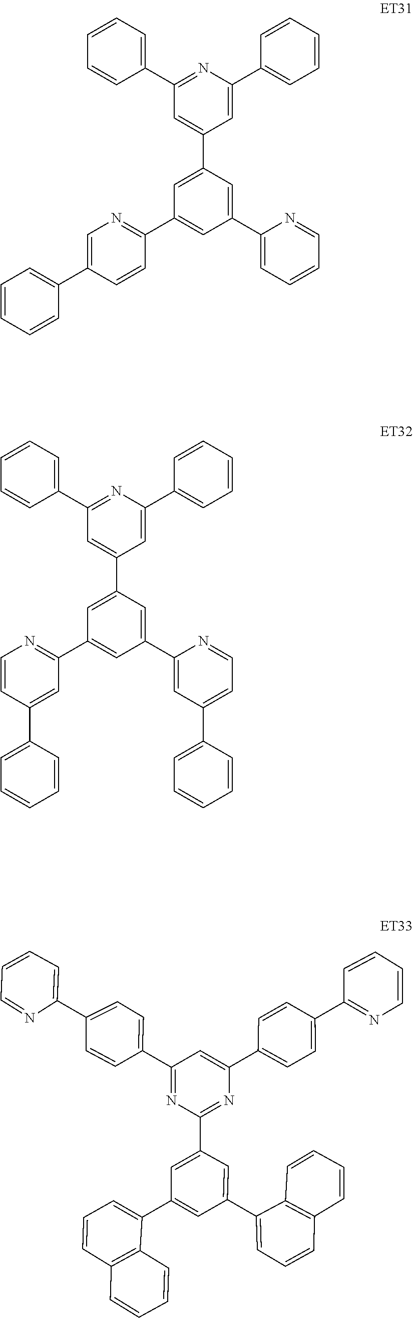 [SMILES: c1ccc(-c2ccc(-c3cc(-c4cc(-c5ccccc5)nc(-c5ccccc5)c4)cc(-c4ccccn4)c3)nc2)cc1 ; c1ccc(-c2ccnc(-c3cc(-c4cc(-c5ccccc5)nc(-c5ccccc5)c4)cc(-c4cc(-c5ccccc5)ccn4)c3)c2)cc1 ; c1ccc(-c2ccc(-c3cc(-c4ccc(-c5ccccn5)cc4)nc(-c4cc(-c5cccc6ccccc56)cc(-c5cccc6ccccc56)c4)n3)cc2)nc1]

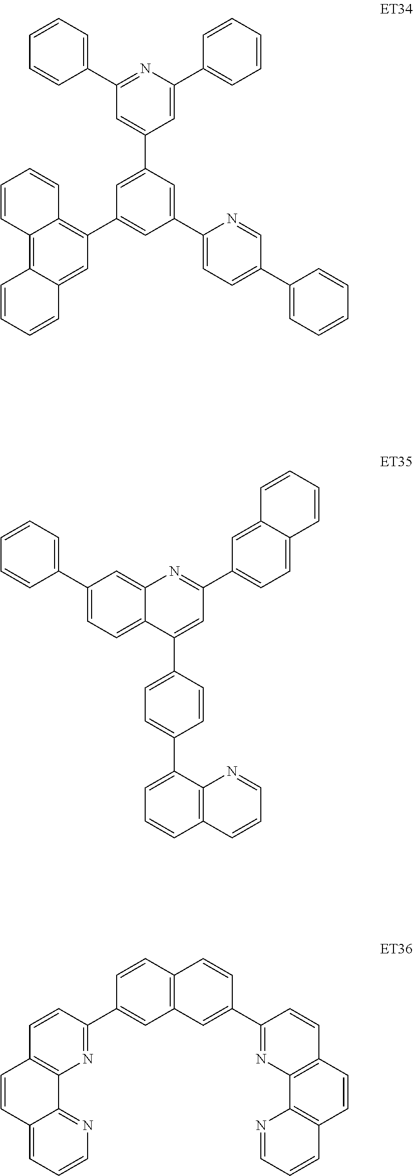 [SMILES: c1ccc(-c2ccc(-c3cc(-c4cc(-c5ccccc5)nc(-c5ccccc5)c4)cc(-c4cc5ccccc5c5ccccc45)c3)nc2)cc1 ; c1ccc(-c2ccc3c(-c4ccc(-c5cccc6cccnc56)cc4)cc(-c4ccc5ccccc5c4)nc3c2)cc1 ; c1cnc2c(c1)ccc1ccc(-c3ccc4ccc(-c5ccc6ccc7cccnc7c6n5)cc4c3)nc12]

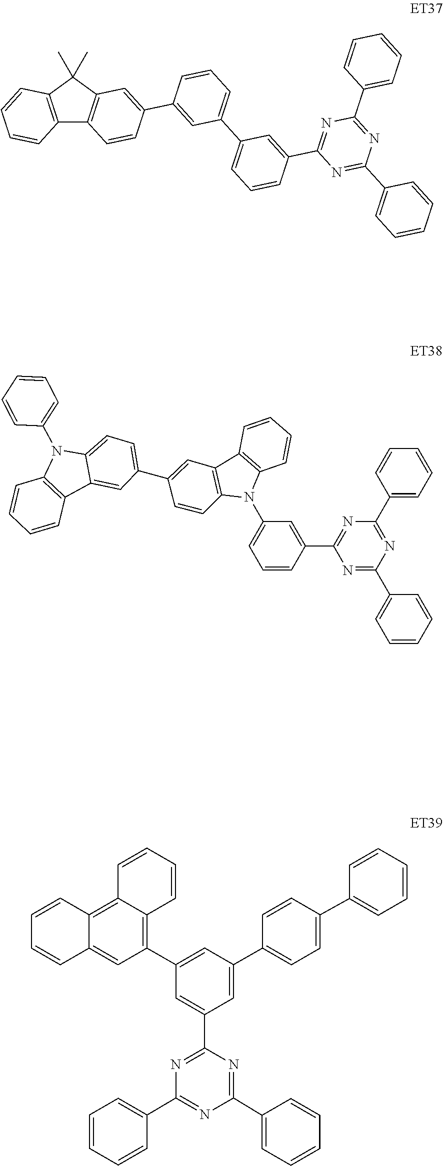 [SMILES: CC1(C)c2ccccc2-c2ccc(-c3cccc(-c4cccc(-c5nc(-c6ccccc6)nc(-c6ccccc6)n5)c4)c3)cc21 ; c1ccc(-c2nc(-c3ccccc3)nc(-c3cccc(-n4c5ccccc5c5cc(-c6ccc7c(c6)c6ccccc6n7-c6ccccc6)ccc54)c3)n2)cc1 ; c1ccc(-c2ccc(-c3cc(-c4nc(-c5ccccc5)nc(-c5ccccc5)n4)cc(-c4cc5ccccc5c5ccccc45)c3)cc2)cc1]

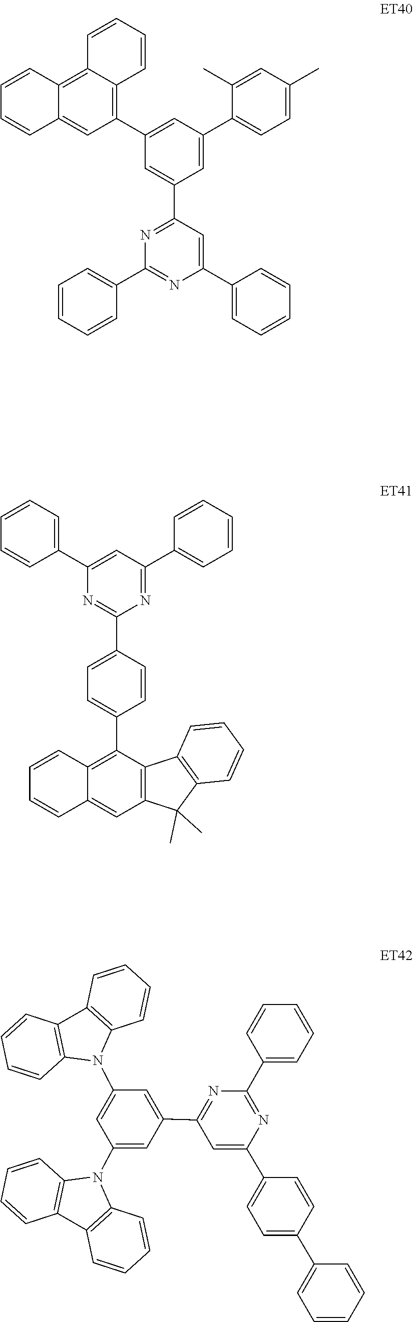 [SMILES: Cc1ccc(-c2cc(-c3cc(-c4ccccc4)nc(-c4ccccc4)n3)cc(-c3cc4ccccc4c4ccccc34)c2)c(C)c1 ; CC1(C)c2ccccc2-c2c1cc1ccccc1c2-c1ccc(-c2nc(-c3ccccc3)cc(-c3ccccc3)n2)cc1 ; c1ccc(-c2ccc(-c3cc(-c4cc(-n5c6ccccc6c6ccccc65)cc(-n5c6ccccc6c6ccccc65)c4)nc(-c4ccccc4)n3)cc2)cc1]

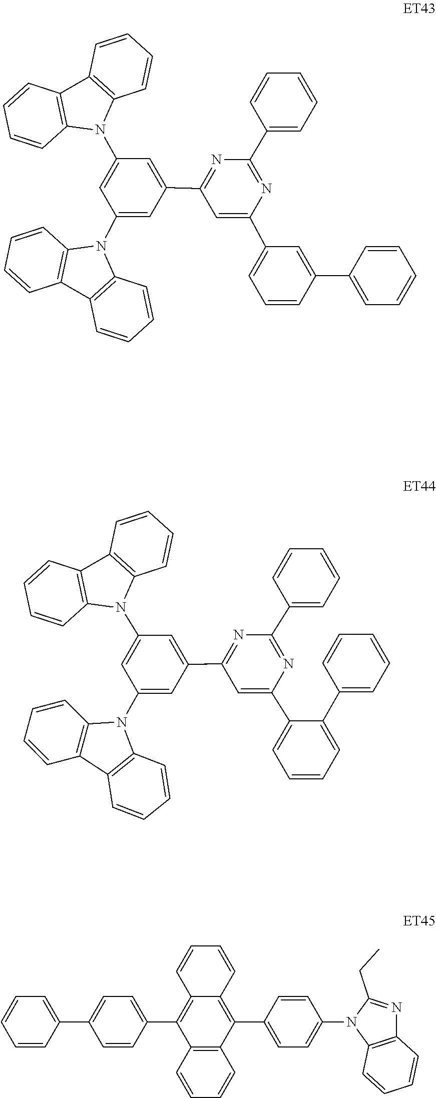 [SMILES: c1ccc(-c2cccc(-c3cc(-c4cc(-n5c6ccccc6c6ccccc65)cc(-n5c6ccccc6c6ccccc65)c4)nc(-c4ccccc4)n3)c2)cc1 ; c1ccc(-c2nc(-c3cc(-n4c5ccccc5c5ccccc54)cc(-n4c5ccccc5c5ccccc54)c3)cc(-c3ccccc3-c3ccccc3)n2)cc1 ; CCc1nc2ccccc2n1-c1ccc(-c2c3ccccc3c(-c3ccc(-c4ccccc4)cc3)c3ccccc23)cc1]

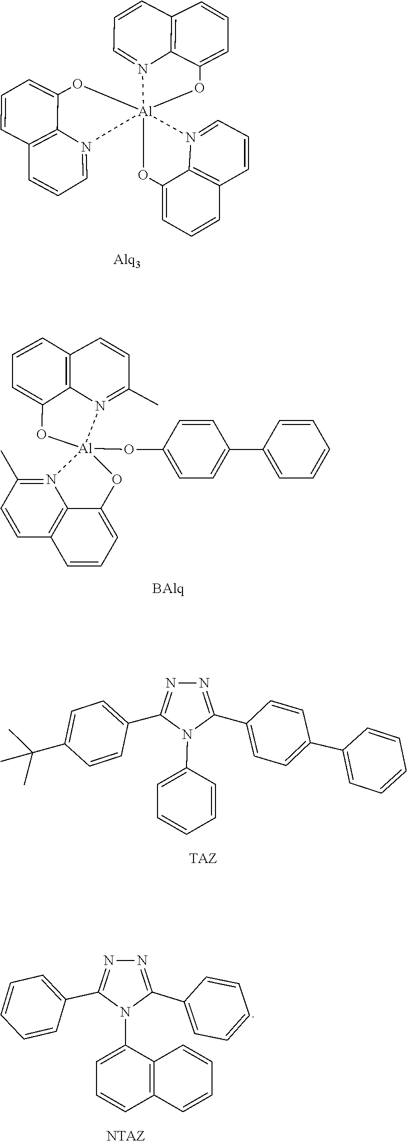

A thickness of the electron transport region may be in a range of about 100 Å to about 5,000 Å, for example, about 160 Å to about 4,000 Å. When the electron transport region includes a buffer layer, a hole blocking layer, an electron control layer, an electron transport layer, or a combination thereof, the thickness of the buffer layer, the hole blocking layer, or the electron control layer may each independently be in a range of about 20 Å to about 1,000 Å, for example, about 30 Å to about 300 Å, and the thickness of the electron transport layer may be in a range of about 100 Å to about 1,000 Å, for example, about 150 Å to about 500 Å. When the thicknesses of the buffer layer, the hole blocking layer, the electron control layer, the electron transport layer, and/or the electron transport region are within these ranges, satisfactory and/or suitable electron transporting characteristics may be obtained without a substantial increase in driving voltage.

In one or more embodiments, the electron transport region (for example, the electron transport layer in the electron transport region) may further include, in addition to the materials described above, a metal-containing material.

The metal-containing material may include an alkali metal complex, an alkaline earth metal complex, or a combination thereof. A metal ion of the alkali metal complex may be a Li ion, a Na ion, a K ion, a Rb ion, or a Cs ion, and a metal ion of the alkaline earth metal complex may be a Be ion, a Mg ion, a Ca ion, a Sr ion, or a Ba ion. A ligand coordinated with the metal ion of the alkali metal complex or the alkaline earth-metal complex may include a hydroxyquinoline, a hydroxyisoquinoline, a hydroxybenzoquinoline, a hydroxyacridine, a hydroxyphenanthridine, a hydroxyphenyloxazole, a hydroxyphenylthiazole, a hydroxyphenyloxadiazole, a hydroxyphenylthiadiazole, a hydroxyphenylpyridine, a hydroxyphenylbenzimidazole, a hydroxyphenylbenzothiazole, a bipyridine, a phenanthroline, a cyclopentadiene, or a combination thereof.

For example, in some embodiments, the metal-containing material may include a L₁ complex. The L₁ complex may include, for example, Compound ET-D1 (LiQ) or ET-D2:

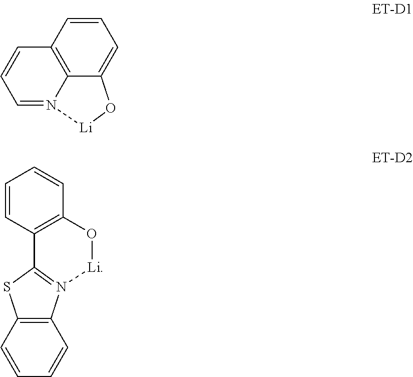

In one or more embodiments, the electron transport region may include an electron injection layer that facilitates the injection of electrons from the second electrode 150. The electron injection layer may directly contact the second electrode 150.

The electron injection layer may have: i) a single-layer structure including (e.g., consisting of) a single layer including (e.g., consisting of) a single material, ii) a single-layer structure including (e.g., consisting of) a single layer including (e.g., consisting of) multiple different materials, or iii) a multi-layer structure including multiple layers including different materials.

The electron injection layer may include an alkali metal, an alkaline earth metal, a rare earth metal, an alkali metal-containing compound, an alkaline earth metal-containing compound, a rare earth metal-containing compound, an alkali metal complex, an alkaline earth metal complex, a rare earth metal complex, or a combination thereof.

The alkali metal may include Li, Na, K, Rb, Cs, or a combination thereof. The alkaline earth metal may include Mg, Ca, Sr, Ba, or a combination thereof. The rare earth metal may include Sc, Y, Ce, Tb, Yb, Gd, or a combination thereof.

The alkali metal-containing compound, the alkaline earth metal-containing compound, and the rare earth metal-containing compound may be oxides, halides (for example, fluorides, chlorides, bromides, iodides, etc.), or tellurides of the alkali metal, the alkaline earth metal, and the rare earth metal, or a combination thereof.

The alkali metal-containing compound may include: an alkali metal oxide, such as Li₂O, Cs₂O, K₂O, and/or the like; an alkali metal halide, such as LiF, NaF, CsF, KF, LiI, NaI, CsI, KI, and/or the like; or a combination thereof. The alkaline earth metal-containing compound may include an alkaline earth metal compound, such as BaO, SrO, CaO, BaXSri-XO (wherein x is a real number satisfying 0<x<1), BaₓCa₁₋ₓO (wherein x is a real number satisfying 0<x<1), and/or the like. The rare earth metal-containing compound may include YbF₃, ScF₃, Sc₂O₃, Y₂O₃, Ce₂O₃, GdF₃, TbF₃, YbI₃, ScI₃, TbI₃, or a combination thereof. In one or more embodiments, the rare earth metal-containing compound may include lanthanide metal telluride. Non-limiting examples of the lanthanide metal telluride may be LaTe, CeTe, PrTe, NdTe, PmTe, SmTe, EuTe, GdTe, TbTe, DyTe, HoTe, ErTe, TmTe, YbTe, LuTe, La₂Te₃, Ce₂Te₃, Pr₂Te₃, Nd₂Te₃, Pm₂Te₃, Sm₂Te₃, Eu₂Te₃, Gd₂Te₃, Tb₂Te₃, Dy₂Te₃, Ho₂Te₃, Er₂Te₃, Tm₂Te₃, Yb₂Te₃, Lu₂Te₃, and/or the like.

The alkali metal complex, the alkaline earth-metal complex, and the rare earth metal complex may include i) one of ions of the alkali metal, the alkaline earth metal, and the rare earth metal, respectively, and ii) a ligand bonded to the metal ion, for example, hydroxyquinoline, hydroxyisoquinoline, hydroxybenzoquinoline, hydroxyacridine, hydroxyphenanthridine, hydroxyphenyloxazole, hydroxyphenylthiazole, hydroxyphenyloxadiazole, hydroxyphenylthiadiazole, hydroxyphenylpyridine, hydroxyphenyl benzimidazole, hydroxyphenylbenzothiazole, bipyridine, phenanthroline, cyclopentadiene, or a combination thereof.

In one or more embodiments, the electron injection layer may include (e.g., consist of) an alkali metal, an alkaline earth metal, a rare earth metal, an alkali metal-containing compound, an alkaline earth metal-containing compound, a rare earth metal-containing compound, an alkali metal complex, an alkaline earth metal complex, a rare earth metal complex, or a combination thereof, as described above. In one or more embodiments, the electron injection layer may further include an organic material (for example, a compound represented by Formula 601).

In one or more embodiments, the electron injection layer may include (e.g., consist of) i) an alkali metal-containing compound (for example, alkali metal halide), ii) a) an alkali metal-containing compound (for example, alkali metal halide); and b) an alkali metal, an alkaline earth metal, a rare earth metal, or a combination thereof. For example, in some embodiments, the electron injection layer may be a KI:Yb co-deposited layer, an RbI:Yb co-deposited layer, a LiF:Yb co-deposited layer, and/or the like.

When the electron injection layer further includes an organic material, an alkali metal, an alkaline earth metal, a rare earth metal, an alkali metal-containing compound, an alkaline earth metal-containing compound, a rare earth metal-containing compound, an alkali metal complex, an alkaline earth-metal complex, a rare earth metal complex, or a combination thereof may be substantially uniformly or non-uniformly dispersed in a matrix including the organic material.

A thickness of the electron injection layer may be in a range of about 1 Å to about 100 Å, and, for example, about 3 Å to about 90 Å. When the thickness of the electron injection layer is within the ranges above, satisfactory and/or suitable electron injection characteristics may be obtained without a substantial increase in driving voltage.

### Second Electrode 150

The second electrode 150 may be arranged on the interlayer 130 having a structure as described above. In one or more embodiments, the second electrode 150 may be a cathode, which is an electron injection electrode, and as a material for forming the second electrode 150, a metal, an alloy, an electrically conductive compound, or a combination thereof, each having a low-work function, may be utilized.

The second electrode 150 may include lithium (Li), silver (Ag), magnesium (Mg), aluminum (Al), aluminum-lithium (Al—Li), calcium (Ca), magnesium-indium (Mg—In), magnesium-silver (Mg—Ag), ytterbium (Yb), silver-ytterbium (Ag—Yb), ITO, IZO, or a combination thereof. The second electrode 150 may be a transmissive electrode, a semi-transmissive electrode, or a reflective electrode.

The second electrode 150 may have a single-layer structure or a multi-layer structure including multiple layers.

### Capping Layer

A first capping layer may be arranged outside the first electrode 110, and/or a second capping layer may be arranged outside the second electrode 150. In one or more embodiments, the light-emitting device 10 may have a structure in which the first capping layer, the first electrode 110, the interlayer 130, and the second electrode 150 are sequentially stacked in the stated order, a structure in which the first electrode 110, the interlayer 130, the second electrode 150, and the second capping layer are sequentially stacked in the stated order, or a structure in which the first capping layer, the first electrode 110, the interlayer 130, the second electrode 150, and the second capping layer are sequentially stacked in the stated order.

In some embodiments, light generated in an emission layer of the interlayer 130 of the light-emitting device 10 may be extracted toward the outside through the first electrode 110 which is a semi-transmissive electrode or a transmissive electrode, and the first capping layer. In some embodiments, light generated in an emission layer of the interlayer 130 of the light-emitting device 10 may be extracted toward the outside through the second electrode 150 which is a semi-transmissive electrode or a transmissive electrode, and the second capping layer.

The first capping layer and/or the second capping layer may increase external emission efficiency according to the principle of constructive interference. Accordingly, the light extraction efficiency of the light-emitting device 10 is increased, so that the luminescence efficiency of the light-emitting device 10 may be improved.

Each of the first capping layer and the second capping layer may include a material having a refractive index of greater than or equal to 1.6 (e.g., at 589 nm).

The first capping layer and the second capping layer may each independently be an organic capping layer including an organic material, an inorganic capping layer including an inorganic material, or an organic-inorganic composite capping layer including an organic material and an inorganic material.

At least one selected from among the first capping layer and the second capping layer may each independently include a carbocyclic compound, a heterocyclic compound, an amine group-containing compound, a porphine derivative, a phthalocyanine derivative, a naphthalocyanine derivative, an alkali metal complex, an alkaline earth metal complex, or a combination thereof. In some embodiments, the carbocyclic compound, the heterocyclic compound, and the amine group-containing compound may optionally be substituted with a substituent including O, N, S, Se, Si, F, Cl, Br, I, or a combination thereof. In one or more embodiments, at least one selected from among the first capping layer and the second capping layer may each independently include an amine group-containing compound.

In one or more embodiments, at least one selected from among the first capping layer and the second capping layer may each independently include a compound represented by Formula 201, a compound represented by Formula 202, or a combination thereof.

In one or more embodiments, at least one selected from among the first capping layer and the second capping layer may each independently include at least one amine-based compound represented by Formula 1.

In one or more embodiments, at least one selected from among the first capping layer and the second capping layer may each independently include: at least one selected from among Compounds HT28 to HT33; at least one selected from among Compounds CP1 to CP6; β-NPB; and/or a combination thereof:

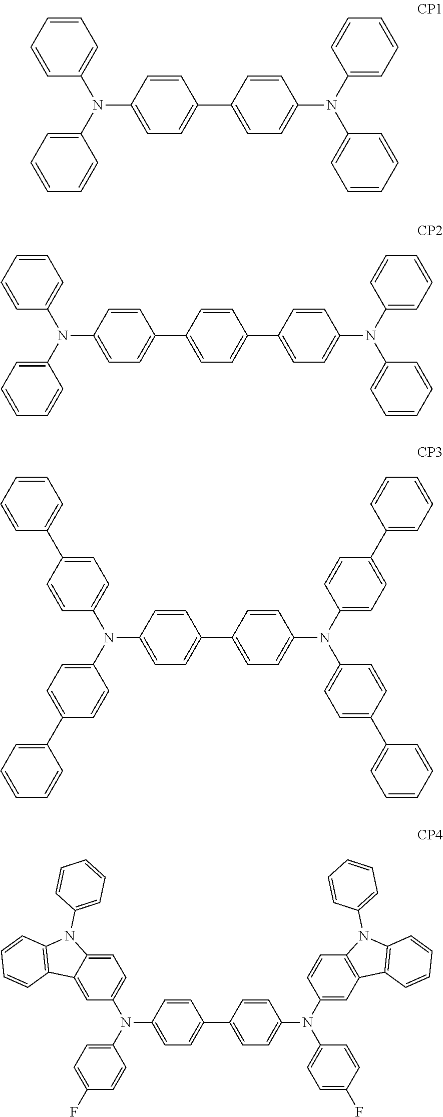 [SMILES: c1ccc(N(c2ccccc2)c2ccc(-c3ccc(N(c4ccccc4)c4ccccc4)cc3)cc2)cc1 ; c1ccc(N(c2ccccc2)c2ccc(-c3ccc(-c4ccc(N(c5ccccc5)c5ccccc5)cc4)cc3)cc2)cc1 ; c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1 ; Fc1ccc(N(c2ccc(-c3ccc(N(c4ccc(F)cc4)c4ccc5c(c4)c4ccccc4n5-c4ccccc4)cc3)cc2)c2ccc3c(c2)c2ccccc2n3-c2ccccc2)cc1]

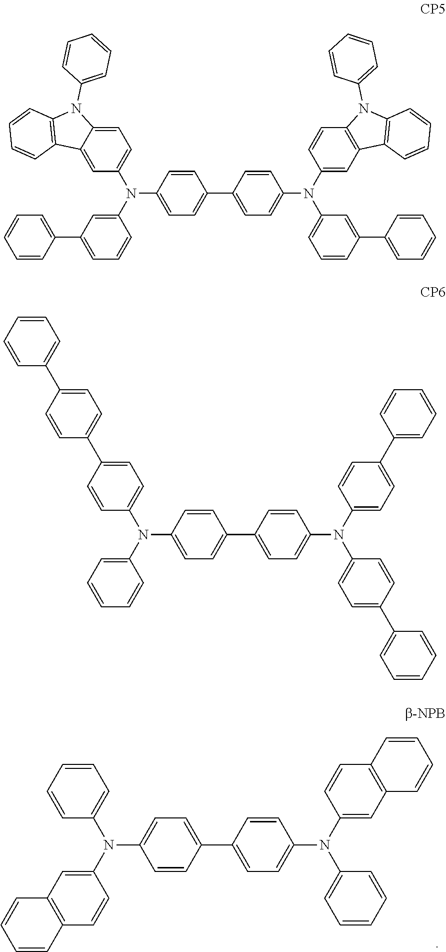 [SMILES: c1ccc(-c2cccc(N(c3ccc(-c4ccc(N(c5cccc(-c6ccccc6)c5)c5ccc6c(c5)c5ccccc5n6-c5ccccc5)cc4)cc3)c3ccc4c(c3)c3ccccc3n4-c3ccccc3)c2)cc1 ; c1ccc(-c2ccc(-c3ccc(N(c4ccccc4)c4ccc(-c5ccc(N(c6ccc(-c7ccccc7)cc6)c6ccc(-c7ccccc7)cc6)cc5)cc4)cc3)cc2)cc1 ; c1ccc(N(c2ccc(-c3ccc(N(c4ccccc4)c4ccc5ccccc5c4)cc3)cc2)c2ccc3ccccc3c2)cc1]

### Film

The amine-based compound represented by Formula 1 may be included in one or more suitable films. Accordingly, in one or more embodiments, a film may include an amine-based compound represented by Formula 1. The film may be, for example, an optical member (or a light control element) (for example, a color filter, a color conversion member, a capping layer, a light extraction efficiency enhancement layer, a selective light absorbing layer, a polarizing layer, a quantum dot-containing layer, and/or like), a light-blocking member (for example, a light reflective layer, a light absorbing layer, and/or the like), and/or a protective member (for example, an insulating layer, a dielectric layer, and/or the like).

### Electronic Apparatus

The light-emitting device may be included in one or more suitable electronic apparatuses. For example, in some embodiments, the electronic apparatus including the light-emitting device may be a light-emitting apparatus, an authentication apparatus, and/or the like.

In one or more embodiments, the electronic apparatus (for example, a light-emitting apparatus) may further include, in addition to the light-emitting device, i) a color filter, ii) a color conversion layer, or iii) a color filter and a color conversion layer. The color filter and/or the color conversion layer may be arranged in at least one travel direction of light emitted from the light-emitting device. For example, in one or more embodiments, the light emitted from the light-emitting device may be blue light or white light (e.g., combined white light). Details on the light-emitting device may be referred to the descriptions provided herein. In one or more embodiments, the color conversion layer may include a quantum dot. The quantum dot may be, for example, the quantum dot as described herein.

The electronic apparatus may include a first substrate. The first substrate may include a plurality of subpixel areas, the color filter may include a plurality of color filter areas respectively corresponding to the subpixel areas, and the color conversion layer may include a plurality of color conversion areas respectively corresponding to the subpixel areas.

A pixel defining layer may be arranged among the subpixel areas to define each of the subpixel areas.

The color filter may further include a plurality of color filter areas and light-shielding patterns arranged among the color filter areas, and the color conversion layer may further include a plurality of color conversion areas and light-shielding patterns arranged among the color conversion areas.

The plurality of color filter areas (or the plurality of color conversion areas) may include a first area to emit first color light, a second area to emit second color light, and/or a third area to emit third color light, wherein the first color light, the second color light, and/or the third color light may have different maximum emission wavelengths from one another. For example, in some embodiments, the first color light may be red light, the second color light may be green light, and the third color light may be blue light. For example, in one or more embodiments, the plurality of color filter areas (or the plurality of color conversion areas) may include quantum dots. In some embodiments, the first area may include a red quantum dot to emit red light, the second area may include a green quantum dot to emit green light, and the third area may not include (e.g., may exclude) a quantum dot. Details on the quantum dot may be referred to the descriptions provided herein. The first area, the second area, and/or the third area may each further include a scatterer.

For example, in one or more embodiments, the light-emitting device may be to emit first light, the first area may be to absorb the first light to emit first-first color light, the second area may be to absorb the first light to emit second-first color light, and the third area may be to absorb the first light to emit third-first color light. Here, the first-first color light, the second-first color light, and the third-first color light may have different maximum emission wavelengths. In some embodiments, the first light may be blue light, the first-first color light may be red light, the second-first color light may be green light, and the third-first color light may be blue light.

In one or more embodiments, the electronic apparatus may further include a thin-film transistor, in addition to the light-emitting device as described above. The thin-film transistor may include a source electrode, a drain electrode, and an activation layer, wherein one selected from the source electrode and the drain electrode may be electrically connected to the first electrode or the second electrode of the light-emitting device.

The thin-film transistor may further include a gate electrode, a gate insulating film, and/or the like.

The activation layer may include crystalline silicon, amorphous silicon, an organic semiconductor, an oxide semiconductor, and/or the like.

In one or more embodiments, the electronic apparatus may further include a sealing portion for sealing the light-emitting device. The sealing portion may be arranged between the color filter and/or the color conversion layer and the light-emitting device. The sealing portion allows light from the light-emitting device to be extracted to the outside, and concurrently (e.g., simultaneously) prevents and/or reduce ambient air and moisture from penetrating into the light-emitting device. The sealing portion may be a sealing substrate including a transparent glass substrate or a plastic substrate. The sealing portion may be a thin-film encapsulation layer including at least one layer of an organic layer and/or an inorganic layer. When the sealing portion is a thin film encapsulation layer, the electronic apparatus may be flexible.

One or more functional layers may be additionally arranged on the sealing portion, in addition to the color filter and/or the color conversion layer, according to the utilization of the electronic apparatus. Non-limiting examples of the functional layers may include a touch screen layer, a polarizing layer, and/or the like. The touch screen layer may be a pressure-sensitive touch screen layer, a capacitive touch screen layer, or an infrared touch screen layer. The authentication apparatus may be, for example, a biometric authentication apparatus that authenticates an individual by utilizing biometric information of a living body (for example, fingertips, pupils, etc.).

The authentication apparatus may further include, in addition to the light-emitting device as described above, a biometric information collector.

The electronic apparatus may be applied to one or more of displays, light sources, lighting, personal computers (for example, a mobile personal computer), mobile phones, digital cameras, electronic organizers, electronic dictionaries, electronic game machines, medical instruments (for example, electronic thermometers, sphygmomanometers, blood glucose meters, pulse measurement devices, pulse wave measurement devices, electrocardiogram displays, ultrasonic diagnostic devices, or endoscope displays), fish finders, one or more suitable measuring instruments, meters (for example, meters for a vehicle, an aircraft, and a vessel), projectors, and/or the like.

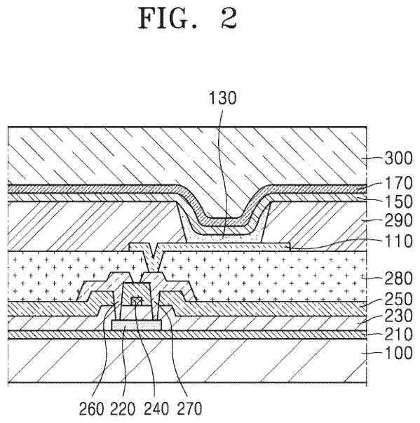

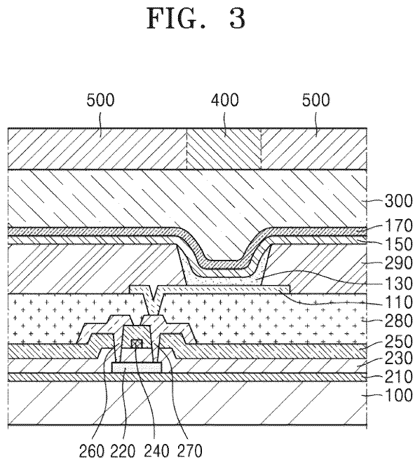

### Description of FIGS. 2 and 3

FIG. 2 is a cross-sectional view showing an electronic apparatus according to one or more embodiments of the present disclosure.

The electronic apparatus of FIG. 2 includes a substrate 100, a thin-film transistor (TFT), a light-emitting device, and an encapsulation portion 300 that seals the light-emitting device.

The substrate 100 may be a flexible substrate, a glass substrate, or a metal substrate. A buffer layer 210 may be on the substrate 100. The buffer layer 210 may prevent or reduce penetration of impurities through the substrate 100 and may provide a flat surface on the substrate 100.

A TFT may be on the buffer layer 210. The TFT may include an activation layer 220, a gate electrode 240, a source electrode 260, and a drain electrode 270.

The activation layer 220 may include an inorganic semiconductor such as silicon or polysilicon, an organic semiconductor, or an oxide semiconductor, and may include a source region, a drain region, and a channel region.

A gate insulating film 230 for insulating the activation layer 220 from the gate electrode 240 may be on the activation layer 220, and the gate electrode 240 may be on the gate insulating film 230.

An interlayer insulating film 250 may be on the gate electrode 240. The interlayer insulating film 250 may be between the gate electrode 240 and the source electrode 260 and between the gate electrode 240 and the drain electrode 270, to insulate from one another.

The source electrode 260 and the drain electrode 270 may be on the interlayer insulating film 250. The interlayer insulating film 250 and the gate insulating film 230 may be formed to expose the source region and the drain region of the activation layer 220, and the source electrode 260 and the drain electrode 270 may be respectively in contact with the exposed portions of the source region and the drain region of the activation layer 220.

The TFT may be electrically connected to a light-emitting device to drive the light-emitting device, and may be covered and protected by a passivation layer 280. The passivation layer 280 may include an inorganic insulating film, an organic insulating film, or a combination thereof. A light-emitting device may be provided on the passivation layer 280. The light-emitting device may include a first electrode 110, an interlayer 130, and a second electrode 150.

The first electrode 110 may be on the passivation layer 280. The passivation layer 280 may be arranged to expose a portion of the drain electrode 270, not fully covering the drain electrode 270, and the first electrode 110 may be arranged to be connected to the exposed portion of the drain electrode 270.

A pixel defining layer 290 including an insulating material may be on the first electrode 110. The pixel defining layer 290 may expose a certain region of the first electrode 110, and the interlayer 130 may be formed in the exposed region of the first electrode 110. The pixel defining layer 290 may be a polyimide-based organic film or a polyacrylic-based organic film. In some embodiments, at least some layers of the interlayer 130 may extend beyond the upper portion of the pixel defining layer 290 to be arranged in the form of a common layer.

The second electrode 150 may be on the interlayer 130, and a capping layer 170 may be additionally formed on the second electrode 150. The capping layer 170 may be formed to cover the second electrode 150.

The encapsulation portion 300 may be on the capping layer 170. The encapsulation portion 300 may be on a light-emitting device to protect the light-emitting device from moisture and/or oxygen. The encapsulation portion 300 may include: an inorganic film including silicon nitride (SiNₓ), silicon oxide (SiOₓ), indium tin oxide, indium zinc oxide, or a combination thereof; an organic film including polyethylene terephthalate, polyethylene naphthalate, polycarbonate, polyimide, polyethylene sulfonate, polyoxymethylene, polyarylate, hexamethyldisiloxane, an acrylic resin (for example, polymethyl methacrylate, polyacrylic acid, and/or the like), an epoxy-based resin (for example, aliphatic glycidyl ether (AGE), and/or the like), or a combination thereof; or a combination of the inorganic films and the organic films.

FIG. 3 shows a cross-sectional view showing an electronic apparatus according to one or more embodiments of the present disclosure.

The electronic apparatus of FIG. 3 may be substantially the same as the electronic apparatus of FIG. 2, except that a light-shielding pattern 500 and a functional region 400 are additionally arranged on the encapsulation portion 300. The functional region 400 may be i) a color filter area, ii) a color conversion area, or iii) a combination of a color filter area and a color conversion area. In one or more embodiments, the light-emitting device included in the electronic apparatus of FIG. 3 may be a tandem light-emitting device.

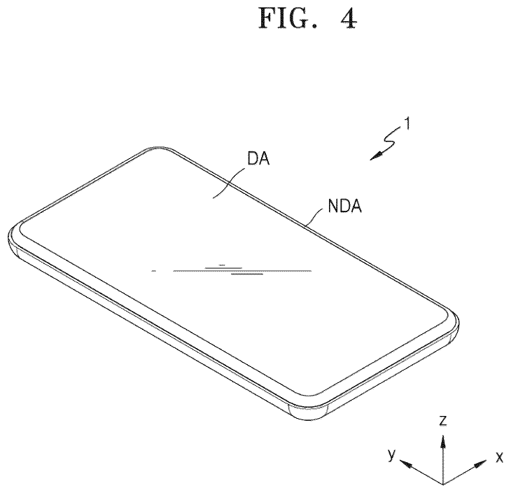

### Description of FIG. 4

FIG. 4 is a schematic perspective view of electronic equipment 1 including a light-emitting device according to one or more embodiments of the present disclosure. The electronic equipment 1 may be, as a device apparatus that displays a moving image or still image, a portable electronic equipment, such as a mobile phone, a smart phone, a tablet personal computer (PC), a mobile communication terminal, an electronic notebook, an electronic book, a portable multimedia player (PMP), a navigation, or a ultra mobile PC (UMPC), as well as one or more suitable products, such as a television, a laptop, a monitor, a billboard, or an Internet of things (IOT). The electronic equipment 1 may be such a product above or a part thereof. In some embodiments, the electronic equipment 1 may be a wearable device, such as a smart watch, a watch phone, a glasses-type or kind display, or a head mounted display (HMD), or a part of the wearable device. However, embodiments of the present disclosure are not limited thereto. For example, in some embodiments, the electron equipment 1 may include a dashboard of a vehicle, a center fascia of a vehicle, a center information display arranged on a dashboard of a vehicle, a room mirror display replacing a side mirror of a vehicle, an entertainment display for the rear seat of a vehicle or a display arranged on the back of the front seat, or a head up display (HUD) installed in the front of a vehicle or projected on a front window glass, a computer generated hologram augmented reality head up display (CGH AR HUD). FIG. 4 illustrates an embodiment in which the electronic equipment 1 is a smart phone for convenience of explanation.

The electronic equipment 1 may include a display area DA and a non-display area NDA outside the display area DA. A display device of the electronic equipment 1 may implement an image through an array of a plurality of pixels that are two-dimensionally arranged in the display area DA.

The non-display area NDA is an area that does not display an image, and may entirely surround the display area DA. At (e.g., in) the non-display area NDA, a driver for providing electrical signals or power to display devices arranged at the display area DA may be arranged. At (e.g., in) the non-display area NDA, a pad, which is an area to which an electronic element or a printing circuit board may be electrically connected, may be arranged.

In the electronic equipment 1, a length in the x-axis direction and a length in the y-axis direction may be different from each other. In one or more embodiments, as shown in FIG. 4, the length in the x-axis direction may be shorter than the length in the y-axis direction. In one or more embodiments, the length in the x-axis direction may be the same as the length in the y-axis direction. In one or more embodiments, the length in the x-axis direction may be longer than the length in the y-axis direction.

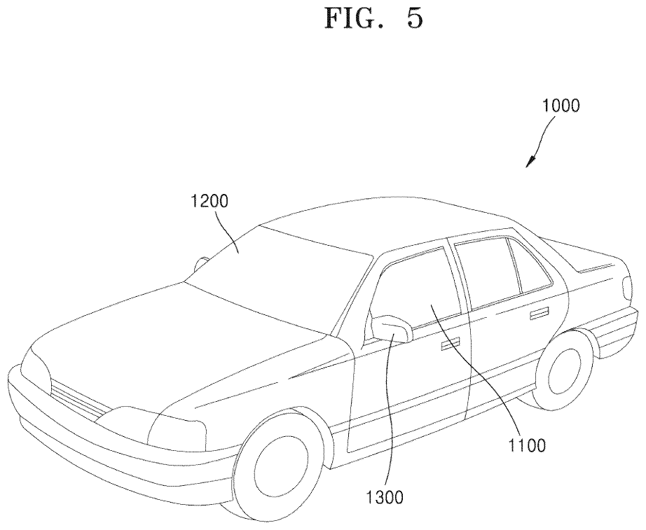

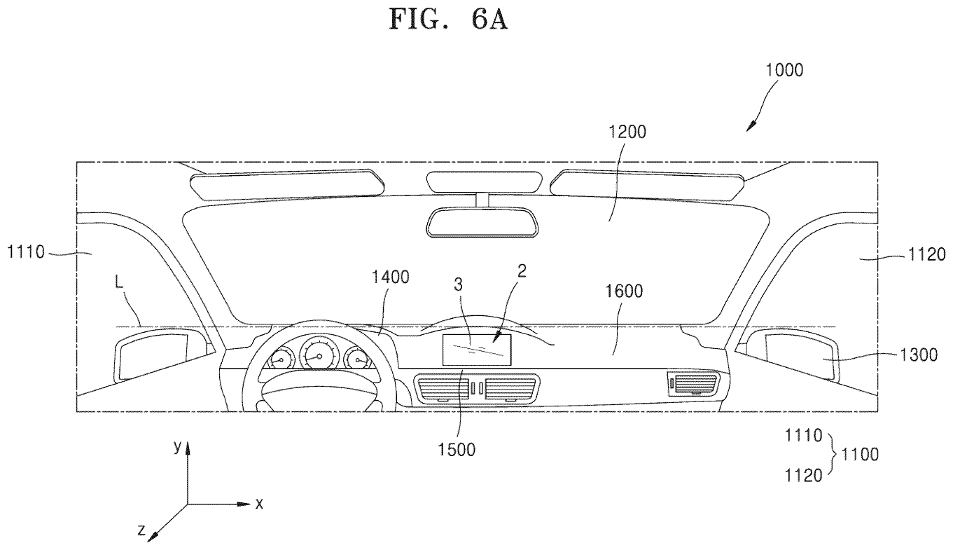

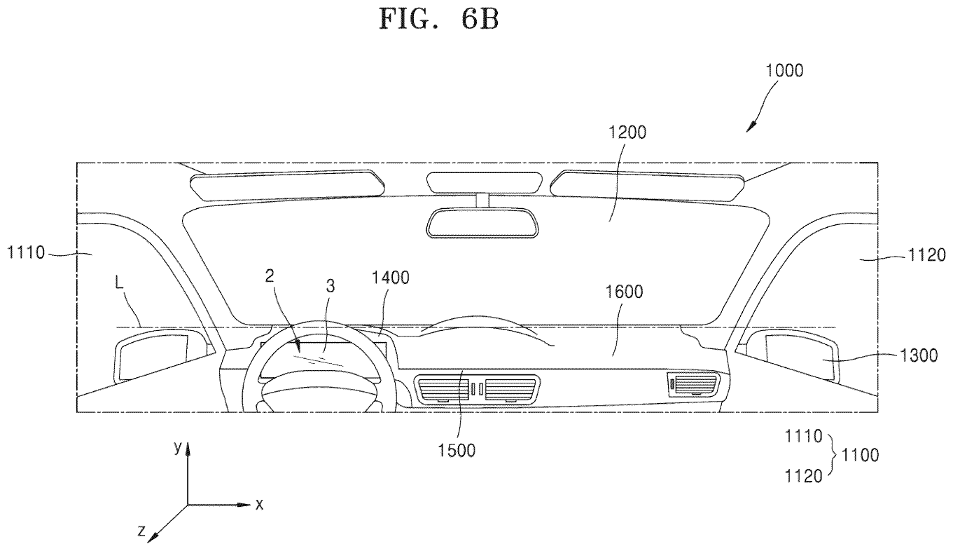

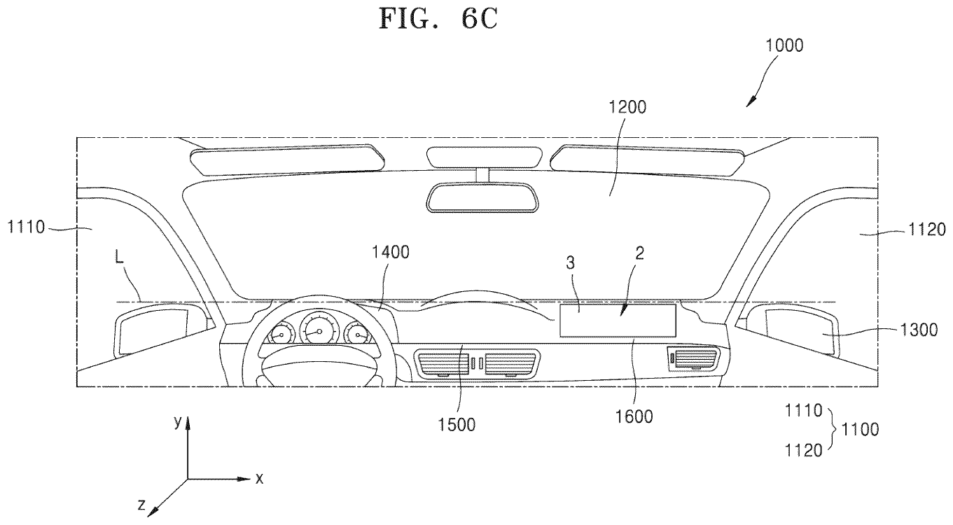

### Description of FIGS. 5 and 6A to 6C

FIG. 5 is a schematic view of an exterior of a vehicle 1000 as an electronic equipment including the light-emitting device according to one or more embodiments of the present disclosure. FIGS. 6A to 6C are each a diagram schematically illustrating an interior of a vehicle 1000 according to one or more embodiments pf the present disclosure.

Referring to FIGS. 5, 6A, 6B, and 6C, the vehicle 1000 may refer to one or more suitable apparatuses for moving an object to be transported, such as a human, an object, or an animal, from a departure point to a destination point. The vehicle 1000 may include a vehicle traveling on a road or a track, a vessel moving over the sea or a river, an airplane flying in the sky utilizing the action of air, and/or the like.

The vehicle 1000 may travel on a road or a track. The vehicle 1000 may move in a set or predetermined direction according to rotation of at least one wheel. For example, the vehicle 1000 may include a three-wheeled or four-wheeled vehicle, a construction machine, a two-wheeled vehicle, a prime mover device, a bicycle, or a train running on a track.

The vehicle 1000 may include a body having an interior and an exterior, and a chassis in which mechanical apparatuses necessary for driving are installed as other parts except for the body. The exterior of the body may include a front panel, a bonnet, a roof panel, a rear panel, a trunk, a filler provided at a boundary between doors, and/or the like. The chassis of the vehicle 1000 may include a power generating device, a power transmitting device, a driving device, a steering device, a braking device, a suspension device, a transmission device, a fuel device, front and rear left and right wheels, and/or the like.

The vehicle 1000 may include a side window glass 1100, a front window glass 1200, a side mirror 1300, a cluster 1400, a center fascia 1500, a passenger seat dashboard 1600, and a display device 2.

The side window glass 1100 and the front window glass 1200 may be partitioned by a pillar arranged between the side window glass 1100 and the front window glass 1200.

The side window glass 1100 may be installed on a side of the vehicle 1000. In some embodiments, the side window glass 1100 may be installed on a door of the vehicle 1000. A plurality of side window glasses 1100 may be provided and may face each other. In some embodiments, the side window glass 1100 may include a first side window glass 1110 and a second side window glass 1120. In some embodiments, the first side window glass 1110 may be arranged adjacent to the cluster 1400. The second side window glass 1120 may be arranged adjacent to the passenger seat dashboard 1600.

In some embodiments, the side window glasses 1100 may be spaced apart from each other in the x-direction or the −x-direction. For example, the first side window glass 1110 and the second side window glass 1120 may be spaced apart from each other in the x direction or the −x direction. In other words, an imaginary straight line L connecting the side window glasses 1100 may extend in the x-direction or the −x-direction. For example, an imaginary straight line L connecting the first side window glass 1110 and the second side window glass 1120 to each other may extend in the x direction or the −x direction.

The front window glass 1200 may be installed in the front of the vehicle 1000. The front window glass 1200 may be arranged between the side window glasses 1100 facing each other.

The side mirror 1300 may provide a rear view of the vehicle 1000. The side mirror 1300 may be installed on the exterior of the vehicle body. In one embodiment, a plurality of side mirrors 1300 may be provided. Any one of the plurality of side mirrors 1300 may be arranged outside the first side window glass 1110. The other one of the plurality of side mirrors 1300 may be arranged outside the second side window glass 1120.

The cluster 1400 may be arranged in front of the steering wheel. The cluster 1400 may include a tachometer, a speedometer, a coolant thermometer, a fuel gauge turn indicator, a high beam indicator, a warning lamp, a seat belt warning lamp, an odometer, an automatic shift selector indicator lamp, a door open warning lamp, an engine oil warning lamp, and/or a low fuel warning light.

The center fascia 1500 may include a control panel on which a plurality of buttons for adjusting an audio device, an air conditioning device, and/or a heater of a seat are disposed. The center fascia 1500 may be arranged on one side of the cluster 1400.

The passenger seat dashboard 1600 may be spaced apart from the cluster 1400 with the center fascia 1500 arranged therebetween. In one embodiment, the cluster 1400 may be arranged to correspond to a driver seat, and the passenger seat dashboard 1600 may be disposed to correspond to a passenger seat. In one embodiment, the cluster 1400 may be adjacent to the first side window glass 1110, and the passenger seat dashboard 1600 may be adjacent to the second side window glass 1120.

In one or more embodiments, the display device 2 may include a display panel 3, and the display panel 3 may display an image. The display device 2 may be arranged inside the vehicle 1000. In one embodiment, the display device 2 may be arranged between the side window glasses 1100 facing each other. The display device 2 may be arranged on at least one selected from among the cluster 1400, the center fascia 1500, and the passenger seat dashboard 1600.

The display device 2 may include an organic light-emitting display device, an inorganic EL display device, a quantum dot display device, and/or the like. Hereinafter, as the display device 2 according to one or more embodiments of the present disclosure, an organic light-emitting display device display including the light-emitting device according to one or more embodiments of the present disclosure will be described as an example, but one or more suitable types (kinds) of display devices as described above may be utilized in embodiments of the present disclosure.

Referring to FIG. 6A, the display device 2 may be arranged on the center fascia 1500. In one embodiment, the display device 2 may display navigation information. In one embodiment, the display device 2 may display information regarding audio, video, and/or vehicle settings.

Referring to FIG. 6B, the display device 2 may be arranged on the cluster 1400. When the display device 2 is arranged on the cluster 1400, the cluster 1400 may display driving information and/or the like through the display device 2. For example, the cluster 1400 may be implemented digitally. The digital cluster 1400 may display vehicle information and driving information as images. For example, in some embodiments, a needle and a gauge of a tachometer and one or more suitable warning light icons may be displayed by digital signals.

Referring to FIG. 6C, the display device 2 may be arranged on the passenger seat dashboard 1600. The display device 2 may be embedded in the passenger seat dashboard 1600 or arranged on the passenger seat dashboard 1600. In some embodiments, the display device 2 arranged on the passenger seat dashboard 1600 may display an image related to information displayed on the cluster 1400 and/or information displayed on the center fascia 1500. In one or more embodiments, the display device 2 arranged on the passenger seat dashboard 1600 may display information different from information displayed on the cluster 1400 and/or information displayed on the center fascia 1500.

### Manufacturing Method

Respective layers included in the hole transport region, the emission layer, and respective layers included in the electron transport region may be formed in a certain region by utilizing one or more suitable methods selected from vacuum deposition, spin coating, casting, Langmuir-Blodgett (LB) deposition, ink-jet printing, laser-printing, laser-induced thermal imaging, and/or the like.

When respective layers included in the hole transport region, the emission layer, and respective layers included in the electron transport region are formed by vacuum deposition, the deposition may be performed at a deposition temperature of about 100° C. to about 500° C., a vacuum degree of about 10⁻⁸ torr to about 10⁻³ torr, and a deposition speed of about 0.01 Å/sec to about 100 Å/sec, depending on a material to be included in a layer to be formed and the structure of a layer to be formed.

### Definition of Terms

The term “C₃-C₆₀ carbocyclic group” as utilized herein refers to a cyclic group consisting of carbon only as a ring-forming atom and having three to sixty carbon atoms, and the term “C₁-C₆₀ heterocyclic group” as utilized herein refers to a cyclic group that has one to sixty carbon atoms and further has, in addition to carbon, a heteroatom as a ring-forming atom. The C₃-C₆₀ carbocyclic group and the C₁-C₆₀ heterocyclic group may each be a monocyclic group consisting of one ring or a polycyclic group in which two or more rings are condensed with each other. For example, the number of ring-forming atoms of the C₁-C₆₀ heterocyclic group may be from 3 to 61.

The “cyclic group” as utilized herein may include both (e.g., simultaneously) the C₃-C₆₀ carbocyclic group and/or the C₁-C₆₀ heterocyclic group.

The term “π electron-rich C₃-C₆₀ cyclic group” as utilized herein refers to a cyclic group that has three to sixty carbon atoms and does not include *—N=* as a ring-forming moiety, and the term “π electron-deficient nitrogen-containing C₁-C₆₀ cyclic group” as utilized herein refers to a heterocyclic group that has one to sixty carbon atoms and includes *—N=*′ as a ring-forming moiety.

For example,
the C₃-C₆₀ carbocyclic group may be i) a T1 group or ii) a condensed cyclic group in which two or more T1 groups are condensed with each other (for example, a cyclopentadiene group, an adamantane group, a norbornane group, a benzene group, a pentalene group, a naphthalene group, an azulene group, an indacene group, an acenaphthylene group, a phenalene group, a phenanthrene group, an anthracene group, a fluoranthene group, a triphenylene group, a pyrene group, a chrysene group, a perylene group, a pentaphene group, a heptalene group, a naphthacene group, a picene group, a hexacene group, a pentacene group, a rubicene group, a coronene group, an ovalene group, an indene group, a fluorene group, a spiro-bifluorene group, a benzofluorene group, an indenophenanthrene group, or an indenoanthracene group),
the C₁-C₆₀ heterocyclic group may be i) a T2 group, ii) a condensed cyclic group in which at least two T2 groups are condensed with each other, or iii) a condensed cyclic group in which at least one T2 group and at least one T1 group are condensed with each other (for example, a pyrrole group, a thiophene group, a furan group, an indole group, a benzoindole group, a naphthoindole group, an isoindole group, a benzoisoindole group, a naphthoisoindole group, a benzosilole group, a benzothiophene group, a benzofuran group, a carbazole group, a dibenzosilole group, a dibenzothiophene group, a dibenzofuran group, an indenocarbazole group, an indolocarbazole group, a benzofurocarbazole group, a benzothienocarbazole group, a benzosilolocarbazole group, a benzoindolocarbazole group, a benzocarbazole group, a benzonaphthofuran group, a benzonaphthothiophene group, a benzonaphthosilole group, a benzofurodibenzofuran group, a benzofurodibenzothiophene group, a benzothienodibenzothiophene group, a pyrazole group, an imidazole group, a triazole group, an oxazole group, an isoxazole group, an oxadiazole group, a thiazole group, an isothiazole group, a thiadiazole group, a benzopyrazole group, a benzimidazole group, a benzoxazole group, a benzoisoxazole group, a benzothiazole group, a benzoisothiazole group, a pyridine group, a pyrimidine group, a pyrazine group, a pyridazine group, a triazine group, a quinoline group, an isoquinoline group, a benzoquinoline group, a benzoisoquinoline group, a quinoxaline group, a benzoquinoxaline group, a quinazoline group, a benzoquinazoline group, a phenanthroline group, a cinnoline group, a phthalazine group, a naphthyridine group, an imidazopyridine group, an imidazopyrimidine group, an imidazotriazine group, an imidazopyrazine group, an imidazopyridazine group, an azacarbazole group, an azafluorene group, an azadibenzosilole group, an azadibenzothiophene group, an azadibenzofuran group, and/or the like),
the π electron-rich C₃-C₆₀ cyclic group may be i) a T1 group, ii) a condensed cyclic group in which at least two T1 groups are condensed with each other, iii) a T3 group, iv) a condensed cyclic group in which at least two T3 groups are condensed with each other, or v) a condensed cyclic group in which at least one T3 group and at least one T1 group are condensed with each other (for example, the C₃-C₆₀ carbocyclic group, a 1H-pyrrole group, a silole group, a borole group, a 2H-pyrrole group, a 3H-pyrrole group, a thiophene group, a furan group, an indole group, a benzoindole group, a naphthoindole group, an isoindole group, a benzoisoindole group, a naphthoisoindole group, a benzosilole group, a benzothiophene group, a benzofuran group, a carbazole group, a dibenzosilole group, a dibenzothiophene group, a dibenzofuran group, an indenocarbazole group, an indolocarbazole group, a benzofurocarbazole group, a benzothienocarbazole group, a benzosilolocarbazole group, a benzoindolocarbazole group, a benzocarbazole group, a benzonaphthofuran group, a benzonaphthothiophene group, a benzonaphthosilole group, a benzofurodibenzofuran group, a benzofurodibenzothiophene group, a benzothienodibenzothiophene group, and/or the like),
the π electron-deficient nitrogen-containing C₁-C₆₀ cyclic group may be i) a T4 group, ii) a condensed cyclic group in which at least two T4 groups are condensed with each other, iii) a condensed cyclic group in which at least one T4 group and at least one T1 group are condensed with each other, iv) a condensed cyclic group in which at least one T4 group and at least one T3 group are condensed with each other, or v) a condensed cyclic group in which at least one T4 group, at least one T1 group, and at least one T3 group are condensed with one another (for example, a pyrazole group, an imidazole group, a triazole group, an oxazole group, an isoxazole group, an oxadiazole group, a thiazole group, an isothiazole group, a thiadiazole group, a benzopyrazole group, a benzimidazole group, a benzoxazole group, a benzoisoxazole group, a benzothiazole group, a benzoisothiazole group, a pyridine group, a pyrimidine group, a pyrazine group, a pyridazine group, a triazine group, a quinoline group, an isoquinoline group, a benzoquinoline group, a benzoisoquinoline group, a quinoxaline group, a benzoquinoxaline group, a quinazoline group, a benzoquinazoline group, a phenanthroline group, a cinnoline group, a phthalazine group, a naphthyridine group, an imidazopyridine group, an imidazopyrimidine group, an imidazotriazine group, an imidazopyrazine group, an imidazopyridazine group, an azacarbazole group, an azafluorene group, an azadibenzosilole group, an azadibenzothiophene group, an azadibenzofuran group, and/or the like),
the T1 group may be a cyclopropane group, a cyclobutane group, a cyclopentane group, a cyclohexane group, a cycloheptane group, a cyclooctane group, a cyclobutene group, a cyclopentene group, a cyclopentadiene group, a cyclohexene group, a cyclohexadiene group, a cycloheptene group, an adamantane group, a norbornane (or bicyclo[2.2.1]heptane) group, a norbornene group, a bicyclo[1.1.1]pentane group, a bicyclo[2.1.1]hexane group, a bicyclo[2.2.2]octane group, or a benzene group,
the T2 group may be a furan group, a thiophene group, a 1H-pyrrole group, a silole group, a borole group, a 2H-pyrrole group, a 3H-pyrrole group, an imidazole group, a pyrazole group, a triazole group, a tetrazole group, an oxazole group, an isoxazole group, an oxadiazole group, a thiazole group, an isothiazole group, a thiadiazole group, an azasilole group, an azaborole group, a pyridine group, a pyrimidine group, a pyrazine group, a pyridazine group, a triazine group, a tetrazine group, a pyrrolidine group, an imidazolidine group, a dihydropyrrole group, a piperidine group, a tetrahydropyridine group, a dihydropyridine group, a hexahydropyrimidine group, a tetrahydropyrimidine group, a dihydropyrimidine group, a piperazine group, a tetrahydropyrazine group, a dihydropyrazine group, a tetrahydropyridazine group, or a dihydropyridazine group,
the T3 group may be a furan group, a thiophene group, a 1H-pyrrole group, a silole group, or a borole group, and
the T4 group may be a 2H-pyrrole group, a 3H-pyrrole group, an imidazole group, a pyrazole group, a triazole group, a tetrazole group, an oxazole group, an isoxazole group, an oxadiazole group, a thiazole group, an isothiazole group, a thiadiazole group, an azasilole group, an azaborole group, a pyridine group, a pyrimidine group, a pyrazine group, a pyridazine group, a triazine group, or a tetrazine group.

The terms “cyclic group,” “C₃-C₆₀ carbocyclic group,” “C₁-C₆₀ heterocyclic group,” “π electron-rich C₃-C₆₀ cyclic group,” or “π electron-deficient nitrogen-containing C₁-C₆₀ cyclic group” as utilized herein may refer to a group condensed to any cyclic group, a monovalent group, or a polyvalent group (for example, a divalent group, a trivalent group, a tetravalent group, etc.) according to the structure of a formula for which the corresponding term is utilized. For example, the “benzene group” may be a benzo group, a phenyl group, a phenylene group, and/or the like, which may be easily understood by one of ordinary skill in the art according to the structure of a formula including the “benzene group.”

Depending on context, in the present disclosure, a divalent group may refer or be a polyvalent group (e.g., trivalent, tetravalent, etc., and not just divalent) per, e.g., the structure of a formula in connection with which of the terms are utilized.

Non-limiting examples of the monovalent C₃-C₆₀ carbocyclic group and the monovalent C₁-C₆₀ heterocyclic group are a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₁-C₆₀ heteroaryl group, a monovalent non-aromatic condensed polycyclic group, and a monovalent non-aromatic condensed heteropolycyclic group. Non-limiting examples of the divalent C₃-C₆₀ carbocyclic group and the divalent C₁-C₆₀ heterocyclic group are a C₃-C₁₀ cycloalkylene group, a C₁-C₁₀ heterocycloalkylene group, a C₃-C₁₀ cycloalkenylene group, a C₁-C₁₀ heterocycloalkenylene group, a C₆-C₆₀ arylene group, a C₁-C₆₀ heteroarylene group, a divalent non-aromatic condensed polycyclic group, and a substituted or unsubstituted divalent non-aromatic condensed heteropolycyclic group.

The term “C₁-C₆₀ alkyl group” as utilized herein refers to a linear or branched aliphatic hydrocarbon monovalent group that has one to sixty carbon atoms, and non-limiting examples thereof may be a methyl group, an ethyl group, an n-propyl group, an isopropyl group, an n-butyl group, a sec-butyl group, an isobutyl group, a tert-butyl group, an n-pentyl group, a tert-pentyl group, a neopentyl group, an isopentyl group, a sec-pentyl group, a 3-pentyl group, a sec-isopentyl group, an n-hexyl group, an isohexyl group, a sec-hexyl group, a tert-hexyl group, an n-heptyl group, an isoheptyl group, a sec-heptyl group, a tert-heptyl group, an n-octyl group, an isooctyl group, a sec-octyl group, a tert-octyl group, an n-nonyl group, an isononyl group, a sec-nonyl group, a tert-nonyl group, an n-decyl group, an isodecyl group, a sec-decyl group, and a tert-decyl group. The term “C₁-C₆₀ alkylene group” as utilized herein refers to a divalent group having the same structure as the C₁-C₆₀ alkyl group.

The term “C₂-C₆₀ alkenyl group” as utilized herein refers to a monovalent hydrocarbon group having at least one carbon-carbon double bond in the middle or at the terminus of the C₂-C₆₀ alkyl group, and non-limiting examples thereof may be an ethenyl group, a propenyl group, a butenyl group, and/or the like. The term “C₂-C₆₀ alkenylene group” as utilized herein refers to a divalent group having the same structure as the C₂-C₆₀ alkenyl group.

The term “C₂-C₆₀ alkynyl group” as utilized herein refers to a monovalent hydrocarbon group having at least one carbon-carbon triple bond in the middle or at the terminus of the C₂-C₆₀ alkyl group, and non-limiting examples thereof may be an ethynyl group, a propynyl group, and/or the like. The term “C₂-C₆₀ alkynylene group” as utilized herein refers to a divalent group having the same structure as the C₂-C₆₀ alkynyl group.

The term “C₁-C₆₀ alkoxy group” as utilized herein refers to a monovalent group represented by -OA₁₀₁ (wherein A₁₀₁ is the C₁-C₆₀ alkyl group), and non-limiting examples thereof may be a methoxy group, an ethoxy group, an isopropyloxy group, and/or the like.

The term “C₃-C₁₀ cycloalkyl group” as utilized herein refers to a monovalent saturated hydrocarbon cyclic group having 3 to 10 carbon atoms, and non-limiting examples thereof may be a cyclopropyl group, a cyclobutyl group, a cyclopentyl group, a cyclohexyl group, a cycloheptyl group, a cyclooctyl group, an adamantanyl group, a norbornanyl group (or bicyclo[2.2.1]heptyl group), a bicyclo[1.1.1]pentyl group, a bicyclo[2.1.1]hexyl group, a bicyclo[2.2.2]octyl group, and/or the like. The term “C₃-C₁₀ cycloalkylene group” as utilized herein refers to a divalent group having the same structure as the C₃-C₁₀ cycloalkyl group.

The term “C₁-C₁₀ heterocycloalkyl group” as utilized herein refers to a monovalent cyclic group of 1 to 10 carbon atoms, further including, in addition to carbon atoms, at least one heteroatom, as ring-forming atoms, and non-limiting examples thereof may be a 1,2,3,4-oxatriazolidinyl group, a tetrahydrofuranyl group, a tetrahydrothiophenyl group, and/or the like. The term “C₁-C₁₀ heterocycloalkylene group” as utilized herein refers to a divalent group having the same structure as the C₁-C₁₀ heterocycloalkyl group.

The term “C₃-C₁₀ cycloalkenyl group” as utilized herein refers to a monovalent cyclic group that has three to ten carbon atoms and at least one carbon-carbon double bond in the ring thereof and no aromaticity, and non-limiting examples thereof may be a cyclopentenyl group, a cyclohexenyl group, a cycloheptenyl group, and/or the like. The term “C₃-C₁₀ cycloalkenylene group” as utilized herein refers to a divalent group having the same structure as the C₃-C₁₀ cycloalkenyl group.

The term “C₁-C₁₀ heterocycloalkenyl group” as utilized herein refers to a monovalent cyclic group of 1 to 10 carbon atoms, further including, in addition to carbon atoms, at least one heteroatom, as ring-forming atoms, and having at least one carbon-carbon double bond in the cyclic structure thereof. Non-limiting examples of the C₁-C₁₀ heterocycloalkenyl group may be a 4,5-dihydro-1,2,3,4-oxatriazolyl group, a 2,3-dihydrofuranyl group, a 2,3-dihydrothiophenyl group, and/or the like. The term “C₁-C₁₀ heterocycloalkenylene group” as utilized herein refers to a divalent group having the same structure as the C₁-C₁₀ heterocycloalkenyl group.

The term “C₆-C₆₀ aryl group” as utilized herein refers to a monovalent group having a carbocyclic aromatic system of 6 to 60 carbon atoms, and the term “C₆-C₆₀ arylene group” as utilized herein refers to a divalent group having a carbocyclic aromatic system of 6 to 60 carbon atoms. Non-limiting examples of the C₆-C₆₀ aryl group may be a phenyl group, a pentalenyl group, a naphthyl group, an azulenyl group, an indacenyl group, an acenaphthyl group, a phenalenyl group, a phenanthrenyl group, an anthracenyl group, a fluoranthenyl group, a triphenylenyl group, a pyrenyl group, a chrysenyl group, a perylenyl group, a pentaphenyl group, a heptalenyl group, a naphthacenyl group, a picenyl group, a hexacenyl group, a pentacenyl group, a rubicenyl group, a coronenyl group, an ovalenyl group, and/or the like. When the C₆-C₆₀ aryl group and the C₆-C₆₀ arylene group each include two or more rings, the rings may be condensed with each other.

The term “C₁-C₆₀ heteroaryl group” as utilized herein refers to a monovalent group having a heterocyclic aromatic system of 1 to 60 carbon atoms, further including, in addition to carbon atoms, at least one heteroatom, as ring-forming atoms. The term “C₁-C₆₀ heteroarylene group” as utilized herein refers to a divalent group having a heterocyclic aromatic system of 1 to 60 carbon atoms, further including, in addition to carbon atoms, at least one heteroatom, as ring-forming atoms. Non-limiting examples of the C₁-C₆₀ heteroaryl group may be a pyridinyl group, a pyrimidinyl group, a pyrazinyl group, a pyridazinyl group, a triazinyl group, a quinolinyl group, a benzoquinolinyl group, an isoquinolinyl group, a benzoisoquinolinyl group, a quinoxalinyl group, a benzoquinoxalinyl group, a quinazolinyl group, a benzoquinazolinyl group, a cinnolinyl group, a phenanthrolinyl group, a phthalazinyl group, and a naphthyridinyl group. When the C₁-C₆₀ heteroaryl group and the C₁-C₆₀ heteroarylene group each include two or more rings, the rings may be condensed with each other.

The term “monovalent non-aromatic condensed polycyclic group” as utilized herein refers to a monovalent group (for example, having 8 to 60 carbon atoms) having two or more rings condensed to each other, only carbon atoms as ring-forming atoms, and no aromaticity in its entire molecular structure as a whole. Non-limiting examples of the monovalent non-aromatic condensed polycyclic group may be an indenyl group, a fluorenyl group, a spiro-bifluorenyl group, a benzofluorenyl group, an indenophenanthrenyl group, an indeno anthracenyl group, and/or the like. The term “divalent non-aromatic condensed polycyclic group” as utilized herein refers to a divalent group having the same structure as the monovalent non-aromatic condensed polycyclic group described above.

The term “monovalent non-aromatic condensed heteropolycyclic group” as utilized herein refers to a monovalent group (for example, having 1 to 60 carbon atoms) having two or more rings condensed to each other, further including, in addition to carbon atoms, at least one heteroatom, as ring-forming atoms, and having non-aromaticity in its entire molecular structure as a whole. Non-limiting examples of the monovalent non-aromatic condensed heteropolycyclic group may be a pyrrolyl group, a thiophenyl group, a furanyl group, an indolyl group, a benzoindolyl group, a naphthoindolyl group, an isoindolyl group, a benzoisoindolyl group, a naphthoisoindolyl group, a benzosilolyl group, a benzothiophenyl group, a benzofuranyl group, a carbazolyl group, a dibenzosilolyl group, a dibenzothiophenyl group, a dibenzofuranyl group, an azacarbazolyl group, an azafluorenyl group, an azadibenzosilolyl group, an azadibenzothiophenyl group, an azadibenzofuranyl group, a pyrazolyl group, an imidazolyl group, a triazolyl group, a tetrazolyl group, an oxazolyl group, an isoxazolyl group, a thiazolyl group, an isothiazolyl group, an oxadiazolyl group, a thiadiazolyl group, a benzopyrazolyl group, a benzimidazolyl group, a benzoxazolyl group, a benzothiazolyl group, a benzoxadiazolyl group, a benzothiadiazolyl group, an imidazopyridinyl group, an imidazopyrimidinyl group, an imidazotriazinyl group, an imidazopyrazinyl group, an imidazopyridazinyl group, an indeno carbazolyl group, an indolocarbazolyl group, a benzofurocarbazolyl group, a benzothienocarbazolyl group, a benzosilolocarbazolyl group, a benzoindolocarbazolyl group, a benzocarbazolyl group, a benzonaphthofuranyl group, a benzonaphthothiophenyl group, a benzonaphthosilolyl group, a benzofurodibenzofuranyl group, a benzofurodibenzothiophenyl group, and a benzothienodibenzothiophenyl group. The term “divalent non-aromatic condensed heteropolycyclic group” as utilized herein refers to a divalent group having the same structure as the monovalent non-aromatic condensed heteropolycyclic group described above.

The term “C₆-C₆₀ aryloxy group” as utilized herein indicates -OA₁₀₂ (wherein A₁₀₂ is the C₆-C₆₀ aryl group), and the term “C₆-C₆₀ arylthio group” as utilized herein indicates -SA₁₀₃ (wherein A₁₀₃ is the C₆-C₆₀ aryl group).

The term “C₇-C₆₀ arylalkyl group” as utilized herein refers to -A₁₀₄A₁₀₅ (wherein A₁₀₄ is a C₁-C₅₄ alkylene group, and A₁₀₅ is a C₆-C₅₉ aryl group), and the term “C₂-C₆₀ heteroarylalkyl group” as utilized herein refers to -A₁₀₆A₁₀₇ (wherein A₁₀₆ is a C₁-C₅₉ alkylene group, and A₁₀₇ is a C₁-C₅₉ heteroaryl group).

The term “R₁₀ₐ” as utilized herein may be:
deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or a combination thereof;
a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, or a C₂-C₆₀ heteroarylalkyl group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or a combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O) (Q₃₁) (Q₃₂).

In the present disclosure, Q₁ to Q₃, Q₁₁ to Q₁₃, Q₂₁ to Q₂₃, and Q₃₁ to Q₃₃ may each independently be: hydrogen; deuterium; —F; —C₁; —Br; —I; a hydroxyl group; a cyano group; a nitro group; a C₁-C₆₀ alkyl group; a C₂-C₆₀ alkenyl group; a C₂-C₆₀ alkynyl group; a C₁-C₆₀ alkoxy group; a C₃-C₆₀ carbocyclic group or a C₁-C₆₀ heterocyclic group, each unsubstituted or substituted with deuterium, —F, a cyano group, a C₁-C₆₀ alkyl group, a C₁-C₆₀ alkoxy group, a phenyl group, a biphenyl group, or a combination thereof; a C₇-C₆₀ arylalkyl group; or a C₂-C₆₀ heteroarylalkyl group.

The term “heteroatom” as utilized herein refers to any atom other than a carbon atom. Non-limiting examples of the heteroatom may be O, S, N, P, Si, B, Ge, Se, and/or a combination thereof.

In the present disclosure, the third-row transition metal may include hafnium (Hf), tantalum (Ta), tungsten (W), rhenium (Re), osmium (Os), iridium (Ir), platinum (Pt), gold (Au), and/or the like.

In the present disclosure, “Ph” refers to a phenyl group, “Me” refers to a methyl group, “Et” refers to an ethyl group, “tert-Bu” or “t-Bu^{t}” refers to a tert-butyl group, and “OMe” refers to a methoxy group.

The term “biphenyl group” as utilized herein refers to “a phenyl group substituted with a phenyl group.” In other words, the “biphenyl group” may be a substituted phenyl group having a C₆-C₆₀ aryl group as a substituent.

The term “terphenyl group” as utilized herein refers to “a phenyl group substituted with a biphenyl group.” In other words, the “terphenyl group” may be a substituted phenyl group having, as a substituent, a C₆-C₆₀ aryl group substituted with a C₆-C₆₀ aryl group.

* and * as utilized herein, unless defined otherwise, each refer to a binding site to a neighboring atom in a corresponding formula or moiety.

In the present disclosure, the x-axis, y-axis, and z-axis are not limited to three axes in an orthogonal coordinate system, and may be interpreted in a broad sense including these axes. For example, the x-axis, y-axis, and z-axis may refer to those orthogonal to each other, or may refer to those in different directions that are not orthogonal to each other.

Hereinafter, compounds according to one or more embodiments and light-emitting devices according to one or more embodiments will be described in more detail with reference to the following Synthesis Examples and Examples. The wording “B was utilized instead of A” utilized in describing Synthesis Examples refers to that an identical molar equivalent of B was utilized in place of A.

### EXAMPLES

The amine-based compounds according to one or more embodiments may be, for example, synthesized as below. However, the synthesis methods of the amine-based compounds according to one or more embodiments are not limited thereto.

### Synthesis Example 1. Synthesis of Compound 2

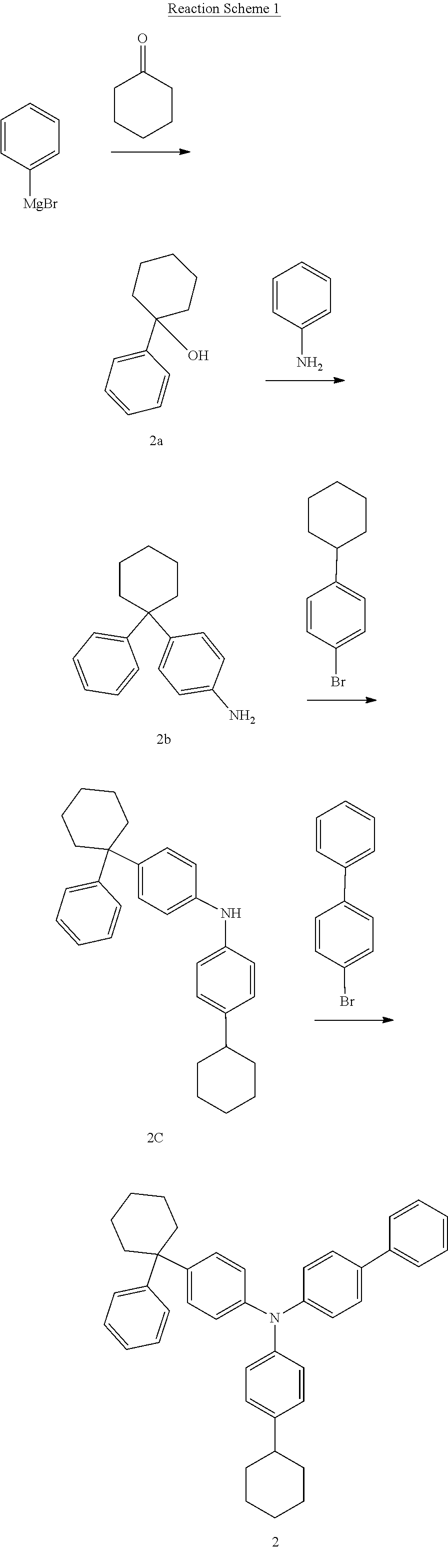

### 1-1. Synthesis of Intermediate 2a

Cyclohexanone (1.0 eq.) was dissolved in 300 mL of tetrahydrofuran, and 3 M of phenylmagnesium bromide (1.2 eq.) was added thereto in a nitrogen atmosphere at 0° C. to prepare a reaction solution. Then, the reaction solution was stirred at room temperature for 1 hour and at 70° C. for 2 hours. After the reaction was completed, the reaction product was washed slowly with ammonium chloride and water, and washed three times with diethyl ether to obtain an organic layer. The organic layer was dried with MgSO₄ and then dried under reduced pressure. The resultant was subjected to column chromatography to obtain 9.27 g of Intermediate 2a. (yield: 67%)

### 1-2. Synthesis of Intermediate 2b

A reaction solution including Intermediate 2a (1.0 eq), aniline (10.0 eq), and methanesulfonic acid (MsOH) (9.0 eq) was prepared in a microwave (Mutiwave 7000, Anton-paar Korea Co., Ltd.). The reaction solution was stirred at 200° C. under a pressure of 63 bar for 30 minutes. After the reaction was completed, the reaction product was washed slowly with sodium hydroxide and water, and washed three times with diethyl ether to obtain an organic layer. The organic layer was dried with MgSO₄ and then dried under reduced pressure. The resultant was subjected to column chromatography to obtain 2.7 g of Intermediate 2b. (yield: 31%)

### 1-3. Synthesis of Intermediate 2c

Intermediate 2b (1.0 eq.), 1-bromo-4-cyclohexylbenzene (1.0 eq.), tris(dibenzylideneacetone)dipalladium (0) (0.05 eq.), tri-tert-butylphosphine (0.10 eq.), and sodium tert-butoxide (1.5 eq.) were dissolved in 50 mL of toluene to prepare a reaction solution. The reaction solution was stirred at 80° C. for 1 hour in a nitrogen atmosphere. After the reaction was completed, the reaction product was washed three times with water and diethyl ether to obtain an organic layer. The organic layer was dried with MgSO₄ and then dried under reduced pressure. The resultant was subjected to column chromatography to obtain 2.0 g of Intermediate 2c. (yield: 46%)

### 1-4. Synthesis of Compound 2

Intermediate 2c (1.0 eq.), 4-bromo-1,1′-biphenyl (1.0 eq.), tris(dibenzylideneacetone)dipalladium (0) (0.05 eq.), tri-tert-butylphosphine (0.10 eq.), and sodium tert-butoxide (1.5 eq.) were dissolved in 50 mL of toluene to prepare a reaction solution. The reaction solution was stirred in a nitrogen atmosphere at 80° C. for 1 hour. After the reaction was completed, the reaction product was washed three times with water and diethyl ether to obtain an organic layer. The organic layer was dried with MgSO₄ and then dried under reduced pressure. The resultant was subjected to column chromatography to obtain 2.8 g of Compound 2. (yield: 84%)

### Synthesis Example 2. Synthesis of Compound 23

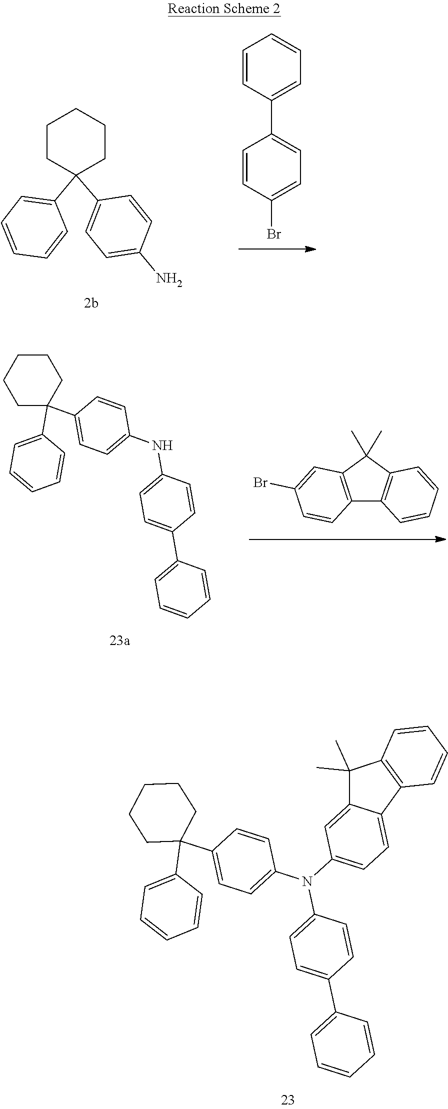 [SMILES: Nc1ccc(C2(c3ccccc3)CCCCC2)cc1 ; Brc1ccc(-c2ccccc2)cc1 ; c1ccc(-c2ccc(Nc3ccc(C4(c5ccccc5)CCCCC4)cc3)cc2)cc1 ; CC1(C)c2ccccc2-c2ccc(Br)cc21 ; CC1(C)c2ccccc2-c2ccc(N(c3ccc(-c4ccccc4)cc3)c3ccc(C4(c5ccccc5)CCCCC4)cc3)cc21]

### 2-1. Synthesis of Intermediate 23a

Intermediate 2b (1.0 eq.), 4-bromo-1,1′-biphenyl (1.0 eq.), tris(dibenzylideneacetone)dipalladium (0) (0.05 eq.), tri-tert-butylphosphine (0.10 eq.), and sodium tert-butoxide (1.5 eq.) were dissolved in 50 mL of toluene to prepare a reaction solution. The reaction solution was stirred in a nitrogen atmosphere at 80° C. for 1 hour. After the reaction was completed, the reaction product was washed three times with water and diethyl ether to obtain an organic layer. The organic layer was dried with MgSO₄ and then dried under reduced pressure. The resultant was subjected to column chromatography to obtain 2.2 g of Intermediate 23a. (yield: 51%)

### 2-2. Synthesis of Compound 23

Intermediate 23a (1.0 eq.), 2-bromo-9,9-dimethyl-9H-fluorene (1.0 eq.), tris(dibenzylideneacetone)dipalladium (0) (0.05 eq.), tri-tert-butylphosphine (0.10 eq.), and sodium tert-butoxide (1.5 eq.) were dissolved in 50 mL of toluene and then stirred for 1 hour at 80° C. in a nitrogen atmosphere. After the reaction was completed, an organic layer obtained by washing three times with water and diethyl ether was dried with MgSO₄ and then dried under reduced pressure. The resultant was subjected to column chromatography to obtain 2.9 g of Compound 23. (yield: 82%)

### Synthesis Example 3. Synthesis of Compound 28

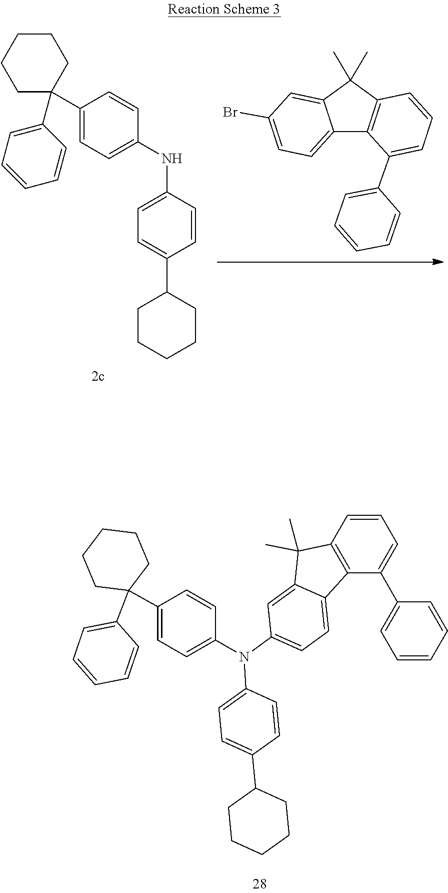 [SMILES: c1ccc(C2(c3ccc(Nc4ccc(C5CCCCC5)cc4)cc3)CCCCC2)cc1 ; CC1(C)c2cc(Br)ccc2-c2c(-c3ccccc3)cccc21 ; CC1(C)c2cc(N(c3ccc(C4CCCCC4)cc3)c3ccc(C4(c5ccccc5)CCCCC4)cc3)ccc2-c2c(-c3ccccc3)cccc21]

### 3-1. Synthesis of Compound 28

Intermediate 2c (1.0 eq.), 2-bromo-9,9-dimethyl-5-phenyl-9H-fluorene (1.0 eq.), tris(dibenzylideneacetone)dipalladium (0) (0.05 eq.), tri-tert-butylphosphine (0.10 eq.), and sodium tert-butoxide (1.5 eq.) were dissolved in 50 mL of toluene to prepare a reaction solution. The reaction solution was stirred at 80° C. for 1 hour in a nitrogen atmosphere. After the reaction was completed, the reaction product was washed three times with water and diethyl ether to obtain an organic layer. The organic layer was dried with MgSO₄ and then dried under reduced pressure. The resultant was subjected to column chromatography to obtain 1.8 g of Compound 28. (yield: 81%)

### Synthesis Example 4. Synthesis of Compound 37

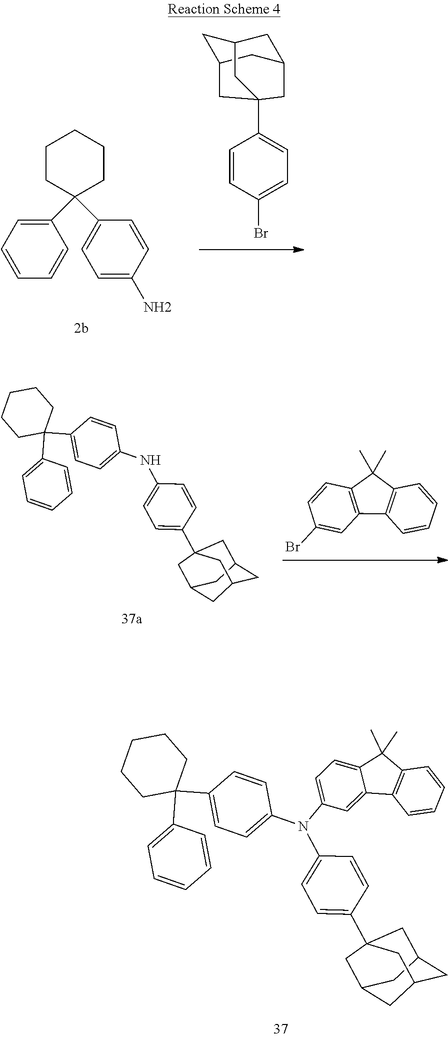 [SMILES: Nc1ccc(C2(c3ccccc3)CCCCC2)cc1 ; Brc1ccc(C23CC4CC(CC(C4)C2)C3)cc1 ; c1ccc(C2(c3ccc(Nc4ccc(C56CC7CC(CC(C7)C5)C6)cc4)cc3)CCCCC2)cc1 ; CC1(C)c2ccccc2-c2cc(Br)ccc21 ; CC1(C)c2ccccc2-c2cc(N(c3ccc(C45CC6CC(CC(C6)C4)C5)cc3)c3ccc(C4(c5ccccc5)CCCCC4)cc3)ccc21]

### 4-1. Synthesis of Intermediate 37

Intermediate 2b (1.0 eq.), (3r,5r,7r)-1-(4-bromophenyl)adamantane (1.0 eq.), tris(dibenzylideneacetone)dipalladium (0) (0.05 eq.), tri-tert-butylphosphine (0.10 eq.), and sodium tert-butoxide (1.5 eq.) were dissolved in 50 mL of toluene to prepare a reaction solution. The reaction solution was stirred at 80° C. for 1 hour in a nitrogen atmosphere. After the reaction was completed, the reaction product was washed three times with water and diethyl ether to obtain an organic layer. The organic layer was dried with MgSO₄ and then dried under reduced pressure. The resultant was subjected to column chromatography to obtain 2.4 g of Intermediate 37a. (yield: 55%)

### 4-2. Synthesis of Compound 37

Intermediate 37a (1.0 eq.), 3-bromo-9,9-dimethyl-9H-fluorene (1.0 eq.), tris(dibenzylideneacetone)dipalladium (0) (0.05 eq.), tri-tert-butylphosphine (0.10 eq.), and sodium tert-butoxide (1.5 eq.) were dissolved in 50 mL of toluene to prepare a reaction solution. The reaction solution was stirred at 80° C. for 1 hour in a nitrogen atmosphere. After the reaction was completed, the reaction product was washed three times with water and diethyl ether to obtain an organic layer. The organic layer was dried with MgSO₄ and then dried under reduced pressure. The resultant was subjected to column chromatography to obtain 1.9 g of Compound 37. (yield: 83%)

### Synthesis Example 5. Synthesis of Compound 78

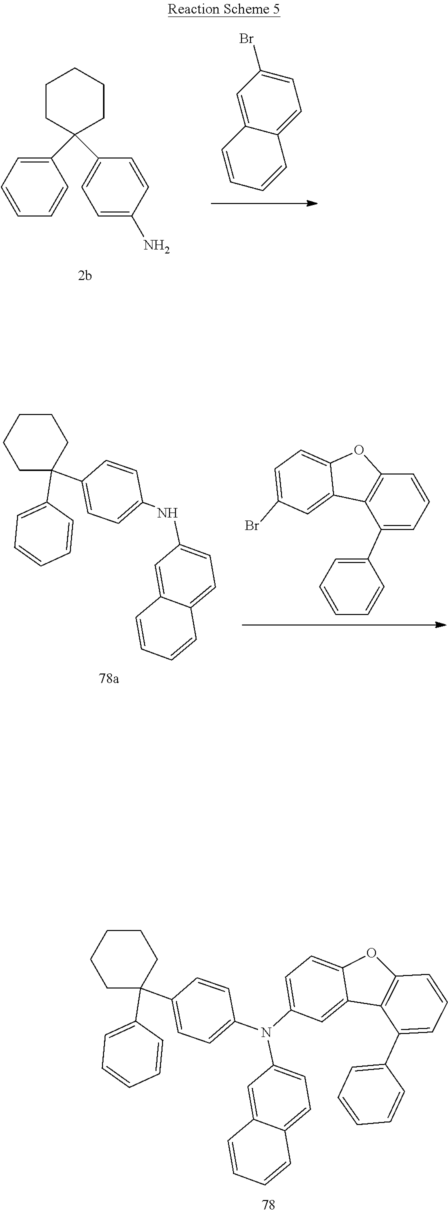 [SMILES: Nc1ccc(C2(c3ccccc3)CCCCC2)cc1 ; Brc1ccc2ccccc2c1 ; c1ccc(C2(c3ccc(Nc4ccc5ccccc5c4)cc3)CCCCC2)cc1 ; Brc1ccc2oc3cccc(-c4ccccc4)c3c2c1 ; c1ccc(-c2cccc3oc4ccc(N(c5ccc(C6(c7ccccc7)CCCCC6)cc5)c5ccc6ccccc6c5)cc4c23)cc1]

### 5-1. Synthesis of Intermediate 78a

Intermediate 2b (1.0 eq.), 2-bromonaphthalene (1.0 eq.), tris(dibenzylideneacetone)dipalladium (0) (0.05 eq.), tri-tert-butylphosphine (0.10 eq.), and sodium tert-butoxide (1.5 eq.) were dissolved in 50 mL of toluene to prepare a reaction solution. The reaction solution was stirred at 80° C. for 1 hour in a nitrogen atmosphere. After the reaction was completed, the reaction product was washed three times with water and diethyl ether to obtain an organic layer. The organic layer was dried with MgSO₄ and then dried under reduced pressure. The resultant was subjected to column chromatography to obtain 2.2 g of Intermediate 78a. (yield: 57%)

### 5-2. Synthesis of Compound 78

Intermediate 78a (1.0 eq.), 8-bromo-1-phenyldibenzo[b,d]furan (1.0 eq.), tris(dibenzylideneacetone)dipalladium (0) (0.05 eq.), tri-tert-butylphosphine (0.10 eq.), and sodium tert-butoxide (1.5 eq.) were dissolved in 50 mL of toluene to prepare a reaction solution. The reaction solution was stirred at 80° C. for 1 hour in a nitrogen atmosphere. After the reaction was completed, the reaction product was washed three times with water and diethyl ether to obtain an organic layer. The organic layer was dried with MgSO₄ and then dried under reduced pressure. The resultant was subjected to column chromatography to obtain 2.0 g of Compound 78. (yield: 78%)

### Synthesis Example 6. Synthesis of Compound 121

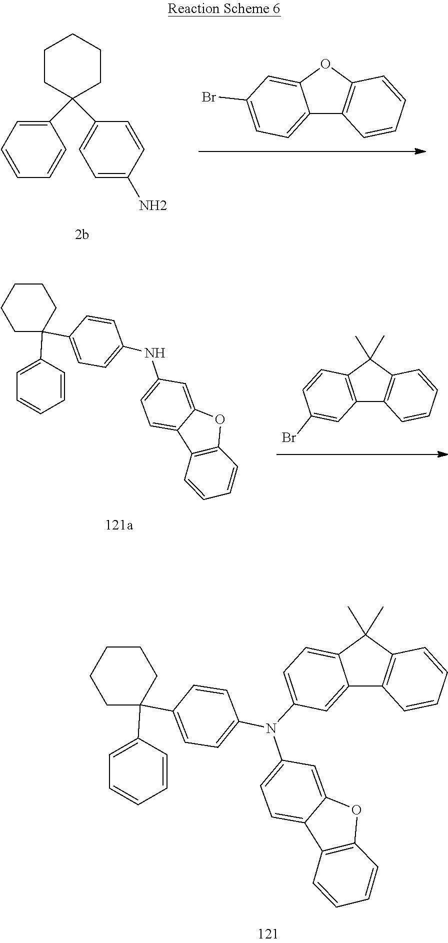 [SMILES: Nc1ccc(C2(c3ccccc3)CCCCC2)cc1 ; Brc1ccc2c(c1)oc1ccccc12 ; c1ccc(C2(c3ccc(Nc4ccc5c(c4)oc4ccccc45)cc3)CCCCC2)cc1 ; CC1(C)c2ccccc2-c2cc(Br)ccc21 ; CC1(C)c2ccccc2-c2cc(N(c3ccc(C4(c5ccccc5)CCCCC4)cc3)c3ccc4c(c3)oc3ccccc34)ccc21]

### 6-1. Synthesis of Intermediate 121a

Intermediate 2b (1.0 eq.), 3-bromodibenzo[b,d]furan (1.0 eq.), tris(dibenzylideneacetone)dipalladium (0) (0.05 eq.), tri-tert-butylphosphine (0.10 eq.), and sodium tert-butoxide (1.5 eq.) were dissolved in 50 mL of toluene to prepare a reaction solution. The reaction solution was stirred at 80° C. for 1 hour in a nitrogen atmosphere. After the reaction was completed, the reaction product was washed three times with water and diethyl ether to obtain an organic layer. The organic layer was dried with MgSO₄ and then dried under reduced pressure. The resultant was subjected to column chromatography to obtain 2.0 g of Intermediate 121a. (yield: 55%)

### 6-2. Synthesis of Compound 121

Intermediate 121a (1.0 eq.), 3-bromo-9,9-dimethyl-9H-fluorene (1.0 eq.), tris(dibenzylideneacetone)dipalladium (0) (0.05 eq.), tri-tert-butylphosphine (0.10 eq.), and sodium tert-butoxide (1.5 eq.) were dissolved in 50 mL of toluene to prepare a reaction solution. The reaction solution was stirred at 80° C. for 1 hour in a nitrogen atmosphere. After the reaction was completed, the reaction product was washed three times with water and diethyl ether to obtain an organic layer. The organic layer was dried with MgSO₄ and then dried under reduced pressure. The resultant was subjected to column chromatography to obtain 1.8 g of Compound 121. (yield: 71%)

### Synthesis Example 7. Synthesis of Compound 129

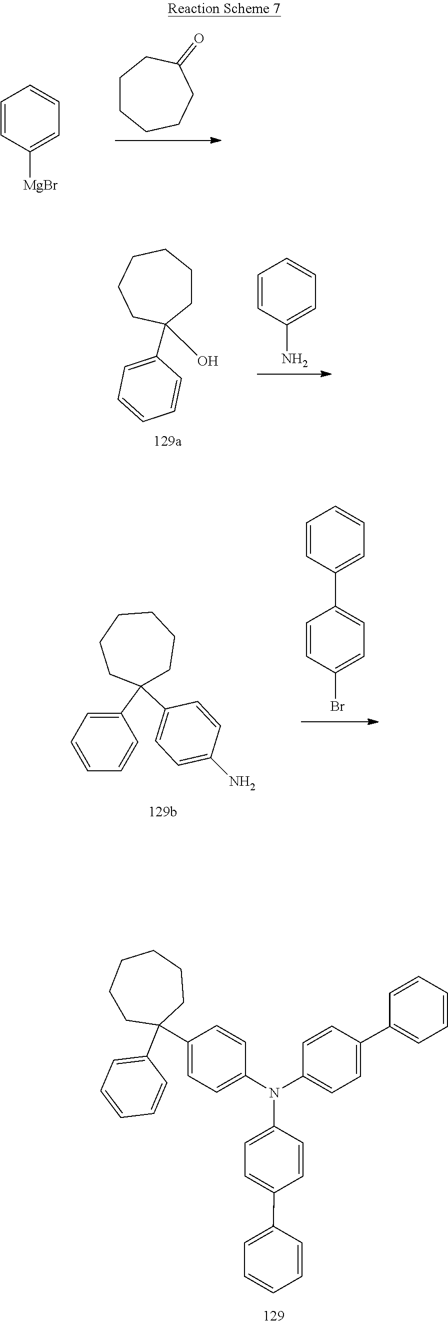

### 7-1. Synthesis of Intermediate 129a

Cycloheptanone (1.0 eq.) was dissolved in 300 mL of tetrahydrofuran, and 3 M of phenylmagnesium bromide (1.2 eq.) was added thereto in a nitrogen atmosphere at 0° C. to prepare a reaction solution. Then, the reaction solution was stirred at room temperature for 1 hour and at 70° C. for 2 hours. After the reaction was completed, the reaction product was washed slowly with ammonium chloride and water, and washed three times with diethyl ether to obtain an organic layer. The organic layer was dried with MgSO₄ and then dried under reduced pressure. The resultant was subjected to column chromatography to obtain 8.5 g of Intermediate 129a. (yield: 61%)

### 7-2. Synthesis of Intermediate 129b

A reaction solution including Intermediate 129a (1.0 eq), aniline (10.0 eq), and MsOH (9.0 eq) was prepared in a microwave (Mutiwave 7000, Anton-paar Korea Co., Ltd.). The reaction solution was stirred at 200° C. under a pressure of 63 bar for 30 minutes. After the reaction was completed, the reaction product was washed slowly with sodium hydroxide and water, and washed three times with diethyl ether to obtain an organic layer. The organic layer was dried with MgSO₄ and then dried under reduced pressure. The resultant was subjected to column chromatography to obtain 2.8 g of Intermediate 129b. (yield: 35%)

### 7-3. Synthesis of Compound 129

Intermediate 129b (1.0 eq.), 4-bromo-1,1′-biphenyl (2.2 eq.), tris(dibenzylideneacetone)dipalladium(0) (0.1 eq.), tri-tert-butylphosphine (0.2 eq.), and sodium tert-butoxide (3.0 eq.) were dissolved in 50 mL of toluene to prepare a reaction solution. The reaction solution was stirred at 80° C. for 1 hour in a nitrogen atmosphere. After the reaction was completed, the reaction product was washed three times with water and diethyl ether to obtain an organic layer. The organic layer was dried with MgSO₄ and then dried under reduced pressure. The resultant was subjected to column chromatography to obtain 2.1 g of Compound 129. (yield: 61%)

### Synthesis Example 8. Synthesis of Compound 184

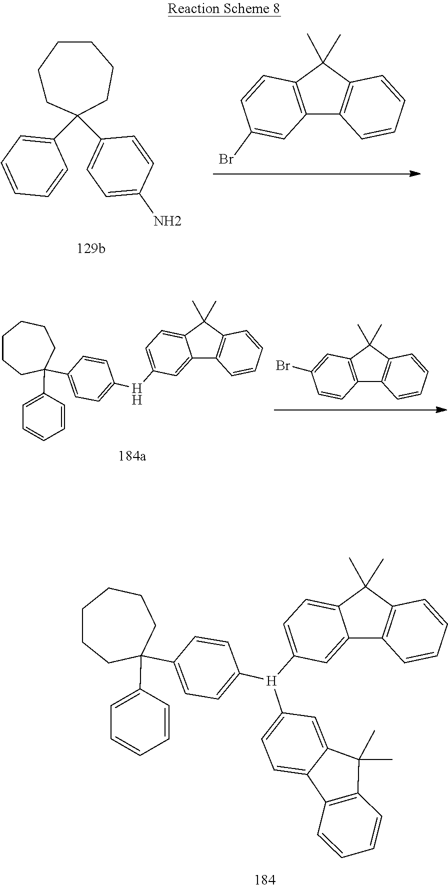

### 8-1. Synthesis of Intermediate 184a

Intermediate 129b (1.0 eq.), 3-bromo-9,9-dimethyl-9H-fluorene (1.0 eq.), tris(dibenzylideneacetone)dipalladium(0) (0.05 eq.), tri-tert-butylphosphine (0.10 eq.), and sodium tert-butoxide (1.5 eq.) were dissolved in 50 mL of toluene to prepare a reaction solution. The reaction solution was stirred at 80° C. for 1 hour in a nitrogen atmosphere. After the reaction was completed, the reaction product was washed three times with water and diethyl ether to obtain an organic layer. The organic layer was dried with MgSO₄ and then dried under reduced pressure. The resultant was subjected to column chromatography to obtain 1.8 g of Intermediate 184a. (yield: 43%)

### 8-2. Synthesis of Compound 184

Intermediate 184a (1.0 eq.), 2-bromo-9,9-dimethyl-9H-fluorene (1.0 eq.), tris(dibenzylideneacetone)dipalladium(0) (0.05 eq.), tri-tert-butylphosphine (0.10 eq.), and sodium tert-butoxide (1.5 eq.) were dissolved in 50 mL of toluene to prepare a reaction solution. The reaction solution was stirred at 80° C. for 1 hour in a nitrogen atmosphere. After the reaction was completed, the reaction product was washed three times with water and diethyl ether to obtain an organic layer. The organic layer was dried with MgSO₄ and then dried under reduced pressure. The resultant was subjected to column chromatography to obtain 1.7 g of Compound 184. (yield: 79%)

### Synthesis Example 9. Synthesis of Compound 290

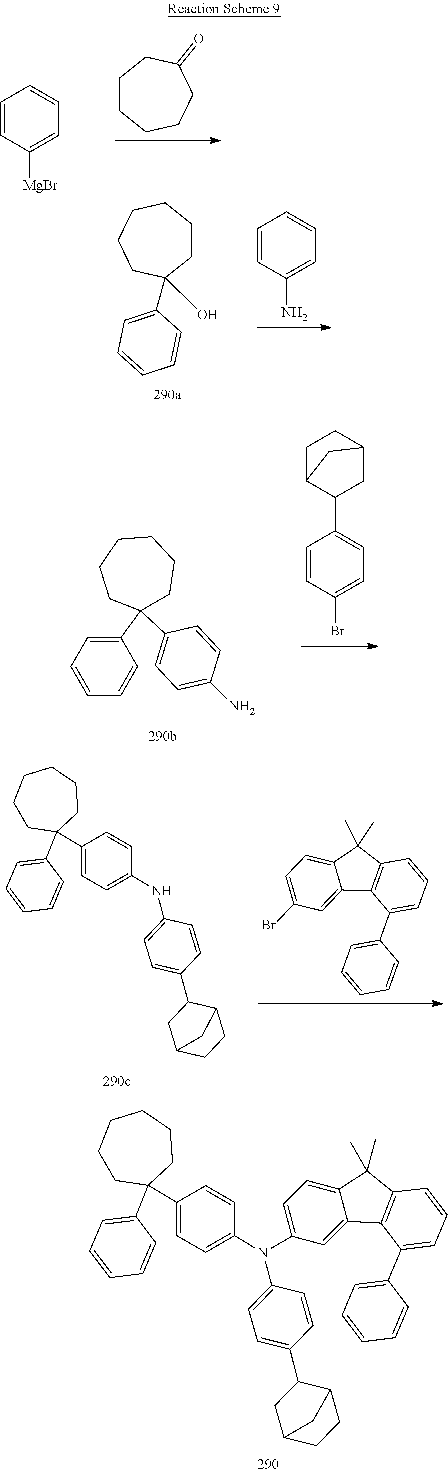

### 9-1. Synthesis of Intermediate 290a

Cyclooctanone (1.0 eq.) was dissolved in 300 mL of tetrahydrofuran, and 3 M of phenylmagnesium bromide (1.2 eq.) was added thereto in a nitrogen atmosphere at 0° C. to prepare a reaction solution. Then, the reaction solution was stirred at room temperature for 1 hour and at 70° C. for 2 hours. After the reaction was completed, the reaction product was washed slowly with ammonium chloride and water, and washed three times with diethyl ether to obtain an organic layer. The organic layer was dried with MgSO₄ and then dried under reduced pressure. The resultant was subjected to column chromatography to obtain 7.8 g of Intermediate 290a. (yield: 58%)

### 9-2. Synthesis of Intermediate 290b

A reaction solution including Intermediate 290a (1.0 eq), aniline (10.0 eq), and MsOH (9.0 eq) was prepared in a microwave (Mutiwave 7000, Anton-paar Korea Co., Ltd.). The reaction solution was stirred at 200° C. under a pressure of 63 bar for 30 minutes. After the reaction was completed, the reaction product was washed slowly with sodium hydroxide and water, and washed three times with diethyl ether to obtain an organic layer. The organic layer was dried with MgSO₄ and then dried under reduced pressure. The resultant was subjected to column chromatography to obtain 2.5 g of Intermediate 290b. (yield: 31%)

### 9-3. Synthesis of Intermediate 290c

Intermediate 290b (1.0 eq.), 2-(4-bromophenyl)bicyclo[2.2.1]heptane (1.0 eq.), tris(dibenzylideneacetone)dipalladium(0) (0.05 eq.), tri-tert-butylphosphine (0.1 eq.), and sodium tert-butoxide (1.5 eq.) were dissolved in 50 mL of toluene to prepare a reaction solution. The reaction solution was stirred at 80° C. for 1 hour in a nitrogen atmosphere. After the reaction was completed, the reaction product was washed three times with water and diethyl ether to obtain an organic layer. The organic layer was dried with MgSO₄ and then dried under reduced pressure. The resultant was subjected to column chromatography to obtain 2.0 g of Intermediate 290c. (yield: 52%)

### 9-4. Synthesis of Compound 290

Intermediate 290c (1.0 eq.), 3-bromo-9,9-dimethyl-5-phenyl-9H-fluorene (1.0 eq.), tris(dibenzylideneacetone)dipalladium(0) (0.05 eq.), tri-tert-butylphosphine (0.1 eq.), and sodium tert-butoxide (1.5 eq.) were dissolved in 50 mL of toluene to prepare a reaction solution. The reaction solution was stirred at 80° C. for 1 hour in a nitrogen atmosphere. After the reaction was completed, the reaction product was washed three times with water and diethyl ether to obtain an organic layer. The organic layer was dried with MgSO₄ and then dried under reduced pressure. The resultant was subjected to column chromatography to obtain 1.8 g of Compound 290. (yield: 63%)

### Synthesis Example 10. Synthesis of Compound 374

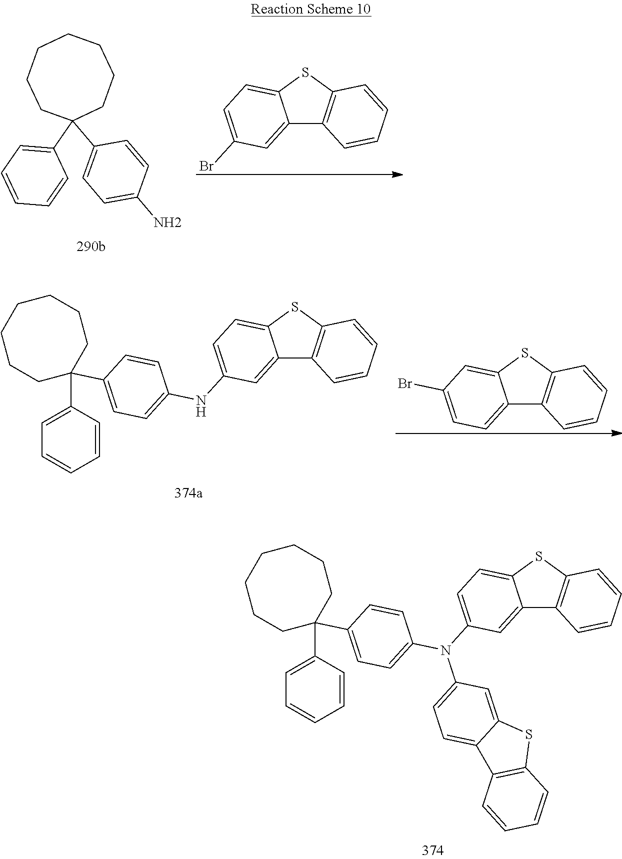

### 10-1. Synthesis of Intermediate 374a

Intermediate 290b (1.0 eq.), 2-bromodibenzo[b,d]thiophene (1.0 eq.), tris(dibenzylideneacetone)dipalladium (0) (0.05 eq.), tri-tert-butylphosphine (0.10 eq.), and sodium tert-butoxide (1.5 eq.) were dissolved in 50 mL of toluene to prepare a reaction solution. The reaction solution was stirred at 80° C. for 1 hour in a nitrogen atmosphere. After the reaction was completed, the reaction product was washed three times with water and diethyl ether to obtain an organic layer. The organic layer was dried with MgSO₄ and then dried under reduced pressure. The resultant was subjected to column chromatography to obtain 2.0 g of Intermediate 374a. (yield: 49%)

### 10-2. Synthesis of Compound 374

Intermediate 374a (1.0 eq.), 3-bromodibenzo[b,d]thiophene (1.0 eq.), tris(dibenzylideneacetone)dipalladium(0) (0.05 eq.), tri-tert-butylphosphine (0.10 eq.), and sodium tert-butoxide (1.5 eq.) were dissolved in 50 mL of toluene to prepare a reaction solution. The reaction solution was stirred at 80° C. for 1 hour in a nitrogen atmosphere. After the reaction was completed, the reaction product was washed three times with water and diethyl ether to obtain an organic layer. The organic layer was dried with MgSO₄ and then dried under reduced pressure. The resultant was subjected to column chromatography to obtain 1.8 g of Compound 374. (yield: 81%)

¹H NMR (Nuclear Magnetic Resonance) and MS/FAB (Mass Spectrometry/Fast Atom Bombardment) of the compounds synthesized according to the Synthesis Examples are shown in Table 1.

TABLE 1

| | | MS/FAB | |
|---|---|---|---|
| Compound | ¹H NMR (δ) | Calc | found |
| Compound | 7.75(d, 2H), 7.55-7.49(m, 4H), 7.41(t, 1H), | 561.34 | 561.21 |
| 2 | 7.27-7.23(m, 4H), 7.15-7.06(m, 11H), 2.72(m, 1H), | | |
| | 2.15-1.90(m, 4H), 1.85-1.43(m, 16H) | | |
| Compound | 7.90(d, 1H), 7.86(d, 1H), 7.75(d, 2H), 7.55(d, 3H), | 595.32 | 595.04 |
| 23 | 7.49(t, 2H), 7.41-7.07(m, 16H), 2.15-1.90(m, 4H), | | |
| | 1.69(s, 6H), 1.53-1.43(m, 6H) | | |
| Compound | 7.86(d, 1H), 7.79-7.78 (m, 3H), 7.65 (d, 1H), | 677.40 | 677.68 |
| 28 | 7.47-7.41 (m, 4H), 7.33(s, 1H), 7.23(t, 2H), | | |
| | 7.16-7.06(m, 12H), 2.72(m, 1H), 2.15-1.90(m, 4H), | | |
| | 1.85(m, 2H), 1.69(s, 6H), 1.60-1.43 (m, 14H) | | |
| Compound | 7.90(d, 1H), 7.62(s, 1H), 7.55 (d, 1H), 7.51 (d, 1H), | 653.40 | 653.28 |
| 37 | 7.38(t, 1H), 7.28-7.23 (m, 3H), 7.15-7.00(m, 12H), | | |
| | 2.15-1.90(m, 4H), 2.05(d, 4H), 1.99(d, 2H), 1.87(m, | | |
| | 3H), 1.76(m, 6H), 1.69(s, 6H), 1.53-1.43(m, 6H) | | |
| Compound | 8.22(s, 1H), 7.79(d, 2H), 7.71(d, 1H), 7.62-7.38(m, | 619.29 | 619.34 |
| 78 | 12H), 7.23-7.07 (m, 10H), 6.97(d, 1H), | | |
| | 2.15-1.90(m, 4H), 1.53-1.43(m, 6H) | | |
| Compound | 8.03(s, 1H), 7.98(d, 1H), 7.90(d, 1H), 7.62(s, 1H), | 609.30 | 609.15 |
| 121 | 7.55-7.51(m, 4H), 7.39-7.23(m, 6H), 7.15-7.06(m, | | |
| | 8H), 6.91(d, 1H), 2.15-1.90(m, 4H), 1.69(s, 6H), | | |
| | 1.53-1.43(m, 6H) | | |
| Compound | 7.75(d, 4H), 7.55-7.41(m, 10H), 7.27-7.23 (m, 6H), | 569.31 | 569.28 |
| 129 | 7.15-7.07(m, 7H), 1.96-1.71(m, 4H), 1.56-1.46(m, | | |
| | 8H) | | |
| Compound | 7.90-7.86(m, 3H), 7.62(s, 1H), 7.55-7.51(m, 3H), | 649.37 | 649.59 |
| 184 | 7.38-7.23(m, 7H), 7.16-7.06(m, 9H), 1.96-1.71(m, | | |
| | 4H), 1.69(s, 12H), 1.56-1.46(m, 8H) | | |
| Compound | 7.79-7.78(m, 3H), 7.65(d, 1H), 7.62(s, 1H), | 717.43 | 717.05 |
| 290 | 7.51-7.41(m, 5H), 7.23(t, 2H), 7.15-7.06 (m, 12H), | | |
| | 2.62(m, 1H), 2.19(m, 1H), 1.96-1.71(m, 4H), | | |
| | 1.82-1.21(m, 9H), 1.69(s, 6H), 1.32-1.20(m, 10H) | | |
| Compound | 8.45(d, 2H), 7.95(s, 1H), 7.86-7.85(m, 3H), 7.74(d, | 643.24 | 643.08 |
| 374 | 1H), 7.64(s, 1H), 7.56(m, 2H), 7.43-7.41(m, 2H), | | |
| | 7.31-7.23(m, 4H), 7.15-7.07(m, 7H), 1.96-1.71(m, | | |
| | 4H), 1.32-1.20(m, 10H) | | |

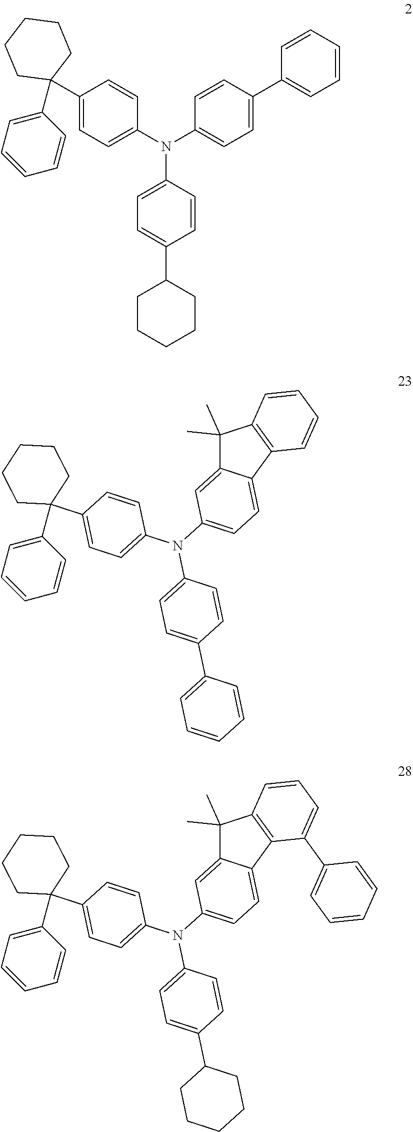 [SMILES: c1ccc(-c2ccc(N(c3ccc(C4CCCCC4)cc3)c3ccc(C4(c5ccccc5)CCCCC4)cc3)cc2)cc1 ; CC1(C)c2ccccc2-c2ccc(N(c3ccc(-c4ccccc4)cc3)c3ccc(C4(c5ccccc5)CCCCC4)cc3)cc21 ; CC1(C)c2cc(N(c3ccc(C4CCCCC4)cc3)c3ccc(C4(c5ccccc5)CCCCC4)cc3)ccc2-c2c(-c3ccccc3)cccc21]

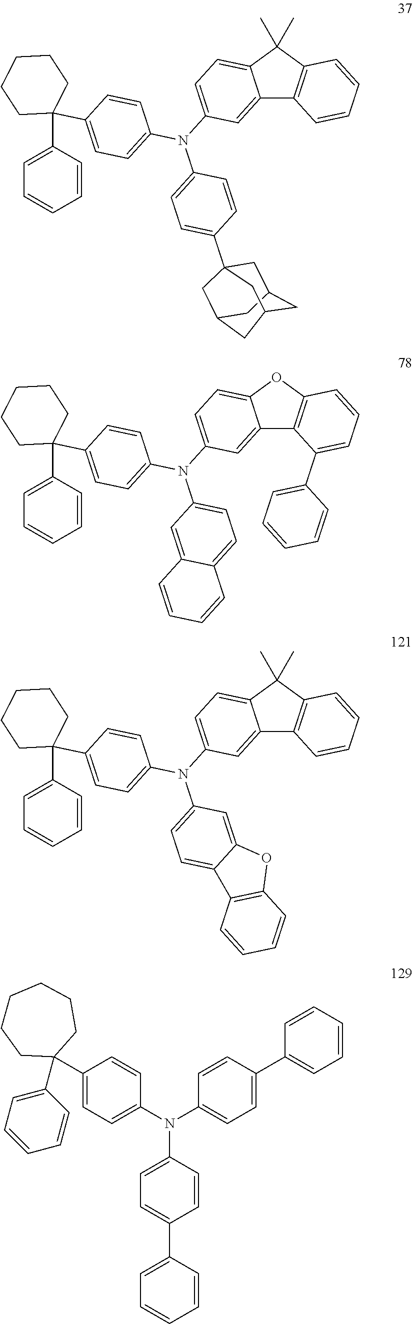 [SMILES: CC1(C)c2ccccc2-c2cc(N(c3ccc(C45CC6CC(CC(C6)C4)C5)cc3)c3ccc(C4(c5ccccc5)CCCCC4)cc3)ccc21 ; c1ccc(-c2cccc3oc4ccc(N(c5ccc(C6(c7ccccc7)CCCCC6)cc5)c5ccc6ccccc6c5)cc4c23)cc1 ; CC1(C)c2ccccc2-c2cc(N(c3ccc(C4(c5ccccc5)CCCCC4)cc3)c3ccc4c(c3)oc3ccccc34)ccc21 ; c1ccc(-c2ccc(N(c3ccc(-c4ccccc4)cc3)c3ccc(C4(c5ccccc5)CCCCCC4)cc3)cc2)cc1]

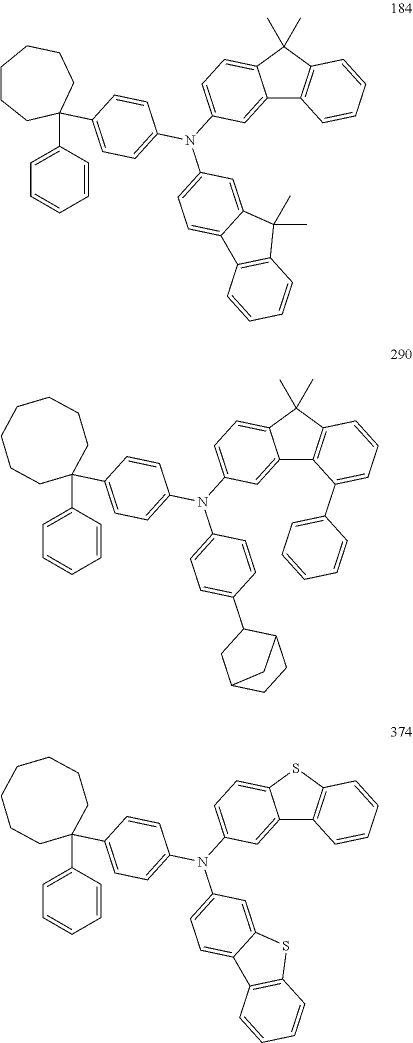 [SMILES: CC1(C)c2ccccc2-c2cc(N(c3ccc(C4(c5ccccc5)CCCCCC4)cc3)c3ccc4c(c3)C(C)(C)c3ccccc3-4)ccc21 ; CC1(C)c2ccc(N(c3ccc(C4CC5CCC4C5)cc3)c3ccc(C4(c5ccccc5)CCCCCCC4)cc3)cc2-c2c(-c3ccccc3)cccc21 ; c1ccc(C2(c3ccc(N(c4ccc5c(c4)sc4ccccc45)c4ccc5sc6ccccc6c5c4)cc3)CCCCCCC2)cc1]

### Example 1

As an anode, a substrate with 15 Ω/cm² (1,200 Å) ITO glass thereon was cut to a size of 50 mm×50 mm×0.7 mm, sonicated in isopropyl alcohol and pure water each for 5 minutes, and then cleaned by exposure to ultraviolet rays and ozone for 30 minutes. The resultant ITO glass substrate was loaded onto a vacuum deposition apparatus.

2-TNATA was vacuum-deposited on the ITO glass substrate to form a hole injection layer having a thickness of 600 Å, and Compound 2 was then vacuum-deposited on the hole injection layer to form a hole transport layer having a thickness of 300 Å.

9,10-di(naphthalen-2-yl)anthracene (DNA) as a host and 4,4′-bis[2-(4-(N,N-diphenylamino)phenyl)vinyl]biphenyl (DPAVBi) as a dopant were co-deposited on the hole transport layer at a weight ratio of 98:2 to form an emission layer having a thickness of 300 Å.

Subsequently, Alq₃ was deposited on the emission layer to form an electron transport layer having a thickness of 300 Å, and LiF, which is a halogenated alkali metal, was deposited on the electron transport layer to form an electron injection layer having a thickness of 10 Å, and Al was vacuum-deposited thereon to form a LiF/AI electrode having a thickness of 3,000 Å, thereby completing the manufacture of a light-emitting device.

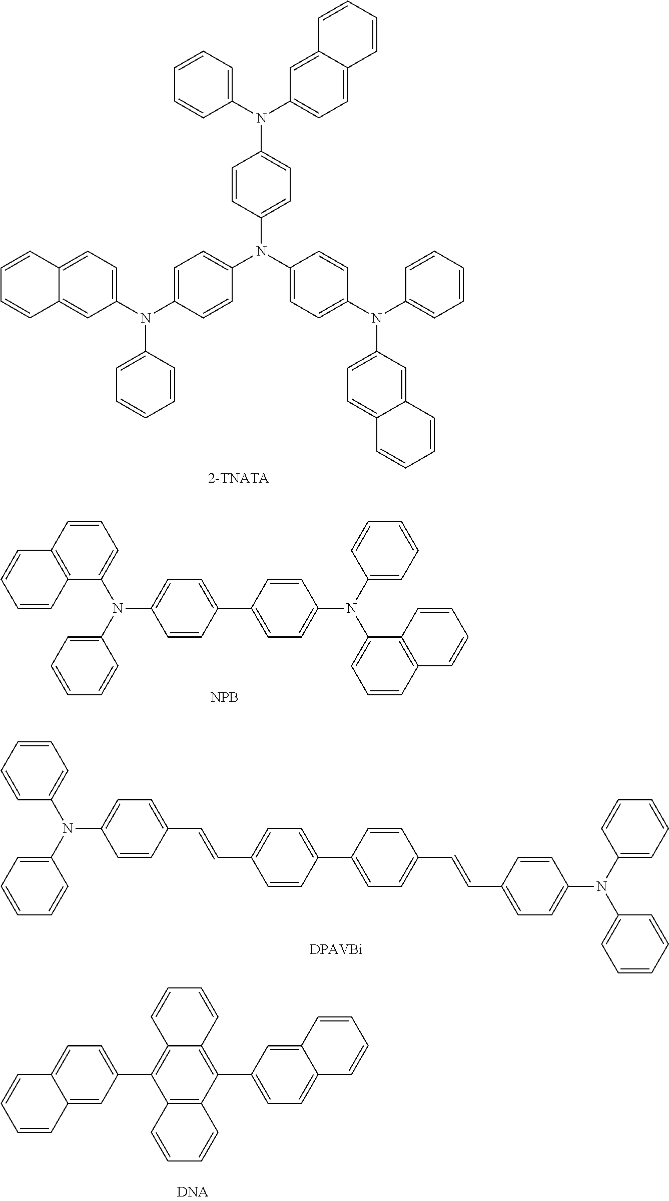

### Examples 2 to 10 and Comparative Examples 1 to 6

Examples 2 to 10 and Comparative Examples 1 to 6 were prepared in substantially the same manner as Example 1, except that different compositions as in Table 2 were utilized in the respective hole transport layers.

A voltage was applied so that a current density of each light-emitting device became 50 mA/cm². A driving voltage (V), a current density (mA/cm²), luminance (cd/m²), a luminescence efficiency (cd/A), an emission color, and a half lifespan (hours (hr) @100 mA/cm²) were each measured by utilizing Keithley SMU 236 and luminance meter PR650, and results thereof are shown in Table 2.

TABLE 2

| | | | | | | | Half |
|---|---|---|---|---|---|---|---|
| Light- | Hole | Driving | Current | | | | lifespan |
| emitting | transport | voltage | density | Luminance | Efficiency | Emission | (hr @100 |
| device | material | (V) | (mA/cm²) | (cd/m²) | (cd/A) | color | mA/cm²) |
| Example 1 | Compound 2 | 4.80 | 50 | 3,200 | 6.40 | Blue | 370 |
| Example 2 | Compound 23 | 4.65 | 50 | 3,100 | 6.20 | Blue | 420 |
| Example 3 | Compound 28 | 4.25 | 50 | 3,350 | 6.70 | Blue | 480 |
| Example 4 | Compound 37 | 4.32 | 50 | 3210 | 6.42 | Blue | 435 |
| Example 5 | Compound 78 | 4.45 | 50 | 3190 | 6.38 | Blue | 440 |
| Example 6 | Compound 121 | 4.52 | 50 | 3010 | 6.02 | Blue | 400 |
| Example 7 | Compound 129 | 5.05 | 50 | 3100 | 6.20 | Blue | 380 |
| Example 8 | Compound 184 | 5.12 | 50 | 3250 | 6.50 | Blue | 410 |
| Example 9 | Compound 290 | 4.96 | 50 | 3110 | 6.22 | Blue | 420 |
| Example 10 | Compound 374 | 4.85 | 50 | 3080 | 6.16 | Blue | 410 |
| Comparative | NPB | 7.05 | 50 | 2510 | 5.02 | Blue | 240 |
| Example 1 | | | | | | | |
| Comparative | Compound A | 4.95 | 50 | 3100 | 6.20 | Blue | 310 |
| Example 2 | | | | | | | |
| Comparative | Compound B | 4.92 | 50 | 3080 | 6.16 | Blue | 340 |
| Example 3 | | | | | | | |
| Comparison | Compound C | 5.01 | 50 | 3020 | 6.04 | Blue | 360 |
| Example 4 | | | | | | | |
| Comparison | Compound D | 5.23 | 50 | 3050 | 6.10 | Blue | 280 |
| Example 5 | | | | | | | |
| Comparative | Compound E | 4.98 | 50 | 3070 | 6.14 | Blue | 350 |
| Example 6 | | | | | | | |

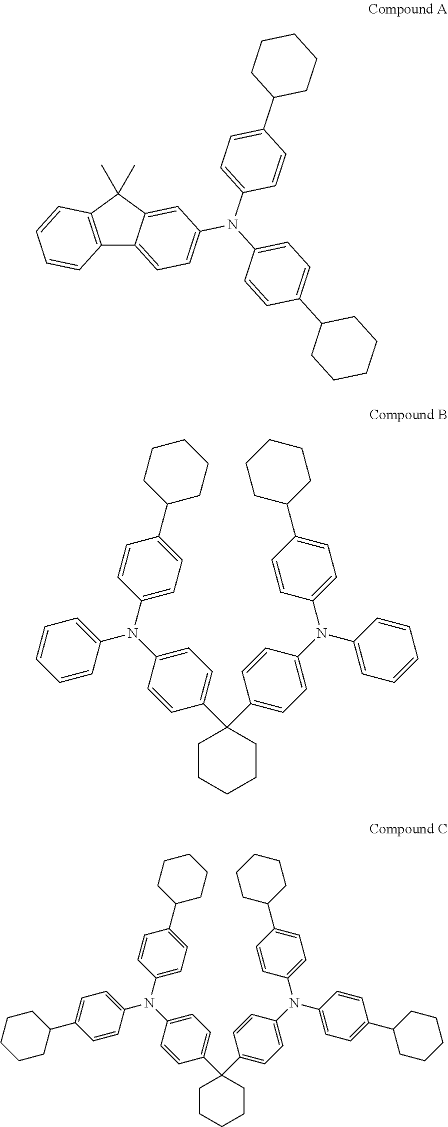 [SMILES: CC1(C)c2ccccc2-c2ccc(N(c3ccc(C4CCCCC4)cc3)c3ccc(C4CCCCC4)cc3)cc21 ; c1ccc(N(c2ccc(C3CCCCC3)cc2)c2ccc(C3(c4ccc(N(c5ccccc5)c5ccc(C6CCCCC6)cc5)cc4)CCCCC3)cc2)cc1 ; c1cc(N(c2ccc(C3CCCCC3)cc2)c2ccc(C3(c4ccc(N(c5ccc(C6CCCCC6)cc5)c5ccc(C6CCCCC6)cc5)cc4)CCCCC3)cc2)ccc1C1CCCCC1]

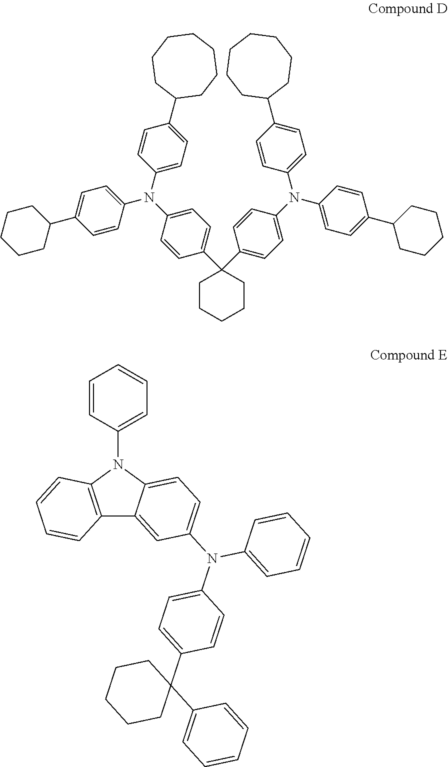 [SMILES: c1cc(N(c2ccc(C3CCCCC3)cc2)c2ccc(C3(c4ccc(N(c5ccc(C6CCCCCCC6)cc5)c5ccc(C6CCCCC6)cc5)cc4)CCCCC3)cc2)ccc1C1CCCCCCC1 ; c1ccc(N(c2ccc(C3(c4ccccc4)CCCCC3)cc2)c2ccc3c(c2)c2ccccc2n3-c2ccccc2)cc1]

From Table 2, the light-emitting devices each including the amine-based compound according to one or more embodiments were found to have excellent or suitable driving voltage (V), luminescence efficiency (cd/A), luminance (cd/m²), and half lifespan (hr @100 mA/cm²), as compared with the light-emitting devices of Comparative Examples 1 to 6. For example, the light-emitting devices of Examples 1 to 10 were found to have improved lifespan as compared with the light-emitting devices of Comparative Examples 1 to 6.

According to the one or more embodiments of the present disclosure, the utilization of the amine-based compound of the present disclosure may enable the manufacture of a light-emitting device having high efficiency and a long lifespan and a high-quality electronic apparatus including the light-emitting device.

In the present disclosure, singular expressions may include plural expressions unless the context clearly indicates otherwise. It will be further understood that the terms “comprise(s),” “include(s),” or “have/has” when utilized in the present disclosure, specify the presence of stated features, integers, steps, operations, elements, and/or components, but do not preclude the presence or addition of one or more other features, integers, steps, operations, elements, components, and/or groups thereof. The ‘T’ utilized herein may be interpreted as “and” or as “or” depending on the situation.

Throughout the present disclosure, when a component such as a layer, a film, a region, or a plate is mentioned to be placed “on” another component, it will be understood that it may be directly on another component or that another component may be interposed therebetween. In some embodiments, “directly on” may refer to that there are no additional layers, films, regions, plates, etc., between a layer, a film, a region, a plate, etc. and the other part. For example, “directly on” may refer to two layers or two members are disposed without utilizing an additional member such as an adhesive member therebetween.

In the present disclosure, although the terms “first,” “second,” etc., may be utilized herein to describe one or more elements, components, regions, and/or layers, these elements, components, regions, and/or layers should not be limited by these terms. These terms are only utilized to distinguish one component from another component.

As utilized herein, the singular forms “a,” “an,” “one,” and “the” are intended to include the plural forms as well, unless the context clearly indicates otherwise. Further, the use of “may” when describing embodiments of the present disclosure refers to “one or more embodiments of the present disclosure”.

In the present disclosure, when particles are spherical, “diameter” indicates a particle diameter or an average particle diameter, and when the particles are non-spherical, the “diameter” indicates a major axis length or an average major axis length. The diameter (or size) of the particles may be measured utilizing a scanning electron microscope or a particle size analyzer. As the particle size analyzer, for example, HORIBA, LA-950 laser particle size analyzer, may be utilized. When the size of the particles is measured utilizing a particle size analyzer, the average particle diameter (or size) is referred to as D50. D50 refers to the average diameter (or size) of particles whose cumulative volume corresponds to 50 vol % in the particle size distribution (e.g., cumulative distribution), and refers to the value of the particle size corresponding to 50% from the smallest particle when the total number of particles is 100% in the distribution curve accumulated in the order of the smallest particle size to the largest particle size.

As utilized herein, the terms “substantially,” “about,” or similar terms are used as terms of approximation and not as terms of degree, and are intended to account for the inherent deviations in measured or calculated values that would be recognized by those of ordinary skill in the art. “About” as used herein, is inclusive of the stated value and means within an acceptable range of deviation for the particular value as determined by one of ordinary skill in the art, considering the measurement in question and the error associated with measurement of the particular quantity (i.e., the limitations of the measurement system). For example, “about” may mean within one or more standard deviations, or within ±30%, 20%, 10%, 5% of the stated value.

Any numerical range recited herein is intended to include all sub-ranges of the same numerical precision subsumed within the recited range. For example, a range of “1.0 to 10.0” is intended to include all subranges between (and including) the recited minimum value of 1.0 and the recited maximum value of 10.0, that is, having a minimum value equal to or greater than 1.0 and a maximum value equal to or less than 10.0, such as, for example, 2.4 to 7.6. Any maximum numerical limitation recited herein is intended to include all lower numerical limitations subsumed therein and any minimum numerical limitation recited in this specification is intended to include all higher numerical limitations subsumed therein. Accordingly, Applicant reserves the right to amend this specification, including the claims, to expressly recite any sub-range subsumed within the ranges expressly recited herein.

The light-emitting device, the display device, the electronic apparatus, the electronic equipment, or any other relevant devices or components according to embodiments of the present disclosure described herein may be implemented utilizing any suitable hardware, firmware (e.g., an application-specific integrated circuit), software, or a combination of software, firmware, and hardware. For example, the various components of the device may be formed on one integrated circuit (IC) chip or on separate IC chips. Further, the various components of the device may be implemented on a flexible printed circuit film, a tape carrier package (TCP), a printed circuit board (PCB), or formed on one substrate. Further, the various components of the device may be a process or thread, running on one or more processors, in one or more computing devices, executing computer program instructions and interacting with other system components for performing the various functionalities described herein. The computer program instructions are stored in a memory which may be implemented in a computing device using a standard memory device, such as, for example, a random access memory (RAM). The computer program instructions may also be stored in other non-transitory computer readable media such as, for example, a CD-ROM, flash drive, or the like. Also, a person of skill in the art should recognize that the functionality of various computing devices may be combined or integrated into a single computing device, or the functionality of a particular computing device may be distributed across one or more other computing devices without departing from the scope of the embodiments of the present disclosure.

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the drawings, it will be understood by those of ordinary skill in the art that one or more suitable changes in form and details may be made therein without departing from the spirit and scope as defined by the following claims and equivalents thereof.

What is claimed is:

1. A light-emitting device comprising:
a first electrode;
a second electrode facing the first electrode;
an interlayer between the first electrode and the second electrode and comprising an emission layer; and
an amine-based compound represented by Formula 1: 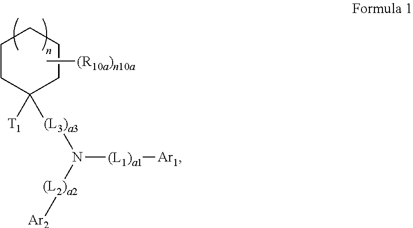
wherein, in Formula 1,
n is an integer from 0 to 5,
L₁ to L₃ are each independently a C₃-C₆₀ carbocyclic group unsubstituted or substituted with at least one R₁₀ₐ or a C₁-C₆₀ heterocyclic group unsubstituted or substituted with at least one R₁₀ₐ,
a1 to a3 are each independently an integer from 0 to 3,
*-(L₁)ₐ₁-* is a single bond when a1 is 0,
*-(L₂)ₐ₂-*′ is a single bond when a2 is 0,
*-(L₃)ₐ₃-*′ is a single bond when a3 is 0,
Ar₁ and Ar₂ each independently comprise a group represented by Formula 1-1, a group represented by Formula 1-2, a C₃-C₆₀ carbocyclic group unsubstituted or substituted with at least one R₁₀ₐ, or a C₁-C₆₀ heterocyclic group unsubstituted or substituted with at least one R₁₀ₐ, 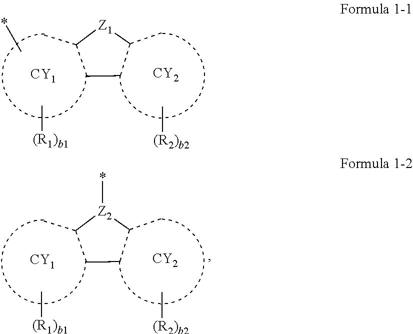
wherein, in Formulae 1-1 and 1-2,
Z₁ is O, S, P(R₃), C(R₃)(R₄), C(R₃)H, CH₂, Si(R₃)(R₄), Si(R₃)H, or SiH₂,
Z₂ is P, C(R₃), CH, Si(R₃), or SiH,
CY₁ and CY₂ are each independently a C₃-C₆₀ carbocyclic group or a C₁-C₆₀ heterocyclic group,
b1 and b2 are each independently an integer from 0 to 5,
* indicates a binding site to a neighboring atom,
T₁, R₁, R₂, R₃, R₄, and R₁₀ₐ are each independently:
deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, —Si(Q₁₁)(Q₁₂)(Q₁₃), —B(Q₁₁)(Q₁₂), —C(═O)(Q₁₁), —S(═O)₂(Q₁₁), —P(═O)(Q₁₁)(Q₁₂), or a combination thereof;
a C₃-C₆₀ carbocyclic group, a C₁-C₆₀ heterocyclic group, a C₆-C₆₀ aryloxy group, or a C₆-C₆₀ arylthio group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, —Si(Q₂₁)(Q₂₂)(Q₂₃), —B(Q₂₁)(Q₂₂), —C(═O)(Q₂₁), —S(═O)₂(Q₂₁), —P(═O)(Q₂₁)(Q₂₂), or a combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂), and
wherein Q₁₁ to Q₁₃, Q₂₁ to Q₂₃, and Q₃₁ to Q₃₃ are each independently:
hydrogen; deuterium; —F; —C₁; —Br; —I; a hydroxyl group; a cyano group; a nitro group; a C₁-C₆₀ alkyl group; a C₂-C₆₀ alkenyl group; a C₂-C₆₀ alkynyl group; a C₁-C₆₀ alkoxy group; or a C₃-C₆₀ carbocyclic group or a C₁-C₆₀ heterocyclic group, each unsubstituted or substituted with deuterium, —F, a cyano group, a C₁-C₆₀ alkyl group, a C₁-C₆₀ alkoxy group, a phenyl group, a biphenyl group, or a combination thereof, and
n10a is an integer from 0 to 10.

2. The light-emitting device of claim 1, wherein the interlayer comprises the amine-based compound represented by Formula 1.

3. The light-emitting device of claim 1, wherein
the first electrode is an anode,
the second electrode is a cathode,
the interlayer further comprises a hole transport region between the first electrode and the emission layer and an electron transport region between the emission layer and the second electrode,
the hole transport region comprises a hole injection layer, a hole transport layer, an emission auxiliary layer, an electron blocking layer, or a combination thereof, and
the electron transport region comprises a hole blocking layer, an electron transport layer, an electron injection layer, or a combination thereof.

4. The light-emitting device of claim 3, wherein the hole transport region comprises the amine-based compound represented by Formula 1.

5. The light-emitting device of claim 3, wherein the hole transport layer comprises the amine-based compound represented by Formula 1.

6. The light-emitting device of claim 3, further comprising
a first capping layer, a second capping layer, or the first capping layer and the second capping layer, wherein
the first capping layer is on a surface of the first electrode, and/or
the second capping layer is on a surface of the second electrode.

7. The light-emitting device of claim 1, wherein the emission layer is to emit blue light.

8. An electronic apparatus comprising the light-emitting device according to claim 1.

9. The electronic apparatus of claim 8, further comprising:
a thin-film transistor; and
a color filter, a color conversion layer, a touch screen layer, a polarizing layer, or
a combination thereof,
wherein the thin-film transistor comprises a source electrode and a drain electrode, and
the first electrode of the light-emitting device is electrically connected to the source electrode or the drain electrode of the thin-film transistor.

10. An electronic equipment comprising the light-emitting device of claim 1, wherein the electronic equipment is at least one selected from a flat panel display, a curved display, a computer monitor, a medical monitor, a television, an advertisement board, an indoor or outdoor lighting and/or signaling light, a head-up display, a fully or partially transparent display, a flexible display, a rollable display, a foldable display, a stretchable display, a laser printer, a telephone, a mobile phone, a tablet, a phablet, a personal digital assistant (FDA), a wearable device, a laptop computer, a digital camera, a camcorder, a viewfinder, a microdisplay, a 3D display, a virtual or augmented reality display, a vehicle, a video wall comprising multiple displays tiled together, a theater or stadium screen, a phototherapy device, and a sign.

11. An amine-based compound represented by Formula 1: 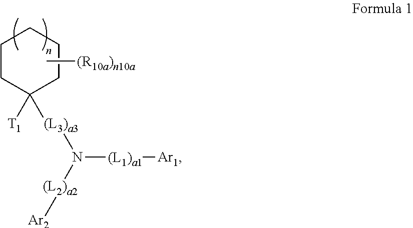
wherein, in Formula 1,
n is an integer from 0 to 5,
L₁ to L₃ are each independently a C₃-C₆₀ carbocyclic group unsubstituted or substituted with at least one R₁₀ₐ or a C₁-C₆₀ heterocyclic group unsubstituted or substituted with at least one R₁₀ₐ,
a1 to a3 are each independently an integer from 0 to 3,
*-(L₁)ₐ₁-* is a single bond when a1 is 0,
*-(L₂)ₐ₂-*′ is a single bond when a2 is 0,
*-(L₃)ₐ₃-*′ is a single bond when a3 is 0,
Ar₁ and Ar₂ each independently comprise a group represented by Formula 1-1, a group represented by Formula 1-2, a C₃-C₆₀ carbocyclic group unsubstituted or substituted with at least one R₁₀ₐ, or a C₁-C₆₀ heterocyclic group unsubstituted or substituted with at least one R₁₀ₐ, 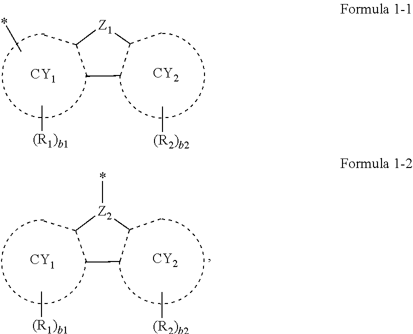
wherein, in Formulae 1-1 and 1-2,
Z₁ is O, S, P(R₃), C(R₃)(R₄), C(R₃)H, CH₂, Si(R₃)(R₄), Si(R₃)H, or SiH₂,
Z₂ is P, C(R₃), CH, Si(R₃), or SiH,
CY₁ and CY₂ are each independently a C₃-C₆₀ carbocyclic group or a C₁-C₆₀ heterocyclic group,
b1 and b2 are each independently an integer from 0 to 5,
* indicates a binding site to a neighboring atom,
T₁, R₁, R₂, R₃, R₄, and R₁₀ₐ are each independently:
deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, —Si(Q₁₁)(Q₁₂)(Q₁₃), —B(Q₁₁)(Q₁₂), —C(═O)(Q₁₁), —S(═O)₂(Q₁₁), —P(═O)(Q₁₁)(Q₁₂), or a combination thereof;
a C₃-C₆₀ carbocyclic group, a C₁-C₆₀ heterocyclic group, a C₆-C₆₀ aryloxy group, or a C₆-C₆₀ arylthio group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, —Si(Q₂₁)(Q₂₂)(Q₂₃), —B(Q₂₁)(Q₂₂), —C(═O)(Q₂₁), —S(═O)₂(Q₂₁), —P(═O)(Q₂₁)(Q₂₂), or a combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂), and
wherein Q₁₁ to Q₁₃, Q₂₁ to Q₂₃, and Q₃₁ to Q₃₃ are each independently:
hydrogen; deuterium; —F; —C₁; —Br; —I; a hydroxyl group; a cyano group; a nitro group; a C₁-C₆₀ alkyl group; a C₂-C₆₀ alkenyl group; a C₂-C₆₀ alkynyl group; a C₁-C₆₀ alkoxy group; or a C₃-C₆₀ carbocyclic group or a C₁-C₆₀ heterocyclic group, each unsubstituted or substituted with deuterium, —F, a cyano group, a C₁-C₆₀ alkyl group, a C₁-C₆₀ alkoxy group, a phenyl group, a biphenyl group, or a combination thereof, and
n10a is an integer from 0 to 10.

12. The amine-based compound of claim 11, wherein n is an integer from 0 to 2.

13. The amine-based compound of claim 11, wherein,
in Formula 1, an A value of a moiety represented by 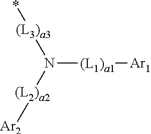
is equal to or less than an A value of T₁.

14. The amine-based compound of claim 11, wherein a sum of a1, a2, and a3 is 1 or more.

15. The amine-based compound of claim 11, wherein at least one selected from among L₁, L₂, and L₃ is a benzene group unsubstituted or substituted with at least one R₁₀ₐ, a naphthalene group unsubstituted or substituted with at least one R₁₀ₐ, a fluorene group unsubstituted or substituted with at least one R₁₀ₐ, a dibenzofuran group unsubstituted or substituted with at least one R₁₀ₐ, or a dibenzothiophene group unsubstituted or substituted with at least one R₁₀ₐ.

16. The amine-based compound of claim 11, wherein at least one selected from among L₁, L₂, and L₃ is a group represented by Formulae 1-6-1 to 1-6-3: 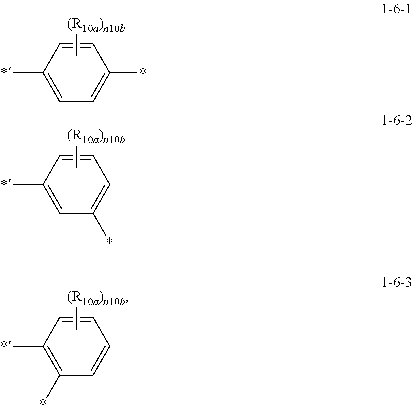
wherein, in Formulae 1-6-1 to 1-6-3,
R₁₀ₐ is the same as defined in Formula 1,
n10b is an integer from 0 to 4, and
* and * are each a binding site to a neighboring atom.

17. The amine-based compound of claim 11, wherein
the amine-based compound satisfies one of Condition i) or ii):
i) at least one selected from among Ar₁ and Ar₂ comprises a group represented by Formula 1-1 or a group represented by Formula 1-2;
ii) Ar₁ and Ar₂ each do not include the group represented by Formula 1-1 and the group represented by Formula 1-2.

18. The amine-based compound of claim 11, wherein Ar₁ and Ar₂ each independently comprise a cyclopentane group, a cyclohexane group, a norbornane group, a bicycloheptane group, an adamantane group, a benzene group, a pentalene group, a naphthalene group, an azulene group, an indacene group, an acenaphthylene group, a phenalene group, a phenanthrene group, an anthracene group, a fluoranthene group, a triphenylene group, a pyrene group, a chrysene group, a perylene group, a pentaphene group, a heptalene group, a naphthacene group, a picene group, a hexacene group, a pentacene group, a rubicene group, a coronene group, an ovalene group, an indene group, a fluorene group, a spiro-bifluorene group, a benzofluorene group, an indenophenanthrene group, an indenoanthracene group, a pyrrole group, a thiophene group, a furan group, an indole group, a benzoindole group, a naphthoindole group, an isoindole group, a benzoisoindole group, a naphthoisoindole group, a benzosilole group, a benzothiophene group, a benzofuran group, a dibenzosilole group, a dibenzothiophene group, a dibenzofuran group, a benzonaphthofuran group, a benzonaphthothiophene group, a benzonaphthosilole group, a benzofurodibenzofuran group, a benzofurodibenzothiophene group, a benzothienodibenzothiophene group, a pyrazole group, an imidazole group, a triazole group, an oxazole group, an isoxazole group, an oxadiazole group, a thiazole group, an isothiazole group, a thiadiazole group, a benzopyrazole group, a benzimidazole group, a benzoxazole group, a benzoisoxazole group, a benzothiazole group, a benzoisothiazole group, a pyridine group, a pyrimidine group, a pyrazine group, a pyridazine group, a triazine group, a quinoline group, an isoquinoline group, a benzoquinoline group, a benzoisoquinoline group, a quinoxaline group, a benzoquinoxaline group, a quinazoline group, a benzoquinazoline group, a phenanthroline group, a cinnoline group, a phthalazine group, a naphthyridine group, an imidazopyridine group, an imidazopyrimidine group, an imidazotriazine group, an imidazopyrazine group, an imidazopyridazine group, an azafluorene group, an azadibenzosilole group, an azadibenzothiophene group, or an azadibenzofuran group.

19. The amine-based compound of claim 11, wherein T₁ and L₃ comprise an identical group.

20. The amine-based compound of claim 11, wherein T₁ comprises an isopropyl group, an isobutyl group, a sec-butyl group, a tert-butyl group, an isopentyl group, a sec-pentyl group, a tert-pentyl group, a neopentyl group, a cyclopentane group, a cyclohexane group, a norbornane group, a bicycloheptane group, an adamantane group, a benzene group, a pentalene group, a naphthalene group, an azulene group, an indacene group, an acenaphthylene group, a phenalene group, a phenanthrene group, an anthracene group, a fluoranthene group, a triphenylene group, a pyrene group, a chrysene group, a perylene group, a pentaphene group, a heptalene group, a naphthacene group, a picene group, a hexacene group, a pentacene group, a rubicene group, a coronene group, an ovalene group, an indene group, a fluorene group, a spiro-bifluorene group, a benzofluorene group, an indenophenanthrene group, or an indenoanthracene group.
